(19) United States
(12) Patent Application Publication
(10) Pub. No.: US 2023/0357406 A1
(43) Pub. Date: Nov. 9, 2023

(54) CTLA-4 BINDING MOLECULES COMPRISING SHIGA TOXIN A SUBUNIT SCAFFOLDS AND USES THEREOF

(71) Applicant: Molecular Templates, Inc., Austin, TX (US)

(72) Inventors: Eric POMA, New York, NY (US); Erin WILLERT, Round Rock, TX (US); Aimee IBERG, Austin, TX (US); Swati KHANNA, Austin, TX (US); Roger WALTZMAN, New York, NY (US); Kogan BAO, Austin, TX (US)

(21) Appl. No.: 18/180,785

(22) Filed: Mar. 8, 2023

Related U.S. Application Data
(60) Provisional application No. 63/483,104, filed on Feb. 3, 2023.
(60) Provisional application No. 63/422,652, filed on Nov. 4, 2022.
(60) Provisional application No. 63/317,764, filed on Mar. 8, 2022.

(51) Int. Cl. C07K 16/28; C07K 14/245; C07K 14/25; A61P 35/00

(52) U.S. Cl. CPC C07K 16/2818; C07K 14/245; C07K 14/25; A61P 35/00; C07K 2317/569; C07K 2319/00; A61K 2039/545

(57) ABSTRACT

Provided herein are binding molecules that each comprise (1) a Shiga toxin A subunit effector polypeptide and (2) a binding region capable of specifically binding CTLA-4 on the surface of cell, such as a tumor cell or an immunosuppressive immune cell. Further provided are methods of using such binding molecules to treat diseases and disorders, such as cancer.

### CROSS-REFERENCE TO RELATED APPLICATIONS

This application claims priority to U.S. Provisional Application No. 63/317,764 filed on Mar. 8, 2022; 63/422,652 filed on Nov. 4, 2022; and 63/483,104 filed on Feb. 3, 2023; each of which are herein incorporated by reference in their entireties.

### REFERENCE TO AN ELECTRONIC SEQUENCE LISTING

The Sequence Listing associated with this application is provided in xml format in lieu of a paper copy, and is hereby incorporated by reference in its entirety. The name of the xml file containing the Sequence Listing is MTEM_018_05 US_SeqList_ST26.xml. The text file is about 359,734 kilobytes in size, was created on Mar. 7, 2023, and was submitted electronically via Patent Center.

### FIELD

The present application relates to compositions and methods for treating cancer. More specifically, the present application relates to binding molecules which comprise (i) a Shiga toxin A subunit effector polypeptide and (ii) a binding region capable of specifically binding a target (e.g., CTLA-4) on the surface of a cell.

### BACKGROUND

In many cancers, non-malignant cells present in the tumor microenvironment (TME) help sustain growth of the tumor. One key component of the TME of certain cancers is immunosuppressive immune cells (IICs), including tumor-associated macrophages (TAMs), myeloid-derived suppressor cells (MDSCs), tumor-associated neutrophils (TANs), cancer-associated fibroblasts (CAFs), and regulatory T-cells (Tregs). IICs promote tumor growth and invasion and protect the tumor from immune surveillance by cytotoxic cells according to various mechanisms. For example, TAMs can produce a variety of chemokines, such as CCL17, CCL18 and CCL22, which attract Tregs to cancer sites, thereby impeding cytotoxic T cell activation. Moreover, TAMs are able to produce angiogenic factors, such as vascular endothelial growth factor (VEGF), platelet-derived growth factor and transforming growth factor β, and also induce angiogenesis by expressing matrix metalloproteinase (MMPs). Thus, inhibition of the pro-cancer activities of IICs is a promising strategy for treatment of some cancers.

Immune checkpoint inhibitors (ICIs) can, in some cases, reinvigorate antitumor immune responses by interrupting co-inhibitory signaling pathways and promote immune-mediated elimination of tumor cells. However, ICIs are not always effective, and checkpoint inhibitor therapy has been associated with severe side effects in patients. Additionally, ICIs are believed to predominantly inhibit the activities of IICs and not eliminate or remove IICs from the TME.

Thus, there is a need in the art for cancer therapies that are more effective and have less side effects compared to checkpoint inhibitors. There is also a need in the art for cancer therapies that can be used once ICI therapy has failed, such as therapies that function by a different mechanism of action, or can be used in combination with ICI therapy.

### BRIEF SUMMARY

The present disclosure provides a CTLA-4 binding molecule comprising a Shiga toxin A subunit effector polypeptide and a binding region capable of specifically binding an extracellular part of CTLA-4; wherein the binding region comprises a VHH domain comprising a HCDR1, a HCDR2, and a HCDR3. In some embodiments, the CTLA-4 binding molecule comprises: (a) the HCDR1 comprises the amino acid sequence of SEQ ID NO: 23, the HCDR2 comprises the amino acid sequence of SEQ ID NO: 24, and the HCDR3 comprises the amino acid sequence of SEQ ID NO: 25; or (b) the HCDR1 comprises the amino acid sequence of SEQ ID NO: 26, the HCDR2 comprises the amino acid sequence of SEQ ID NO: 27, and the HCDR3 comprises the amino acid sequence of SEQ ID NO: 28. In some embodiments, the VHH domain comprises the amino acid sequence of SEQ ID NO: 21 or SEQ ID NO: 22.

In some embodiments, the present disclosure provides a CTLA-4 binding molecule comprising a Shiga toxin A subunit effector polypeptide and a binding region capable of specifically binding an extracellular part of CTLA-4; wherein the binding region comprises a first VHH domain comprising a first HCDR1, a first HCDR2, and a first HCDR3 and a second VHH domain comprising a second HCDR1, a second HCDR2, and a second HCDR3. In some embodiments, the CTLA-4 binding molecule comprises a linker that links the first VHH domain and the second VHH domain. In some embodiments, the first HCDR1 comprises the amino acid sequence of SEQ ID NO: 23, the first HCDR2 comprises the amino acid sequence of SEQ ID NO: 24, and the first HCDR3 comprises the amino acid sequence of SEQ ID NO: 25. In some embodiments, the first VHH domain comprises the amino acid sequence of SEQ ID NO: 21. In some embodiments, the second HCDR1 comprises the amino acid sequence of SEQ ID NO: 26, the second HCDR2 comprises the amino acid sequence of SEQ ID NO: 27, and the second HCDR3 comprises the amino acid sequence of SEQ ID NO: 28. In some embodiments, the second VHH domain comprises the amino acid sequence of SEQ ID NO: 22. In some embodiments, the linker comprises the amino acid sequence of SEQ ID NO: 29.

In some embodiments, the Shiga toxin A subunit effector polypeptide comprises a polypeptide having the sequence of: (i) amino acids 1 to 251 of any one of SEQ ID NOs: 1-18; or (ii) amino acids 1 to 261 of any one of SEQ ID NOs: 1-18; or a polypeptide having a sequence that is at least 90% or at least 95% identical thereto. In some embodiments, the Shiga toxin A subunit effector polypeptide comprises or consists of a polypeptide having the sequence of any one of SEQ ID NO: 40 to 68. In some embodiments, the Shiga toxin A subunit effector polypeptide comprises the amino acid sequence of SEQ ID NO: 41.

In some embodiments, the CTLA-4 binding molecule comprises a linker that links the Shiga toxin A subunit effector polypeptide and the binding region. In some embodiments, the linker that links the Shiga toxin A subunit effector polypeptide and the binding region comprises the amino acid sequence of SEQ ID NO: 218.

In some embodiments, the CTLA-4 binding molecule comprises, from N-terminus to C-terminus or from C-terminus to N-terminus, the Shiga toxin A subunit effector polypeptide, the linker, and the binding region.

In some embodiments, the CTLA-4 binding molecule comprises, from N-terminus to C-terminus, the Shiga toxin A subunit effector polypeptide, the binding region linker, the first VHH domain, the linker, and the second VHH domain.

In some embodiments, the CTLA-4 binding molecule comprises the amino acid sequence of SEQ ID NO: 329, or an amino acid sequence at least 95% identical to SEQ ID NO: 329.

In some embodiments, the CTLA-4 binding molecule is a single continuous polypeptide. In some embodiments, the CTLA-4 binding molecule comprises two polypeptides. In some embodiments, the polypeptides are non-covalently linked. In some embodiments, the polypeptides are covalently linked.

In some embodiments, the CTLA-4 binding molecule is cytotoxic. In some embodiments, the CTLA-4 binding molecule is non-cytotoxic.

In some embodiments, the present disclosure provides a pharmaceutical composition comprising the CTLA-4 binding molecule of the present disclosure, and at least one pharmaceutically acceptable excipient or carrier. In some embodiments, the pharmaceutical composition comprises about 0.1 mg/mL to about 100.0 mg/mL of the CTLA-4 binding molecule. In some embodiments, the pharmaceutical composition comprises about 0.5 mg/mL of the CTLA-4 binding molecule. In some embodiments, the pharmaceutical composition comprises the CTLA-4 binding molecule in a buffer comprising one or more of sodium acetate, sucrose, sodium chloride, and poloxamer 188. In some embodiments, the sodium acetate is at a concentration of about 1 mM to about 50 mM. In some embodiments, the sodium acetate is at a concentration of about 20 mM. In some embodiments, the sucrose is at a concentration of about 1% w/v to about 10% w/v. In some embodiments, the sucrose is at a concentration of about 6% w/v. In some embodiments, the sodium chloride is at a concentration of about 50 mM to about 100 mM. In some embodiments, the sodium chloride is at a concentration of about 75 mM. In some embodiments, the poloxamer 188 is at a concentration of about 0.01% w/v to about 1% w/v. In some embodiments, the poloxamer 188 is at a concentration of about 0.1% w/v. In some embodiments, the buffer has a pH of about 4.0 to about 7.0. In some embodiments, the buffer has a pH of about 5.0.

In some embodiments, the present disclosure provides a polynucleotide encoding any one of the CTLA-4 binding molecules described herein, or a complement thereof. In some embodiments, the present disclosure provides an expression vector comprising the polynucleotide. In some embodiments, the present disclosure provides a host cell comprising the polynucleotide or the expression vector.

In some embodiments, the present disclosure provides a method of treating cancer, the method comprising administering to a subject in need thereof an effective amount of any one of the CTLA-4 binding molecules described herein, or any one of the pharmaceutical compositions described herein.

In some embodiments, the effective amount of the CTLA-4 binding molecule is a dose in a range of about 1 μg/kg to about 250 μg/kg. In some embodiments, the dose is about 1 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 125 μg/kg, about 150 μg/kg, about 175 μg/kg, about 200 μg/kg, about 250 μg/kg, or any value therebetween. In some embodiments, the dose is about 32 μg/kg.

In some embodiments, the CTLA-4 binding molecule is administered to the subject by intravenous, subcutaneous, or intramuscular injection. In some embodiments, the CTLA-4 binding molecule is administered to the subject by intravenous injection. In some embodiments, the CTLA-4 binding molecule is administered to the subject over a period of about 10 minutes to about 1 hour. In some embodiments, the CTLA-4 binding molecule is administered to the subject over a period of about 30 minutes.

In some embodiments, the CTLA-4 binding molecule is administered to the subject once. In some embodiments, the CTLA-4 binding molecule is administered to the subject more than once. In some embodiments, the CTLA-4 binding molecule is administered to the subject once every seven days.

In some embodiments, the CTLA-4 binding molecule is administered to the subject over a 28 day cycle. In some embodiments, the CTLA-4 binding molecule is administered to the subject on days 1, 8, 15, and 22 of the 28 day cycle. In some embodiments, the CTLA-4 binding molecule is administered to the subject on days 1 and 15 of the 28 day cycle. In some embodiments, the CTLA-4 binding molecule is administered to the subject on days 1, 8, and 15 of the 28 day cycle.

In some embodiments, the subject is administered a dose in the range of about 1 μg/kg to about 250 μg/kg at each administration. In some embodiments, the subject is administered a dose of about 1 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 125 μg/kg, about 150 μg/kg, about 175 μg/kg, about 200 μg/kg, about 250 μg/kg, or any value therebetween at each administration. In some embodiments, the subject is administered a dose of about 32 μg/kg at each administration.

In some embodiments, the CTLA-4 binding molecule is administered to the subject over at least one 28 day cycle. In some embodiments, the CTLA-4 binding molecule is administered to the subject over one 28 day cycle, two 28 day cycles, three 28 day cycles, four 28 day cycles, five 28 day cycles, or six 28 day cycles.

In some embodiments, the method comprises administering to the subject a second anti-cancer agent. In some embodiments, the CTLA-4 binding molecule is administered to the subject before the second anti-cancer agent. In some embodiments, the CTLA-4 binding molecule is administered to the subject after the additional anti-cancer agent. In some embodiments, the CTLA-4 binding molecule is administered to the subject at the same time as the additional anti-cancer agent.

In some embodiments, the second anti-cancer agent is a PD-1 or PD-L1 inhibitor. In some embodiments, the PD-1 inhibitor is an anti-PD-1 antibody or anti-PD-1 antibody-drug conjugate (ADC). In some embodiments, the anti-PD-1 antibody is nivolumab, pembrolizumab, dostarlimab, tislelizumab, or cemiplimab. In some embodiments, the anti-PD-1 antibody is nivolumab. In some embodiments, the PD-1 inhibitor is administered to the subject at a dose of about 250 mg to about 750 mg. In some embodiments, the PD-1 inhibitor is administered to the subject at a dose of about 480 mg. In some embodiments, the PD-1 inhibitor is administered to the subject by intravenous injection. In some embodiments, the PD-1 inhibitor is administered to the subject once. In some embodiments, the PD-1 inhibitor is administered to the subject more than once. In some embodiments, the PD-1 inhibitor is administered to the subject once in a 28 day cycle. In some embodiments, the PD-1 inhibitor is administered to the subject on day 1 of the 28 day cycle. In some embodiments, the PD-1 inhibitor is administered to the subject over at least one 28 day cycle. In some embodiments, the PD-1 inhibitor is administered to the subject starting on day 1 of a second 28 day cycle.

In some embodiments, the PD-L1 inhibitor is an anti-PD-L1 antibody or anti-PD-L1 ADC. In some embodiments, the anti-PD-L1 antibody is atezolizumab, durvalumab, or avelumab.

In some embodiments, the subject receives at least one pre-medication prior to the administration of the CTLA-4 binding molecule. In some embodiments, the at least one pre-medication is an H1/H2 blocker-containing agent and/or anti-pyrectic agent.

In some embodiments, the cancer is a solid tumor. In some embodiments, the cancer is bladder cancer, breast cancer, cervical cancer, colon cancer, endometrial cancer, esophageal cancer, fallopian tube cancer, gastrointestinal cancer, glioma, head and neck cancer, kidney cancer, liver cancer, lung cancer, lymphoma, Merkel cell carcinoma, mesothelioma, myeloma, nasopharyngeal neoplasm, ovarian cancer, pancreatic cancer, peritoneal neoplasm, prostate cancer, skin cancer, transitional cell carcinoma, soft tissue sarcoma, microsatellite instability-high (MSI-H) or mismatch repair deficient (dMMR), or urothelial cancer. In some embodiments, the cancer is bladder cancer, and the bladder cancer is urothelial carcinoma. In some embodiments, the cancer is breast cancer, and the breast cancer is HER2 positive breast cancer or triple negative breast cancer. In some embodiments, the cancer is colon cancer, and the colon cancer is colorectal cancer. In some embodiments, the cancer is gastrointestinal cancer, and the gastrointestinal cancer is gastric cancer, biliary tract neoplasm, or gastroesophageal junction cancer. In some embodiments, the cancer is glioma, and the glioma is glioblastoma. In some embodiments, the cancer is head and neck cancer, and the head and neck cancer is squamous cell carcinoma of the head and neck. In some embodiments, the cancer is kidney cancer, and the kidney cancer is renal cell carcinoma. In some embodiments, the cancer is liver cancer, and the liver cancer is hepatocellular carcinoma. In some embodiments, the cancer is lung cancer, and the lung cancer is non-small cell lung cancer or small-cell lung cancer. In some embodiments, the non-small cell lung cancer is metastatic non-small cell lung cancer. In some embodiments, the cancer is lymphoma, and the lymphoma is Hodgkin lymphoma, non-Hodgkin lymphoma, primary mediastinal large B-cell lymphoma, or diffuse large B-cell lymphoma. In some embodiments, the cancer is mesothelioma, and the mesothelioma is pleural mesothelioma. In some embodiments, the pleural mesothelioma is malignant pleural mesothelioma. In some embodiments, the cancer is myeloma, and the myeloma is multiple myeloma. In some embodiments, the cancer is skin cancer, and the skin cancer is squamous cell cancer of the skin or melanoma. In some embodiments, the melanoma is unresectable melanoma or metastatic melanoma. In some embodiments, the cancer is cervical cancer, and the cervical cancer is cervical carcinoma. In some embodiments, the cancer is esophageal cancer, and the esophageal cancer is esophageal squamous cell carcinoma. In some embodiments, the cancer is MSI-H or dMMR cancer. In some embodiments, the cancer is metastatic.

In some embodiments, the cancer is relapsed or refractory to a treatment involving at least one other cancer therapy, or the subject is known to be intolerant of at least one other cancer therapy. In some embodiments, the at least one other cancer therapy is ipilimumab, nivolumab, pembrolizumab, atezolizumab, durvalumab, avelumab, tremelimumab, cemiplimab, relatlimab, tiragolumab, ociperlimab, vibostolimab, domvanalimab, sacituzumab, sacituzumab govitecan, datopotamab, or datopotamab deruxtecan.

In some embodiments, the present disclosure provides a CTLA-4 binding molecule comprising a Shiga toxin A subunit effector polypeptide and a binding region capable of specifically binding an extracellular part of CTLA-4; wherein the CTLA-4 binding molecule comprises the amino acid sequence of SEQ ID NO: 329, or an amino acid sequence at least 95% identical thereto. In some embodiments, the CTLA-4 binding molecule comprises the amino acid sequence of SEQ ID NO: 329.

In some embodiments, the present disclosure provides a pharmaceutical composition comprising any one of the CTLA-4 binding molecules described herein, and at least one pharmaceutically acceptable excipient or carrier.

In some embodiments, the present disclosure provides a method of treating cancer, the method comprising administering to a subject in need thereof an effective amount of any one of the CTLA-4 binding molecules described herein, or any one of the pharmaceutical compositions described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts an illustrative CTLA-4-binding molecule monomer as described herein, comprising, from N-terminus to C-terminus or from C-terminus to N-terminus, a Shiga toxin A subunit effector polypeptide and a CTLA-4 binding region. The CTLA-4-binding molecule can further comprise a binding region linker (depicted in black) that links the Shiga toxin A subunit effector polypeptide and the CTLA-4 binding region.

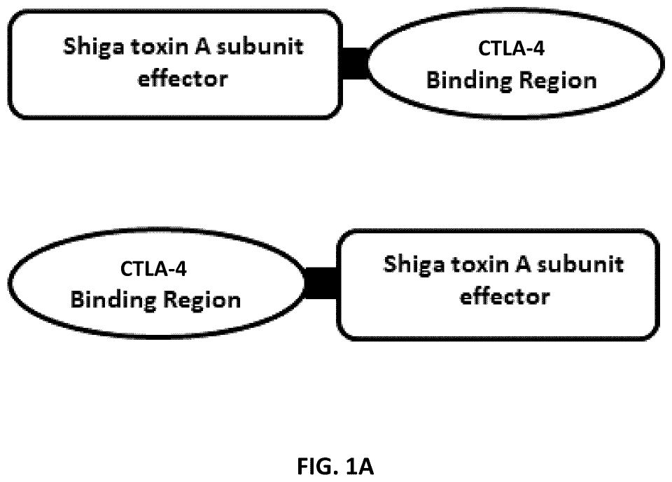

FIG. 1B depicts illustrative dimers of CTLA-4-binding molecules as described herein, wherein each CTLA-4 binding molecule comprises from N-terminus to C-terminus or from C-terminus to N-terminus, a Shiga toxin A subunit effector polypeptide and a CTLA-4 binding region. The CTLA-4-binding molecules can further comprise a binding region linker (depicted in black) that links the Shiga toxin A subunit effector polypeptide and the CTLA-4-binding region. The dimers can further comprise a second linker (each depicted as a black curve) that links the two binding molecules. In some embodiments, for example as shown in FIG. 1B (bottom left), the CTLA-4-binding region is an scFv, and the scFv participates in intermolecular variable domain exchange (i.e., non-covalent association) with a neighboring scFv.

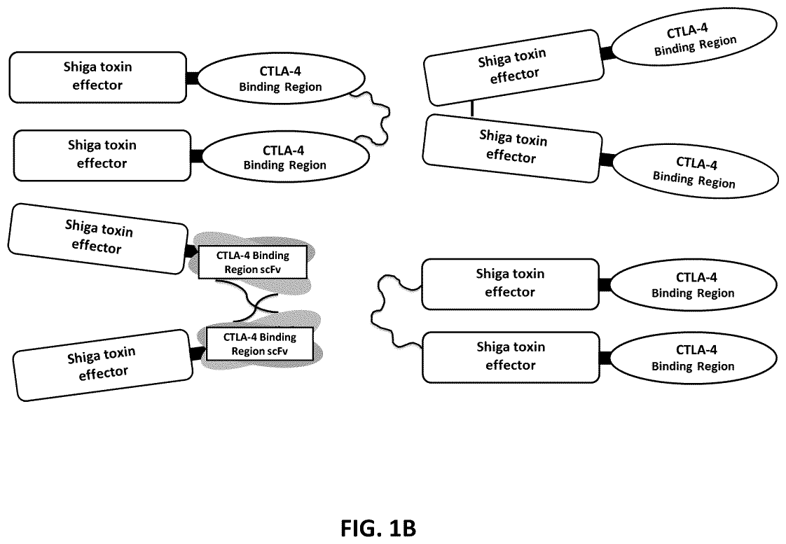

FIG. 1C depicts illustrative CTLA-4-binding molecules as described herein, comprising from N-terminus to C-terminus: (i) a Shiga toxin A subunit effector polypeptide, (ii) a CTLA-4 binding region, and (iii) a T-cell epitope for delivery to the interior of a target cell and subsequent cell-surface presentation; or (i) a CTLA-4 binding region, (ii) a Shiga toxin A subunit effector polypeptide, and (iii) a T-cell epitope for delivery to the interior of a target cell and subsequent cell-surface presentation.

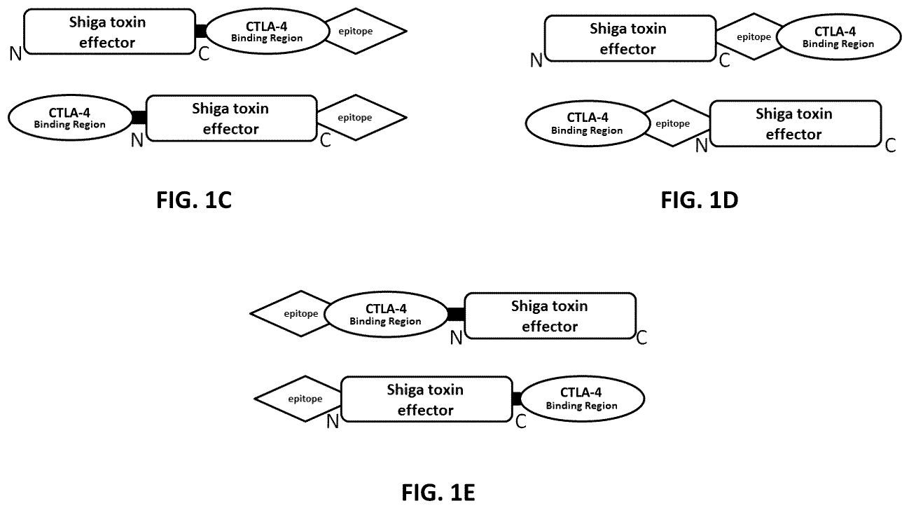

FIG. 1D depicts illustrative CTLA-4 binding molecules as described herein, comprising from N-terminus to C-terminus or from C-terminus to N-terminus, (i) a Shiga toxin A subunit effector polypeptide, (ii) a T-cell epitope for delivery to the interior of a target cell and subsequent cell-surface presentation, and (iii) a CTLA-4 binding region; or comprising from N-terminus to C-terminus or from C-terminus to N-terminus, (i) a CTLA-4 binding region, (ii) a T-cell epitope for delivery to the interior of a target cell and subsequent cell-surface presentation, and (iii) a Shiga toxin A subunit effector polypeptide.

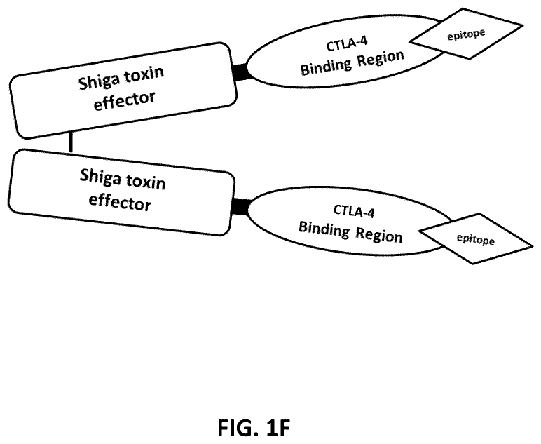

FIG. 1E depicts illustrative CTLA-4 binding molecules as described herein, comprising: (i) a T-cell epitope for delivery to the interior of a target cell and subsequent cell-surface presentation, (ii) a CTLA-4 binding region, and (iii) a Shiga toxin A subunit effector polypeptide; or (i) a T-cell epitope for delivery to the interior of a target cell and subsequent cell-surface presentation, (ii) a Shiga toxin A subunit effector, and (iii) a CTLA-4 binding region.

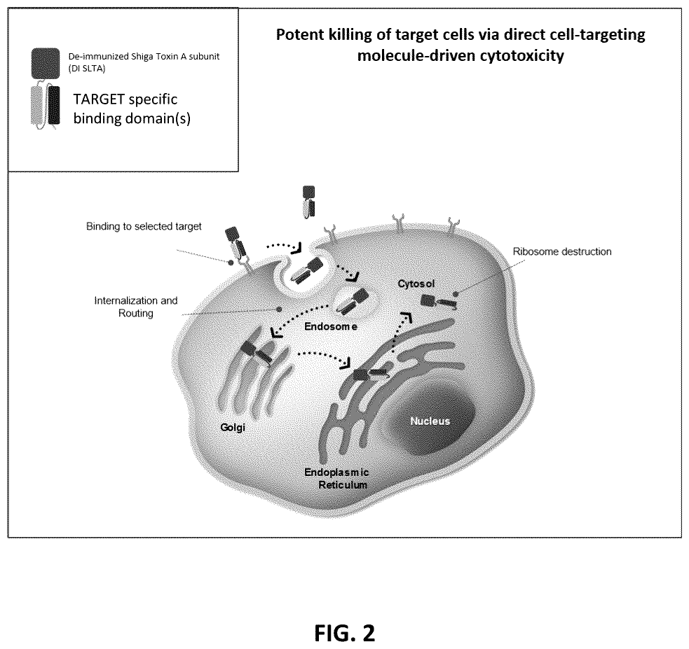

FIG. 1F depicts an illustrative CTLA-4 binding molecule dimer. The dimer comprises two T-cell epitopes for delivery to the interior of a target cell and subsequent cell-surface presentation. The epitopes can be the same or different.

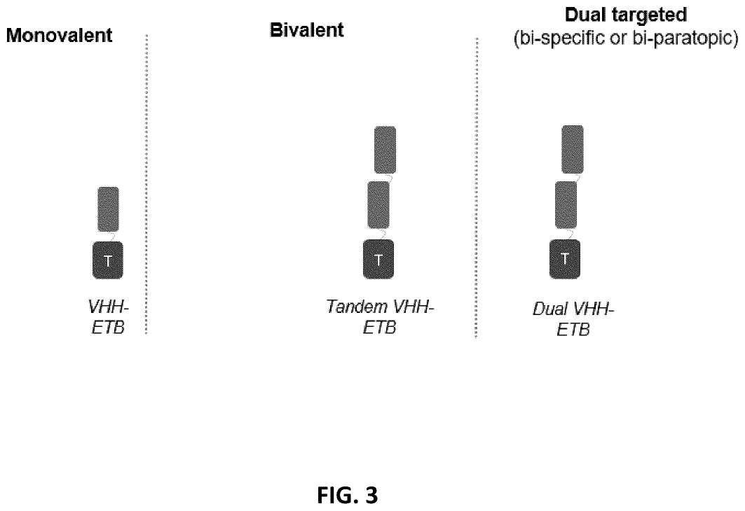

FIG. 2 (top left panel) is a schematic drawing of an illustrative binding molecule comprising an engineered deimmunized (DI) Shiga toxin subunit A effector polypeptide (DI-SLTA) fused via a binding region linker to a CTLA-4 binding region comprising a CTLA-4-specific binding domain or domains. FIG. 2 also provides a schematic drawing showing a potential mechanism by which the binding molecule may directly kill a target cell, which involves specific binding to the CTLA-4-expressing cells, internalization into these target cells; intracellular self-routing in a retrograde pathway from the endosome to the Golgi, then to the endoplasmic reticulum, and then to the cytosol; and once in the cytosol irreversible and enzymatic inactivation of ribosomes to resulting in target cell death. This putative mechanism of action for the binding molecule is predicted to be independent of patient's immune function status.

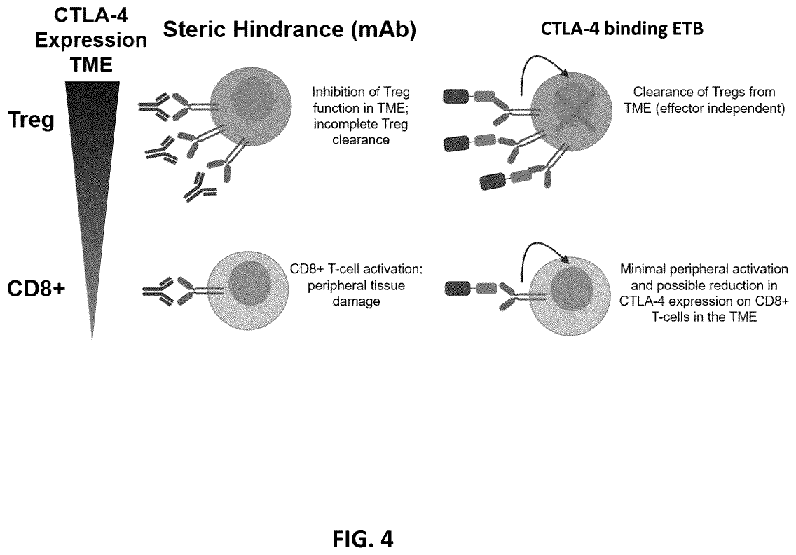

FIG. 3 illustrates the structure of exemplary binding molecules. In the left panel, a monovalent binding molecule is provided which comprises a VHH linked to a Shiga toxin A subunit effector peptide (labeled with a “T”). An illustrative bivalent binding molecule is depicted in the center panel. For example, the bivalent binding molecule can be a non-covalent dimer. In some embodiments, the binding molecule may comprise two VHH domains in tandem, linked to a Shiga toxin A subunit effector peptide. In the right panel, an illustrative dual-targeted binding molecule is shown, wherein the binding molecule comprises a first binding domain capable of binding CTLA-4, a second binding domain capable of binding a second and different CTLA-4 epitope, and a Shiga toxin A subunit effector peptide.

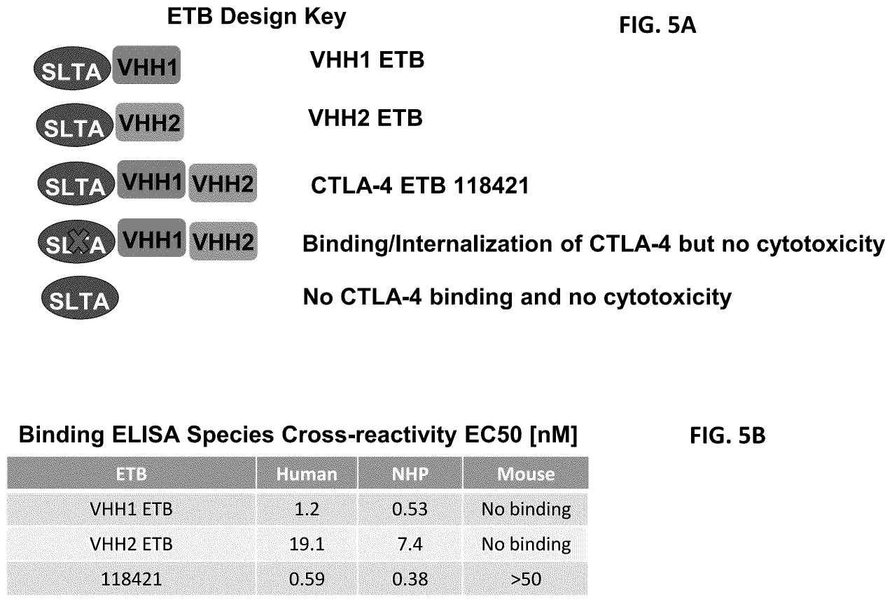

FIG. 4 is a schematic comparing the binding of monoclonal anti-CTLA-4 antibodies (mAb) and CTLA-4 binding ETBs (engineered toxin bodies) as described herein to Tregs and CD8+ T-cells. Because CTLA-4 is highly expressed on Tregs in the TME in a subject with cancer but expressed at low levels on CD8+ T-cells, the ETBs are predicted to have greater potency on Tregs in the TME over CD8+ T-cells in the periphery. Anti-CTLA-4 mAbs inhibit Treg function at least in part by blocking the ability of CTLA-4 to interact with its ligands (i.e., by steric hindrance). Some CTLA-4 antibody-bound Tregs may be cleared from the TME by an effector cell-dependent mechanism. Anti-CTLA-4 antibodies bind to and activate CD8+ T-cells, resulting in peripheral tissue damage. In contrast, and without being bound by any particular theory, it is hypothesized that the CTLA-4 binding ETBs described herein bind to Tregs in the TME and are subsequently internalized, leading to death and clearance of the Tregs according to an effector-independent mechanism. When CTLA-4 binding ETBs bind to CD8+ T-cells in the periphery, there might be minimal peripheral activation, and it is possible that CTLA-4 expression on CD8+ T-cells in the TME may be reduced. This may further limit the binding of CTLA-4 binding ETBs thereto, as long as there is a competitive binding “sink” present, such as a higher CTLA-4 expressing cell type (e.g., a Treg).

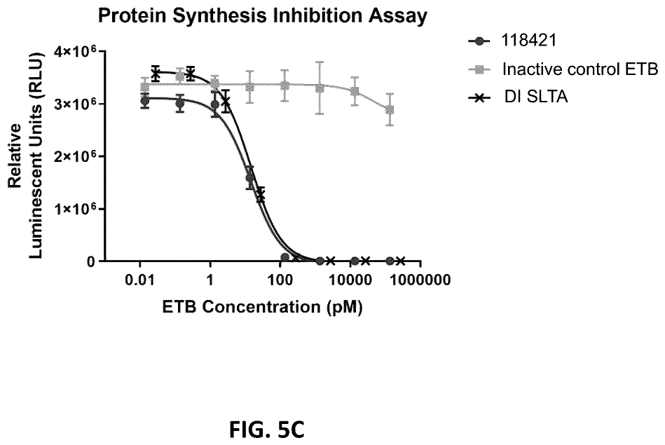

FIG. 5A is a schematic depicting the design of CTLA-4 binding molecules as described herein. VHH1 and VHH2 are unique CTLA-4 binding domains.

FIG. 5B is a table showing the species cross-reactivity EC50 of exemplary CTLA-4 binding molecules.

FIG. 5C depicts the results from a protein synthesis inhibition assay with the CTLA-4 ETB 118421. Inactive ETB was used as a negative control and SLT-I A1 V1 was used as a positive control.

FIG. 6A-FIG. 6B depict results from a CTLA-4 blockade bioassay of exemplary CTLA-4 binding molecules tested in a cellular system.

FIG. 7 depicts the structure of CTLA-4 with critical contact residues for binding by CTLA-4 ETB 118421 mapped on the CTLA-4 structure. Left: VHH1 (Red) and VHH2 (Blue) critical CTLA-4 contact residues identified through shotgun mutagenesis and high-throughput flow cytometry (Integral Molecular) Right: Docked structure is superimposed on crystal structure of complex of CTLA-4 with Fv of ipilimumab (PDB: 5TRU, cyan) and B7-1 (PDB: 1I8L, blue). CDR3 loop of VHHs are colored black.

FIG. 8A, FIG. 8B, and FIG. 8C depict results from assays measuring CTLA-4 expression on T cells from melanoma patient samples.

FIG. 9A, FIG. 9B, and FIG. 9C depict results from cell viability assays analyzing CTLA-4 ETB 118421 potency on gain-of-function cell lines expressing different CTLA-4 levels. FIG. 9C shows cell viability of gain-of-function cell lines upon treatment with 60 nM CTLA-4 ETB 118421.

FIG. 10A and FIG. 10B depict results from assays analyzing CTLA-4 ETB 118421 reduction of Treg-mediated T cell suppression.

FIG. 11A shows phenotyping of ex-vivo expanded Tregs from healthy donor (Donor 8316). FIG. 11B depicts results from a cytotoxicity assay analyzing CTLA-4 ETB 118421 induction of apoptosis in primary Tregs shown in FIG. 11A.

FIG. 11C shows phenotyping of ex-vivo expanded Tregs from healthy donor (Donor 110040210). FIG. 11D depicts results from a cytotoxicity assay analyzing CTLA-4 ETB 118421 induction of apoptosis in primary Tregs shown in FIG. 11C.

FIG. 11E depicts results from a cytotoxicity assay analyzing CTLA-4 ETB 118421 induction of apoptosis in primary CD8+ T cells.

FIG. 12 depicts results from assays analyzing CTLA-4 ETB 118421 and/or anti-PD-1 antibody inhibition of Treg-mediated T cell suppression.

FIG. 13A depicts the results for the pro-inflammatory cytokine IL-6 in PBMCs stimulated with LPS, anti-CD3/anti-CD28 beads, deimmunized (DI) SLTA, enzymatically inactive CTLA-4 ETB, or CTLA-4 ETB 118421. LPS and anti-CD3/anti-CD28 beads were used as a positive control. Each of the symbols on the bar graph represent the average value obtained from 3 individual donor PBMCs.

FIG. 13B depicts the results for the pro-inflammatory cytokine TNF-α in PBMCs stimulated with LPS, anti-CD3/anti-CD28 beads, deimmunized (DI) SLTA, enzymatically inactive CTLA-4 ETB, or CTLA-4 ETB 118421. LPS and anti-CD3/anti-CD28 beads were used as a positive control. Each of the symbols on the bar graph represent the average value obtained from 3 individual donor PBMCs.

FIG. 13C depicts the results for the pro-inflammatory cytokine IL-6 in PBMCs stimulated with LPS, anti-CD3/anti-CD28 beads, deimmunized (DI) SLTA, IgG4 isotype control, anti-PD-1, and/or CTLA-4 ETB 118421. LPS and anti-CD3/anti-CD28 beads were used as a positive control.

FIG. 13D depicts the results for the pro-inflammatory cytokine TNF-α in PBMCs stimulated with LPS, anti-CD3/anti-CD28 beads, deimmunized (DI) SLTA, IgG4 isotype control, anti-PD-1, and/or CTLA-4 ETB 118421. LPS and anti-CD3/anti-CD28 beads were used as a positive control.

FIG. 14A is a timeline depicting intravenous treatments with CTLA-4 ETB 118421 in a study with a human CTLA-4 knock-in HuGEMM mouse model inoculated with MC38 tumors. On day 4, tumor and spleen tissue was harvested and processed for immunophenotyping.

FIG. 14B depicts results from a study analyzing the effect of CTLA-4 ETB 118421 on Tregs in the tumor microenvironment (top panel) and spleen (bottom panel) in a study with a human CTLA-4 knock-in HuGEMM mouse model inoculated with MC38 tumors.

FIG. 15A shows the study design for a non-human primate toxicology study with CTLA-4 ETB 118421. FIG. 15B depicts a graph showing serum concentration-time profiles following intravenous administration of CTLA-4 ETB 118421 in non-human primates. FIG. 15C is a table summarizing individual pharmacokinetic parameters following intravenous administration of CTLA-4 ETB 118421 in non-human primates.

FIG. 16A shows the study design for a non-human primate pharmacokinetics study with CTLA-4 ETB 118421. FIG. 16B and FIG. 16C depict serum concentration-time profiles following intravenous administration of CTLA-4 ETB 118421 in non-human primates.

FIG. 17A and FIG. 17B show the study designs for the phase 1 clinical trial examining the CTLA-4 ETB 118421 as a monotherapy and in combination with nivolumab in patients with advanced solid cancer types.

FIG. 18 shows the dose escalation/de-escalation rule used for the CTLA-4 ETB 118421 in the phase 1 clinical trial.

FIG. 19 shows the statistical design for Part B expansion cohorts.

FIG. 20 shows cytotoxicity of CTLA-4 ETB 118421 in human CTLA-4 expressing CHO-K1 cells in the presence of ipilimumab.

### DETAILED DESCRIPTION

In some embodiments, the CTLA-4 binding molecules described herein comprise (i) a Shiga toxin A subunit effector polypeptide and (ii) a binding region capable of specifically binding CTLA-4 on the surface of a cell, such as an IIC. Such CTLA-4 binding molecules are also referred to as ETBs (engineered toxin bodies). In some embodiments, upon binding to CTLA-4 on the cell, the CTLA-4 binding molecules are internalized and the activity of the Shiga toxin A subunit effector polypeptide effectively and specifically kills the cell. In some embodiments, this direct cell kill activity depletes immunosuppressive immune cells, such as Tregs in the TME. In some embodiments, the CTLA-4 binding molecules bind and inhibit CTLA-4 signaling to or from immunosuppressive immune cells. Once immunosuppression in the TME is lifted, non-suppressive immune cells (e.g., cytotoxic T cells) can attack the tumor. The CTLA-4 binding molecules described herein can be used in combination therapy with at least one additional anti-cancer agent.

In some embodiments, the binding molecules described herein bind to CTLA-4 that is on an IIC and on a tumor cell. Thus, in some embodiments, in addition to depleting immunosuppressive immune cells in the TME, the binding molecules also bind to and directly kill tumor cells. This dual mechanism of action can enhance effectiveness of the disclosed binding molecules in cancer therapy.

In some embodiments, the binding molecules modulate expression of CTLA-4 to which the binding molecules' binding region binds. In some embodiments, the binding molecules reduce or downregulate expression of CTLA-4. In some embodiments, the binding molecules reduce cell-surface density of CTLA-4. In some embodiments, modulation of expression of CTLA-4 reduces immunosuppression. In some embodiments, modulation of expression of CTLA-4 leads to cell death.

Thus, the disclosed binding molecules are useful (1) for selectively killing a cell type(s) expressing CTLA-4 amongst other cells, and (2) as therapeutic molecules for treating a variety of diseases, disorders, and conditions, including cancers.

The present invention is described more fully hereinafter using illustrative, non-limiting embodiments, and references to the accompanying figures. This invention can, however, be embodied in many different forms and should not be construed as to be limited to the embodiments set forth below. Rather, these embodiments are provided so that this invention is thorough and conveys the scope of the invention to those skilled in the art.

All references, articles, publications, patents, patent publications, and patent applications cited herein are incorporated by reference in their entireties for all purposes. However, disclosure of any reference, article, publication, patent, patent publication, and patent application cited herein is not, and should not be taken as, an acknowledgment or any form of suggestion that it constitutes valid prior art or form part of the common general knowledge in any country in the world.

### I. Definitions

Unless otherwise noted, the terms used herein have definitions as ordinarily used in the art. Some terms are defined below, and additional definitions can be found within the detailed description.

As used in the specification and the appended claims, the terms “a,” “an” and “the” include both singular and plural referents unless the context clearly dictates otherwise.

As used in the specification and the appended claims, the term “and/or” when referring to two species, A and B, means at least one of A and B. As used in the specification and the appended claims, the term “and/or” when referring to greater than two species, such as A, B, and C, means at least one of A, B, or C, or at least one of any combination of A, B, or C (with each species in singular or multiple possibility).

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

The term “amino acid residue” or “amino acid” includes reference to an amino acid that is incorporated into a protein, polypeptide, or peptide. The term “polypeptide” includes any polymer of amino acids or amino acid residues. The term “polypeptide sequence” refers to a series of amino acids or amino acid residues which physically comprise a polypeptide.

A “protein” is a macromolecule comprising one or more polypeptides or polypeptide “chains.” For example, a protein can comprise one, two, three, four, five, six, seven, eight, nine, ten, or more polypeptides. In embodiments wherein the protein comprises more than one polypeptide, the polypeptides of the protein can either be the same or different from one another. A protein can be a monomer, or a multimer, such as a dimer, trimer, tetramer, etc.

A “peptide” is a small polypeptide of a size less than about a total of 15 to 20 amino acid residues. The term “amino acid sequence” refers to a series of amino acids or amino acid residues which physically comprise a peptide or polypeptide depending on the length. Sometimes “residue” as used herein is meant to indicate a position in a protein and its associated amino acid identity. Unless otherwise indicated, polypeptide and protein sequences disclosed herein are written from left to right representing their order from an amino-terminus to a carboxy-terminus.

The terms “amino acid,” “amino acid residue,” “amino acid sequence,” or polypeptide sequence include naturally occurring amino acids (including L and D stereoisomers) and, unless otherwise limited, also include known analogs of natural amino acids that can function in a similar manner as naturally occurring amino acids, such as selenocysteine, pyrrolysine, N-formylmethionine, gamma-carboxyglutamate, hydroxyprolinehypusine, pyroglutamic acid, and selenomethionine. The amino acids referred to herein are described by shorthand designations as follows in Table 1:

TABLE 1
Amino Acid Nomenclature

| Name | 3-letter | 1-letter |
|---|---|---|
| Alanine | Ala | A |
| Arginine | Arg | R |
| Asparagine | Asn | N |
| Aspartic Acid or Aspartate | Asp | D |
| Cysteine | Cys | C |
| Glutamic Acid or Glutamate | Glu | E |
| Glutamine | Gln | Q |
| Glycine | Gly | G |
| Histidine | His | H |
| Isoleucine | Ile | — |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, “modification” refers to an amino acid substitution, insertion, and/or deletion in a polypeptide sequence or an alteration to a moiety chemically linked to a protein. For example, a modification can be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

As used herein, “amino acid substitution” or “substitution” refer to the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution N297A refers to a variant polypeptide, in this case an Fc variant, in which the asparagine at position 297 is replaced with alanine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

The phrase “conservative substitution” with regard to an amino acid residue of a peptide, peptide region, polypeptide region, protein, or molecule refers to a change in the amino acid composition of the peptide, peptide region, polypeptide region, protein, or molecule that does not substantially alter the function and structure of the overall peptide, peptide region, polypeptide region, protein, or molecule.

As used herein, “amino acid insertion” or “insertion” refer to the addition of an amino acid sequence at a particular position in a parent polypeptide sequence. For example,−233E or 233E designates an insertion of glutamic acid after position 233 and before position 234. Additionally, 233ADE or A233ADE designates an insertion of AlaAspGlu after position 233 and before position 234.

As used herein, “amino acid deletion” or “deletion” refer to the removal of an amino acid sequence at a particular position in a parent polypeptide sequence. For example, E233− or E233 #, E233( ) or E233del designates a deletion of glutamic acid at position 233. Additionally, EDA233− or EDA233 #designates a deletion of the sequence GluAspAla that begins at position 233.

As used herein, the terms “modified protein,” “mutant protein,” “variant protein” or “protein variant”, or “variant” mean a protein that differs from that of a parent protein by virtue of at least one amino acid modification. Protein variant can refer to the protein itself, a composition comprising the protein, or the amino sequence that encodes it. In some embodiments, the protein variant has at least one amino acid modification compared to the parent protein, e.g., from about one to about seventy amino acid modifications, and in some embodiments, from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4, although human sequences with variants can also serve as “parent polypeptides”. In some embodiments, the protein variant sequence herein will possess at least about 80% identity with a parent protein sequence, and in some embodiments will possess at least about 90% identity, at least about 95%, at least about 95%, or at least about 99% identity to the parent protein sequences. Variant protein can refer to the variant protein itself, compositions comprising the protein variant, or the DNA sequence that encodes it.

The terms “binding molecule” or “protein fusion” are used herein to describe a protein comprising at least two domains that are encoded by separate genes and have been joined so that they are transcribed and translated as a single unit, producing a single polypeptide. In some embodiments, a binding molecule can be a homodimeric binding molecule (comprising two identical binding molecule monomers) or a heterodimeric binding molecule (comprising two different binding molecule monomers). In some embodiments, a binding molecule is a multimeric binding molecule (comprising at least two binding molecule monomers.)

“Specific binding” or “specifically binds to” or is “specific for” a particular target or an epitope means binding that is measurably different from a non-specific interaction, e.g., binds preferentially to one target relative to another. Specific binding can be measured, for example, by determining binding of a first molecule, e.g., binding molecule, or binding domain thereof, compared to binding of a second, control molecule or binding domain thereof. In some embodiments, the control molecule that has a structure that is similar to that of the first molecule, but that does not bind to the particular target. For example, specific binding can be determined by competition with a control molecule that is similar to the target. Specific binding can include binding having an equilibrium dissociation constant (K_{D}) of at least 10⁶ M⁻¹, at least 10⁷ M⁻¹, at least 10⁸ M⁻¹, at least 10⁹ M⁻¹, or at least 10¹⁰ M⁻¹, or an affinity in the range of, for example, about 10⁶ M⁻¹ to about 10¹⁰ M⁻¹, about 10⁷ M⁻¹ to about 10¹⁰ M⁻¹, or about 10⁸ M⁻¹ to about 10¹⁰ M.

By “binding region” herein is meant a polypeptide capable of specifically binding to a target (e.g., CTLA-4). In some embodiments, a binding region comprises a set of six Complementary Determining Regions (CDRs) that, when present as part of a polypeptide sequence, specifically binds a target antigen. Thus, an “anti-CTLA-4 binding region” binds a CTLA-4 target as outlined herein. As is known in the art, these CDRs are generally present as a first set of variable heavy CDRs (HCDRs or VHCDRs) and a second set of variable light CDRs (LCDRs or VLCDRs), each comprising three CDRs: HCDR1, HCDR2, HCDR3 for the heavy chain and LCDR1, LCDR2 and LCDR3 for the light chain. As is understood in the art, the CDRs are separated by framework regions in each of the heavy variable and light variable regions: for the light variable region, these are (VL)FR1-LCDR1-(VL)FR2-LCDR2-(VL)FR3-LCDR3-(VL)FR4, and for the heavy variable region, these are (VH)FR1-HCDR1-(VH)FR2-HCDR2-(VH)FR3-HCDR3-(VH)FR4.

Binding regions can be embodied in multiple formats, for example, in Fab, Fv and scFv. In an “Fab” format, the set of 6 CDRs are contributed by two different polypeptide sequences, the heavy variable region (vh or VH; containing the HCDR1, HCDR2 and HCDR3) and the light variable region (v1 or VL; containing the LCDR1, LCDR2 and LCDR3), with the C-terminus of the VH being attached to the N-terminus of the CH1 domain of the heavy chain and the C-terminus of the VL being attached to the N-terminus of the constant light domain (and thus forming the light chain). Heavy variable regions and light variable regions together form Fvs, which can be either scFvs or Fabs, as outlined herein. Thus, in some cases, the six CDRs of the antigen binding domain are contributed by a VH and VL. In an scFv format, the VH and VL are covalently attached, generally through the use of a linker as outlined herein, into a single polypeptide sequence, which can be either (starting from the N-terminus) VH-linker-VL or VL-linker-VH.

By “Fab” or “Fab region” as used herein is meant the polypeptide that comprises the VH, CH1, VL, and CL immunoglobulin domains. Fab can refer to this region in isolation, or this region in the context of a full-length antibody or antibody fragment.

By “Fv” or “Fv fragment” or “Fv region” as used herein is meant a polypeptide that comprises the VL and VH domains of a single antibody. As will be appreciated by those in the art, these are made up of two domains, a variable heavy domain and a variable light domain.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (VH-linker-VL or VL-linker-VH). In general, the linker is a scFv linker as is generally known in the art, and discussed above.

The term “VHH” is used herein to describe a single domain antibody, an autonomous heavy domain antibody variable domain, or a binding region having structural and/or sequence similarity to, for example, a variable antigen-binding domain heavy-chain antibody from a camelid (camel, dromedary, llama, alpaca, etc.) or to an immunoglobulin new antigen receptor (IgNAR) of a cartilaginous fish (e.g., a shark). In some embodiments, a VHH may be very small in size, for example about 12 to about 15 kDa. A VHH may also be referred to herein as a “nanobody.”

By “linker” herein is meant a domain linker that joins two protein domains together, such as are used in scFv and/or other protein and protein fusion structures. For example, a linker may be used to link a Shiga Toxin A subunit effector polypeptide with a binding region, and a “scFv linker” may be used to link the VH and the VL in an scFv. Generally, there are a number of suitable linkers that can be used, including traditional peptide bonds, generated by recombinant techniques that allows for recombinant attachment of the two domains with sufficient length and flexibility to allow each domain to retain its biological function. In some embodiments, the linker peptide can predominantly include the following amino acid residues: Gly, Ser, Ala, or Thr. The linker peptide should have a length that is adequate to link two molecules in such a way that they assume the correct conformation relative to one another so that they retain the desired activity. In some embodiments, the linker is from about 1 to about 50 amino acids in length. In some embodiments, the linker is from about 1 to about 30 amino acids in length. In one embodiment, linkers of 1 to 20 amino acids in length can be used, with from about 5 to about 10 amino acids finding use in some embodiments. Useful linkers include glycine-serine polymers, including for example (GS)n (SEQ ID NO: 187), (GSGGS)n (SEQ ID NO: 188), (GGGGS)n (SEQ ID NO: 189), and (GGGS)n (SEQ ID NO: 190), where n is an integer of at least one (and generally from 3 to 4), glycine-alanine polymers, alanine-serine polymers, and other flexible linkers. Alternatively, a variety of non-proteinaceous polymers, including but not limited to polyethylene glycol (PEG), polypropylene glycol, polyoxyalkylenes, or copolymers of polyethylene glycol and polypropylene glycol, can find use as linkers. Other linker sequences can include any sequence of any length of CL/CH1 domain but not all residues of CL/CH1 domain; for example, the first 5-12 amino acid residues of the CL/CH1 domains. Linkers can also be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences can also be derived from other proteins such as Ig-like proteins (e.g., TCR, FcR, KIR), hinge region-derived sequences, and other natural sequences from other proteins. While any suitable linker can be used, some embodiments utilize a glycine-serine polymer, including for example (GS)n (SEQ ID NO: 187), (GSGGS)n (SEQ ID NO: 188), (GGGGS)n (SEQ ID NO: 189), and (GGGS)n (SEQ ID NO: 190), where n is an integer of at least one (and generally from 2 to 3 to 4 to 5). “scFv linkers” generally include these glycine-serine polymers.

The term “antibody” is used in the broadest sense and includes, for example, an intact immunoglobulin or an antigen binding portion of an immunoglobulin or an antigen binding protein related or derived from an immunoglobulin. Intact antibody structural units often comprise a tetrameric protein. Each tetramer is typically composed of two identical pairs of polypeptide chains, each pair having one “light” chain (typically having a molecular weight of about 25 kDa) and one “heavy” chain (typically having a molecular weight of about 50- to 70 kDa). Human immunoglobulin light chains can be classified as having kappa or lambda light chains. In some embodiments, provided herein are antibody structures comprising antigen binding domains (e.g., antibody heavy and/or light chains) that generally are based on the IgG class, which has several subclasses, including, but not limited to IgG1, IgG2, IgG3, and IgG4. In general, IgG1 has different allotypes with polymorphisms at 356 (D or E), IgG2 and 358 (L or M). The sequences depicted herein use the 356D/358M allotype, however the other allotype is included herein. That is, any sequence inclusive of an IgG1 Fc domain included herein can have 356E/358L replacing the 356D/358M allotype. IgG4 are used more frequently than IgG3.

It should be noted that IgG1 has different allotypes with polymorphisms at 356 (D or E) and 358 (L or M). The sequences depicted herein use the 356D/358M allotype, however the other allotype is included herein. That is, any sequence inclusive of an IgG1 Fc domain included herein can have 356E/358L replacing the 356D/358M allotype.

Thus, “isotype” as used herein is meant any of the subclasses of immunoglobulins defined by the chemical and antigenic characteristics of their constant regions. By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification. Similarly, because IgG1 has a proline at position 241 and IgG4 has a serine there, an IgG4 molecule with a S241 P is considered an IgG subclass modification. Note that subclass modifications are considered amino acid substitutions herein.

By “Fc” or “Fc region” or “Fc domain” as used herein is meant the polypeptide comprising the constant region of an antibody excluding the first constant region immunoglobulin domain (e.g., CH1) and in some cases, part of the hinge. For IgG, the Fc domain comprises immunoglobulin domains CH2 and CH3 (Cγ2 and Cγ3) and the lower hinge region between CH1 (Cγ1) and CH2 (Cγ2). Although the boundaries of the Fc region can vary, the human IgG heavy chain Fc region is usually defined to include residues C226 or P230 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Thus, the “Fc domain” includes the —CH2-CH3 domain, and optionally a hinge domain (hinge-CH2-CH3). In some embodiments, as is more fully described below, amino acid modifications are made to the Fc region, for example to alter binding to one or more FcγR receptors or to the FcRn receptor.

By “heavy constant region” herein is meant the CH1-hinge-CH2-CH3 portion of a human IgG antibody.

By “light constant region” is meant the CL domain from kappa or lambda.

By “variable domain” as used herein is meant the region of an immunoglobulin that comprises one or more Ig domains substantially encoded by any of the Vκ (V·kappa), Vλ (V·lambda), and/or VH genes that make up the kappa, lambda, and heavy chain immunoglobulin genetic loci respectively. Thus a “variable heavy domain” comprises (VH)FR1-HCDR1-(VH)FR2-HCDR2-(VH)FR3-HCDR3-(VH)FR4 and a “variable light domain” comprises (VL)FR1-LCDR1-(VL)FR2-LCDR2-(VL)FR3-LCDR3-(VL)FR4.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that some segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs are described below.

As will be appreciated by those in the art, the exact numbering and placement of the CDRs can be different among different numbering systems. A useful comparison of CDR numbering is as below (Table 2), see Lafranc et al., Dev. Comp. Immunol. 27(1):55-77 (2003):

TABLE 2
Antibody CDR Nomenclature

| | Kabat + | | | | | |
|---|---|---|---|---|---|---|
| | Chothia | IMGT | Kabat | AbM | Chothia | Contact |
| HCDR1 | 26-35 | 27-38 | 31-35 | 26-35 | 26-32 | 30-35 |
| HCDR2 | 50-65 | 56-65 | 50-65 | 50-58 | 52-56 | 47-58 |
| HCDR3 | 95-102 | 105-117 | 95-102 | 95-102 | 95-102 | 93-101 |
| LCDR1 | 24-34 | 27-38 | 24-34 | 24-34 | 24-34 | 30-36 |
| LCDR2 | 50-56 | 56-65 | 50-56 | 50-56 | 50-56 | 46-55 |
| LCDR3 | 89-97 | 105-117 | 89-97 | 89-97 | 89-97 | 89-96 |

In the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

Provided herein are a number of different CDR sets. In this case, a “full CDR set” can comprise the three variable light and three variable heavy CDRs, e.g. a LCDR1, LCDR2, LCDR3, HCDR1, HCDR2 and HCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies.

“Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen can have more than one epitope. The epitope can comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide. Epitopes can be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes can be distinguished in that the binding to the former but not the latter is lost in the presence of denaturing solvents.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. In some embodiments, the method for determining the K_{D} of an antibody is by using surface plasmon resonance (SPR), for example, by using a biosensor system such as a BIACORE® system. In some embodiments, the K_{D} of an antibody is determined by Bio-Layer Interferometry. In some embodiments, the K_{D} is measured using flow cytometry with antigen-expressing cells. In some embodiments, the K_{D} value is measured with the antigen immobilized. In other embodiments, the K_{D} value is measured with the antibody (e.g., parent mouse antibody, chimeric antibody, or humanized antibody variants) immobilized. In some embodiments, the K_{D} value is measured in a bivalent binding mode. In other embodiments, the K_{D} value is measured in a monovalent binding mode. Specific binding for a particular antigen or an epitope can be exhibited, for example, by an antibody having a K_{D} for an antigen or epitope of at least about 10⁻⁵ M, at least about 10⁻⁶ M, at least about 10⁻⁷ M, at least about 10⁻⁸ M, at least about 10⁻⁸ M, at least about 10⁻¹⁰ M, at least about 10⁻¹¹ M, at least about 10⁻¹² M, at least about 10⁻¹³ M, at least about 10⁻¹⁴ M. Typically, an antibody that specifically binds an antigen will have a K_{D} that is about 20-, about 50-, about 100-, about 500-, about 1000-, about 5,000-, about 10,000- or more times greater for a control molecule relative to the antigen or epitope.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity. In some embodiments, the percent sequence identity is at least about 90%, at least about 95%, at least about 96%, at least about 97%, at least about 98%, or at least about 99%.

Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full-length of the sequences being compared. One particular program is the ALIGN-2 program outlined at paragraphs [0279] to [0280] of US Pre-Grant Pub. No. 2016/0244525, hereby incorporated by reference. Another approximate alignment for nucleic acid sequences is provided by the local homology algorithm of Smith and Waterman, Advances in Applied Mathematics, 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986).

An example of an implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). Another method of establishing percent identity is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the internet address located by placing http:// in front of blast.ncbi.nlm.nih.gov/Blast.cgi.

The term “nucleic acid” includes RNA or DNA molecules having more than one nucleotide in any form including single-stranded, double-stranded, oligonucleotide or polynucleotide. The term “nucleotide sequence” includes the ordering of nucleotides in an oligonucleotide or polynucleotide in a single-stranded form of nucleic acid.

The term “promoter” as used herein includes a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors.

A “vector” is capable of transferring gene sequences to a target cell. Typically, “vector construct,” “expression vector,” and “gene transfer vector,” mean any nucleic acid construct capable of directing the expression of a gene of interest and which can transfer a gene sequence to a target cell, which can be accomplished by genomic integration of all or a portion of the vector, or transient or inheritable maintenance of the vector as an extrachromosomal element. Thus, the term includes cloning, and expression vehicles, as well as integrating vectors.

The term “regulatory element” as used herein includes a nucleotide sequence which controls some aspect of the expression of a nucleic acid sequence. Examples of regulatory elements illustratively include an enhancer, an internal ribosome entry site (IRES), an intron, an origin of replication, a polyadenylation signal (pA), a promoter, an enhancer, a transcription termination sequence, and an upstream regulatory domain, which contribute to the replication, transcription, and/or post-transcriptional processing of a nucleic acid sequence. In some embodiments, regulatory elements can also include cis-regulatory DNA elements as well as transposable elements (TEs). Those of ordinary skill in the art are capable of selecting and using these and other regulatory elements in an expression construct with no more than routine experimentation. Expression constructs can be generated using a genetic recombinant approach or synthetically using well-known methodology.

A “control element” or “control sequence” is a nucleotide sequence involved in an interaction of molecules contributing to the functional regulation of a polynucleotide, including replication, duplication, transcription, splicing, translation, or degradation of the polynucleotide. The regulation can affect the frequency, speed, or specificity of the process, and can be enhancing or inhibitory in nature. Control elements known in the art include, for example, transcriptional regulatory sequences such as promoters and enhancers. A promoter is a DNA region capable under some conditions of binding RNA polymerase and initiating transcription of a coding region usually located downstream (in the 3′ direction) from the promoter.

As used herein, the phrase “derived from” when referring to a polypeptide or polypeptide region means that the polypeptide or polypeptide region comprises amino acid sequences originally found in a “parental” protein and which may now comprise some amino acid residue additions, deletions, truncations, rearrangements, or other alterations relative to the original polypeptide or polypeptide region as long as a some function(s) and a structure(s) of the “parental” molecule are substantially conserved. The skilled worker will be able to identify a parental molecule from which a polypeptide or polypeptide region was derived using techniques known in the art, e.g., protein sequence alignment software.

As used herein, and with regard to a Shiga toxin polypeptide sequence or Shiga toxin derived polypeptide, the term “wild-type” generally refers to a naturally occurring, Shiga toxin protein sequence(s) found in a living species, such as, e.g., a pathogenic bacterium, wherein that Shiga toxin protein sequence(s) is one of the most frequently occurring variants. This is in contrast to infrequently occurring Shiga toxin protein sequences that, while still naturally occurring, are found in less than one percent of individual organisms of a given species when sampling a statistically powerful number of naturally occurring individual organisms of that species which comprise at least one Shiga toxin protein variant. A clonal expansion of a natural isolate outside its natural environment (regardless of whether the isolate is an organism or molecule comprising biological sequence information) does not alter the naturally occurring requirement as long as the clonal expansion does not introduce new sequence variety not present in naturally occurring populations of that species and/or does not change the relative proportions of sequence variants to each other.

As used herein, the term “linked” refer to two or more molecular components associated by one or more atomic interactions such that a single molecule is formed and wherein the atomic interactions include at least one covalent bond. As used herein, the term “linking” or “links” refers to the act of creating a linked molecule as described above.

As used herein, the terms “expressed,” “expressing,” or “expresses,” and grammatical variants thereof, refer to translation of a polynucleotide or nucleic acid into a protein. The expressed protein can remain intracellular, become a component of the cell surface membrane or be secreted into an extracellular space.

As used herein, cells which express a significant amount of CTLA-4 on at least one cellular surface are “CTLA-4 positive cells” or “CTLA-4+ cells.” These cells are considered to be “physically coupled” to the CTLA-4.

The term “CTLA-4,” as used herein, stands for cytotoxic T-lymphocyte-associated protein 4. It can also be referred to as CD152. CTLA-4 is a protein receptor that binds CD80 or CD86 on the surface of antigen-presenting cells. CTLA-4 downregulates immune responses and is constitutively expressed on various immunosuppressive immune cells such as regulatory T cells (Tregs). CTLA-4 comprises an extracellular V domain, a transmembrane domain, and a cytoplasmic tail. Alternatively splice variants, encoding different isoforms, have been characterized. The membrane-bound isoform functions as a homodimer interconnected by a disulfide bond, while the soluble isoform acts as a monomer. An illustrative sequence for human CTLA-4 is provided as SEQ ID NO: 19. See also Uniprot Accession No. P16410.

(SEQ ID NO: 19)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET

As used herein, the term “effector” means providing a biological activity, such as cytotoxicity, biological signaling, enzymatic catalysis, subcellular routing, and/or intermolecular binding resulting in an allosteric effect(s) and/or the recruitment of one or more factors.

The term “Shiga toxin” herein refers to two families of related toxins: Shiga toxin (Stx)/Shiga-like toxin 1 (SLT-1/Stx1) and Shiga-like toxin 2 (SLT-2/Stx2). Stx is produced by Shigella dysenteriae, while SLT-1 and SLT-2 are derived from Escherichia coli. Members of the Shiga toxin family share the same overall structure and mechanism of action (Engedal N et al., Microbial Biotech 4: 32-46 (2011)). For example, Stx, SLT-1 and SLT-2 display indistinguishable enzymatic activity in cell free systems (Head S et al., J Biol Chem 266: 3617-21 (1991); Tesh V et al., Infect Immun 61: 3392-402 (1993); Brigotti M et al., Toxicon 35:1431-1437 (1997)).

Stx, SLT-1, and SLT-2 are multimeric molecules comprised of two polypeptide subunits, A and B. The B Subunit is a pentamer that binds the toxin to glycolipids on host cell membranes and enters the cell via endocytosis. Once inside the cell, the A Subunit undergoes proteolytic cleavage and the reduction of an internal disulfide bond to generate the A1 Subunit and the A2 Subunit. The Shiga toxin or Shiga-like toxin A1 Subunits (e.g., SLT-1-A1) are N-glycosidases that catalytically inactivate the 28S ribosomal RNA subunit to inhibit protein synthesis.

As described herein, the phrase “Shiga toxin effector region” refers to a polypeptide derived from a Shiga toxin A Subunit or Shiga-like toxin A Subunit of the Shiga toxin family, which exhibits at least one Shiga toxin effector function. In some embodiments, the Shiga toxin effector region of the CTLA-4-binding molecule is a Shiga toxin A Subunit, such as StxA. In some embodiments, the Shiga toxin effector region of the CTLA-4-binding molecule is a Shiga-like toxin A Subunit, such as SLT-1A or SLT-2A. In some embodiments, the Shiga toxin effector region of the CTLA-4-binding molecule is an A1 Subunit of SLT-1 (e.g., SLT-1-A1). In some embodiments, the Shiga toxin effector region of the CTLA-4-binding molecule is an enzymatically active, de-immunized Shiga-like toxin A1 Subunit of SLT-1 (e.g., SLT-1-A1 V1). In some embodiments, the Shiga toxin effector region has a sequence of SEQ ID NO: 41, or a sequence at least 85%, at least 90%, at least 95%, at least 96%, at least 97%, at least 98%, or at least 99% identical thereto. In some embodiments, the Shiga toxin effector region has a sequence of SEQ ID NO: 41 with 1-10, 10-20, 20-30, 30-40, 40-50 or more amino acid substitutions.

As used herein, a Shiga toxin effector function is a biological activity conferred by a polypeptide region derived from a Shiga toxin A Subunit. Non-limiting examples of Shiga toxin effector functions include promoting cell entry; lipid membrane deformation; promoting cellular internalization; stimulating clathrin-mediated endocytosis; directing intracellular routing to various intracellular compartments such as, e.g., the Golgi, endoplasmic reticulum, and cytosol; directing intracellular routing with a cargo; inhibiting a ribosome function(s); catalytic activities, such as, e.g., N-glycosidase activity and catalytically inhibiting ribosomes; reducing protein synthesis, inducing caspase activity, activating effector caspases, effectuating cytostatic effects, and cytotoxicity. Shiga toxin catalytic activities include, for example, ribosome inactivation, protein synthesis inhibition, N-glycosidase activity, polynucleotide:adenosine glycosidase activity, RNAase activity, and DNAase activity. Shiga toxins are ribosome inactivating proteins (RIPs). RIPs can depurinate nucleic acids, polynucleosides, polynucleotides, rRNA, ssDNA, dsDNA, mRNA (and polyA), and viral nucleic acids (see e.g., Barbieri L et al., Biochem J 286: 1-4 (1992); Barbieri L et al., Nature 372: 624 (1994); Ling J et al., FEBS Lett 345: 143-6 (1994); Barbieri L et al., Biochem J 319: 507-13 (1996); Roncuzzi L, Gasperi-Campani A, FEBS Lett 392: 16-20 (1996); Stirpe F et al., FEBS Lett 382: 309-12 (1996); Barbieri L et al., Nucleic Acids Res 25: 518-22 (1997); Wang P, Tumer N, Nucleic Acids Res 27: 1900-5 (1999); Barbieri L et al., Biochim Biophys Acta 1480: 258-66 (2000); Barbieri L et al., J Biochem 128: 883-9 (2000); Brigotti M et al., Toxicon 39: 341-8 (2001); Brigotti M et al., FASEB J 16: 365-72 (2002); Bagga S et al., J Biol Chem 278: 4813-20 (2003); Picard D et al., J Biol Chem 280: 20069-75 (2005)). Some RIPs show antiviral activity and superoxide dismutase activity (Erice A et al., Antimicrob Agents Chemother 37: 835-8 (1993); Au T et al., FEBS Lett 471: 169-72 (2000); Parikh B, Tumer N, Mini Rev Med Chem 4: 523-43 (2004); Sharma N et al., Plant Physiol 134: 171-81 (2004)). Shiga toxin catalytic activities have been observed both in vitro and in vivo. Non-limiting examples of assays for Shiga toxin effector activity measure various activities, such as, e.g., protein synthesis inhibitory activity, depurination activity, inhibition of cell growth, cytotoxicity, supercoiled DNA relaxation activity, and nuclease activity.

As used herein, the retention of Shiga toxin effector function refers to being capable of exhibiting a level of Shiga toxin functional activity, as measured by an appropriate quantitative assay with reproducibility, comparable to a wild-type, Shiga toxin effector polypeptide control (e.g. a Shiga toxin A1 fragment) or a binding molecule comprising a wild-type Shiga toxin effector polypeptide (e.g. a Shiga toxin A1 fragment) under the same conditions. For the Shiga toxin effector function of ribosome inactivation or ribosome inhibition, retained Shiga toxin effector function is exhibiting an IC₅₀ of 10,000 pM or less in an in vitro setting, such as, e.g., by using an assay known to the skilled worker and/or described herein. For the Shiga toxin effector function of cytotoxicity in a target positive cell-kill assay, retained Shiga toxin effector function is exhibiting a CD₅₀ of 1,000 nM or less, depending on the cell type and its expression of the appropriate extracellular target biomolecule, as shown, e.g., by using an assay known to the skilled worker and/or described herein.

The term “selective cytotoxicity” with regard to the cytotoxic activity of a molecule refers to the relative level of cytotoxicity between a target positive cell population (e.g. a CTLA-4+ cell-type) and a non-targeted bystander cell population (e.g. a CTLA-4 negative cell-type), which can be expressed as a ratio of the half-maximal cytotoxic concentration (CD₅₀) for a targeted cell type over the CD₅₀ for an untargeted cell type to provide a metric of cytotoxic selectivity or indication of the selectivity of killing of a targeted cell versus an untargeted cell.

The cell surface representation and/or density of CTLA-4 (or extracellular epitope thereof) can influence the applications for which some binding molecules can be most suitably used. Differences in cell surface representation and/or density of CTLA-4 between cells can alter, both quantitatively and qualitatively, the efficiency of cellular internalization and/or cytotoxicity potency of a given binding molecule. The cell surface representation and/or density of CTLA-4 can vary greatly among CTLA-4 positive cells or even on the same cell at different points in the cell cycle or cell differentiation. The total cell surface representation of CTLA-4 and/or of some extracellular epitopes of CTLA-4 on a particular cell or population of cells can be determined using methods known to the skilled worker, such as methods involving fluorescence-activated cell sorting (FACS) flow cytometry.

As used herein, the terms “disrupted,” “disruption,” or “disrupting,” and grammatical variants thereof, with regard to a polypeptide region or feature within a polypeptide refers to an alteration of at least one amino acid within the region or composing the disrupted feature. Amino acid alterations include various mutations, such as, e.g., a deletion, inversion, insertion, or substitution which alter the amino acid sequence of the polypeptide. Amino acid alterations also include chemical changes, such as, e.g., the alteration one or more atoms in an amino acid functional group or the addition of one or more atoms to an amino acid functional group.

As used herein, “de-immunized” means reduced antigenic and/or immunogenic potential after administration to a subject (e.g., a human subject) as compared to a reference molecule, such as, e.g., a wild-type peptide region, polypeptide region, or polypeptide. This includes a reduction in overall antigenic and/or immunogenic potential despite the introduction of one or more, de novo, antigenic and/or immunogenic epitopes as compared to a reference molecule. For some embodiments, “de-immunized” means a molecule exhibited reduced antigenicity and/or immunogenicity after administration to a mammal as compared to a “parental” molecule from which it was derived, such as, e.g., a wild-type Shiga toxin A1 fragment or binding molecule comprising the aforementioned. In some embodiments, the de-immunized, Shiga toxin effector polypeptide is capable of exhibiting a relative antigenicity compared to a reference “parental” molecule which is reduced by about 10%, about 20%, about 30%, about 40%, about 50%, about 60%, about 70%, about 80%, about 90%, or greater than the antigenicity of the reference molecule under the same conditions measured by the same assay, such as, e.g., an assay known to the skilled worker and/or described herein like a quantitative ELISA or Western blot analysis. In some embodiments, the de-immunized, Shiga toxin effector polypeptide is capable of exhibiting a relative immunogenicity compared to a reference “parental” molecule which is reduced by about 10%, about 20%, about 30%, about 40%, about 50%, about 60%, about 70%, about 80%, about 90%, about 95%, about 97%, about 99%, or greater than the immunogenicity of the reference molecule under the same conditions measured by the same assay, such as, e.g., an assay known to the skilled worker and/or described herein like a quantitative measurement of anti-molecule antibodies produced in a mammal(s) after receiving parenteral administration of the molecule at a given time-point.

As used herein, the phrase “B-cell and/or CD4+ T-cell de-immunized” means that the molecule has a reduced antigenic and/or immunogenic potential after administration to a mammal regarding either B-cell antigenicity or immunogenicity and/or CD4+ T-cell antigenicity or immunogenicity. For some embodiments, “B-cell de-immunized” means a molecule exhibited reduced B-cell antigenicity and/or immunogenicity after administration to a mammal as compared to a “parental” molecule from which it was derived, such as, e.g., a wild-type Shiga toxin A1 fragment. For some embodiments, “CD4+ T-cell de-immunized” means a molecule exhibited reduced CD4 T-cell antigenicity and/or immunogenicity after administration to a mammal as compared to a “parental” molecule from which it was derived, such as, e.g., a wild-type Shiga toxin A1 fragment.

The term “endogenous” with regard to a B-cell epitope, CD4+ T-cell epitope, B-cell epitope region, or CD4+ T-cell epitope region in a Shiga toxin effector polypeptide refers to an epitope present in a wild-type Shiga toxin A Subunit.

As used herein, the term “heterologous” means of a different source, e.g., a heterologous Shiga A Subunit polypeptide is not naturally found as part of any A Subunit of a native Shiga toxin.

The term “embedded” and grammatical variants thereof with regard to a binding molecule refers to the internal replacement of one or more amino acids within a polypeptide region with different amino acids in order to generate a new polypeptide sequence sharing the same total number of amino acid residues with the starting polypeptide region. Thus, the term “embedded” does not include any external, terminal fusion of any additional amino acid, peptide, or polypeptide component to the starting polypeptide nor any additional internal insertion of any additional amino acid residues, but rather includes only substitutions for existing amino acids. The internal replacement can be accomplished merely by amino acid residue substitution or by a series of substitutions, deletions, insertions, and/or inversions. If an insertion of one or more amino acids is used, then the equivalent number of proximal amino acids must be deleted next to the insertion to result in an embedded peptide. This is in contrast to use of the term “inserted” with regard to a binding molecule to refer to the insertion of one or more amino acids internally within a polypeptide resulting in a new polypeptide having an increased number of amino acids residues compared to the starting polypeptide.

The term “inserted” and grammatical variants thereof with regard to a binding molecule refers to the insertion of one or more amino acids within a polypeptide resulting in a new polypeptide sequence having an increased number of amino acids residues compared to the starting polypeptide. The phrases “partially inserted,” “embedded and inserted,” and grammatical variants thereof with regard to a polypeptide component of a binding molecule, refers to when the resulting polypeptide increased in length, but by less than the number of amino acid residues equivalent to the length of the entire, inserted polypeptide. Insertions, whether “pure” or “partial,” include any of the previously described insertions even if other regions of the polypeptide not proximal to the insertion site within the polypeptide are deleted thereby resulting in a decrease in the total length of the final polypeptide because the final polypeptide still comprises an internal insertion of one or more amino acids of a T-cell epitope-peptide within a polypeptide region.

As used herein, the phrase “proximal to an amino-terminus” with reference to the position of a Shiga toxin effector polypeptide region of a binding molecule refers to a distance wherein at least one amino acid residue of the Shiga toxin effector polypeptide region is within 1, 2, 3, 4, 5, 6, 7, 8, 9, 10, 11, 12 or more, e.g., up to 18-20 amino acid residues, of an amino-terminus of the binding molecule as long as the binding molecule is capable of exhibiting the appropriate level of Shiga toxin effector functional activity noted herein (e.g., a some level of cytotoxic potency). Thus, for some embodiments, any amino acid residue(s) fused amino-terminal to the Shiga toxin effector polypeptide should not reduce any Shiga toxin effector function (e.g., by sterically hindering a structure(s) near the amino-terminus of the Shiga toxin effector polypeptide region) such that a functional activity of the Shiga toxin effector polypeptide is reduced below the appropriate activity level required herein.

As used herein, the phrase “more proximal to an amino-terminus” with reference to the position of a Shiga toxin effector polypeptide region within a binding molecule as compared to another component (e.g., a cell-targeting, binding region, molecular moiety, and/or additional exogenous material) refers to a position wherein at least one amino acid residue of the amino-terminus of the Shiga toxin effector polypeptide is closer to the amino-terminus of a linear, polypeptide component of the binding molecule as compared to the other referenced component.

As used herein, the phrase “active enzymatic domain derived from one A Subunit of a member of the Shiga toxin family” refers to having the ability to inhibit protein synthesis via a catalytic ribosome inactivation mechanism. The enzymatic activities of naturally occurring Shiga toxins can be defined by the ability to inhibit protein translation using assays known to the skilled worker, such as, e.g., in vitro assays involving RNA translation in the absence of living cells or in vivo assays involving RNA translation in a living cell. Using assays known to the skilled worker and/or described herein, the potency of a Shiga toxin enzymatic activity can be assessed directly by observing N-glycosidase activity toward ribosomal RNA (rRNA), such as, e.g., a ribosome nicking assay, and/or indirectly by observing inhibition of ribosome function and/or protein synthesis.

As used herein, the term “Shiga toxin A1 fragment region” refers to a polypeptide region consisting essentially of a Shiga toxin A1 fragment and/or derived from a Shiga toxin A1 fragment of a Shiga toxin.

As used herein, the terms “terminus,” “amino-terminus,” or “carboxy-terminus” with regard to a polypeptide region refers to the regional boundaries of that region, regardless of whether additional amino acid residues are linked by peptide bonds outside of that region. In other words, the terminals of the polypeptide region regardless of whether that region is fused to other peptides or polypeptides. For example, a binding molecule comprising two proteinaceous regions, e.g., a binding region comprising a peptide or polypeptide and a Shiga toxin effector polypeptide, can have a Shiga toxin effector polypeptide region with a carboxy-terminus ending at amino acid residue 251 of the Shiga toxin effector polypeptide region despite a peptide bond involving residue 251 to an amino acid residue at position 252 representing the beginning of another proteinaceous region, e.g., the binding region. In this example, the carboxy-terminus of the Shiga toxin effector polypeptide region refers to residue 251, which is not a terminus of the binding molecule but rather represents an internal, regional boundary. Thus, for polypeptide regions, the terms “terminus,” “amino-terminus,” and “carboxy-terminus” are used to refer to the boundaries of polypeptide regions, whether the boundary is a physically terminus or an internal, position embedded within a larger polypeptide chain.

As used herein, the phrase “carboxy-terminus region of a Shiga toxin A1 fragment” refers to a polypeptide region derived from a naturally occurring Shiga toxin A1 fragment, the region beginning with a hydrophobic residue (e.g., V236 of StxA-A1 and SLT-1A1, and V235 of SLT-2A1) that is followed by a hydrophobic residue and the region ending with the furin-cleavage site conserved among Shiga toxin A1 fragment polypeptides and ending at the junction between the A1 fragment and the A2 fragment in native, Shiga toxin A Subunits. In some embodiments, the carboxy-terminal region of a Shiga toxin A1 fragment includes a peptidic region derived from the carboxy-terminus of a Shiga toxin A1 fragment polypeptide, such as, e.g., a peptidic region comprising or consisting essentially of the carboxy-terminus of a Shiga toxin A1 fragment. Non-limiting examples of peptidic regions derived from the carboxy-terminus of a Shiga toxin A1 fragment include the amino acid residue sequences natively positioned from position 236 to position 239, 240, 241, 242, 243, 244, 245, 246, 247, 248, 249, 250, or 251 in Stx1A (SEQ ID NO:2) or SLT-1A (SEQ ID NO:1); and from position 235 to position 239, 240, 241, 242, 243, 244, 245, 246, 247, 248, 249, or 250 in SLT-2A (SEQ ID NO:3).

As used herein, the phrase “proximal to the carboxy-terminus of an A1 fragment polypeptide” with regard to a linked molecular moiety and/or binding region refers to being within 1, 2, 3, 4, 5, 6, 7, 8, 9, 10, 11, or 12 amino acid residues from the amino acid residue defining the last residue of the Shiga toxin A1 fragment polypeptide.

As used herein, the phrase “sterically covers the carboxy-terminus of the A1 fragment-derived region” includes any molecular moiety of a size of 4.5 kDa or greater (e.g., an immunoglobulin-type binding region) linked and/or fused to an amino acid residue in the carboxy-terminus of the A1 fragment-derived region, such as, e.g., the amino acid residue derived from the amino acid residue natively positioned at any one of positions 236 to 251 in Stx1A (SEQ ID NO:2) or SLT-1A (SEQ ID NO:1) or from 235 to 250 in SLT-2A (SEQ ID NO:3). As used herein, the phrase “sterically covers the carboxy-terminus of the A1 fragment-derived region” also includes any molecular moiety of a size of 4.5 kDa or greater (e.g., an immunoglobulin-type binding region) linked and/or fused to an amino acid residue in the carboxy-terminus of the A1 fragment-derived region, such as, e.g., the amino acid residue carboxy-terminal to the last amino acid A1 fragment-derived region and/or the Shiga toxin effector polypeptide. As used herein, the phrase “sterically covers the carboxy-terminus of the A1 fragment-derived region” also includes any molecular moiety of a size of 4.5 kDa or greater (e.g., an immunoglobulin-type binding region) physically preventing cellular recognition of the carboxy-terminus of the A1 fragment-derived region, such as, e.g., recognition by the ERAD machinery of a eukaryotic cell.

In some embodiments, a binding region, such as, e.g., an immunoglobulin-type binding region, that comprises a polypeptide comprising at least forty amino acids and that is linked (e.g., fused) to the carboxy-terminus of the Shiga toxin effector polypeptide region comprising an A1 fragment-derived region is a molecular moiety which is “sterically covering the carboxy-terminus of the A1 fragment-derived region.”

In some embodiments, a binding region, such as, e.g., an immunoglobulin-type binding region, that comprises a polypeptide comprising at least forty amino acids and that is linked (e.g., fused) to the carboxy-terminus of the Shiga toxin effector polypeptide region comprising an A1 fragment-derived region is a molecular moiety “encumbering the carboxy-terminus of the A1 fragment-derived region.”

As used herein, the term “A1 fragment of a member of the Shiga toxin family” refers to the remaining amino-terminal fragment of a Shiga toxin A Subunit after proteolysis by furin at the furin-cleavage site conserved among Shiga toxin A Subunits and positioned between the A1 fragment and the A2 fragment in wild-type Shiga toxin A Subunits.

As used herein, the phrase “furin-cleavage site at the carboxy-terminus of the A1 fragment region” refers to a specific, furin-cleavage site conserved among Shiga toxin A Subunits and bridging the junction between the A1 fragment and the A2 fragment in naturally occurring, Shiga toxin A Subunits.

As used herein, the phrase “furin-cleavage site proximal to the carboxy-terminus of the A1 fragment region” refers to any identifiable, furin-cleavage site having an amino acid residue within a distance of less than 1, 2, 3, 4, 5, 6, 7, or more amino acid residues of the amino acid residue defining the last amino acid residue in the A1 fragment region or A1 fragment derived region, including a furin-cleavage site located carboxy-terminal of an A1 fragment region or A1 fragment derived region, such as, e.g., at a position proximal to the linkage of the A1 fragment-derived region to another component of the molecule, such as, e.g., a molecular moiety of a binding molecule as described herein.

As used herein, the term “additional therapeutic agent” means an additional therapeutic agent (e.g., a molecule) that targets the cell to produce a therapeutic effect or benefit. This additional therapeutic agent is complementary to the binding molecule and does not compete directly with the binding molecule in its targeting activity. The additional therapeutic agent can comprise, consist essentially of, or consist of an antibody or small molecule inhibitor that interferes with signaling. For example, additional therapeutic agents can comprise, consist essentially of, or consist of an antibody that binds to an antigenic determinant that does not overlap with the antigenic determinant bound by the binding molecule.

As used herein, the phrase “disrupted furin-cleavage site” refers to (i) a specific furin-cleavage site as described herein in Section I-B and (ii) which comprises a mutation and/or truncation that can confer a molecule with a reduction in furin-cleavage as compared to a reference molecule, such as, e.g., a reduction in furin-cleavage reproducibly observed to be 30%, 40%, 50%, 60%, 70%, 80%, 90%, 95%, 97%, 98%, 99%, or less (including 100% for no cleavage) than the furin-cleavage of a reference molecule observed in the same assay under the same conditions. The percentage of furin-cleavage as compared to a reference molecule can be expressed as a ratio of cleaved:uncleaved material of the molecule of interest divided by the cleaved:uncleaved material of the reference molecule (see e.g. WO 2015/191764; WO 2016/196344). Non-limiting examples of suitable reference molecules include some molecules comprising a wild-type Shiga toxin furin-cleavage site as described herein.

As used herein, the phrase “furin-cleavage resistant” means a molecule or specific polypeptide region thereof exhibits reproducibly less furin cleavage than (i) the carboxy-terminus of a Shiga toxin A1 fragment in a wild-type Shiga toxin A Subunit or (ii) the carboxy-terminus of the Shiga toxin A1 fragment derived region of construct wherein the naturally occurring furin-cleavage site natively positioned at the junction between the A1 and A2 fragments is not disrupted; as assayed by any available means to the skilled worker, including by using a method described herein.

As used herein, the phrase “active enzymatic domain derived from an A Subunit of a member of the Shiga toxin family” refers to a polypeptide structure having the ability to inhibit protein synthesis via catalytic inactivation of a ribosome based on a Shiga toxin enzymatic activity. The ability of a molecular structure to exhibit inhibitory activity of protein synthesis and/or catalytic inactivation of a ribosome can be observed using various assays known to the skilled worker, such as, e.g., in vitro assays involving RNA translation assays in the absence of living cells or in vivo assays involving the ribosomes of living cells. For example, using assays known to the skilled worker, the enzymatic activity of a molecule based on a Shiga toxin enzymatic activity can be assessed directly by observing N-glycosidase activity toward ribosomal RNA (rRNA), such as, e.g., a ribosome nicking assay, and/or indirectly by observing inhibition of ribosome function, RNA translation, and/or protein synthesis.

As used herein with respect to a Shiga toxin effector polypeptide, a “combination” describes a Shiga toxin effector polypeptide comprising two or more sub-regions wherein each sub-region comprises at least one of the following, e.g., (1) a disruption in an endogenous epitope or epitope region; and (2) a disrupted furin-cleavage site at the carboxy-terminus of an A1 fragment region.

As used herein, the term “directly kill” refers to the ability of a binding molecule comprising a Shiga toxin A subunit effector polypeptide and a binding region to kill the CTLA-4-positive cell to which it binds. A binding molecule can directly kill the cell by one or more of: promoting cell entry; lipid membrane deformation; promoting cellular internalization; stimulating clathrin-mediated endocytosis; directing intracellular routing to various intracellular compartments such as, e.g., the Golgi, endoplasmic reticulum, and cytosol; directing intracellular routing with a cargo; inhibiting a ribosome function(s); catalytic activities, such as, e.g., N-glycosidase activity and catalytically inhibiting ribosomes; reducing protein synthesis, inducing caspase activity, activating effector caspases, effectuating cytostatic effects, and cytotoxicity. In some embodiments, the binding molecule causes ribosome inactivation, protein synthesis inhibition, N-glycosidase activity, polynucleotide:adenosine glycosidase activity, RNAase activity, and/or DNAase activity in the CTLA-4-positive cell, leading to the cell's death.

As used herein, the terms “does not directly kill” or “indirectly kills” refers to a process wherein a binding molecule comprising a Shiga toxin A subunit effector polypeptide and a binding region binds to a target cell (e.g., a ICC), which leads to the downstream killing of a second cell (e.g., a cancer cell). For example, a binding molecule can indirectly kill a tumor cell by binding to and killing an immunosuppressive immune cell in the tumor microenvironment (TME). Once immunosuppression is lifted in the TME, the cancer cell can be killed by non-suppressive immune cells (e.g., cytotoxic T cells, etc).

### II. Binding Regions

As used herein, the term “binding region” refers to a molecular moiety (e.g. a proteinaceous molecule) or agent capable of specifically binding an extracellular part of a target molecule with high affinity, such as, e.g., having a dissociation constant with regard to the target of about 10⁻⁵ to about 10⁻¹² moles per liter. In some embodiments, the binding region comprises a cell-targeting component, such as, e.g., a domain, molecular moiety, or agent, capable of binding specifically to an extracellular part of a target on a cell surface (i.e., an extracellular target biomolecule) with high affinity.

An extracellular part of a target (e.g., a target biomolecule such as CTLA-4) refers to a portion of its structure exposed to the extracellular environment when the target is physically coupled to a cell, such as, e.g., when the target is expressed at a cellular surface by the cell. In this context, exposed to the extracellular environment means that part of the target is accessible by, e.g., an antibody or at least a binding moiety smaller than an antibody such as a single-domain antibody domain, a nanobody, a heavy-chain antibody domain derived from camelids or cartilaginous fishes, a single-chain variable fragment, or any number of engineered alternative scaffolds to immunoglobulins. The exposure to the extracellular environment of or accessibility to a part of target biomolecule physically coupled to a cell can be empirically determined by the skilled worker using methods well known in the art.

The binding molecules described herein comprise a binding region capable of specifically binding CTLA-4 on the surface of a cell, e.g., a CTLA-4 expressing cell (also referred to herein as a CTLA-4 positive cell). In some embodiments, the CTLA-4 positive cell is a tumor cell. In some embodiments, the CTLA-4 positive cell is an immunosuppressive immune cell, such as an immunosuppressive T cell, an immunosuppressive B cell, an immunosuppressive plasma cell, or an immunosuppressive myeloid cell. In some embodiments, the immunosuppressive immune cell is a Treg, an MDSC, or a TAM. In some embodiments, the immunosuppressive immune cell is a TAN or a CAF. In some embodiments, the binding region does not specifically bind to a resident memory T cell, a tumor-excluded dendritic cell, and/or a CD14+ monocyte.

In some embodiments, the binding region is an immunoglobulin-type binding region. In some embodiments, the immunoglobulin-type binding region is derived from an immunoglobulin binding region, such as an antibody paratope. This engineered polypeptide can optionally include polypeptide scaffolds comprising or consisting essentially of complementary determining regions and/or antigen binding regions from immunoglobulins as described herein.

In some embodiments, the binding region can be, e.g., a monoclonal antibody or engineered antibody derivative. In some embodiments, the binding region is an antibody fragment, e.g., a Fv, Fab, Fab′, single chain variable fragment (scFv), diabody, Fab′-SH, or F(ab′)2 fragment. In some embodiments, the binding region is a scFv. In some embodiments, the binding region is a diabody. In some embodiments, the binding region is a full-length antibody, e.g., an intact IgG1 antibody or other antibody class or isotype as defined herein and/or known to the skilled worker. The “class” of an antibody refers to the type of constant domain or constant region present in the heavy chain. There are five major classes of antibodies: IgA, IgD, IgE, IgG, and IgM, and several of these can be further divided into isotypes, e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

In some embodiments, the binding region is a synthetically engineered antibody derivative, such as, e.g. a single-domain antibody fragment, single-chain variable fragment, antibody variable fragment, Fd fragment, Fab (antigen-binding fragment), an autonomous VH domain, single domain immunoglobulin-derived region VHH, heavy-chain antibody domain derived from a camelid VHH fragment or VH domain fragment, heavy-chain antibody domain derived from cartilaginous fish, VHH fragment or VH domain fragment, immunoglobulin new antigen receptor (IgNAR), VNAR fragment, disulfide stabilized antibody variable (Fv) fragment, Armadillo repeat polypeptide, fibronectin-derived 10^{th} fibronectin type III domain, tenascin type III domain, ankyrin repeat motif domain, low-density-lipoprotein-receptor-derived A-domain, lipocalin, Kunitz domain, Protein-A-derived Z domain, gamma-B crystalline-derived domain, ubiquitin-derived domain, Sac7d-derived polypeptide, Fyn-derived SH2 domain, miniprotein, C-type lectin-like domain scaffold and so forth.

In some embodiments, the binding region comprises a multivalent antibody format, such as a multimerizing scFv fragment such as a diabody, triabody, tetrabody, bispecific tandem scFv fragment, bispecific tandem VHH fragment, bispecific minibody or bivalent minibody.

In some embodiments, the binding region is an autonomous V_{H} domain (such as, e.g., from camelids, murine, or human sources), single-domain antibody domain (sdAb), heavy-chain antibody domain derived from a camelid (V_{H}H fragment or V_{H} domain fragment), heavy-chain antibody domain derived from a camelid V_{H}H fragments or V_{H} domain fragments, heavy-chain antibody domain derived from a cartilaginous fish (e.g., a shark), immunoglobulin new antigen receptor (IgNAR), V_{NAR} fragment, single-chain variable (scFv) fragment, nanobody, “camelized” scaffolds comprising a VH domain, Fd fragment consisting of the heavy chain and C_{H}1 domains, single chain Fv-C_{H}3 minibody, Fc antigen binding domain (Fcabs), scFv-Fc fusion, multimerizing scFv fragment (diabodies, triabodies, tetrabodies), disulfide-stabilized antibody variable (Fv) fragment (dsFv), disulfide-stabilized antigen-binding (Fab) fragment consisting of the V_{L}, V_{H}, C_{L} and C_{H}1 domains, single-chain variable-region fragment comprising a disulfide-stabilized heavy and light chain (sc-dsFvs), bivalent nanobody, bivalent minibody, bivalent F(ab′)₂ fragment (Fab dimers), bispecific tandem V_{H}H fragment, bispecific tandem scFv fragment, bispecific nanobody, bispecific minibody, Fab-FCabs (mAb²'s), and any genetically manipulated counterparts of the foregoing that retain its paratope and binding function, such as, e.g., wherein the relative orientation or order of the heavy and light chains is reversed or “flipped”.

In some embodiments, the binding region can comprise an immunoglobulin-type binding region. The term “immunoglobulin-type binding region” as used herein refers to a polypeptide region capable of binding one or more targets, such as an antigen or epitope. Binding regions can be functionally defined by their ability to bind to target molecules. Immunoglobulin-type binding regions are commonly derived from antibody or antibody-like structures.

Immunoglobulin (Ig) proteins have a structural domain known as an Ig domain. Ig domains range in length from about 70-110 amino acid residues and possess a characteristic Ig-fold, in which typically 7 to 9 antiparallel beta strands arrange into two beta sheets which form a sandwich-like structure. The Ig fold is stabilized by hydrophobic amino acid interactions on inner surfaces of the sandwich and highly conserved disulfide bonds between cysteine residues in the strands. Ig domains can be variable (IgV or V-set), constant (IgC or C-set) or intermediate (IgI or I-set). Some Ig domains can be associated with a complementarity determining region (CDR), also called a “complementary determining region,” which is important for the specificity of antibodies binding to their epitopes. Ig-like domains are also found in non-immunoglobulin proteins and are classified on that basis as members of the Ig superfamily of proteins. The HUGO Gene Nomenclature Committee (HGNC) provides a list of members of the Ig-like domain containing family.

An immunoglobulin-type binding region can be a polypeptide sequence of an antibody or antigen-binding fragment thereof wherein the amino acid sequence has been varied from that of a native antibody or an Ig-like domain of a non-immunoglobulin protein, for example by molecular engineering or selection by library screening. Because of the relevance of recombinant DNA techniques and in vitro library screening in the generation of immunoglobulin-type binding regions, antibodies can be redesigned to obtain desired characteristics, such as smaller size, cell entry, or other improvements for in vivo and/or therapeutic applications. The possible variations are many and can range from the changing of just one amino acid to the complete redesign of, for example, a variable region. Typically, changes in the variable region will be made in order to improve the antigen-binding characteristics, improve variable region stability, or reduce the potential for immunogenic responses.

There are numerous immunoglobulin-type binding regions useful as components of the binding molecules described herein. In some embodiments, the immunoglobulin-type binding region is derived from an immunoglobulin binding region, such as an antibody paratope capable of binding an extracellular part of a target such as CTLA-4. In some embodiments, the immunoglobulin-type binding region comprises an engineered polypeptide not derived from any immunoglobulin domain but which functions like an immunoglobulin binding region by providing high-affinity binding to an extracellular part of a target, such as CTLA-4. This engineered polypeptide can optionally include polypeptide scaffolds comprising or consisting essentially of complementary determining regions from immunoglobulins as described herein.

In some embodiments, the binding region is useful for targeting binding molecules to specific cell-types via their high-affinity binding characteristics. In some embodiments, the binding region is an autonomous V_{H} domain, a single-domain antibody domain (sdAbs), a heavy-chain antibody domain derived from camelids (V_{H}H fragments or V_{H} domain fragments), a heavy-chain antibody domain derived from camelid V_{H}H fragments or V_{H} domain fragments, a heavy-chain antibody domain derived from cartilaginous fishes (e.g., a shark), an immunoglobulin new antigen receptor (IgNAR), a V_{NAR} fragment, a single-chain variable (scFv) fragment, a nanobody, a Fd fragment consisting of the heavy chain and C_{H}1 domains, a single chain Fv-C_{H}3 minibody, a dimeric C_{H}2 domain fragment (C_{H}2D), a Fc antigen binding domain (Fcab), an isolated complementary determining region 3 (CDR3) fragment, a constrained framework region 3, a CDR3, a framework region 4 (FR3-CDR3-FR4) polypeptide, a small modular immunopharmaceutical (SMIP) domain, a scFv-Fc fusion, a multimerizing scFv fragment (e.g., a diabody, a triabody, a tetrabody), a disulfide stabilized antibody variable (Fv) fragment, a disulfide stabilized antigen-binding (Fab) fragment consisting of V_{L}, V_{H}, C_{L} and C_{H}1 domains, a bivalent nanobody, a bivalent minibody, a bivalent F(ab′)₂ fragment (Fab dimers), a bispecific tandem V_{H}H fragment, a bispecific tandem scFv fragment, a bispecific nanobody, a bispecific minibody, and any genetically manipulated counterpart of the foregoing that retains its paratope and binding function, such as, e.g., wherein the relative orientation or order of the heavy and light chains is reversed or flipped (see Ward E et al., Nature 341: 544-6 (1989); Davies J, Riechmann L, Biotechnology (NY) 13: 475-9 (1995); Reiter Y et al., Mol Biol 290: 685-98 (1999); Riechmann L, Muyldermans S, J Immunol Methods 231: 25-38 (1999); Tanha J et al., J Immunol Methods 263: 97-109 (2002); Vranken W et al., Biochemistry 41: 8570-9 (2002); Jespers L et al., J Mol Biol 337: 893-903 (2004); Jespers L et al., Nat Biotechnol 22: 1161-5 (2004); To R et al., J Biol Chem 280: 41395-403 (2005); Saerens D et al., Curr Opin Pharmacol 8: 600-8 (2008); Dimitrov D, MAbs 1:26-8 (2009); Weiner L, Cell 148: 1081-4 (2012); Ahmad Z et al., Clin Dev Immunol 2012: 980250 (2012)).

In some embodiments, the binding region comprises a VHH domain comprising a HCDR1, a HCDR2, and a HCDR3.

In some embodiments the HCDR1 comprises the amino acid sequence of SEQ ID NO: 23, the HCDR2 comprises the amino acid sequence of SEQ ID NO: 24, and the HCDR3 comprises the amino acid sequence of SEQ ID NO: 25. In some embodiments, the VHH domain comprises the amino acid sequence of SEQ ID NO: 21.

In some embodiments, the HCDR1 comprises the amino acid sequence of SEQ ID NO: 26, the HCDR2 comprises the amino acid sequence of SEQ ID NO: 27, and the HCDR3 comprises the amino acid sequence of SEQ ID NO: 28. In some embodiments, the VHH domain comprises the amino acid sequence of SEQ ID NO: 22.

In some embodiments, the binding region comprises two VHH domains in tandem. In some embodiments, each VHH domain binds a different CTLA-4 epitope.

In some embodiments, the binding region comprises a first VHH domain comprising a first HCDR1, a first HCDR2, and a first HCDR3 and a second VHH domain comprising a second HCDR1, a second HCDR2, and a second HCDR3. In some embodiments, the first HCDR1 comprises the amino acid sequence of SEQ ID NO: 23, the first HCDR2 comprises the amino acid sequence of SEQ ID NO: 24, and the first HCDR3 comprises the amino acid sequence of SEQ ID NO: 25. In some embodiments, the first VHH domain comprises the amino acid sequence of SEQ ID NO: 21. In some embodiments, the second HCDR1 comprises the amino acid sequence of SEQ ID NO: 26, the second HCDR2 comprises the amino acid sequence of SEQ ID NO: 27, and the second HCDR3 comprises the amino acid sequence of SEQ ID NO: 28. In some embodiments, the second VHH domain comprises the amino acid sequence of SEQ ID NO: 22.

In some embodiments, the binding region comprises a linker that links the first VHH domain and the second VHH domain. In some embodiments, the linker is any of the linkers described herein. In some embodiments, the linker comprises the amino acid sequence of (GₓS)ₙ, wherein x is 1 to 6 and n is 1 to 30 (SEQ ID NO: 203). In some embodiments, the linker comprises the amino acid sequence of SEQ ID NO: 29.

In some embodiments, the binding region comprises, from N-terminus to C-terminus, a first VHH domain comprising the amino acid sequence of SEQ ID NO: 21, a linker comprising the amino acid sequence of SEQ ID NO: 29, and a second VHH domain comprises the amino acid sequence of SEQ ID NO: 22.

In some embodiments, the binding region comprises one or more polypeptides derived from the constant regions of immunoglobulins, such as, e.g., engineered dimeric Fc domains, monomeric Fcs (mFcs), scFv-Fcs, V_{H}H-Fcs, C_{H}2 domains, monomeric C_{H}3s domains (mC_{H}3s), synthetically reprogrammed immunoglobulin domains, and/or hybrid fusions of immunoglobulin domains with ligands (Hofer T et al., Proc Natl Acad Sci U.S.A. 105: 12451-6 (2008); Xiao J et al., J Am Chem Soc 131: 13616-13618 (2009); Xiao X et al., Biochem Biophys Res Commun 387: 387-92 (2009); Wozniak-Knopp G et al., Protein Eng Des Sel 23 289-97 (2010); Gong R et al., PLoS ONE 7: e42288 (2012); Wozniak-Knopp G et al., PLoS ONE 7: e30083 (2012); Ying T et al., J Biol Chem 287: 19399-408 (2012); Ying T et al., J Biol Chem 288: 25154-64 (2013); Chiang M et al., J Am Chem Soc 136: 3370-3 (2014); Rader C, Trends Biotechnol 32: 186-97 (2014); Ying T et al., Biochimica Biophys Acta 1844: 1977-82 (2014)). In some embodiments, the binding region is an intact antibody and/or comprises an Fc region.

In some embodiments, the binding region comprises an engineered, alternative scaffold to immunoglobulin domains. Engineered alternative scaffolds which exhibit similar functional characteristics to immunoglobulin-derived structures, such as high-affinity and specific binding of CTLA-4, are known in the art and might provide improved characteristics to certain immunoglobulin domains, such as, e.g., greater stability or reduced immunogenicity. Generally, alternative scaffolds to immunoglobulins are less than 20 kilodaltons, consist of a single polypeptide chain, lack cysteine residues, and exhibit relatively high thermodynamic stability.

In some embodiments, a binding molecule comprises a binding region comprising a polypeptide capable of selectively and/or specifically binding an extracellular part of CTLA-4. In some embodiments, the binding region is a VHH or a fragment thereof. In some embodiments, the binding region is an immunoglobulin-type binding region. In some embodiments, the binding region is derived from an anti-CTLA-4 antibody, such as any one of the antibodies listed in Table 3.

In some embodiments, the binding region comprises (i) a heavy chain variable domain (VH) comprising a HCDR1 of SEQ ID NO: 72, a HCDR2 of SEQ ID NO: 73, and a HCDR3 of SEQ ID NO: 74; and (2) a light chain comprising a light chain variable domain (VL) comprising a LCDR1 of SEQ ID NO: 69, a LCDR2 of SEQ ID NO: 70, and a LCDR3 of SEQ ID NO: 71. In some embodiments, the binding region comprises (i) a heavy chain variable domain (VH) comprising a HCDR1 of SEQ ID NO: 78, a HCDR2 of SEQ ID NO: 79, and a HCDR3 of SEQ ID NO: 80; and (2) a light chain comprising a light chain variable domain (VL) comprising a LCDR1 of SEQ ID NO: 75, a LCDR2 of SEQ ID NO: 76, and a LCDR3 of SEQ ID NO: 77. In some embodiments, the CDRs comprise one, two, or three mutations compared to the sequences shown in Table 3.

TABLE 3
CDRs of anti-CTLA-4 antibodies

| Name | CDR Sequences | SEQ ID NO |
|---|---|---|
| Ipilimumab | LCDR1: QSVGSSY | 69 |
| (Yervoy, | LCDR2: GAF | 70 |
| MDX-010) | LCDR3: QQYGSSPWT | 71 |
| | HCDR1: GFTFSSYT | 72 |
| | HCDR2: TFISYDGNNK | 73 |
| | HCDR3: ARTGWLGPFDY | 74 |
| Tremelimumab | LCDR1: QSINSY | 75 |
| (CP-675, 206) | LCDR2: AAS | 76 |
| | LCDR3: QQYYSTPFT | 77 |
| | HCDR1: GFTFSSYG | 78 |
| | HCDR2: AVIWYDGSNK | 79 |
| | HCDR3: ARDPRGATLYYYYYGMDV | 80 |

### III. Shiga Toxin A Subunit Effector Polypeptides

The binding molecules described herein comprise a Shiga toxin A subunit effector polypeptide. Shiga toxin A Subunit effector polypeptides provide robust and powerful scaffolds for engineering novel, binding molecules.

In some embodiments, the binding molecules comprise a Shiga toxin effector polypeptide derived from a Shiga toxin A Subunit. A Shiga toxin effector polypeptide is a polypeptide derived from a Shiga toxin A Subunit member of the Shiga toxin family that is capable of exhibiting a Shiga toxin function (see e.g., Cheung M et al., Mol Cancer 9: 28 (2010); WO 2014/164680, WO 2014/164693, WO 2015/138435, WO 2015/138452, WO 2015/113005, WO 2015/113007, WO 2015/191764, WO 2016/196344, WO 2017/019623, WO 2018/106895, and WO 2018/140427). Shiga toxin functions include, for example, increasing cellular internalization, directing subcellular routing from an endosomal compartment to the cytosol, avoiding intracellular degradation, catalytically inactivating ribosomes, and effectuating cytostatic and/or cytotoxic effects.

The Shiga toxin family of protein toxins includes various naturally-occurring toxins which are structurally and functionally related, such as Shiga toxin, Shiga-like toxin 1 (SLT-1), and Shiga-like toxin 2 (SLT-2) (Johannes L, Römer W, Nat Rev Microbiol 8: 105-16 (2010)). Holotoxin members of the Shiga toxin family contain targeting domains that preferentially bind a specific glycosphingolipid present on the surface of some host cells and an enzymatic domain capable of permanently inactivating ribosomes once inside a cell (Johannes L, Römer W, Nat Rev Microbiol 8: 105-16 (2010)). Members of the Shiga toxin family share the same overall structure and mechanism of action (Engedal N et al., Microbial Biotech 4: 32-46 (2011)). For example, Stx, SLT-1 and SLT-2 display indistinguishable enzymatic activity in cell free systems (Head S et al., J Biol Chem 266: 3617-21 (1991); Tesh V et al., Infect Immun 61: 3392-402 (1993); Brigotti M et al., Toxicon 35:1431-1437 (1997)).

The Shiga toxin family encompasses true Shiga toxin (Stx) isolated from S. dysenteriae serotype 1, Shiga-like toxin 1 variants (SLT1 or Stx1 or SLT-1 or Slt-I) isolated from serotypes of enterohemorrhagic E. coli, and Shiga-like toxin 2 variants (SLT2 or Stx2 or SLT-2) isolated from serotypes of enterohemorrhagic E. coli. SLT1 differs by only one amino acid residue from Stx, and both have been referred to as Verocytotoxins or Verotoxins (VTs) (O'Brien A, Curr Top Microbiol Immunol 180: 65-94 (1992)). Although SLT1 and SLT2 variants are only about 53-60% similar to each other at the primary amino acid sequence level, they share mechanisms of enzymatic activity and cytotoxicity common to the members of the Shiga toxin family (Johannes L, Römer W, Nat Rev Microbiol 8: 105-16 (2010)). Over 39 different Shiga toxins have been described, such as the defined subtypes Stx1a, Stx1c, Stx1d, and Stx2a-g (Scheutz F et al., J Clin Microbiol 50: 2951-63 (2012)). Members of the Shiga toxin family are not naturally restricted to any bacterial species because Shiga-toxin-encoding genes can spread among bacterial species via horizontal gene transfer (Strauch E et al., Infect Immun 69: 7588-95 (2001); Bielaszewska M et al., Appl Environ Micrbiol 73: 3144-50 (2007); Zhaxybayeva O, Doolittle W, Curr Biol 21: R242-6 (2011)). As an example of interspecies transfer, a Shiga toxin was discovered in a strain of A. haemolyticus isolated from a patient (Grotiuz G et al., J Clin Microbiol 44: 3838-41 (2006)). Once a Shiga toxin encoding polynucleotide enters a new subspecies or species, the Shiga toxin amino acid sequence is presumed to be capable of developing slight sequence variations due to genetic drift and/or selective pressure while still maintaining a mechanism of cytotoxicity common to members of the Shiga toxin family (see Scheutz F et al., J Clin Microbiol 50: 2951-63 (2012)).

In some embodiments, the binding molecules described herein comprise a Shiga toxin A subunit effector polypeptide that comprises or consists of a polypeptide having the sequence of: (i) amino acids 1 to 251 of any one of SEQ ID NOs: 1-18; or (ii) amino acids 1 to 261 of any one of SEQ ID NOs: 1-18; or a polypeptide having a sequence that is at least 95%, at least 96%, at least 97%, at least 98%, or at least 99% identical thereto. In some embodiments, the Shiga toxin A subunit effector polypeptide comprises or consists of a polypeptide having the sequence of any one of SEQ ID NO: 40 to 68; or a polypeptide having a sequence that is at least 95%, at least 96%, at least 97%, at least 98%, or at least 99% identical thereto.

In some embodiments, the binding molecules described herein comprise a Shiga toxin A subunit effector polypeptide that comprises or consists of a polypeptide comprising the amino acid sequence of SEQ ID NO: 41.

As described in further detail below, in some embodiments, the binding molecules comprise a Shiga toxin A Subunit effector polypeptide that comprises two or more of the following Shiga toxin effector polypeptide sub-regions: (1) a de-immunized sub-region, (2) a protease-cleavage resistant sub-region near the carboxy-terminus of a Shiga toxin A1 fragment region, and (3) a T-cell epitope-peptide embedded or inserted sub-region.

### 1. De-Immunized, Shiga Toxin A Subunit Effector Polypeptides

In some embodiments, the Shiga toxin A subunit effector polypeptides are de-immunized. For example, the Shiga toxin A subunit effector polypeptide can be de-immunized as compared to a wild-type Shiga toxin, wild-type Shiga toxin polypeptide, and/or a Shiga toxin effector polypeptide comprising only wild-type polypeptide sequences. A Shiga toxin effector polypeptide and/or Shiga toxin A Subunit polypeptide, whether naturally occurring or not, can be de-immunized by a method either described herein, described in WO 2015/113005, WO 2015/113007, WO 2016/196344, and WO 2018/140427, and/or known to the skilled worker, wherein the resulting molecule retains a Shiga toxin A Subunit function. The de-immunized, Shiga toxin effector polypeptide can comprise a disruption of at least one, putative endogenous epitope region in order to reduce the antigenic and/or immunogenic potential of the Shiga toxin effector polypeptide after administration of the polypeptide to a subject.

In some embodiments, the Shiga toxin effector polypeptide comprises a disruption of an endogenous epitope or epitope region, such as, e.g., a B-cell and/or CD4+ T-cell epitope. In certain embodiments, the Shiga toxin effector polypeptide comprises a disruption of at least one endogenous epitope region, wherein the disruption reduces the antigenic and/or immunogenic potential of the Shiga toxin effector polypeptide after administration of the polypeptide to a subject, and wherein the Shiga toxin effector polypeptide is capable of exhibiting a Shiga toxin A Subunit function, such as, e.g., a significant level of Shiga toxin cytotoxicity.

The term “disrupted” or “disruption” as used herein with regard to an epitope region refers to the deletion of at least one amino acid residue in an epitope region, inversion of two or more amino acid residues wherein at least one of the inverted amino acid residues is in an epitope region, insertion of at least one amino acid into an epitope region, and/or a substitution of at least one amino acid residue in an epitope region. An epitope region disruption by mutation includes amino acid substitutions with non-standard amino acids and/or non-natural amino acids. Epitope regions can alternatively be disrupted by mutations comprising the modification of an amino acid by the addition of a covalently-linked chemical structure which masks at least one amino acid in an epitope region, see, e.g. PEGylation (see Zhang C et al., BioDrugs 26: 209-15 (2012), small molecule adjuvants (Flower D, Expert Opin Drug Discov 7: 807-17 (2012), and site-specific albumination (Lim S et al., J Control Release 207-93 (2015)).

Certain epitope regions and disruptions are indicated herein by reference to specific amino acid positions of native Shiga toxin A Subunits provided in the Sequence Listing, noting that naturally occurring Shiga toxin A Subunits can comprise precursor forms containing signal sequences of about 22 amino acids at their amino-termini which are removed to produce mature Shiga toxin A Subunits and are recognizable to the skilled worker. Further, certain epitope region disruptions are indicated herein by reference to specific amino acids (e.g. S for a serine residue) natively present at specific positions within native Shiga toxin A Subunits (e.g. S33 for the serine residue at position 33 from the amino-terminus) followed by the amino acid with which that residue has been substituted in the particular mutation under discussion (e.g. S33I represents the amino acid substitution of isoleucine for serine at amino acid residue 33 from the amino-terminus).

In some embodiments, the de-immunized, Shiga toxin effector polypeptide comprises a disruption of at least one epitope region provided herein. In certain embodiments, the de-immunized, Shiga toxin effector polypeptide comprises a disruption of at least one epitope region described in WO 2015/113005 or WO 2015/113007.

In some embodiments, the de-immunized, Shiga toxin effector polypeptide comprises or consists of a full-length Shiga toxin A Subunit (e.g. SLT-1A (SEQ ID NO:1), StxA (SEQ ID NO:2), or SLT-2A (SEQ ID NO:3)) comprising at least one disruption of the amino acid sequence of the following natively positioned amino acids: 1-15 of SEQ ID NO:1 or SEQ ID NO:2; 3-14 of SEQ ID NO:3; 26-37 of SEQ ID NO:3; 27-37 of SEQ ID NO:1 or SEQ ID NO:2; 39-48 of SEQ ID NO:1 or SEQ ID NO:2; 42-48 of SEQ ID NO:3; 53-66 of SEQ ID NO:1, SEQ ID NO:2, or SEQ ID NO:3; 94-115 of SEQ ID NO:1, SEQ ID NO:2, or SEQ ID NO:3; 141-153 of SEQ ID NO:1 or SEQ ID NO:2; 140-156 of SEQ ID NO:3; 179-190 of SEQ ID NO:1 or SEQ ID NO:2; 179-191 of SEQ ID NO:3; 204 of SEQ ID NO:3; 205 of SEQ ID NO:1 or SEQ ID NO:2; 210-218 of SEQ ID NO:3; 240-258 of SEQ ID NO:3; 243-257 of SEQ ID NO:1 or SEQ ID NO:2; 254-268 of SEQ ID NO:1 or SEQ ID NO:2; 262-278 of SEQ ID NO:3; 281-297 of SEQ ID NO:3; or 285-293 of SEQ ID NO:1 or SEQ ID NO:2, or the equivalent position in a Shiga toxin A Subunit polypeptide, conserved Shiga toxin effector polypeptide sub-region, and/or non-native, Shiga toxin effector polypeptide sequence.

In some embodiments, the Shiga toxin effector polypeptide comprises or consists of a truncated Shiga toxin A Subunit. Truncations of Shiga toxin A Subunits might result in the deletion of an entire epitope region(s) without affecting Shiga toxin effector function(s). The smallest, Shiga toxin A Subunit fragment shown to exhibit significant enzymatic activity was a polypeptide composed of residues 75-247 of StxA (Al-Jaufy A et al., Infect Immun 62: 956-60 (1994)). Truncating the carboxy-terminus of SLT-1A, StxA, or SLT-2A to amino acids 1-251 removes two predicted B-cell epitope regions, two predicted CD4 positive (CD4+) T-cell epitopes, and a predicted, discontinuous, B-cell epitope. Truncating the amino-terminus of SLT-1A, StxA, or SLT-2A to 75-293 removes at least three, predicted, B-cell epitope regions and three predicted CD4+ T-cell epitopes. Truncating both amino- and carboxy-terminals of SLT-1A, StxA, or SLT-2A to 75-251 deletes at least five, predicted, B-cell epitope regions; four, putative, CD4+ T-cell epitopes; and one, predicted, discontinuous, B-cell epitope.

In some embodiments, a Shiga toxin effector polypeptide comprises or consists essentially of a full-length or truncated Shiga toxin A Subunit with at least one mutation, e.g. deletion, insertion, inversion, or substitution, in a provided epitope region. In some embodiments, the polypeptides comprise a disruption which comprises a deletion of at least one amino acid within the epitope region. In some embodiments, the polypeptides comprise a disruption which comprises an insertion of at least one amino acid within the epitope region. In some embodiments, the polypeptides comprise a disruption which comprises an inversion of amino acids, wherein at least one inverted amino acid is within the epitope region. In some embodiments, the polypeptides comprise a disruption which comprises a mutation, such as an amino acid substitution to a non-standard amino acid or an amino acid with a chemically modified side chain.

In some embodiments, the Shiga toxin effector polypeptides comprise or consist of a full-length or truncated Shiga toxin A Subunit with at least one mutation as compared to the native sequence which comprises at least one amino acid substitution of the following group: A, G, V, L, I, P, C, M, F, S, D, N, Q, H, and K. In some embodiments, the polypeptide comprises or consists essentially of a full-length or truncated Shiga toxin A Subunit with a single mutation as compared to the native sequence wherein the substitution is: D to A, D to G, D to V, D to L, D to I, D to F, D to S, D to Q, E to A, E to G, E to V, E to L, E to I, E to F, E to S, E to Q, E to N, E to D, E to M, E to R, G to A, H to A, H to G, H to V, H to L, H to I, H to F, H to M, K to A, K to G, K to V, K to L, K to I, K to M, K to H, L to A, L to G, N to A, N to G, N to V, N to L, N to I, N to F, P to A, P to G, P to F, R to A, R to G, R to V, R to L, R to I, R to F, R to M, R to Q, R to S, R to K, R to H, S to A, S to G, S to V, S to L, S to I, S to F, S to M, T to A, T to G, T to V, T to L, T to I, T to F, T to M, T to S, Y to A, Y to G, Y to V, Y to L, Y to I, Y to F, or Y to M.

In some embodiments, the Shiga toxin effector polypeptides comprise or consist of a full-length or truncated Shiga toxin A Subunit with at least one mutation as compared to the native amino acid residue sequence which comprises an amino acid substitution of an immunogenic residue and/or within an epitope region, wherein at least one substitution occurs at the natively positioned following group of amino acids: 1 of SEQ ID NO:1 or SEQ ID NO:2; 4 of SEQ ID NO:1, SEQ ID NO:2, or SEQ ID NO:3; 8 of SEQ ID NO:1, SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1 or SEQ ID NO:2; 47 of SEQ ID NO:1 or SEQ ID NO:2; 48 of SEQ ID NO:1, SEQ ID NO:2, or SEQ ID NO:3; 49 of SEQ ID NO:1 or SEQ ID NO:2; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1 or SEQ ID NO:2; 55 of SEQ ID NO:1 or SEQ ID NO:2; 56 of SEQ ID NO:1 or SEQ ID NO:2; 57 of SEQ ID NO:1 or SEQ ID NO:2; 58 of SEQ ID NO:1, SEQ ID NO:2, or SEQ ID NO:3; 59 of SEQ ID NO:1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1 or SEQ ID NO:2; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1 or SEQ ID NO:2; 108 of SEQ ID NO:1 or SEQ ID NO:2; 109 of SEQ ID NO:1, SEQ ID NO:2, or SEQ ID NO:3; 110 of SEQ ID NO:1 or SEQ ID NO:2; 111 of SEQ ID NO:1 or SEQ ID NO:2; 112 of SEQ ID NO:1, SEQ ID NO:2, or SEQ ID NO:3; 141 of SEQ ID NO:1 or SEQ ID NO:2; 147 of SEQ ID NO:1, SEQ ID NO:2, or SEQ ID NO:3; 154 of SEQ ID NO:1 or SEQ ID NO:2; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 198 of SEQ ID NO:1 or SEQ ID NO:2; 204 of SEQ ID NO:3; 205 of SEQ ID NO:1 or SEQ ID NO:2; 241 of SEQ ID NO:3; 242 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; or 286 of SEQ ID NO:1 or SEQ ID NO:2.

In some embodiments, the Shiga toxin effector polypeptides comprise or consist of a full-length or truncated Shiga toxin A Subunit with at least one substitution of an immunogenic residue and/or within an epitope region, wherein at least one amino acid substitution is to a non-conservative amino acid relative to a natively occurring amino acid positioned at one of the following native positions: 1 of SEQ ID NO:1 or SEQ ID NO:2; 4 of SEQ ID NO:1, SEQ ID NO:2, or SEQ ID NO:3; 8 of SEQ ID NO:1, SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1 or SEQ ID NO:2; 47 of SEQ ID NO:1 or SEQ ID NO:2; 48 of SEQ ID NO:1, SEQ ID NO:2, or SEQ ID NO:3; 49 of SEQ ID NO:1 or SEQ ID NO:2; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1 or SEQ ID NO:2; 55 of SEQ ID NO:1 or SEQ ID NO:2; 56 of SEQ ID NO:1 or SEQ ID NO:2; 57 of SEQ ID NO:1 or SEQ ID NO:2; 58 of SEQ ID NO:1, SEQ ID NO:2, or SEQ ID NO:3; 59 of SEQ ID NO:1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1 or SEQ ID NO:2; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1 or SEQ ID NO:2; 108 of SEQ ID NO:1 or SEQ ID NO:2; 109 of SEQ ID NO:1, SEQ ID NO:2, or SEQ ID NO:3; 110 of SEQ ID NO:1 or SEQ ID NO:2; 111 of SEQ ID NO:1 or SEQ ID NO:2; 112 of SEQ ID NO:1, SEQ ID NO:2, or SEQ ID NO:3; 141 of SEQ ID NO:1 or SEQ ID NO:2; 147 of SEQ ID NO:1, SEQ ID NO:2, or SEQ ID NO:3; 154 of SEQ ID NO:1 or SEQ ID NO:2; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 198 of SEQ ID NO:1 or SEQ ID NO:2; 204 of SEQ ID NO:3; 205 of SEQ ID NO:1 or SEQ ID NO:2; 241 of SEQ ID NO:3; 242 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2.

In some embodiments, the Shiga toxin effector polypeptides comprise or consist of a full-length or truncated Shiga toxin A Subunit with at least one amino acid substitution selected from: K1 to A, G, V, L, I, F, M and H; T4 to A, G, V, L, I, F, M, and S; D6 to A, G, V, L, I, F, S, and Q; S8 to A, G, V, I, L, F, and M; T8 to A, G, V, I, L, F, M, and S; T9 to A, G, V, I, L, F, M, and S; S9 to A, G, V, L, I, F, and M; K11 to A, G, V, L, I, F, M and H; T12 to A, G, V, I, L, F, M, and S; S33 to A, G, V, L, I, F, and M; S43 to A, G, V, L, I, F, and M; G44 to A and L; S45 to A, G, V, L, I, F, and M; T45 to A, G, V, L, I, F, and M; G46 to A and P; D47 to A, G, V, L, I, F, S, and Q; N48 to A, G, V, L, and M; L49 to A or G; F50; A51 to V; D53 to A, G, V, L, I, F, S, and Q; V54 to A, G, and L; R55 to A, G, V, L, I, F, M, Q, S, K, and H; G56 to A and P; 157 to A, G, M, and F; 157 to A, G, M, and F; D58 to A, G, V, L, I, F, S, and Q; P59 to A, G, and F; E60 to A, G, V, L, I, F, S, Q, N, D, M, and R; E61 to A, G, V, L, I, F, S, Q, N, D, M, and R; G62 to A; D94 to A, G, V, L, I, F, S, and Q; R84 to A, G, V, L, I, F, M, Q, S, K, and H; V88 to A and G; 188 to A, G, and V; D94; S96 to A, G, V, I, L, F, and M; T104 to A, G, V, I, L, F, M, and S; A105 to L; T107 to A, G, V, I, L, F, M, and S; S107 to A, G, V, L, I, F, and M; L108 to A, G, and M; S109 to A, G, V, I, L, F, and M; T109 to A, G, V, I, L, F, M, and S; G110 to A; D111 to A, G, V, L, I, F, S, and Q; S112 to A, G, V, L, I, F, and M; D141 to A, G, V, L, I, F, S, and Q; G147 to A; V154 to A and G; R179 to A, G, V, L, I, F, M, Q, S, K, and H; T180 to A, G, V, L, I, F, M, and S; T181 to A, G, V, L, I, F, M, and S; D183 to A, G, V, L, I, F, S, and Q; D184 to A, G, V, L, I, F, S, and Q; L185 to A, G, and V; 5186 to A, G, V, I, L, F, and M; G187 to A; R188 to A, G, V, L, I, F, M, Q, S, K, and H; 5189 to A, G, V, I, L, F, and M; D197 to A, G, V, L, I, F, S, and Q; D198 to A, G, V, L, I, F, S, and Q; R204 to A, G, V, L, I, F, M, Q, S, K, and H; R205 to A, G, V, L, I, F, M, Q, S, K and H; C242 to A, G, V, and S; S247 to A, G, V, I, L, F, and M; Y247 to A, G, V, L, I, F, and M; R248 to A, G, V, L, I, F, M, Q, S, K, and H; R250 to A, G, V, L, I, F, M, Q, S, K, and H; R251 to A, G, V, L, I, F, M, Q, S, K, and H; C262 to A, G, V, and S; D264 to A, G, V, L, I, F, S, and Q; G264 to A; and T286 to A, G, V, L, I, F, M, and S, wherein the amino acid numbering is relative to SEQ ID NO:1 or SEQ ID NO:2, or the equivalent position in a Shiga toxin A Subunit polypeptide, conserved Shiga toxin effector polypeptide sub-region, and/or non-native, Shiga toxin effector polypeptide sequence.

In some embodiments, the Shiga toxin effector polypeptides comprise or consist of a full-length or truncated Shiga toxin A Subunit with at least one of the following amino acid substitutions K1A, K1 M, T4I, D6R, S8I, TBV, T9I, S9I, K11A, K11H, T12K, S33I, S33C, S43N, G44L, S45V, S451, T45V, T451, G46P, D47M, D47G, N48V, N48F, L49A, F50T, A51V, D53A, D53N, D53G, V54L, V541, R55A, R55V, R55L, G56P, 157F, 157M, D58A, D58V, D58F, P59A, P59F, E60I, E60T, E60R, E61A, E61V, E61L, G62A, R84A, V88A, D94A, S961, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I. These epitope disrupting substitutions can be combined to form a de-immunized, Shiga toxin effector polypeptide with multiple substitutions per epitope region and/or multiple epitope regions disrupted while still retaining Shiga toxin effector function. For example, substitutions at the natively positioned K1A, K1 M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I can be combined, where possible, with substitutions at the natively positioned residues K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61 L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I to create de-immunized, Shiga toxin effector polypeptides, wherein the amino acid numbering is relative to SEQ ID NO:1 or SEQ ID NO:2, or the equivalent position in a Shiga toxin A Subunit polypeptide, conserved Shiga toxin effector polypeptide sub-region, and/or non-native, Shiga toxin effector polypeptide sequence.

Any of the de-immunized, Shiga toxin effector polypeptide sub-regions and/or epitope disrupting mutations described herein can be used alone or in combination with each individual embodiment described herein, including methods described herein.

### 2. Protease-Cleavage Resistant, Shiga Toxin A Subunit Effector Polypeptides

In some embodiments, the Shiga toxin effector polypeptide of a binding molecule as described herein comprises (1) a Shiga toxin A1 fragment derived region having a carboxy-terminus and (2) a disrupted furin-cleavage site at the carboxy-terminus of the Shiga toxin A1 fragment region. Improving the stability of connections between the Shiga toxin component and other components of the binding molecules, e.g., CTLA-4 binding regions, can improve their toxicity profiles after administration to subjects by reducing non-specific toxicities caused by the breakdown of the connection and loss of CTLA-4-binding, such as, e.g., as a result of proteolysis.

Shiga toxin A Subunits of members of the Shiga toxin family comprise a conserved, furin-cleavage site at the carboxy-terminal of their A1 fragment regions important for Shiga toxin function. Furin-cleavage sites can be identified by the skilled worker using standard techniques and/or by using the information herein.

The model of Shiga toxin cytotoxicity is that intracellular proteolytic processing of Shiga toxin A Subunits by furin in intoxicated cells is essential for 1) liberation of the A1 fragment from the rest of the Shiga holotoxin, 2) escape of the A1 fragment from the endoplasmic reticulum by exposing a hydrophobic domain in the carboxy-terminus of the A1 fragment, and 3) enzymatic activation of the A1 fragment (see Johannes L, Römer W, Nat Rev Microbiol 8: 105-16 (2010)). The efficient liberation of the Shiga toxin A1 fragment from the A2 fragment and the rest of the components of the Shiga holotoxin in the endoplasmic reticulum of intoxicated cells is important for efficient intracellular routing to the cytosol, maximal enzymatic activity, efficient ribosome inactivation, and achieving optimal cytotoxicity, i.e. comparable to a wild-type Shiga toxin (see e.g. WO 2015/191764 and references therein).

During Shiga toxin intoxication, the A Subunit is proteolytically cleaved by furin at the carboxy bond of a conserved arginine residue (e.g. the arginine residue at position 251 in StxA and SLT-1A and the arginine residue at position 250 in Stx2A and SLT-2A). Furin cleavage of Shiga toxin A Subunits occurs in endosomal and/or Golgi compartments. Furin is a specialized serine endoprotease which is expressed by a wide variety of cell types, in all human tissues examined, and by most animal cells. Furin cleaves polypeptides comprising accessible motifs often centered on the minimal, dibasic, consensus motif R-x-(R/K/x)-R (SEQ ID NO: 193). The A Subunits of members of the Shiga toxin family comprise a conserved, surface-exposed, extended loop structure (e.g. 242-261 in StxA and SLT-1A, and 241-260 in SLT-2) with a conserved S-R/Y-x-x-R (SEQ ID NO: 194) motif which is cleaved by furin. The surface exposed, extended loop structure positioned at amino acid residues 242-261 in StxA is required for furin-induced cleavage of StxA, including features flanking the minimal, furin-cleavage site R-x-x-R (SEQ ID NO: 195).

Furin-cleavage sites in Shiga toxin A Subunits and Shiga toxin effector polypeptides can be identified by the skilled worker using standard methods and/or by using the information herein. Furin cleaves the minimal, consensus site R-x-x-R (SEQ ID NO: 195) (Schalken J et al., J Clin Invest 80: 1545-9 (1987); Bresnahan P et al., J Cell Biol 111: 2851-9 (1990); Hatsuzawa K et al., J Biol Chem 265: 22075-8 (1990); Wise R et al., Proc Natl Acad Sci USA 87: 9378-82 (1990); Molloy S et al., J Biol Chem 267: 16396-402 (1992)). Consistent with this, many furin inhibitors comprise peptides comprising the site R-x-x-R (SEQ ID NO: 195). An example of a synthetic inhibitor of furin is a molecule comprising the peptide R-V-K-R (SEQ ID NO: 196) (Henrich S et al., Nat Struct Biol 10: 520-6 (2003)). In general, a peptide or protein comprising a surface accessible, dibasic amino acid motif with two positively charged, amino acids separated by two amino acid residues can be predicted to be sensitive to furin-cleavage with cleavage occurring at the carboxy bond of the last basic amino acid in the motif.

Consensus sites in substrates cleaved by furin have been identified with some degree of specificity. A furin-cleavage site has been described that comprises a region of twenty, continuous, amino acid residues, which can be labeled P14 through P6′ (Tian S et al., Int J Mol Sci 12: 1060-5 (2011)) using the nomenclature described in Schechter I, Berger, A, Biochem Biophys Res Commun 32: 898-902 (1968). According to this nomenclature, the furin-cleavage site is at the carboxy bond of the amino acid residue designated P1, and the amino acid residues of the furin-cleavage site are numbered P2, P3, P4, etc., in the direction going toward the amino-terminus from this reference P1 residue. The amino acid residues of the motif going toward the carboxy-terminus from the P1 reference residue are numbered with the prime notation P2′, P3′, P4′, etc. Using this nomenclature, the P6 to P2′ region delineates the core substrate of the furin cleavage site which is bound by the enzymatic domain of furin. The two flanking regions P14 to P7 and P3′ to P6′ are often rich in polar, amino acid residues to increase the accessibility to the core furin cleavage site located between them.

A general, furin-cleavage site is often described by the consensus site R-x-x-R (SEQ ID NO: 195) which corresponds to P4-P3-P2-P1; where “R” represents an arginine residue, a dash “-” represents a peptide bond, and a lowercase “x” represents any amino acid residue. However, other residues and positions can help to further define furin-cleavage sites. A slightly more refined furin-cleavage site is often reported as the consensus motif R-x-[K/R]-R (SEQ ID NO: 197) (where a forward slash “I” means “or” and divides alternative amino acid residues at the same position), which corresponds to P4-P3-P2-P1, because it was observed that furin has a strong preference for cleaving substrates containing this motif.

In addition to the minimal, furin-cleavage site R-x-x-R (SEQ ID NO: 195), a larger, furin-cleavage site has been described with certain amino acid residue preferences at certain positions. By comparing various known furin substrates, certain physicochemical properties have been characterized for the amino acid residues in a 20 amino acid residue long, furin-cleavage site. The P6 to P2′ region of the furin-cleavage site delineates the core furin-cleavage site which physically interacts with the enzymatic domain of furin. The two flanking regions P14 to P7 and P3′ to P6′ are often hydrophilic being rich in polar, amino acid residues to increase the surface accessibility of the core furin-cleavage site located between them.

In general, the furin-cleavage site region from position P5 to P1 tends to comprise amino acid residues with a positive charge and/or high isoelectric points. In particular, the P1 position, which marks the position of furin proteolysis, is generally occupied by an arginine but other positively charged, amino acid residues can occur in this position. Positions P2 and P3 tend to be occupied by flexible, amino acid residues, and in particular P2 tends to be occupied by arginine, lysine, or sometimes by very small and flexible amino acid residues like glycine. The P4 position tends to be occupied by positively charged, amino acid residues in furin substrates. However, if the P4 position is occupied by an aliphatic, amino acid residue, then the lack of a positively charged, functional group can be compensated for by a positively charged residue located at position(s) P5 and/or P6. Positions P1′ and P2′ are commonly occupied by aliphatic and/or hydrophobic amino acid residues, with the P1′ position most commonly being occupied by a serine.

The two, hydrophilic, flanking regions tend to be occupied by amino acid residues which are polar, hydrophilic, and have smaller amino acid functional groups; however, in certain verified furin substrates, the flanking regions do not contain any hydrophilic, amino acid residues (see Tian S, Biochem Insights 2: 9-20 (2009)).

The twenty amino acid residue, furin-cleavage site found in native, Shiga toxin A Subunits at the junction between the Shiga toxin A1 fragment and A2 fragment is well characterized in certain Shiga toxins. For example, in StxA (SEQ ID NO:2) and SLT-1A (SEQ ID NO:1), this furin-cleavage site is natively positioned from L238 to F257, and in SLT-2A (SEQ ID NO:3), this furin-cleavage site is natively positioned from V237 to Q256. Based on amino acid homology, experiment, and/or furin-cleavage assays described herein, the skilled worker can identify furin-cleavage sites in other native, Shiga toxin A Subunits or Shiga toxin effector polypeptides, where the sites are actual furin-cleavage sites or are predicted to result in the production of A1 and A2 fragments after furin cleavage of those molecules within a eukaryotic cell.

In some embodiments, the Shiga toxin effector polypeptide comprises (1) a Shiga toxin A1 fragment derived polypeptide having a carboxy-terminus and (2) a disrupted furin-cleavage site at the carboxy-terminus of the Shiga toxin A1 fragment derived polypeptide. The carboxy-terminus of a Shiga toxin A1 fragment derived polypeptide can be identified by the skilled worker by using techniques known in the art, such as, e.g., by using protein sequence alignment software to identify (i) a furin-cleavage site conserved with a naturally occurring Shiga toxin, (ii) a surface exposed, extended loop conserved with a naturally occurring Shiga toxin, and/or (iii) a stretch of amino acid residues which are predominantly hydrophobic (i.e. a hydrophobic “patch”) that can be recognized by the ERAD system.

A protease-cleavage resistant, Shiga toxin effector polypeptide of the binding molecules (1) can be completely lacking any furin-cleavage site at a carboxy-terminus of its Shiga toxin A1 fragment region and/or (2) comprise a disrupted furin-cleavage site at the carboxy-terminus of its Shiga toxin A1 fragment region and/or region derived from the carboxy-terminus of a Shiga toxin A1 fragment. A disruption of a furin-cleavage site includes various alterations to an amino acid residue in the furin-cleavage site, such as, e.g., a post-translation modification(s), an alteration of an atom in an amino acid functional group, the addition of an atom to an amino acid functional group, the association to a non-proteinaceous moiety(ies), and/or the linkage to an amino acid residue, peptide, polypeptide such as resulting in a branched proteinaceous structure.

Protease-cleavage resistant, Shiga toxin effector polypeptides can be created from a Shiga toxin effector polypeptide and/or Shiga toxin A Subunit polypeptide, whether naturally occurring or not, using a method described herein, described in WO 2015/191764, and/or known to the skilled worker, wherein the resulting molecule still retains a Shiga toxin A Subunit function.

As used herein with regard to a furin-cleavage site, the term “disruption” or “disrupted” refers to an alteration from the naturally occurring furin-cleavage site and/or furin-cleavage site, such as, e.g., a mutation, that results in a reduction in furin-cleavage proximal to the carboxy-terminus of a Shiga toxin A1 fragment region, or identifiable region derived thereof, as compared to the furin-cleavage of a wild-type Shiga toxin A Subunit or a polypeptide derived from a wild-type Shiga toxin A Subunit comprising only wild-type polypeptide sequences. An alteration to an amino acid residue in the furin-cleavage site includes a mutation in the furin-cleavage site, such as, e.g., a deletion, insertion, inversion, substitution, and/or carboxy-terminal truncation of the furin-cleavage site, as well as a post-translational modification, such as, e.g., as a result of glycosylation, albumination, and the like which involve conjugating or linking a molecule to the functional group of an amino acid residue. Because the furin-cleavage site is comprised of about twenty, amino acid residues, in theory, alterations, modifications, mutations, deletions, insertions, and/or truncations involving amino acid residues of any one of these twenty positions might result in a reduction of furin-cleavage sensitivity (Tian S et al., Sci Rep 2: 261 (2012)). The disruption of a furin-cleavage site and/or furin-cleavage site might or might not increase resistance to cleavage by other proteases, such as, e.g., trypsin and extracellular proteases common in the vascular system of mammals. The effects of a given disruption to cleavage sensitivity of a given protease can be tested by the skilled worker using techniques known in the art.

As described herein, a “disrupted furin-cleavage site” is furin-cleavage site comprising an alteration to an amino acid residue derived from the 20 amino acid residue region representing a conserved, furin-cleavage site found in native, Shiga toxin A Subunits at the junction between the Shiga toxin A1 fragment and A2 fragment regions and positioned such that furin cleavage of a Shiga toxin A Subunit results in the production of the A1 and A2 fragments; wherein the disrupted furin-cleavage site exhibits reduced furin cleavage in an experimentally reproducible way as compared to a reference molecule comprising a wild-type, Shiga toxin A1 fragment region fused to a carboxy-terminal polypeptide of a size large enough to monitor furin cleavage using the appropriate assay known to the skilled worker and/or described herein.

Examples of types of mutations which can disrupt a furin-cleavage site and furin-cleavage site are amino acid residue deletions, insertions, truncations, inversions, and/or substitutions, including substitutions with non-standard amino acids and/or non-natural amino acids. In addition, furin-cleavage sites can be disrupted by mutations comprising the modification of an amino acid by the addition of a covalently-linked structure which masks at least one amino acid in the site, such as, e.g., as a result of PEGylation, the coupling of small molecule adjuvants, and/or site-specific albumination.

If a furin-cleavage site has been disrupted by mutation and/or the presence of non-natural amino acid residues, certain disrupted furin-cleavage sites cannot be easily recognizable as being related to any furin-cleavage site; however, the carboxy-terminus of the Shiga toxin A1 fragment derived region will be recognizable and will define where the furin-cleavage site would be located were it not disrupted. For example, a disrupted furin-cleavage site can comprise less than the twenty amino acid residues of the furin-cleavage site due to a carboxy-terminal truncation as compared to a Shiga toxin A Subunit and/or Shiga toxin A1 fragment.

In some embodiments, the Shiga toxin effector polypeptide comprises (1) a Shiga toxin A1 fragment derived polypeptide having a carboxy-terminus and (2) a disrupted furin-cleavage site at the carboxy-terminus of the Shiga toxin A1 fragment polypeptide region; wherein the Shiga toxin effector polypeptide (and any binding molecule comprising it) is more furin-cleavage resistant as compared to a reference molecule, such as, e.g., a wild-type Shiga toxin polypeptide comprising the carboxy-terminus of an A1 fragment and/or the conserved, furin-cleavage site between A1 and A2 fragments. For example, a reduction in furin cleavage of one molecule compared to a reference molecule can be determined using an in vitro, furin-cleavage assay described in WO 2015/191764, conducted using the same conditions, and then performing a quantitation of the band density of any fragments resulting from cleavage to quantitatively measure in change in furin cleavage.

In some embodiments, the Shiga toxin effector polypeptide is more resistant to furin-cleavage in vitro and/or in vivo as compared to a wild-type, Shiga toxin A Subunit.

In general, the protease-cleavage sensitivity of a binding molecule is tested by comparing it to the same molecule having its furin-cleavage resistant, Shiga toxin effector polypeptide replaced with a wild-type, Shiga toxin effector polypeptide comprising a Shiga toxin A1 fragment. In certain embodiments, the binding molecules comprising a disrupted furin-cleavage site exhibit a reduction in in vitro furin cleavage of about 30%, about 40%, about 50%, about 60%, about 70%, about 80%, about 90%, about 95%, about 97%, about 98% or greater compared to a reference molecule comprising a wild-type, Shiga toxin A1 fragment fused at its carboxy-terminus to a peptide or polypeptide.

Several furin-cleavage site disruptions have been described. For example, mutating the two conserved arginines to alanines in the minimal R-x-x-R (SEQ ID NO: 195) site completely blocked processing by furin and/or furin-like proteases (see e.g Duda A et al., J Virology 78: 13865-70 (2004)). Because the furin-cleavage site is comprised of about twenty amino acid residues, in theory, certain mutations involving any one of these twenty, amino acid residue positions might abolish furin cleavage or reduce furin cleavage efficiency (see e.g., Tian S et al., Sci Rep 2: 261 (2012)).

In some embodiments, the binding molecules comprise a Shiga toxin effector polypeptide derived from at least one A Subunit of a member of the Shiga toxin family wherein the Shiga toxin effector polypeptide comprises a disruption in at least one amino acid derived from the conserved, highly accessible, protease-cleavage sensitive loop of Shiga toxin A Subunits. For example, in StxA and SLT-1A, this highly accessible, protease-sensitive loop is natively positioned from amino acid residues 242 to 261, and in SLT-2A, this conserved loop is natively positioned from amino acid residues 241 to 260. Based on polypeptide sequence homology, the skilled worker can identify this conserved, highly accessible loop structure in other Shiga toxin A Subunits. Certain mutations to the amino acid residues in this loop can reduce the accessibility of certain amino acid residues within the loop to proteolytic cleavage and this might reduce furin-cleavage sensitivity.

In some embodiments, a binding molecule comprises a Shiga toxin effector polypeptide comprising a disrupted furin-cleavage site comprising a mutation in the surface-exposed, protease sensitive loop conserved among Shiga toxin A Subunits. In some embodiments, a binding molecule comprises a Shiga toxin effector polypeptide comprising a disrupted furin-cleavage site comprising a mutation in this protease-sensitive loop of Shiga toxin A Subunits, wherein the mutation reduces the surface accessibility of certain amino acid residues within the loop such that furin-cleavage sensitivity is reduced.

In some embodiments, the disrupted furin-cleavage site of a Shiga toxin effector polypeptide comprises a disruption in terms of existence, position, or functional group of one or both of the consensus amino acid residues P1 and P4, such as, e.g., the amino acid residues in positions 1 and 4 of the minimal furin-cleavage site R/Y-x-x-R (SEQ ID NO: 198). For example, mutating one or both of the two arginine residues in the minimal, furin consensus site R-x-x-R (SEQ ID NO: 195) to alanine will disrupt a furin-cleavage site and prevent furin-cleavage at that site. Similarly, amino acid residue substitutions of one or both of the arginine residues in the minimal furin-cleavage site R-x-x-R (SEQ ID NO: 195) to any non-conservative amino acid residue known to the skilled worker will reduced the furin-cleavage sensitivity of the site. In particular, amino acid residue substitutions of arginine to any non-basic amino acid residue which lacks a positive charge, such as, e.g., A, G, P, S, T, D, E, Q, N, C, I, L, M, V, F, W, and Y, will result in a disrupted furin-cleavage site.

In some embodiments, the disrupted furin-cleavage site of a Shiga toxin effector polypeptide comprises a disruption in the spacing between the consensus amino acid residues P4 and P1 in terms of the number of intervening amino acid residues being other than two, and, thus, changing either P4 and/or P1 into a different position and eliminating the P4 and/or P1 designations. For example, deletions within the furin-cleavage site of the minimal furin-cleavage site or the core, furin-cleavage site will reduce the furin-cleavage sensitivity of the furin-cleavage site.

In some embodiments, the disrupted furin-cleavage site comprises amino acid residue substitutions, as compared to a wild-type, Shiga toxin A Subunit. In some embodiments, the disrupted furin-cleavage site comprises amino acid residue substitutions within the minimal furin-cleavage site R/Y-x-x-R (SEQ ID NO: 198), such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In some embodiments, the disrupted furin-cleavage site comprises an un-disrupted, minimal furin-cleavage site R/Y-x-x-R (SEQ ID NO: 198) but instead comprises a disrupted flanking region, such as, e.g., amino acid residue substitutions in amino acid residues in the furin-cleavage site flanking regions natively position at, e.g., 241-247 and/or 252-259. In some embodiments, the disrupted furin cleavage site comprises a substitution of at least one of the amino acid residues located in the P1-P6 region of the furin-cleavage site; mutating P1′ to a bulky amino acid, such as, e.g., R, W, Y, F, and H; and mutating P2′ to a polar and hydrophilic amino acid residue; and substituting at least one of the amino acid residues located in the P1′-P6′ region of the furin-cleavage site with bulky and hydrophobic amino acid residue(s).

In some embodiments, the disruption of the furin-cleavage site comprises a deletion, insertion, inversion, and/or mutation of at least one amino acid residue within the furin-cleavage site. In certain embodiments, a protease-cleavage resistant, Shiga toxin effector polypeptide comprises a disruption of the amino acid sequence natively positioned at 249-251 of the A Subunit of Shiga-like toxin 1 (SEQ ID NO:1) or Shiga toxin (SEQ ID NO:2), or at 247-250 of the A Subunit of Shiga-like toxin 2 (SEQ ID NO:3) or the equivalent position in a conserved Shiga toxin effector polypeptide and/or non-native Shiga toxin effector polypeptide sequence. In some embodiments, protease-cleavage resistant, Shiga toxin effector polypeptides comprise a disruption which comprises a deletion of at least one amino acid within the furin-cleavage site. In some embodiments, protease-cleavage resistant, Shiga toxin effector polypeptides comprise a disruption which comprises an insertion of at least one amino acid within the protease-cleavage motif region. In some embodiments, the protease-cleavage resistant, Shiga toxin effector polypeptides comprise a disruption which comprises an inversion of amino acids, wherein at least one inverted amino acid is within the protease motif region. In some embodiments, the protease-cleavage resistant, Shiga toxin effector polypeptides comprise a disruption which comprises a mutation, such as an amino acid substitution to a non-standard amino acid or an amino acid with a chemically modified side chain.

In some embodiments, the disrupted furin-cleavage site comprises the deletion of nine, ten, eleven, or more of the carboxy-terminal amino acid residues within the furin-cleavage site. In these embodiments, the disrupted furin-cleavage site will not comprise a furin-cleavage site or a minimal furin-cleavage site. In other words, some embodiments lack a furin-cleavage site at the carboxy-terminus of the A1 fragment region.

In some embodiments, the disrupted furin-cleavage site comprises both an amino acid residue deletion and an amino acid residue substitution as compared to a wild-type, Shiga toxin A Subunit. In some embodiments, the disrupted furin-cleavage site comprises amino acid residue deletions and substitutions within the minimal furin-cleavage site R/Y-x-x-R (SEQ ID NO: 198), such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In some embodiments, the disrupted furin-cleavage site comprises an amino acid residue deletion and an amino acid residue substitution as well as a carboxy-terminal truncation as compared to a wild-type, Shiga toxin A Subunit. In some embodiments, the disrupted furin-cleavage site comprises amino acid residue deletions and substitutions within the minimal furin-cleavage site R/Y-x-x-R (SEQ ID NO: 198), such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In some embodiments, the disrupted furin-cleavage site comprises both an amino acid substitution within the minimal furin-cleavage site R/Y-x-x-R (SEQ ID NO: 198) and a carboxy-terminal truncation as compared to a wild-type, Shiga toxin A Subunit, such as, e.g., for StxA and SLT-1A derived Shiga toxin effector polypeptides, truncations ending at the natively amino acid position 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue R248 and/or R251 substituted with any non-positively charged, amino acid residue where appropriate; and for SLT-2A derived Shiga toxin effector polypeptides, truncations ending at the natively amino acid position 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue Y247 and/or R250 substituted with any non-positively charged, amino acid residue where appropriate.

In some embodiments, the disrupted furin-cleavage site comprises an insertion of amino acid residues as compared to a wild-type, Shiga toxin A Subunit as long as the inserted amino residue(s) does not create a de novo furin-cleavage site. In some embodiments, the insertion of at least one amino acid residues disrupts the natural spacing between the arginine residues in the minimal, furin-cleavage site R/Y-x-x-R (SEQ ID NO: 198), such as, e.g., StxA and SLT-1A derived polypeptides comprising an insertion of at least one amino acid residues at 249 or 250 and thus between R248 and R251; or SLT-2A derived polypeptides comprising an insertion of at least one amino acid residues at 248 or 249 and thus between Y247 and R250.

In some embodiments, the disrupted furin-cleavage site comprises both an amino acid residue insertion and a carboxy-terminal truncation as compared to a wild-type, Shiga toxin A Subunit. In some embodiments, the disrupted furin-cleavage site comprises both an amino acid residue insertion and an amino acid residue substitution as compared to a wild-type, Shiga toxin A Subunit. In certain embodiments, the disrupted furin-cleavage site comprises both an amino acid residue insertion and an amino acid residue deletion as compared to a wild-type, Shiga toxin A Subunit.

In some embodiments, the disrupted furin-cleavage site comprises an amino acid residue deletion, an amino acid residue insertion, and an amino acid residue substitution as compared to a wild-type, Shiga toxin A Subunit.

In some embodiments, the disrupted furin-cleavage site comprises an amino acid residue deletion, insertion, substitution, and carboxy-terminal truncation as compared to a wild-type, Shiga toxin A Subunit.

In some embodiments, the Shiga toxin effector polypeptide comprising a disrupted furin-cleavage site is directly fused by a peptide bond to a molecular moiety comprising an amino acid, peptide, and/or polypeptide wherein the fused structure involves a single, continuous polypeptide. In these fusion embodiments, the amino acid sequence following the disrupted furin-cleavage site should not create a de novo, furin-cleavage site at the fusion junction.

Any of the above protease-cleavage resistant, Shiga toxin effector polypeptide sub-regions and/or disrupted furin-cleavage sites can be used alone or in combination with each individual embodiment described herein, including the methods described herein.

### 3. T-Cell Hyper-Immunized, Shiga Toxin A Subunit Effector Polypeptides

In some embodiments, the Shiga toxin effector polypeptides comprise an embedded or inserted epitope-peptide. In some embodiments, the epitope-peptide is a heterologous, T-cell epitope-peptide, such as, e.g., an epitope considered heterologous to Shiga toxin A Subunits. In certain further embodiments, the epitope-peptide is a CD8+ T-cell epitope. In certain further embodiments, the CD8+ T-cell epitope-peptide has a binding affinity to a MHC class I molecule characterized by a dissociation constant (K_{D}) of 10⁻⁴ molar or less (e.g., 10⁻⁴ molar to 10⁻⁶ molar) and/or the resulting MHC class I-epitope-peptide complex has a binding affinity to a T-cell receptor (TCR) characterized by a dissociation constant (K_{D}) of 10⁻⁴ molar or less (e.g., 10⁻⁴ molar to 10⁻⁶ molar).

In some embodiments, the Shiga toxin effector polypeptide comprises an embedded or inserted, heterologous, T-cell epitope, such as, e.g., a human CD8+ T-cell epitope. In some embodiments, the heterologous, T-cell epitope is embedded or inserted so as to disrupt an endogenous epitope or epitope region (e.g. a B-cell epitope and/or CD4+ T-cell epitope) identifiable in a naturally occurring Shiga toxin polypeptide or parental Shiga toxin effector polypeptide from which the Shiga toxin effector polypeptide is derived.

In some embodiments, the Shiga toxin effector polypeptide (and any binding molecule comprising it) is CD8+ T-cell hyper-immunized, such as, e.g., as compared to a wild-type Shiga toxin polypeptide. Each CD8+ T-cell hyper-immunized, Shiga toxin effector polypeptide comprises an embedded or inserted T-cell epitope-peptide. Hyper-immunized, Shiga toxin effector polypeptides can be created from Shiga toxin effector polypeptides and/or Shiga toxin A Subunit polypeptides, whether naturally occurring or not, using a method described herein, described in WO 2015/113005, and/or known to the skilled worker, wherein the resulting molecule still retains at least one Shiga toxin A Subunit function.

As described herein, a T-cell epitope is a molecular structure which is comprised by an antigenic peptide and can be represented by a linear, amino acid sequence. Commonly, T-cell epitopes are peptides of sizes of eight to eleven amino acid residues (Townsend A, Bodmer H, Annu Rev Immunol 7: 601-24 (1989)); however, certain T-cell epitope-peptides have lengths that are smaller than eight or larger than eleven amino acids long (see e.g. Livingstone A, Fathman C, Annu Rev Immunol 5: 477-501 (1987); Green K et al., Eur J Immunol 34: 2510-9 (2004)). In some embodiments, the embedded or inserted epitope is at least seven amino acid residues in length. In some embodiments, the embedded or inserted epitope is bound by a TCR with a binding affinity characterized by a K_{D} less than 10 mM (e.g. 1-100 μM) as calculated using the formula in Stone J et al., Immunology 126: 165-76 (2009). However, it should be noted that the binding affinity within a given range between the MHC-epitope and TCR might not correlate with antigenicity and/or immunogenicity (see e.g. Al-Ramadi B et al., J Immunol 155: 662-73 (1995)), such as due to factors like MHC-peptide-TCR complex stability, MHC-peptide density and MHC-independent functions of TCR cofactors such as CD8 (Baker B et al., Immunity 13: 475-84 (2000); Hornell T et al., J Immunol 170: 4506-14 (2003); Woolridge L et al., J Immunol 171: 6650-60 (2003)).

A heterologous, T-cell epitope is an epitope not already present in a wild-type Shiga toxin A Subunit; a naturally occurring Shiga toxin A Subunit; and/or a parental, Shiga toxin effector polypeptide used as a source polypeptide for modification by a method described herein, described in WO 2015/113005, and/or known to the skilled worker.

A heterologous, T-cell epitope-peptide can be incorporated into a source polypeptide via numerous methods known to the skilled worker, including, e.g., the processes of creating amino acid substitutions within the source polypeptide, fusing at least one amino acid to the source polypeptide, inserting at least one amino acid into the source polypeptide, linking a peptide to the source polypeptide, and/or a combination of the aforementioned processes. The result of such a method is the creation of a modified variant of the source polypeptide which comprises at least one embedded or inserted, heterologous, T-cell epitope-peptides.

T-cell epitopes can be chosen or derived from a number of source molecules for use in the binding molecules described herein. T-cell epitopes can be created or derived from various naturally occurring proteins. T-cell epitopes can be created or derived from various naturally occurring proteins foreign to mammals, such as, e.g., proteins of microorganisms. T-cell epitopes can be created or derived from mutated human proteins and/or human proteins aberrantly expressed by malignant human cells. T-cell epitopes can be synthetically created or derived from synthetic molecules (see e.g., Carbone F et al., J Exp Med 167: 1767-9 (1988); Del Val M et al., J Virol 65: 3641-6 (1991); Appella E et al., Biomed Pept Proteins Nucleic Acids 1: 177-84 (1995); Perez S et al., Cancer 116: 2071-80 (2010)).

Although many T-cell epitope-peptides are useful as a heterologous, T-cell epitope, certain epitopes can be selected based on desirable properties. For example, in many species, the MHC alleles in its genome encode multiple MHC-I molecular variants. Because MHC class I protein polymorphisms can affect antigen-MHC class I complex recognition by CD8+ T-cells, T-cell epitopes can be chosen for use in the binding molecules based on knowledge about certain MHC class I polymorphisms and/or the ability of certain antigen-MHC class I complexes to be recognized by T-cells having different genotypes.

In some embodiments, the binding molecules comprise CD8+ T-cell hyper-immunized, Shiga toxin effector polypeptides, meaning that the heterologous, T-cell epitope is highly immunogenic and can elicit robust immune responses in vivo when displayed complexed with a MHC class I molecule on the surface of a cell. In some embodiments, the Shiga toxin effector polypeptide comprises at least one embedded or inserted, heterologous, T-cell epitopes which are CD8+ T-cell epitopes. A Shiga toxin effector polypeptide that comprises a heterologous, CD8+ T-cell epitope is considered a CD8+ T-cell hyper-immunized, Shiga toxin effector polypeptide.

T-cell epitope components can be obtained or derived from a number of source molecules already known to be capable of eliciting a vertebrate immune response. T-cell epitopes can be derived from various naturally occurring proteins foreign to vertebrates, such as, e.g., proteins of pathogenic microorganisms and non-self, cancer antigens. In particular, infectious microorganisms can contain numerous proteins with known antigenic and/or immunogenic properties. Further, infectious microorganisms can contain numerous proteins with known antigenic and/or immunogenic sub-regions or epitopes.

In some embodiments, the proteins of intracellular pathogens with mammalian hosts are sources for T-cell epitopes. There are numerous intracellular pathogens, such as viruses, bacteria, fungi, and single-cell eukaryotes, with well-studied antigenic proteins or peptides. T-cell epitopes can be selected or identified from human viruses or other intracellular pathogens, such as, e.g., bacteria like mycobacterium, fungi like toxoplasmae, and protists like trypanosomes.

In some embodiments, there are many immunogenic, viral peptide components of viral proteins from viruses that are infectious to humans. Numerous, human T-cell epitopes have been mapped to peptides within proteins from influenza A viruses, such as peptides in the proteins HA glycoproteins FE17, S139/1, CH65, C05, hemagglutinin 1 (HA1), hemagglutinin 2 (HA2), nonstructural protein 1 and 2 (NS1 and NS 2), matrix protein 1 and 2 (M1 and M2), nucleoprotein (NP), neuraminidase (NA)), and many of these peptides have been shown to elicit human immune responses, such as by using ex vivo assay. Similarly, numerous, human T-cell epitopes have been mapped to peptide components of proteins from human cytomegaloviruses (HCMV), such as peptides in the proteins pp65 (UL83), UL128-131, immediate-early 1 (IE-1; UL123), glycoprotein B, tegument proteins, and many of these peptides have been shown to elicit human immune responses, such as by using ex vivo assays.

Another example is there are many immunogenic cancer antigens in humans. The CD8+ T-cell epitopes of cancer and/or tumor cell antigens can be identified by the skilled worker using techniques known in the art, such as, e.g., differential genomics, differential proteomics, immunoproteomics, prediction then validation, and genetic approaches like reverse-genetic transfection (see e.g., Admon A et al., Mol Cell Proteomics 2: 388-98 (2003); Purcell A, Gorman J, Mol Cell Proteomics 3: 193-208 (2004); Comber J, Philip R, Ther Adv Vaccines 2: 77-89 (2014)). There are many antigenic and/or immunogenic T-cell epitopes already identified or predicted to occur in human cancer and/or tumor cells. For example, T-cell epitopes have been predicted in human proteins commonly mutated or overexpressed in neoplastic cells, such as, e.g., ALK, CEA, N-acetylglucosaminyl-transferase V (GnT-V), HCA587, PD-L1/neu, MAGE, Melan-A/MART-1, MUC-1, p53, and TRAG-3 (see e.g., van der Bruggen P et al., Science 254: 1643-7 (1991); Kawakami Y et al., J Exp Med 180: 347-52 (1994); Fisk B et al., J Exp Med 181: 2109-17 (1995); Guilloux Y et al., J Exp Med 183: 1173 (1996); Skipper J et al., J Exp Med 183: 527 (1996); Brossart P et al., 93: 4309-17 (1999); Kawashima I et al., Cancer Res 59: 431-5 (1999); Papadopoulos K et al., Clin Cancer Res 5: 2089-93 (1999); Zhu B et al., Clin Cancer Res 9: 1850-7 (2003); Li B et al., Clin Exp Immunol 140: 310-9 (2005); Ait-Tahar K et al., Int J Cancer 118: 688-95 (2006); Akiyama Y et al., Cancer Immunol Immunother 61: 2311-9 (2012)). In addition, synthetic variants of T-cell epitopes from human cancer cells have been created (see e.g., Lazoura E, Apostolopoulos V, Curr Med Chem 12: 629-39 (2005); Douat-Casassus C et al., J Med Chem 50: 1598-609 (2007)).

There are well-defined peptide-epitopes that are known to be immunogenic, MHC class I restricted, and/or matched with a specific human leukocyte antigen (HLA) variant(s). For administration to humans for applications involving human target cells, HLA-class I-restricted epitopes can be selected or identified by the skilled worker using standard techniques known in the art. The ability of peptides to bind to human MHC class I molecules can be used to predict the immunogenic potential of putative T-cell epitopes. The ability of peptides to bind to human MHC class I molecules can be scored using software tools. T-cell epitopes can be chosen for use as a heterologous, T-cell epitope component based on the peptide selectivity of the HLA variants encoded by the alleles more prevalent in certain human populations. For example, the human population is polymorphic for the alpha chain of MHC class I molecules due to the varied alleles of the HLA genes from individual to individual. In certain T-cell epitopes can be more efficiently presented by a specific HLA molecule, such as, e.g., the commonly occurring HLA variants encoded by the HLA-A allele groups HLA-A2 and HLA-A3.

Multiple factors can be considered that can influence epitope generation and transport to receptive MHC class I molecules, such as, e.g., the presence and epitope specificity of the following factors in the target cell: proteasome, ERAAP/ERAP1, tapasin, and TAPs.

In some embodiments, the particular epitope is that which best matches the MHC class I molecules present in the cell-type or cell populations to be targeted. Different MHC class I molecules exhibit preferential binding to particular peptide sequences, and particular peptide-MHC class I variant complexes are specifically recognized by the t-cell receptors (TCRs) of effector T-cells. The skilled worker can use knowledge about MHC class I molecule specificities and TCR specificities to optimize the selection of heterologous, T-cell epitopes used in the binding molecules described herein.

In addition, multiple, immunogenic, T-cell epitopes for MHC class I presentation can be embedded in the same Shiga toxin effector polypeptide for use, such as, e.g., in the targeted delivery of a plurality of T-cell epitopes simultaneously.

In some embodiments, the binding molecules described herein comprise a Shiga toxin A subunit effector polypeptide comprising: (i) amino acids 75 to 251 of any one of SEQ ID NOs: 1-18; (ii) amino acids 1 to 241 of any one of SEQ ID NOs: 1-18; (iii) amino acids 1 to 251 of any one of SEQ ID NOs: 1-18; or (iv) amino acids 1 to 261 of any one of SEQ ID NOs: 1-18. In some embodiments, the binding molecules comprise a polypeptide having a sequence that is at least 95%, at least 96%, at least 97%, at least 98%, or at least 99% identical to a Shiga toxin A subunit effector polypeptide comprising: (i) amino acids 75 to 251 of any one of SEQ ID NOs: 1-18; (ii) amino acids 1 to 241 of any one of SEQ ID NOs: 1-18; (iii) amino acids 1 to 251 of any one of SEQ ID NOs: 1-18; or (iv) amino acids 1 to 261 of any one of SEQ ID NOs: 1-18. In some embodiments, the Shiga toxin A subunit effector polypeptide comprises or consists of a polypeptide having the sequence of any one of SEQ ID NO: 40 to 68. In some embodiments, the Shiga toxin A subunit effector polypeptide comprises or consists of a polypeptide having a sequence that is at least 95%, at least 96%, at least 97%, at least 98%, or at least 99% identical to the sequence of any one of SEQ ID NO: 40 to 68.

In some embodiments, the binding molecules described herein comprise a Shiga toxin effector polypeptide SEQ ID NO: 41. In some embodiments, the binding molecules described herein comprise a Shiga toxin effector polypeptide that is a variant of SEQ ID NO: 41. In some embodiments, the binding molecules described herein comprise a Shiga toxin effector polypeptide comprising a sequence with at least 90%, at least 95%, at least 96%, at least 97%, at least 98%, or at least 99% identity to SEQ ID NO: 41. In some embodiments, the binding molecules described herein comprise a Shiga toxin effector polypeptide comprising SEQ ID NO: 41 with one or more mutations, such as 2, 3, 4, 5, 6, 7, 8, or 10 mutations. In some embodiments, the Shiga toxin effector comprises SEQ ID NO: 41, with 1-5, 5-10, 11-5, 15-20, 10-25, 25-30, or more than 30 mutations. In some embodiments, mutations in the Shiga toxin effector polypeptide render the polypeptide catalytically inactive. In some embodiments, mutations in the Shiga toxin effector polypeptide do not affect the catalytic activity of the polypeptide. In some embodiments, mutations in the Shiga toxin effector polypeptide increase the catalytic activity of the polypeptide. In some embodiments, mutations in the Shiga toxin effector polypeptide decrease the catalytic activity of the polypeptide.

In some embodiments, the binding molecules described herein comprise a Shiga toxin A subunit effector polypeptide that comprises or consists of a polypeptide having the sequence of any one of the sequences in Table 4 below, or a sequence that is at least 85%, at least 90%, at least 95%, at least 96%, at least 97%, at least 98%, at least 99% identical thereto. In some embodiments, the binding molecules comprise a Shiga toxin A subunit effector polypeptide that comprises or consists of a polypeptide having the sequence of any one of the sequences in Table 4 below, or a sequence that has at least 1, at least 2, at least 3, at least 4, at least 5, at least 6, at least 7, at least 8, at least 9, or at least 10 amino acids relative thereto.

TABLE 4
Shiga-like toxin subunits

| ID Number | Text Description | Biological Sequence |
|---|---|---|
| SEQ ID | Shiga-like toxin 1 | KEFTLDFSTAKTYVDSLNVIRSAIGTPLQTISSGGTSLLMIDSGS |
| NO: 1 | Subunit A (SLT-1A) | GDNLFAVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHASRVARMASDEFP |
| | | SMCPADGRVRGITHNKILWDSSTLGAILMRRTISS |
| SEQ ID | Shiga toxin Subunit A | KEFTLDFSTAKTYVDSLNVIRSAIGTPLQTISSGGTSLLMIDSGT |
| NO: 2 | (StxA) | GDNLFAVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHASRVARMASDEFP |
| | | SMCPADGRVRGITHNKILWDSSTLGAILMRRTISS |
| SEQ ID | Shiga-like toxin 2 | DEFTVDFSSQKSYVDSLNSIRSAISTPLGNISQGGVSVSVINHVL |
| NO: 3 | Subunit A (SLT-2A) | GGNYISLNVRGLDPYSERFNHLRLIMERNNLYVAGFINTETNIF |
| | | YRFSDFSHISVPDVITVSMTTDSSYSSLQRIADLERTGMQIGRH |
| | | SLVGSYLDLMEFRGRSMTRASSRAMLRFVTVIAEALRFRQIQR |
| | | GFRPALSEASPLYTMTAQDVDLTLNWGRISNVLPEYRGEEGV |
| | | RIGRISFNSLSAILGSVAVILNCHSTGSYSVRSVSQKQKTECQIV |
| | | GDRAAIKVNNVLWEANTIAALLNRKPQDLTEPNQ |
| SEQ ID | Shiga toxin subtype c | KEFTLDFSTAKTYVDSLNVIRSAIGTPLQTISSGGTSLLMIDSGT |
| NO: 4 | Subunit A (Stx1cA) | GDNLFAVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSVNAILGSVALILNCHHHASRVAR |
| SEQ ID | Shiga toxin subtype d | KEFTLDFSTAKKYVDSLNVIRSAIGTPLQTISSGGTSLLMIDSGT |
| NO: 5 | Subunit A (Stx1dA) | GDNLFAVDIMGLEPEEERFNNLRLIVERNNLYVTGFVNRTNN |
| | | VFYRFADFSHVTFPGTRAVTLSGDSSYTTLQRVAGISRTGMQI |
| | | NRHSLTTSYLDLMSYSGTSLTQSVARAMLRFVTVTAEALRFR |
| | | QIQRGFRTTLDDLSGRSYVMTAEDVDLTLNWGRLSSILPDYH |
| | | GQDSVRVGRISFGSINAILGSVALILNCHHHASRVAR |
| SEQ ID | Shiga toxin subtype e | QDFTVDFSTAKKYVDSLNAIRSAIGTPLHSISSGGTSLLMIDNG |
| NO: 6 | Subunit A (Stx1eA) | TGDNLFAVDIRGLDPEEERFDNLRLIIERNNLYVTGFVNRTSNI |
| | | FYRFADFSHVTFPGTRAVTLSGDSSYTTLQRVAGIGRTGMQIN |
| | | RHSLTTSYLDLMSYSGSSLTQPVARAMLRFVTVTAEALRFRQI |
| | | QRGFRTTLDDVSGHSYTMTVEDVDLTLNWGRLSSVLPDYHG |
| | | QDSVRVGRISFGGVNAILGSVALILNCHHHTSRVSR |
| SEQ ID | Shiga toxin subtype 2c | REFTIDFSTQQSYVSSLNSIRTEISTPLEHISQGTTSVSVINHTPP |
| NO: 7 | Subunit A (Stx2cA) | GSYFAVDIRGLDVYQARFDHLRLIIEQNNLYVAGFVNTATNTF |
| | variant 1 | YRFSDFTHISVPGVTTVSMTTDSSYTTLQRVAALERSGMQISR |
| | | HSLVSSYLALMEFSGNTMTRDASRAVLRFVTVTAEALRFRQIQ |
| | | REFRQALSETAPVYTMTPGDVDLTLNWGRISNVLPEYRGEDG |
| | | VRVGRISFNNISAILGTVAVILNCHHQGARSVR |
| SEQ ID | Shiga toxin subtype 2c | REFTIDFSTQQSYVSSLNSIRTEISTPLEHISQGTTSVSVINHTPP |
| NO: 8 | Subunit A (Stx2cA) | GSYFAVDIRGLDVYQARFDHLRLIIEQNNLYVAGFVNTATNTF |
| | variant 2 | YRFSDFAHISVPGVTTVSMTTDSSYTTLQRVAALERSGMQISR |
| | | HSLVSSYLALMEFSGNTMTRDASRAVLRFVTVTAEALRFRQIQ |
| | | REFRQALSETAPVYTMTPGDVDLTLNWGRISNVLPEYRGEDG |
| | | VRVGRISFNNISAILGTVAVILNCHHQGARSVR |
| SEQ ID | Shiga toxin subtype 2c | REFTIDFSTQQSYVSSLNSIRTEISTPLEHISQGTTSVSVINHTPP |
| NO: 9 | Subunit A (Stx2cA) | GSYFAVDIRGLDIYQARFDHLRLIIEQNNLYVAGFVNTATNTF |
| | variant 3 | YRFSDFTHISVPGVTTVSMTTDSSYTTLQRVAALERSGMQISR |
| | | HSLVSSYLALMEFSGNTMTRDASRAVLRFVTVTAEALRFRQIQ |
| | | REFRQALSETAPVYTMTPGDVDLTLNWGRISNVLPEYRGEDG |
| | | VRVGRISFNNISAILGTVAVILNCHHQGARSVR |
| SEQ ID | Shiga toxin subtype 2c | REFTIDFSTQQSYVSSLNSIRTEISTPLEHISQGTTSVSVINHTPP |
| NO: 10 | Subunit A (Stx2cA) | GSYFAVDIRGLDVYQARFDHLRLIIEQNNLYVAGFVNTATNTF |
| | variant 4 | YRFSDFTHISVPSVTTVSMTTDSSYTTLQRVAALERSGMQISRH |
| | | SLVSSYLALMEFSGNTMTRDASRAVLRFVTVTAEALRFRQIQR |
| | | EFRQALSETAPVYTMTPGDVDLTLNWGRISNVLPEYRGEDGV |
| | | RVGRISFNNISAILGTVAVILNCHHQGARSVR |
| SEQ ID | Shiga toxin subtype 2c | REFTIDFSTQQSYVSSLNSIRTEISTPLEHISQGTTSVSVINHTPP |
| NO: 11 | Subunit A (Stx2cA) | GSYFAVDIRGLDVYQARFDHLRLIIEQNNLYMAGFVNTATNTF |
| | variant 5 | YRFSDFTHISVPSVTTVSMTTDSSYTTLQRVAALERSGMQISRH |
| | | SLVSSYLALMEFSGNTMTRDASRAVLRFVTVTAEALRFRQIQR |
| | | EFRQALSETAPVYTMTPGDVDLTLNWGRISNVLPEYRGEDGV |
| | | RVGRISFNNISAILGTVAVILNCHHQGARSVR |
| SEQ ID | Shiga toxin subtype 2c | REFTIDFSTQQSYVSSLNSIRTEISTPLEHISQGTTSVSVINHTPP |
| NO: 12 | Subunit A (Stx2cA) | GSYFAVDIRGLDVYQARFDHLRLIIEQNNLYVAGFVNTATNTF |
| | variant 6 | YRFSDFTHISVPGVTTVSMTTDSSYTTLQRVAALERSGMQISR |
| | | HSLVSSYLALMEFSGNTMTRDASRAVLRFVTVTAEALRFRQIQ |
| | | REFRQVLSETAPVYTMTPGDVDLTLNWGRISNVLPEYRGEDG |
| | | VRVGRISFNNISAILSTVAVILNCHHQGARSVR |
| SEQ ID | Shiga toxin subtype 2d | REFTIDFSTQQSYVSSLNSIRTEISTPLEHISQGTTSVSVINHTPP |
| NO: 13 | Subunit A (Stx2dA) | GSYFAVDIRGLDVYQARFDHLRLIIEQNNLYVAGFVNTATNTF |
| | variant 1 | YRFSDFAHISVPGVTTVSMTTDSSYTTLQRVAALERSGMQISR |
| | | HSLVSSYLALMEFSGNTMTRDASRAVLRFVTVTAEALRFRQIQ |
| | | REFRQALSETAPVYTMTPGDVDLTLNWGRISNVIPEYRGEDG |
| | | VRVGRISFNNISAILGTVAVILNCHHQGARSVR |
| SEQ ID | Shiga toxin subtype 2d | REFMIDFSTQQSYVSSLNSIRTEISTPLEHISQGTTSVSVINHTPP |
| NO: 14 | Subunit A (Stx2dA) | GSYFAVDIRGLDVYQARFDHLRLIIEQNNLYVAGFVNTATNTF |
| | variant 2 | YRFSDFTHISVPGVTTVSMTTDSSYTTLQRVAALERSGMQISR |
| | | HSLVSSYLALMEFSGNTMTRDASRAVLRFVTVTAEALRFRQIQ |
| | | REFRQALSETAPVYTMTPEEVDLTLNWGRISNVLPEFRGEGGV |
| | | RVGRISFNNISAILGTVAVILNCHHQGARSVR |
| SEQ ID | Shiga toxin subtype 2d | REFTIDFSTQQSYVSSLNSIRTEISTPLEHISQGTTSVSVINHTPP |
| NO: 15 | Subunit A (Stx2dA) | GSYFAVDIRGLDVYQARFDHLRLIIEQNNLYVAGFVNTATNTF |
| | variant 3 | YRFSDFTHISVPGVTTVSMTTDSSYTTLQRVAALERSGMQISR |
| | | HSLVSSYLALMEFSGNTMTRDASRAVLRFVTVTAEALRFRQIQ |
| | | REFRQALSETAPVYTMTPGDVDLTLNWGRISNVIPEYRGEDG |
| | | VRVGRISFNNISAILSTVAVILNCHHQGARSVR |
| SEQ ID | Shiga toxin subtype 2e | QEFTIDFSTQQSYVSSLNSIRTAISTPLEHISQGATSVSVINHTPP |
| NO: 16 | Subunit A (Stx2eA) | GSYISVGIRGLDVYQERFDHLRLIIERNNLYVAGFVNTTTNTFY |
| | variant 1 | RFSDFAHISLPGVTTISMTTDSSYTTLQRVAALERSGMQISRHS |
| | | LVSSYLALMEFSGNTMTRDASRAVLRFVTVTAEALRFRQIQRE |
| | | FRLALSETAPVYTMTPEDVDLTLNWGRISNVLPEYRGEAGVR |
| | | VGRISFNNISAILGTVAVILNCHHQGARSVR |
| SEQ ID | Shiga toxin subtype 2e | QEFTIDFSTQQSYVSSLNSIRTAISTPLEHISQGATSVSVINHTPP |
| NO: 17 | Subunit A (Stx2eA) | GSYISVGIRGLDVYQAHFDHLRLIIEQNNLYVAGFVNTATNTF |
| | variant 2 | YRFSDFAHISLPGVTTISMTTDSSYTTLQRVAALERSGMQISRH |
| | | SLVSSYLALMEFSGNTMTREASRAVLRFVTVTAEALRFRQIQR |
| | | EFRQALSETAPVYTMTPEDVDLTLNWGRISNVLPEYRGEDGV |
| | | RVGRISFNNISAILGTVAVILNCHHQGARSVR |
| SEQ ID | Shiga toxin subtype 2f | DEFTVDFSSQKSYVDSLNSIRSAISTPLGNISQGGVSVSVINHVP |
| NO: 18 | Subunit A (Stx2fA) | GGNYISLNVRGLDPYSERFNHLRLIMERNNLYVAGFINTETNT |
| | | FYRFSDFSHISVPDVITVSMTTDSSYSSLQRIADLERTGMQIGR |
| | | HSLVGSYLDLMEFRGRSMTRASSRAMLRFVTVIAEALRFRQIQ |
| | | RGFRPALSEASPLYTMTAQDVDLTLNWGRISNVLPEYRGEEG |
| | | VRIGRISFNSLSAILGSVAVILNCHSTGSYSVR |
| SEQ ID | Shiga toxin effector | AEFTLDFSTAKTYVDSLNVIRSAIGTPLQTISSGGTSLLMIDSGI |
| NO: 40 | polypeptide 1 | GDNLFAVDILGFDFTLGRFNNLRLIVERNNLYVTGFVNRTNNV |
| | | FYRFADFSHVTFPGTTAVTLSADSSYTTLQRVAGISRTGMQIN |
| | | RHSLTTSYLDLMSHSATSLTQSVARAMLRFVTVTAEALRFRQI |
| | | QRGFRTTLDDLSGRSYVMTAEDVDLTLNWGRLSSVLPDYHG |
| | | QDSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGI |
| NO: 41 | polypeptide 2 | GDNLFAVDILGFDFTLGRFNNLRLIVERNNLYVTGFVNRTNNV |
| | | FYRFADFSHVTFPGTTAVTLSADSSYTTLQRVAGISRTGMQIN |
| | | RHSLTTSYLDLMSHSGTSLTQSVARAMLRFVTVTAEALRFRQI |
| | | QRGFRTTLDDLSGASYVMTAEDVDLTLNWGRLSSVLPDYHG |
| | | QDSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGI |
| NO: 42 | polypeptide 3 | GDNLFAVDILGFDFTLGRFNNLRLIVERNNLYVTGFVNRTNNV |
| | | FYRFADFSHVTFPGTTAVTLSADSSYTTLQRVAGISRTGMQIN |
| | | RHSLTTSYLALMSHSGTSLTQSVARAMLRFVTVTAEALRFRQI |
| | | QRGFRTTLDDLSGASYVMTAEDVDLTLNWGRLSSVLPDYHG |
| | | QDSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGI |
| NO: 43 | polypeptide 4 | GDNLFAVDILGFDFTLGRFNNLRLIVERNNLYVTGFVNRTNNA |
| | | FYRFADFSHVTFPGTTAVTLSADSSYTTLQRVAGISRTGMQIN |
| | | RHSLTTSYLALMSHSGTSLTQSAARAMLRFVTVTAEALRFRQI |
| | | QRGFRTTLDDLSGASYVMTAEDVDLTLNWGRLSSVLPDYHG |
| | | QDSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGS |
| NO: 44 | polypeptide 5 | GDNLFAVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHASAVAA |
| SEQ ID | Shiga toxin effector | AEFTLDFSTAKTYVDSLNVIRSAIGTPLQTISSGGTSLLMIDSGI |
| NO: 45 | polypeptide 6 | GDNLFAVDILGFDFTLGRFNNLRLIVERNNLYVTGFVNRTNNV |
| | | FYRFADFSHVTFPGTTAVTLSADSSYTTLQRVAGISRTGMQIN |
| | | RHSLTTSYLDLMSHSATSLTQSVARAMLRFVTVTADALRFRQI |
| | | QRGFRTTLDDLSGRSYVMTAEDVDLTLNWGRLSSVLPDYHG |
| | | QDSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGI |
| NO: 46 | polypeptide 7 | GDNLFAVDILGFDFTLGRFNNLRLIVERNNLYVTGFVNRTNNV |
| | | FYRFADFSHVTFPGTTAVTLSADSSYTTLQRVAGISRTGMQIN |
| | | RHSLTTSYLDLMSHSGTSLTQSVARAMLRFVTVTADALRFRQI |
| | | QRGFRTTLDDLSGASYVMTAEDVDLTLNWGRLSSVLPDYHG |
| | | QDSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGI |
| NO: 47 | polypeptide 8 | GDNLFAVDILGFDFTLGRFNNLRLIVERNNLYVTGFVNRTNNV |
| | | FYRFADFSHVTFPGTTAVTLSADSSYTTLQRVAGISRTGMQIN |
| | | RHSLTTSYLALMSHSGTSLTQSVARAMLRFVTVTADALRFRQI |
| | | QRGFRTTLDDLSGASYVMTAEDVDLTLNWGRLSSVLPDYHG |
| | | QDSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGI |
| NO: 48 | polypeptide 9 | GDNLFAVDILGFDFTLGRFNNLRLIVERNNLYVTGFVNRTNNA |
| | | FYRFADFSHVTFPGTTAVTLSADSSYTTLQRVAGISRTGMQIN |
| | | RHSLTTSYLALMSHSGTSLTQSAARAMLRFVTVTADALRFRQI |
| | | QRGFRTTLDDLSGASYVMTAEDVDLTLNWGRLSSVLPDYHG |
| | | QDSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGS |
| NO: 49 | polypeptide 10 | GDNLFAVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTADALRFR |
| | | QIQRGFRTTLDDLSGRSYVMTAEDVDLTLNWGRLSSVLPDYH |
| | | GQDSVRVGRISFGSINAILGSVALILNCHHHASAVAA |
| SEQ ID | Shiga toxin effector | KEFILRFSVAHKYVDSLNVIRSAIGTPLQTISSGGTSLLMIDSGS |
| NO: 50 | polypeptide 11 | GDNLFAVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDNLV |
| NO: 51 | polypeptide 12 | PMVATVVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDSNL |
| NO: 52 | polypeptide 13 | VPMVATVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDSGI |
| NO: 53 | polypeptide 14 | LGFVFTLDVRGIDPEEGRFNNLRLIVERNNLYVTGFVNRTNNV |
| | | FYRFADFSHVTFPGTTAVTLSGDSSYTTLQRVAGISRTGMQIN |
| | | RHSLTTSYLDLMSHSGTSLTQSVARAMLRFVTVTAEALRFRQI |
| | | QRGFRTTLDDLSGRSYVMTAEDVDLTLNWGRLSSVLPDYHG |
| | | QDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDSGS |
| NO: 54 | polypeptide 15 | GDNLFAVGILGFDFTLGRFNNLRLIVERNNLYVTGFVNRTNNV |
| | | FYRFADFSHVTFPGTTAVTLSGDSSYTTLQRVAGISRTGMQIN |
| | | RHSLTTSYLDLMSHSGTSLTQSVARAMLRFVTVTAEALRFRQI |
| | | QRGFRTTLDDLSGRSYVMTAEDVDLTLNWGRLSSVLPDYHG |
| | | QDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDSGS |
| NO: 55 | polypeptide 16 | GDNLFAVDILGFVFTLGRFNNLRLIVERNNLYVTGFVNRTNNV |
| | | FYRFADFSHVTFPGTTAVTLSGDSSYTTLQRVAGISRTGMQIN |
| | | RHSLTTSYLDLMSHSGTSLTQSVARAMLRFVTVTAEALRFRQI |
| | | QRGFRTTLDDLSGRSYVMTAEDVDLTLNWGRLSSVLPDYHG |
| | | QDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDSGS |
| NO: 56 | polypeptide 17 | GDNLFAVDILGFDFTLGRFNNLRLIVERNNLYVTGFVNRTNNV |
| | | FYRFADFSHVTFPGTTAVTLSGDSSYTTLQRVAGISRTGMQIN |
| | | RHSLTTSYLDLMSHSGTSLTQSVARAMLRFVTVTAEALRFRQI |
| | | QRGFRTTLDDLSGRSYVMTAEDVDLTLNWGRLSSVLPDYHG |
| | | QDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDSGS |
| NO: 57 | polypeptide 18 | GDNLFAVGILGFVFTLGRFNNLRLIVERNNLYVTGFVNRTNNV |
| | | FYRFADFSHVTFPGTTAVTLSGDSSYTTLQRVAGISRTGMQIN |
| | | RHSLTTSYLDLMSHSGTSLTQSVARAMLRFVTVTAEALRFRQI |
| | | QRGFRTTLDDLSGRSYVMTAEDVDLTLNWGRLSSVLPDYHG |
| | | QDSVRVGRISFGSINAILGSVALILNCHHHASAVAA |
| SEQ ID | Shiga toxin effector | KEFILDFSTAKTYVDSLNVIRSAIGTPLQTISSGGTSLLMIDSGIG |
| NO: 58 | polypeptide 19 | DNLFAVDVRGIAPIEARFNNLRLIVERNNLYVTGFVNRTNNVF |
| | | YRFADFSHVTFPGTTAVTLSADSSYTTLQRVAGISRTGMQINR |
| | | HSLTTSYLDLMSHSATSLTQSVARAMLRFVTVTAEALRFRQIQ |
| | | RGFRTTLAALSGASYVMTAEDVDLTLNWGRLSSVLPDYHGQ |
| | | DSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGS |
| NO: 59 | polypeptide 20 | GDNLFAVGILGFVFTLEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDSGS |
| NO: 60 | polypeptide 21 | GDNLFAVNLVPMVATVGRENNLRLIVERNNLYVTGFVNRTN |
| | | NVFYRFADFSHVTFPGTTAVTLSGDSSYTTLQRVAGISRTGMQ |
| | | INRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDSGS |
| NO: 61 | polypeptide 22 | GDNLFAVDVRGIDPEEGRFNNLRLIVERNNLYVTGFVNRTNN |
| | | VFYRFADFSHVTFPGTNLVPMVATVSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDSGS |
| NO: 62 | polypeptide 23 | GDNLFAVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRGILGDVFTLSYVMTAEDVDLTLNWGRLSSVLPDYH |
| | | GQDSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGS |
| NO: 63 | polypeptide 24 | GDNLFAVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHILRFSVAHKASAV |
| | | AA |
| SEQ ID | Shiga toxin effector | KEFTLDFSTAKTYVDSLNVIRSAIGTPLQTISSGGTSLLMIDSGS |
| NO: 64 | polypeptide 25 | GDNLFAVDILGFDFTLGRFNNLRLIVERNNLYVTGFVNRTNNV |
| | | FYRFADFSHVTFPGTTAVTLSGDSSYTTLQRVAGISRTGMQIN |
| | | RHSLTTSYLDLMSHSGTSLTQSVARAMLRFVTVTAEALRFRQI |
| | | QRGFRTTLDDLSGRSYVMTAEDVDLTLNWGRLSSVLPDYHG |
| | | QDSVRVGRISFGSINAILGSVALILNCHHHARNLVPMVATVAS |
| | | AVAA |
| SEQ ID | Shiga toxin effector | KEFTLDFSTAKTYVDSLNVIRSAIGTPLQTISSGGTSLLMIDSGS |
| NO: 65 | polypeptide 26 | GDNLFAVDVRGIDPEEGRFNNLRLIVERNNLYVTGFVNRTNN |
| | | VFYRFADFSHVTFPGTNLVPMVATVSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHASAVAA |
| SEQ ID | Shiga toxin effector | KEFTLDFSTAKTYVDSLNVIRSAIGTPLQTISSGGTSLLMIDSGS |
| NO: 66 | polypeptide 27 | GDNLFAVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRGILGDVFTLSYVMTAEDVDLTLNWGRLSSVLPDYH |
| | | GODSVRVGRISFGSINAILGSVALILNSHHHASAVAA |
| SEQ ID | Shiga toxin effector | KEFTLDFSTAKTYVDSLNVIRSAIGTPLQTISSGGTSLLMIDSGS |
| NO: 67 | polypeptide 28 | GDNLFAVDVRGIDPEEGRFNNLRLIVERNNLYVTGFVNRTNN |
| | | VFYRFADFSHVTFPGTTAVTLSGDSSYTTLQRVAGISRTGMQI |
| | | NRHSLTTSYLDLMSHSGTSLTQSVARAMLRFVTVTAEALRFR |
| | | QIQRGFRTTLDDLSGRSYVMTAEDVDLTLNWGRLSSVLPDYH |
| | | GQDSVRVGRISFGSINAILGSVALILNCHHHILRFSVAHKASAV |
| | | AA |
| SEQ ID | Shiga toxin effector | KEFTLDFSTAKTYVDSLNVIRSAIGTPLQTISSGGTSLLMIDSGS |
| NO: 68 | polypeptide 29 | GDNLFAVDILGFDFTLGRFNNLRLIVERNNLYVTGFVNRTNNV |
| | | FYRFADFSHVTFPGTTAVTLSGDSSYTTLQRVAGISRTGMQIN |
| | | RHSLTTSYLDLMSHSGTSLTQSVARAMLRFVTVTAEALRFRQI |
| | | QRGFRTTLDDLSGRSYVMTAEDVDLTLNWGRLSSVLPDYHG |
| | | QDSVRVGRISFGSINAILGSVALILNCHHHARNLVPMVATVAS |
| | | AVAA |

Any of the protease-cleavage resistant, Shiga toxin effector polypeptide sub-regions and/or disrupted furin-cleavage sites described herein can be used alone or in combination with each individual embodiment described herein, including methods described herein.

### IV. Binding Molecules

The following embodiments describe in more detail the structures of illustrative binding molecules which bind CTLA-4 on the surface of an immunosuppressive cell, such as an immunosuppressive immune cell that expresses CTLA-4.

In some embodiments, the binding molecules are fusion proteins that comprise or consist of, from N-terminus to C-terminus or from C-terminus to N-terminus, a Shiga toxin A subunit effector polypeptide and a CTLA-4-binding region. The binding molecule can further comprise a linker that links the Shiga toxin A subunit effector polypeptide and the CTLA-4-binding region. In some embodiments, the CTLA-4-binding region comprises a VHH domain. In some embodiments, the VHH domain comprises the amino acid sequence of SEQ ID NO: 21 or SEQ ID NO: 22.

In some embodiments, the binding molecule comprises or consists of, from N-terminus to C-terminus or from C-terminus to N-terminus, a Shiga toxin A subunit effector polypeptide, a linker, a first CTLA-4-binding domain, a linker, and a second CTLA-4-binding domain. In some embodiments, the first CTLA4 binding domain is a first VHH domain and the second CTLA4 binding domain is a second VHH domain.

In some embodiments, the binding molecule comprises or consists of, from N-terminus to C-terminus, a Shiga toxin A subunit effector polypeptide, a linker, a first VHH domain, a linker, and a second VHH domain. In some embodiments, the Shiga toxin A subunit effector polypeptide comprises the amino acid sequence of SEQ ID NO: 41. In some embodiments, the linker between the Shiga toxin A subunit effector polypeptide and the first VHH domain comprises the amino acid sequence of SEQ ID NO: 218. In some embodiments, the first VHH domain comprises the amino acid sequence of SEQ ID NO: 21. In some embodiments, the linker between the first VHH domain and the second VHH domain comprises the amino acid sequence of SEQ ID NO: 29. In some embodiments, the second VHH domain comprises the amino acid sequence of SEQ ID NO: 22.

In some embodiments, the CTLA-4 binding molecule comprises the amino acid sequence of SEQ ID NO: 329. In some embodiments, the CTLA-4 binding molecule comprises an amino acid sequence at least 90%, at least 91%, at least 92%, at least 93%, at least 94%, at least 95%, at least 96%, at least 97%, at least 98%, or at least 99% identical to the amino acid sequence of SEQ ID NO: 329.

In some embodiments, the CTLA-4 binding molecule comprises any one of the amino acid sequences of SEQ ID NOs: 286-396. In some embodiments, the CTLA-4 binding molecule comprises an amino acid sequence at least 90%, at least 91%, at least 92%, at least 93%, at least 94%, at least 95%, at least 96%, at least 97%, at least 98%, or at least 99% identical to any one of the amino acid sequences of SEQ ID NOs: 286-396.

In some embodiments, the binding molecule comprises, from N-terminus to C-terminus or from C-terminus to N-terminus, a Shiga toxin A subunit effector polypeptide, a linker, and a binding region.

In some embodiments, the binding molecule is a single continuous polypeptide. In some embodiments, the binding molecule is a fusion protein.

In some embodiments, the binding molecule comprises two polypeptides. For example, in some embodiments, the binding molecule is a dimeric binding molecule (e.g., a homodimeric binding molecule) that comprises two monomeric binding molecules, wherein each monomer comprises or consists of, from N-terminus to C-terminus or from C-terminus to N-terminus, a Shiga toxin A subunit effector polypeptide and a binding region. The monomers can each comprise a linker that links the Shiga toxin A subunit effector polypeptide and the binding region. The monomers can be covalently or non-covalently linked. For example, the dimeric binding molecule can further comprise a linker that links the two monomers. In some embodiments, the dimer comprises two or more T-cell epitopes for delivery to the interior of a target cell and subsequent cell-surface presentation. In embodiments wherein the dimeric binding molecule comprises two or more epitopes for delivery, the epitopes may be the same or different.

In some embodiments, a binding molecule comprises from N-terminus to C-terminus or from C-terminus to N-terminus: (i) a Shiga toxin A subunit effector polypeptide, (ii) a binding region, and (iii) a T-cell epitope for delivery to the interior of a target cell and subsequent cell-surface presentation; or (i) a binding region, (ii) a Shiga toxin A subunit effector polypeptide, and (iii) a T-cell epitope for delivery to the interior of a target cell and subsequent cell-surface presentation.

In some embodiments, a binding molecule comprises from N-terminus to C-terminus or from C-terminus to N-terminus, (i) a Shiga toxin A subunit effector polypeptide, (ii) a T-cell epitope for delivery to the interior of a target cell and subsequent cell-surface presentation, and (iii) a binding region.

In some embodiments, a binding molecule comprises from N-terminus to C-terminus: (i) a T-cell epitope for delivery to the interior of a target cell and subsequent cell-surface presentation, (ii) a binding region, and (iii) a Shiga toxin A subunit effector polypeptide; or (i) a T-cell epitope for delivery to the interior of a target cell and subsequent cell-surface presentation, (ii) a Shiga toxin A subunit effector, and (iii) a binding region.

In some embodiments, the CTLA-4 binding molecule is cytotoxic. In some embodiments, the CTLA-4 binding molecule is non-cytotoxic. For example, the CTLA-4 binding molecule may be non-cytotoxic if the Shiga toxin subunit effector polypeptide is truncated or comprises one or more mutations which eliminate its cytotoxic activity.

In some embodiments, the linker that links the Shiga toxin subunit effector polypeptide to the binding region comprises or consists of the sequence SSGGGGSGGGGSGGGGSGGGGSGGGGS (SEQ ID NO: 199). In some embodiments, the linker comprises or consists of the sequence GSGSG (SEQ ID NO: 200).

Linkers of variable length can be used in the binding molecules described herein. In some embodiments, linkers of 1 to 50 amino acids in length are used. In some embodiments, a linker of 3 to 12 amino acids in length is used. In some embodiments, linkers of 5 amino acids in length are used. In some embodiments, linkers of longer than 12 (e.g., 13, 14, 15, 16, 17, 18, 19, or 20) amino acids in length are used.

An illustrative CLTA-4 binding molecule is provided below in Table 5. In some embodiments, the binding molecule comprises or consists of the polypeptide of SEQ ID NO: 329. Optionally, the binding molecule comprises an amino-terminal methionine residue. In some embodiments, the binding molecule has at least 90%, at least 91%, at least 92%, at least 93%, at least 94%, at least 95%, at least 96%, at least 97%, at least 98%, at least 99% amino acid sequence identity to SEQ ID NO: 329. In some embodiments, the binding molecule comprises or consists of two identical polypeptides, each polypeptide comprising a sequence at least 90%, at least 91%, at least 92%, at least 93%, at least 94%, at least 95%, at least 96%, at least 97%, at least 98%, at least 99% amino acid sequence identity to SEQ ID NO: 329. In some embodiments, the binding molecule comprises or consists of two identical polypeptides, each polypeptide comprising the sequence of SEQ ID NO: 329. In some embodiments, the binding molecule consists of two identical polypeptides, each polypeptide consisting of the sequence of SEQ ID NO: 329. In some embodiments, the CTLA-4 binding molecule comprises an amino acid sequence of SEQ ID NO: 329 with one or more mutations, such as 2, 3, 4, 5, 6, 7, 8, or 10, or more mutations. In some embodiments, the CTLA-4 binding molecule comprises an amino acid sequence of SEQ ID NO: 329 with 1-5, 5-10, 11-5, 15-20, 10-25, 25-30, or more than 30 mutations.

TABLE 5
Illustrative CTLA-4 binding molecule (CTLA-4 ETB 118421)

| Molecule or | | SEQ ID |
|---|---|---|
| region | Sequence | NO |
| CTLA-4 ETB | MKEFTLDFSTAKTYVDSLNVIRSAIGTPLQTISSGGTSLLMIDSGIGDNL | 329 |
| 118421 | FAVDILGFDFTLGRFNNLRLIVERNNLYVTGFVNRTNNVFYRFADFSH | |
| | VTFPGTTAVTLSADSSYTTLQRVAGISRTGMQINRHSLTTSYLDLMSH | |
| | SGTSLTQSVARAMLRFVTVTAEALRFRQIQRGFRTTLDDLSGASYVM | |
| | TAEDVDLTLNWGRLSSVLPDYHGQDSVRVGRISFGSINAILGSVALILN | |
| | SHHHASAVAAEFPKPSTPPGSSGGAP [symbol] | |
| | [symbol] [symbol] | |
| | [symbol] [symbol] | |
| | [symbol] GGGGSGGGSQVQLVESGGGLVQPGGSLRLSCEGTG | |
| | SIFSPNAMGWYRQGPGKQRELVASVTSSGTTNYSDSVKGRFTISMD | |
| | NYKHLAYLQMNSLKPEDTAVYYCNLKELGTFYRRDFWGQGTQVTVSS | |
| SLTA | KEFTLDFSTAKTYVDSLNVIRSAIGTPLQTISSGGTSLLMIDSGIGDNLF | 41 |
| | AVDILGFDFTLGRFNNLRLIVERNNLYVTGFVNRTNNVFYRFADFSHV | |
| | TFPGTTAVTLSADSSYTTLQRVAGISRTGMQINRHSLTTSYLDLMSHS | |
| | GTSLTQSVARAMLRFVTVTAEALRFRQIQRGFRTTLDDLSGASYVMT | |
| | AEDVDLTLNWGRLSSVLPDYHGQDSVRVGRISFGSINAILGSVALILN | |
| | SHHHASAVAA | |
| VHH1 domain | QVKLVESGGGSVQAGGSLRLSCAASGDSYSVKYMGWFRRAPGKQR | 21 |
| | DQREEVASIYPTGGTFYTDSVKGRFTISRDNAKSTLYLQMNSLKPEDT | |
| | AMYYCAAGKWGTDYWGQGTQVTVSS | |
| VHH2 domain | QVQLVESGGGLVQPGGSLRLSCEGTGSIFSPNAMGWYRQGPGKQR | 22 |
| | ELVASVTSSGTTNYSDSVKGRFTISMDNYKHLAYLQMNSLKPEDTAV | |
| | YYCNLKELGTFYRRDFWGQGTQVTVSS | |
| VHH1 HCDR1 | GDSYSVKYMG | 23 |
| VHH1 HCDR2 | SIYPTGGTFYTDSVKGR | 24 |
| VHH1 HCDR3 | AGKWGTDY | 25 |
| VHH2 HCDR1 | PNAMG | 26 |
| VHH2 HCDR2 | SVTSSGTTNYSDSVKG | 27 |
| VHH2 HCDR3 | KELGTFYRRDF | 28 |
| Binding region | EFPKPSTPPGSSGGAP | 218 |
| linker | | |
| VHH linker | GGGGSGGGS | 29 |
| G4SG3S | | |

In the CTLA-4 ETB 118421 sequence, the SLTA is italicized, the binding region linker is underlined, the VHH1 domain is italicized and bolded, the VHH linker is italicized and underlined, and the VHH2 domain is bolded. In the VHH1 and VHH2 domains, the CDR sequences are underlined.

Many variations of the CTLA-4 binding molecule of Table 5 may also be prepared, as described herein. For example, the length of one or more linkers in the molecule may be increased or decreased. In some embodiments, the CTLA-4 binding molecule may also be linked to an antigen as described herein.

In some embodiments, the CTLA-4 binding molecules of the invention are monomers. In some embodiments, the CTLA-4 binding proteins are dimers, such as homodimers or heterodimers. In some embodiments, the CTLA-4 binding proteins are homodimers comprising two identical polypeptides. In some embodiments, the CTLA-4 binding proteins are multimers comprising, for example, two, three, four, five, six, seven, eight, nine, ten, or more CTLA-4-binding polypeptides.

As described above, individual binding regions, toxin components, and/or other components of the binding molecules described herein may be suitably linked to each other, such as, e.g., fused directly or indirectly linked to each other via linkers well known in the art and/or described herein. In some embodiments, a linker can be used to link a Shiga toxin A subunit effector polypeptide to a binding region. In some embodiments, a linker can be used to link a first binding molecule monomer to a second binding molecule monomer, to form a dimeric binding molecule (e.g., a homodimeric binding molecule).

Suitable linkers are generally those which allow each polypeptide component of the binding molecules described herein to fold with a three-dimensional structure very similar to the polypeptide components produced individually without any linker or other component. Suitable linkers include single amino acids, peptides, polypeptides, and linkers lacking any of the aforementioned, such as various non-proteinaceous carbon chains, whether branched or cyclic.

Suitable linkers can be proteinaceous and comprise amino acids, peptides, and/or polypeptides. Proteinaceous linkers are suitable for both recombinant binding molecules and chemically linked conjugates. A proteinaceous linker typically has from about 2 to about 50 amino acid residues, such as, e.g., from about 5 to about 30 or from about 6 to about 25 amino acid residues. The length of the linker selected will depend upon a variety of factors, such as, e.g., the desired property or properties for which the linker is being selected. In some embodiments, the linker is proteinaceous and is linked near the terminus of a protein component of the binding molecules described herein, typically within about 20 amino acids of the terminus.

Suitable linkers can be non-proteinaceous, such as, e.g. chemical linkers. Various non-proteinaceous linkers known in the art can be used to link cell-targeting binding regions to the Shiga toxin effector polypeptide components of the binding molecules, such as linkers commonly used to conjugate immunoglobulin polypeptides to heterologous polypeptides. For example, polypeptide regions can be linked using the functional side chains of their amino acid residues and carbohydrate moieties such as, e.g., a carboxy, amine, sulfhydryl, carboxylic acid, carbonyl, hydroxyl, and/or cyclic ring group. For example, disulfide bonds and thioether bonds can be used to link two or more polypeptides. In addition, non-natural amino acid residues can be used with other functional side chains, such as ketone groups (see e.g. Axup J et al., Proc Natl Acad Sci U.S.A. 109: 16101-6 (2012); Sun S et al., Chembiochem July 18 (2014); Tian F et al., Proc Natl Acad Sci USA 111: 1766-71 (2014)). In addition, non-natural amino acid residues can be used with other functional side chains, such as ketone groups, alkyne groups, or azides (see e.g. the use of antibodies engineered to comprise p-acetyl-L-phenylalanine or p-azidomethyl-N-phenylalanine residues for conjugation to cargos (US 2016/0082119, US 2017/0362334)). Examples of non-proteinaceous chemical linkers include but are not limited to N-hydroxysuccinimide esters (NHS esters) such as sulfo-NHS esters, isothiocyanates, isocyanates, acyl azides, sulfonyl chlorides, aldehydes, glyoxals, epoxides, oxiranes, carbonates, aryl halides, imidoesters, carbodiimides, anhydrides, and fluorophenyl esters. Further examples of non-proteinaceous chemical linkers include but are not limited to N-succinimidyl (4-iodoacetyl)-aminobenzoate, S-(N-succinimidyl) thioacetate (SATA), N-succinimidyl-oxycarbonyl-cu-methyl-α-(2-pyridyldithio) toluene (SMPT), N-succinimidyl 4-(2-pyridyldithio)-pentanoate (SPP), succinimidyl 4-(N-maleimidomethyl) cyclohexane carboxylate (SMCC or MCC), sulfosuccinimidyl (4-iodoacetyl)-aminobenzoate, 4-succinimidyl-oxycarbonyl-a-(2-pyridyldithio) toluene, sulfosuccinimidyl-6-(α-methyl-α-(pyridyldithiol)-toluamido) hexanoate, N-succinimidyl-3-(−2-pyridyldithio)-proprionate (SPDP), succinimidyl 6(3(-(−2-pyridyldithio)-proprionamido) hexanoate, sulfosuccinimidyl 6(3(-(−2-pyridyldithio)-propionamido) hexanoate, maleimidocaproyl (MC), maleimidocaproyl-valine-citrulline-p-aminobenzyloxycarbonyl (MC-vc-PAB), 3-maleimidobenzoic acid N-hydroxysuccinimide ester (MBS), alpha-alkyl derivatives, sulfoNHS-ATMBA (sulfosuccinimidyl N-[3-(acetylthio)-3-methylbutyryl-beta-alanine]), sulfodichlorophenol, 2-iminothiolane, 3-(2-pyridyldithio)-propionyl hydrazide, Ellman's reagent, dichlorotriazinic acid, and S-(2-thiopyridyl)-L-cysteine.

Suitable linkers, whether proteinaceous or non-proteinaceous, can include, e.g., protease sensitive, environmental redox potential sensitive, pH sensitive, acid cleavable, photocleavable, and/or heat sensitive linkers.

Proteinaceous linkers can be chosen for incorporation into recombinant binding molecules. For recombinant binding molecules, linkers typically comprise about 2 to 50 amino acid residues, preferably about 5 to 30 amino acid residues. Commonly, proteinaceous linkers comprise a majority of amino acid residues with polar, uncharged, and/or charged residues, such as, e.g., threonine, proline, glutamine, glycine, and alanine. Non-limiting examples of proteinaceous linkers include alanine-serine-glycine-glycine-proline-glutamate (ASGGPE, SEQ ID NO: 201), valine-methionine (VM), alanine-methionine (AM), AM(G_{2 to 4}S)ₓAM where G is glycine, S is serine, and x is an integer from 1 to 10 (SEQ ID NO: 202).

Proteinaceous linkers can be selected based upon the properties desired. Proteinaceous linkers can be chosen by the skilled worker with specific features in mind, such as to optimize the binding molecule's folding, stability, expression, solubility, pharmacokinetic properties, pharmacodynamic properties, and/or the activity of the fused domains in the context of a fusion construct as compared to the activity of the same domain by itself. For example, proteinaceous linkers can be selected based on flexibility, rigidity, and/or cleavability. The skilled worker can use databases and linker design software tools when choosing linkers. In certain linkers can be chosen to optimize expression. In certain linkers can be chosen to promote intermolecular interactions between identical polypeptides or proteins to form homomultimers or different polypeptides or proteins to form heteromultimers. For example, proteinaceous linkers can be selected which allow for desired non-covalent interactions between polypeptide components of the binding molecules, such as, e.g., interactions related to the formation dimers and other higher order multimers.

Flexible proteinaceous linkers are often greater than 12 amino acid residues long and rich in small, non-polar amino acid residues, polar amino acid residues, and/or hydrophilic amino acid residues, such as, e.g., glycines, serines, and threonines. Flexible proteinaceous linkers can be chosen to increase the spatial separation between components and/or to allow for intramolecular interactions between components. For example, various “GS” linkers are known to the skilled worker and are composed of multiple glycines and/or serines, sometimes in repeating units, such as, e.g., (GₓS)ₙ, (SEQ ID NO: 203), (SₓG)ₙ (SEQ ID NO: 204), (GGGGS)ₙ (SEQ ID NO: 205), and (G)ₙ (SEQ ID NO: 206), in which x is 1 to 6 and n is 1 to 30. Non-limiting examples of flexible proteinaceous linkers include GKSSGSGSESKS (SEQ ID NO: 207), EGKSSGSGSESKEF (SEQ ID NO: 208), GSTSGSGKSSEGKG (SEQ ID NO: 209), GSTSGSGKSSEGSGSTKG (SEQ ID NO: 210), GSTSGSGKPGSGEGSTKG (SEQ ID NO: 211), SRSSG (SEQ ID NO: 212), and SGSSC (SEQ ID NO: 213).

Rigid proteinaceous linkers are often stiff alpha-helical structures and rich in proline residues and/or strategically placed prolines. Rigid linkers can be chosen to prevent intramolecular interactions between linked components. In some embodiments, a rigid linker is EAAAK (SEQ ID NO: 285).

Additional examples of suitable linkers are provided in Table 6.

TABLE 6
Linkers

| | | SEQ ID |
|---|---|---|
| Linker name | Sequence | NO |
| linker 1 | GGGGSGGGGSGGGGSGGGGSGGGGS | 214 |
| linker 2 | GGGGSGGGGSGGGGSGGGGS | 215 |
| linker 3 | GSTSGSGKPGSGEGSTKG | 216 |
| linker 4 | GGGGS | 217 |
| linker 5 | EFPKPSTPPGSSGGAP | 218 |
| linker 6 | EFPKPSTPPGSSGGAPGILGFVFTL | 219 |
| linker 7 | GSTSGSGKPGSGEGS | 220 |
| linker 8 | SPSTPPTPSPSTPPAS | 221 |
| linker 9 | AHHSEDPSSKAPKAP | 222 |

Suitable linkers can allow for in vivo separation of components, such as, e.g., due to cleavage and/or environment-specific instability. In vivo cleavable proteinaceous linkers are capable of unlinking by proteolytic processing and/or reducing environments often at a specific site within an organism or inside a certain cell type. In vivo cleavable proteinaceous linkers often comprise protease sensitive motifs and/or disulfide bonds formed by cysteine pairs. In vivo cleavable proteinaceous linkers can be designed to be sensitive to proteases that exist only at certain locations in an organism, compartments within a cell, and/or become active only under certain physiological or pathological conditions (such as, e.g., involving proteases with abnormally high levels, proteases overexpressed at certain disease sites, and proteases specifically expressed by a pathogenic microorganism). For example, there are proteinaceous linkers known in the art which are cleaved by proteases present only intracellularly, proteases present only within specific cell types, and proteases present only under pathological conditions like cancer or inflammation, such as, e.g., R-x-x-R motif (SEQ ID NO: 195) and AMGRSGGGCAGNRVGSSLSCGGLNLQAM (SEQ ID NO: 223).

In some embodiments, a linker can comprise a protease sensitive site to provide for cleavage by a protease present within a target cell. In some embodiments, the linker is not cleavable, so as to reduce unwanted toxicity after administration to a vertebrate organism.

Suitable linkers include, e.g., protease sensitive, environmental redox potential sensitive, pH sensitive, acid cleavable, photocleavable, and/or heat sensitive linkers, whether proteinaceous or non-proteinaceous (see e.g., Doronina S et al., Bioconjug Chem 17: 114-24 (2003); Saito G et al., Adv Drug Deliv Rev 55: 199-215 (2003); Jeffrey S et al., J Med Chem 48: 1344-58 (2005); Sanderson R et al., Clin Cancer Res 11: 843-52 (2005); Erickson H et al., Cancer Res 66: 4426-33 (2006); Chen X et al., Adv Drug Deliv Rev 65: 1357-69 (2013)). Suitable cleavable linkers can include linkers comprising cleavable groups which are known in the art.

Suitable linkers can include pH sensitive linkers. For example, certain suitable linkers can be chosen for their instability in lower pH environments to provide for dissociation inside a subcellular compartment of a target cell (see e.g., van Der Velden V et al., Blood 97: 3197-204 (2001); Ulbrich K, Subr V, Adv Drug Deliv Rev 56: 1023-50 (2004)). For example, linkers that comprise trityl groups, derivatized trityl groups, bismaleimideothoxy propane groups, adipic acid dihydrazide groups, and/or acid labile transferrin groups, can provide for release of components of the binding molecules, e.g. a polypeptide component, in environments with specific pH ranges. In certain linkers can be chosen which are cleaved in pH ranges corresponding to physiological pH differences between tissues, such as, e.g., the pH of tumor tissue is lower than in healthy tissues.

Photocleavable linkers are linkers that are cleaved upon exposure to electromagnetic radiation of certain wavelength ranges, such as light in the visible range. Photocleavable linkers can be used to release a component of a binding molecule, e.g. a polypeptide component, upon exposure to light of certain wavelengths. Non-limiting examples of photocleavable linkers include a nitrobenzyl group as a photocleavable protective group for cysteine, nitrobenzyloxycarbonyl chloride cross-linkers, hydroxypropylmethacrylamide copolymer, glycine copolymer, fluorescein copolymer, and methylrhodamine copolymer. Photocleavable linkers can have particular uses in linking components to form binding molecules designed for treating diseases, disorders, and conditions that can be exposed to light using fiber optics.

In some embodiments, a CTLA-4 binding region is linked to a Shiga toxin effector polypeptide using any number of means known to the skilled worker, including both covalent and noncovalent linkages.

In some embodiments, the binding molecules comprise a binding region which is a scFv with a linker connecting a heavy chain variable (V_{H}) domain and a light chain variable (V_{L}) domain. There are numerous linkers known in the art suitable for this purpose, such as, e.g., the 15-residue (Gly4Ser)₃ peptide (SEQ ID NO: 215). Suitable scFv linkers which can be used in forming non-covalent multivalent structures include GGS (SEQ ID NO: 224), GGGS (SEQ ID NO: 225), GGGGS (SEQ ID NO: 226), GGGGSGGG (SEQ ID NO: 227), GGSGGGG (SEQ ID NO: 228), GSTSGGGSGGGSGGGGSS (SEQ ID NO: 229), and GSTSGSGKPGSSEGSTKG (SEQ ID NO: 230).

In some embodiments, the binding molecules, or a polypeptide component thereof, comprise a carboxy-terminal, endoplasmic reticulum retention/retrieval signal motif of a member of the KDEL family. In some embodiments, the carboxy-terminal endoplasmic reticulum retention/retrieval signal motif is: KDEL (SEQ ID NO: 231), HDEF (SEQ ID NO: 232), HDEL (SEQ ID NO: 233), RDEF (SEQ ID NO: 234), RDEL (SEQ ID NO: 235), WDEL (SEQ ID NO: 236), YDEL (SEQ ID NO: 237), HEEF (SEQ ID NO: 238), HEEL (SEQ ID NO: 239), KEEL (SEQ ID NO: 240), REEL (SEQ ID NO: 241), KAEL (SEQ ID NO: 242), KCEL (SEQ ID NO: 243), KFEL (SEQ ID NO: 244), KGEL (SEQ ID NO: 245), KHEL (SEQ ID NO: 246), KLEL (SEQ ID NO: 247), KNEL (SEQ ID NO: 248), KQEL (SEQ ID NO: 249), KREL (SEQ ID NO: 250), KSEL (SEQ ID NO: 251), KVEL (SEQ ID NO: 252), KWEL (SEQ ID NO: 253), KYEL (SEQ ID NO: 254), KEDL (SEQ ID NO: 255), KIEL (SEQ ID NO: 256), DKEL (SEQ ID NO: 257), FDEL (SEQ ID NO: 258), KDEF (SEQ ID NO: 259), KKEL (SEQ ID NO: 260), HADL (SEQ ID NO: 261), HAEL (SEQ ID NO: 262), HIEL (SEQ ID NO: 263), HNEL (SEQ ID NO: 264), HTEL (SEQ ID NO: 265), KTEL (SEQ ID NO: 266), HVEL (SEQ ID NO: 267), NDEL (SEQ ID NO: 268), QDEL (SEQ ID NO: 269), REDL (SEQ ID NO: 270), RNEL (SEQ ID NO: 271), RTDL (SEQ ID NO: 272), RTEL (SEQ ID NO: 273), SDEL (SEQ ID NO: 274), TDEL (SEQ ID NO: 275), SKEL (SEQ ID NO: 276), STEL (SEQ ID NO: 277), and EDEL (SEQ ID NO: 278). In some embodiments, the binding molecule when introduced into a cell is capable cytotoxicity that is greater than that of a binding molecule consisting of the binding molecule except for it does not comprise any carboxy-terminal, endoplasmic reticulum retention/retrieval signal motif of the KDEL family. In some embodiments, the binding molecule is capable of exhibiting a cytotoxicity with better optimized, cytotoxic potency, such as, e.g., 4-fold, 5-fold, 6-fold, 9-fold, or greater cytotoxicity as compared to a reference molecule.

In some embodiments, the binding molecules comprise a T-cell epitope peptide. The T-cell epitope peptide can be embedded in the Shiga toxin A subunit effector polypeptide, in the binding region, in the linker, or otherwise incorporated into the binding molecule. In some embodiments, the epitope peptide is a CD8+ T-cell epitope. In some embodiments, the CD8+ T-cell epitope peptide has a binding affinity to a MHC class I molecule characterized by a dissociation constant (KD) of 10⁻⁴ molar or less (e.g., 10⁻⁴ molar to 10⁻⁶ molar) and/or the resulting MHC class I-epitope-peptide complex has a binding affinity to a T-cell receptor (TCR) characterized by a dissociation constant (KD) of 10⁻⁴ molar or less (e.g., 10⁻⁴ molar to 10⁻⁶ molar).

T-cell epitopes can be obtained or derived from various naturally occurring proteins. T-cell epitopes can be created or derived from various naturally occurring proteins foreign to mammals, such as, e.g., proteins of microorganisms. T-cell epitopes can be created or derived from mutated human proteins and/or human proteins aberrantly expressed by malignant human cells. T-cell epitopes can be synthetically created or derived from synthetic molecules (see e.g., Carbone F et al., J Exp Med 167: 1767-9 (1988); Del Val M et al., J Virol 65: 3641-6 (1991); Appella E et al., Biomed Pept Proteins Nucleic Acids 1: 177-84 (1995); Perez S et al., Cancer 116: 2071-80 (2010)). T-cell epitope components can be chosen or derived from a number of source molecules already known to be capable of eliciting a vertebrate immune response. T-cell epitopes can be derived from various naturally occurring proteins foreign to vertebrates, such as, e.g., proteins of pathogenic microorganisms and non-self, cancer antigens. In particular, infectious microorganisms can contain numerous proteins with known antigenic and/or immunogenic properties. Further, infectious microorganisms can contain numerous proteins with known antigenic and/or immunogenic sub-regions or epitopes.

In some embodiments, proteins of intracellular pathogens with mammalian hosts are sources for T-cell epitopes. There are numerous intracellular pathogens, such as viruses, bacteria, fungi, and single-cell eukaryotes, with well-studied antigenic proteins or peptides. T-cell epitopes can be selected or identified from human viruses or other intracellular pathogens, such as, e.g., bacteria like mycobacterium, fungi like toxoplasmae, and protists like trypanosomes. In some embodiments, the T-cell epitope is isolated or derived from proteins from influenza A viruses, such as peptides in the proteins HA glycoproteins FE17, S139/1, CH65, C05, hemagglutinin 1 (HA1), hemagglutinin 2 (HA2), nonstructural protein 1 and 2 (NS1 and NS 2), matrix protein 1 and 2 (M1 and M2), nucleoprotein (NP), neuraminidase (NA)). In some embodiments, the T-cell epitope is isolated or derived from proteins from human cytomegaloviruses (HCMV), such as peptides in the proteins pp65 (UL83), UL128-131, immediate-early 1 (IE-1; UL123), glycoprotein B, tegument proteins.

In some embodiments, the T-cell epitope is isolated or derived from an immunogenic cancer antigen in humans. The CD8+ T-cell epitopes of cancer and/or tumor cell antigens can be identified by the skilled worker using techniques known in the art, such as, e.g., differential genomics, differential proteomics, immunoproteomics, prediction then validation, and genetic approaches like reverse-genetic transfection (see e.g., Admon A et al., Mol Cell Proteomics 2: 388-98 (2003); Purcell A, Gorman J, Mol Cell Proteomics 3: 193-208 (2004); Comber J, Philip R, Ther Adv Vaccines 2: 77-89 (2014)). There are many antigenic and/or immunogenic T-cell epitopes already identified or predicted to occur in human cancer and/or tumor cells. For example, T-cell epitopes have been predicted in human proteins commonly mutated or overexpressed in neoplastic cells, such as, e.g., ALK, CEA, N-acetylglucosaminyl-transferase V (GnT-V), HCA587, PD-L1/neu, MAGE, Melan-A/MART-1, MUC-1, p53, and TRAG-3 (see e.g., van der Bruggen P et al., Science 254: 1643-7 (1991); Kawakami Y et al., J Exp Med 180: 347-52 (1994); Fisk B et al., J Exp Med 181: 2109-17 (1995); Guilloux Y et al., J Exp Med 183: 1173 (1996); Skipper J et al., J Exp Med 183: 527 (1996); Brossart P et al., 93: 4309-17 (1999); Kawashima I et al., Cancer Res 59: 431-5 (1999); Papadopoulos K et al., Clin Cancer Res 5: 2089-93 (1999); Zhu B et al., Clin Cancer Res 9: 1850-7 (2003); Li B et al., Clin Exp Immunol 140: 310-9 (2005); Ait-Tahar K et al., Int J Cancer 118: 688-95 (2006); Akiyama Y et al., Cancer Immunol Immunother 61: 2311-9 (2012)). In addition, synthetic variants of T-cell epitopes from human cancer cells have been created (see e.g., Lazoura E, Apostolopoulos V, Curr Med Chem 12: 629-39 (2005); Douat-Casassus C et al., J Med Chem 50: 1598-609 (2007)).

There are well-defined peptide-epitopes that are known to be immunogenic, MHC class I restricted, and/or matched with a specific human leukocyte antigen (HLA) variant(s). For applications in humans or involving human target cells, HLA-class !-restricted epitopes can be selected or identified by the skilled worker using standard techniques known in the art. The ability of peptides to bind to human MHC class I molecules can be used to predict the immunogenic potential of putative T-cell epitopes. The ability of peptides to bind to human MHC class I molecules can be scored using software tools. T-cell epitopes can be chosen for use as a heterologous, T-cell epitope component based on the peptide selectivity of the HLA variants encoded by the alleles more prevalent in certain human populations. For example, the human population is polymorphic for the alpha chain of MHC class I molecules due to the varied alleles of the HLA genes from individual to individual. In some embodiments, T-cell epitopes can be more efficiently presented by a specific HLA molecule, such as, e.g., the commonly occurring HLA variants encoded by the HLA-A allele groups HLA-A2 and HLA-A3.

In some embodiments, the particular epitope is that which best matches the MHC class I molecules present in the cell-type or cell populations to be targeted. Different MHC class I molecules exhibit preferential binding to particular peptide sequences, and particular peptide-MHC class I variant complexes are specifically recognized by the t-cell receptors (TCRs) of effector T-cells. The skilled worker can use knowledge about MHC class I molecule specificities and TCR specificities to optimize the selection of heterologous, T-cell epitopes used in the binding molecules.

Also provided are polynucleotides encoding one or more of the CTLA-4 binding molecules described thereof, or a complement thereof. Also provided are expression vectors comprising the polynucleotides described herein.

### Other Structural Variations

In some embodiments, the Shiga toxin effector polypeptides or binding molecules described herein, and polynucleotides encoding any of the former, include one or more variations that do not diminish the polypeptides', binding molecules' or polynucleotides' biological activities, e.g., by maintaining the overall structure and function of the Shiga toxin effector polypeptide, such as in conjunction with 1) endogenous epitope disruptions which reduce antigenic and/or immunogenic potential and/or 2) furin-cleavage site disruptions which reduce proteolytic cleavage. For example, some modifications can facilitate expression, facilitate purification, improve pharmacokinetic properties, and/or improve immunogenicity. Such modifications are well known to the skilled worker and include, for example, a methionine added at the amino-terminus to provide an initiation site, additional amino acids placed on either terminus to create conveniently located restriction sites or termination codons, and biochemical affinity tags fused to either terminus to provide for convenient detection and/or purification. A common modification to improve the immunogenicity of a polypeptide produced using a microbial system (e.g. a prokaryotic cell) is to remove, after the production of the polypeptide, the starting methionine residue, which can be formylated during production, such as, e.g., in a bacterial host system, because, e.g., the presence of N-formylmethionine (fMet) might induce undesirable immune responses in subjects such as in human subjects.

In some embodiments, the binding molecules described herein are modified by the inclusion of additional amino acid residues at the amino and/or carboxy termini, such as sequences for epitope tags or other moieties. The additional amino acid residues can be used for various purposes including, e.g., facilitating cloning, facilitating expression, post-translational modification, facilitating synthesis, purification, facilitating detection, and administration. Non-limiting examples of epitope tags and moieties are chitin binding protein domains, enteropeptidase cleavage sites, Factor Xa cleavage sites, FlAsH tags, FLAG tags, green fluorescent proteins (GFP), glutathione-S-transferase moieties, HA tags, maltose binding protein domains, myc tags, polyhistidine tags, ReAsH tags, strep-tags, strep-tag II, TEV protease sites, thioredoxin domains, thrombin cleavage site, and V5 epitope tags.

In some of the above embodiments, the polypeptide sequence of the Shiga toxin effector polypeptides and/or binding molecules are varied by conservative amino acid substitutions introduced into the polypeptide region(s) as long as all required structural features are still present and the Shiga toxin effector polypeptide is capable of exhibiting any required function(s), either alone or as a component of a binding molecule. As used herein, the term “conservative substitution” denotes that at least one amino acid is replaced by another, biologically similar amino acid residue. Examples include substitution of amino acid residues with similar characteristics, e.g. small amino acids, acidic amino acids, polar amino acids, basic amino acids, hydrophobic amino acids and aromatic amino acids (see, for example, Table 7). An example of a conservative substitution with a residue normally not found in endogenous, mammalian peptides and proteins is the conservative substitution of an arginine or lysine residue with, for example, ornithine, canavanine, aminoethylcysteine, or another basic amino acid. For further information concerning phenotypically silent substitutions in peptides and proteins see, e.g., Bowie J et al., Science 247: 1306-10 (1990).

TABLE 7
Examples of Conservative Amino Acid
Substitutions

| I | II | III | IV | V | VI | VII | VIII | IX | X | XI | XII | XIII | XIV |
|---|---|---|---|---|---|---|---|---|---|---|---|---|---|
| A | D | H | C | F | N | A | C | F | A | C | A | A | D |
| G | E | K | I | W | Q | G | M | H | C | D | C | C | E |
| P | Q | R | L | Y | S | I | P | W | F | E | D | D | G |
| S | N | | M | | T | L | | Y | G | H | G | E | K |
| T | | | V | | | V | | | H | K | N | G | P |
| | | | | | | | | | I | N | P | H | Q |
| | | | | | | | | | L | Q | S | K | R |
| | | | | | | | | | M | R | T | N | S |
| | | | | | | | | | R | S | V | Q | T |
| | | | | | | | | | T | T | | R | |
| | | | | | | | | | V | | | S | |
| | | | | | | | | | W | | | P | |
| | | | | | | | | | Y | | | T | |

In the conservative substitution scheme in Table 7, illustrative conservative substitutions of amino acids are grouped by physicochemical properties—I: neutral, hydrophilic; II: acids and amides; Ill: basic; IV: hydrophobic; V: aromatic, bulky amino acids, VI hydrophilic uncharged, VII aliphatic uncharged, VIII non-polar uncharged, IX cycloalkenyl-associated, X hydrophobic, XI polar, XII small, XIII turn-permitting, and XIV flexible. For example, conservative amino acid substitutions include the following: 1) S can be substituted for C; 2) M or L can be substituted for F; 3) Y can be substituted for M; 4) Q or E can be substituted for K; 5) N or Q can be substituted for H; and 6) H can be substituted for N.

Additional conservative amino acid substitutions include the following: 1) S can be substituted for C; 2) M or L can be substituted for F; 3) Y can be substituted for M; 4) Q or E can be substituted for K; 5) N or Q can be substituted for H; and 6) H can be substituted for N.

In some embodiments, the Shiga toxin effector polypeptides and binding molecules can comprise functional fragments or variants of a polypeptide region described herein that have, at most, 20, 15, 10, 9, 8, 7, 6, 5, 4, 3, 2, or 1 amino acid substitutions compared to a polypeptide sequence recited herein, as long as it comprises (1) a disrupted furin-cleavage site at the carboxy-terminus of a Shiga toxin A1 fragment derived region and (2) at least one amino acid disrupted in an endogenous, B-cell and/or CD4+ T-cell epitope region, wherein the disrupted amino acid does not overlap with the disrupted furin-cleavage site. Variants of the Shiga toxin effector polypeptides and binding molecules can be prepared by changing a polypeptide described herein by altering at least one amino acid residue or deleting or inserting at least one amino acid residue, such as within the binding region or Shiga toxin effector polypeptide region, in order to achieve desired properties, such as changed cytotoxicity, changed cytostatic effects, changed immunogenicity, and/or changed serum half-life. The Shiga toxin effector polypeptides and binding molecules described herein further be with or without a signal sequence.

Accordingly, in some embodiments, the Shiga toxin effector polypeptide comprises or consists essentially of amino acid sequences having at least 55%, at least 60%, at least 65%, at least 70%, at least 75%, at least 80%, at least 85%, at least 90%, at least 95%, at least 97%, at least 98%, or at least 99%, overall sequence identity to a naturally occurring Shiga toxin A Subunit or fragment thereof, such as, e.g., Shiga toxin A Subunit, such as SLT-1A (SEQ ID NO:1), StxA (SEQ ID NO:2), and/or SLT-2A (SEQ ID NO:3), wherein the Shiga toxin effector polypeptide has an activity associated with a naturally occurring SLT-1A subunit, e.g., target mediated internalization, catalytic activity and/or cytotoxic activity.

In some embodiments, the Shiga toxin effector polypeptide has at least one amino acid residue which is mutated, inserted, or deleted in order to increase the enzymatic activity of the Shiga toxin effector polypeptide. In some embodiments, the Shiga toxin effector polypeptide has at least one amino acid residue which are mutated or deleted in order to reduce or eliminate catalytic and/or cytotoxic activity of the Shiga toxin effector polypeptide. For example, the catalytic and/or cytotoxic activity of the A Subunits of members of the Shiga toxin family can be diminished or eliminated by mutation or truncation.

The cytotoxicity of the A Subunits of members of the Shiga toxin family can be altered, reduced, or eliminated by mutation and/or truncation. The positions labeled tyrosine-77, glutamate-167, arginine-170, tyrosine-114, and tryptophan-203 have been shown to be important for the catalytic activity of Stx, Stx1, and Stx2 (Hovde C et al., Proc Natl Acad Sci USA 85: 2568-72 (1988); Deresiewicz R et al., Biochemistry 31: 3272-80 (1992); Deresiewicz R et al., Mol Gen Genet 241: 467-73 (1993); Ohmura M et al., Microb Pathog 15: 169-76 (1993); Cao C et al., Microbiol Immunol 38: 441-7 (1994); Suhan M, Hovde C, Infect Immun 66: 5252-9 (1998)). Mutating both glutamate-167 and arginine-170 eliminated the enzymatic activity of Slt-I A1 in a cell-free ribosome inactivation assay (LaPointe P et al., J Biol Chem 280: 23310-18 (2005)). In another approach using de novo expression of Slt-I A1 in the endoplasmic reticulum, mutating both glutamate-167 and arginine-170 eliminated Slt-I A1 fragment cytotoxicity at that expression level (LaPointe P et al., J Biol Chem 280: 23310-18 (2005)). A truncation analysis demonstrated that a fragment of StxA from residues 75 to 268 still retains significant enzymatic activity in vitro (Haddad J et al., J Bacteriol 175: 4970-8 (1993)). A truncated fragment of Slt-I A1 containing residues 1-239 displayed significant enzymatic activity in vitro and cytotoxicity by de novo expression in the cytosol (LaPointe P et al., J Biol Chem 280: 23310-18 (2005)). Expression of a Slt-I A1 fragment truncated to residues 1-239 in the endoplasmic reticulum was not cytotoxic because it could not retrotranslocate to the cytosol (LaPointe P et al., J Biol Chem 280: 23310-18 (2005)).

Significant residues for enzymatic activity and/or cytotoxicity in the Shiga toxin A Subunits were mapped to the following residue-positions: asparagine-75, tyrosine-77, tyrosine-114, glutamate-167, arginine-170, arginine-176, and tryptophan-203 among others (Di R et al., Toxicon 57: 525-39 (2011)). In particular, a double-mutant construct of Stx2A containing glutamate-E167-to-lysine and arginine-176-to-lysine mutations was completely inactivated; whereas, many single mutations in Stx1 and Stx2 showed a 10-fold reduction in cytotoxicity. Further, truncation of Stx1A to 1-239 or 1-240 reduced its cytotoxicity, and similarly, truncation of Stx2A to a conserved hydrophobic residue reduced its cytotoxicity. Significant residues for binding eukaryotic ribosomes and/or eukaryotic ribosome inhibition in the Shiga toxin A Subunit have been mapped to the following residue-positions arginine-172, arginine-176, arginine-179, arginine-188, tyrosine-189, valine-191, and leucine-233 among others (McCluskey A et al., PLoS One 7: e31191 (2012). However, certain modification can increase a Shiga toxin functional activity exhibited by a Shiga toxin effector polypeptide. For example, mutating residue-position alanine-231 in Stx1A to glutamate increased Stx1A's enzymatic activity in vitro (Suhan M, Hovde C, Infect Immun 66: 5252-9 (1998)).

In some embodiments, the Shiga toxin effector polypeptide derived from SLT-1A (SEQ ID NO:1) or StxA (SEQ ID NO:2) has at least one amino acid residue mutated include substitution of the asparagine at position 75, tyrosine at position 77, tyrosine at position 114, glutamate at position 167, arginine at position 170, arginine at position 176, and/or substitution of the tryptophan at position 203. Examples of such substitutions will be known to the skilled worker based on the prior art, such as asparagine at position 75 to alanine, tyrosine at position 77 to serine, substitution of the tyrosine at position 114 to serine, substitution of the glutamate position 167 to glutamate, substitution of the arginine at position 170 to alanine, substitution of the arginine at position 176 to lysine, substitution of the tryptophan at position 203 to alanine, and/or substitution of the alanine at 231 with glutamate. Other mutations which either enhance or reduce Shiga toxin enzymatic activity and/or cytotoxicity may be used and can be determined using well known techniques and assays disclosed herein.

In some embodiments, a CTLA-4 binding molecule comprises an albumin binding domain or a portion of an albumin binding domain. In some embodiments, a CTLA-4 binding molecule comprises an albumin moiety (e.g., a serum albumin moiety). In other embodiments, a CTLA-4 binding molecule comprises an albumin binding domain, such as those derived from streptococcal protein G, e.g., ABD1, ABD2 and/or ABD3. Such albumination may extend the half-life of the binding molecule (Seijsing, J. et al., Front. Microbiol. (2018) 9 (2927): 1-9.

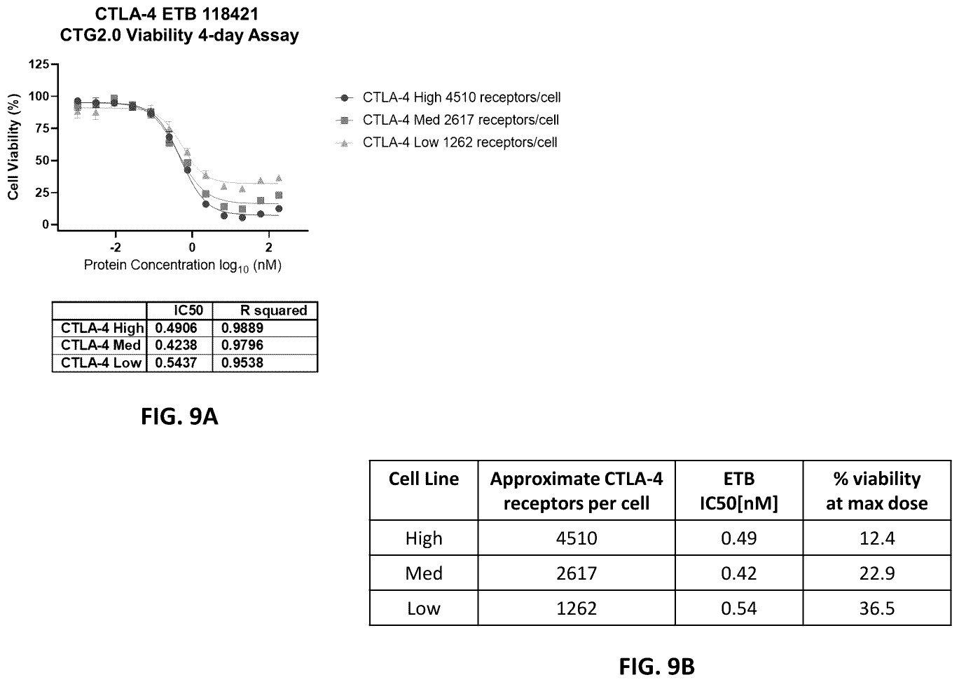

The critical contact residues on CTLA-4 for the VHH1 domain (SEQ ID NO: 21) and the VHH2 domain (SEQ ID NO: 22) were determined (see FIG. 7). The CTLA-4 critical contact residues for VHH1 are R at position 70, Q at position 76, K at position 130, and L at position 141. The CTLA-4 critical contact residues for VHH2 are E at position 59, K at position 65, N at position 110, and N at position 113.

### V. Methods of Making and Purifying

Also provided herein are methods for making and/or purifying binding molecules described herein. In some embodiments, the binding molecules are produced by recombinant expression in a host cell. The term “host cell” refers to a cell which can support the replication or expression of a nucleic acid (such as an expression vector) encoding a binding molecule. Host cells can be prokaryotic cells, such as E. coli or eukaryotic cells (e.g., yeast, insect, amphibian, bird, or mammalian cells). For example, immortalized cell lines such as Sf9, HEK293, CHO-K1, HeLa are often used as host cells. Creation and isolation of host cell lines comprising a nucleic acid or capable of producing a polypeptide and/or binding molecule can be accomplished using standard techniques known in the art.

In some embodiments, the methods comprise preparing a nucleic acid (e.g., an expression vector) encoding a binding molecule. In some embodiments, the methods comprise contacting a host cell with the nucleic acid (e.g., an expression vector) encoding the binding molecule. In some embodiments, the methods comprise introducing the nucleic acid into the host cell, for example by transfection, viral transduction (e.g., using a lentiviral or AAV vector), direct microinjection, particle bombardment, etc.

The binding molecule can be produced by culturing a host cell under conditions under which the binding molecule is expressed, and recovering the binding molecule. Culture conditions for producing recombinant proteins using various host cells are known to those of skill in the art. For example, in some embodiments, the host cell can be maintained in culture medium at 95° C. with 5% CO₂ atmosphere for a period of time sufficient to express the protein.

When a binding molecule is expressed using recombinant techniques in a host cell, it is advantageous to separate (or purify) the desired protein away from other components, such as host cell factors, in order to obtain preparations that are of high purity or are substantially homogeneous. Purification can be accomplished by methods well known in the art, such as centrifugation techniques, extraction techniques, chromatographic and fractionation techniques (e.g. size separation by gel filtration, charge separation by ion-exchange column, hydrophobic interaction chromatography, reverse phase chromatography, chromatography on silica or cation-exchange resins such as DEAE and the like, chromatofocusing, and Protein A Sepharose chromatography to remove contaminants), and precipitation techniques (e.g. ethanol precipitation or ammonium sulfate precipitation). Any number of biochemical purification techniques can be used to increase the purity of a polypeptide and/or binding molecule. In some embodiments, the polypeptides and binding molecules can optionally be purified in homo-multimeric forms (e.g., a molecular complex comprising two or more polypeptides or binding molecules).

In some embodiments, a method for making a CTLA-4 binding molecule as described herein comprises (a) expressing a CTLA-4 binding molecule in and (b) recovering the CTLA-4 binding molecule. In some embodiments, expressing the CTLA-4 binding molecule comprises culturing a host cell of claim under conditions wherein the CTLA-4 binding molecule is expressed. The host cell may comprise, for example, a nucleic acid or an expression vector encoding the CTLA-4 binding molecule or a fragment or variant thereof.

In embodiments, a method of making a CTLA-4 binding molecule comprises culturing a host cell under conditions wherein the CTLA-4 binding molecule is expressed, and recovering the protein. In some embodiments, the binding molecules comprise an epitope that allows them to be purified using affinity chromatography. In some embodiments, the binding molecules comprise an Ig binding domain, such as a bacterial Ig binding domain, or a fragment or functional variant thereof.

In some embodiments, the Ig binding domain used in the purification methods described herein is Protein L, or a derivative or binding domain fragments thereof. Protein L, which was first isolated from Finegoldia magna (formerly Peptostreptococcus magnus), is an immunoglobulin-binding protein that has the unique ability to bind to bind through kappa light chain interactions without interfering with the antigen-binding site of an antibody, scFv, Fab fragment, or other binding protein. Protein L binds native kappa light chain subtypes I, Ill and IV. Protein L does not bind to native kappa light chain subtypes II or native lambda light chains. Protein L binds to human IgG, IgA, IgM, IgE, and IgD. In some embodiments, the protein L is isolated or derived from F. magna. Protein L can be produced recombinantly in, for example, E. coli. In some embodiments, Protein L comprises the sequence of SEQ ID NO: 20, or a sequence at least 90%, at least 95%, at least 96%, at least 97%, at least 98%, or at least 99% identical thereto.

(SEQ ID NO: 20)
MKINKKLLMAALAGAIVVGGGANAYAAEEDNTDNNLSMDEISDAYFDYHG
DVSDSVDPVEEEIDEALAKALAEAKETAKKHIDSLNHLSETAKKLAKNDI
DSATTINAINDIVARADVMERKTAEKEEAEKLAAAKETAKKHIDELKHLA
DKTKELAKRDIDSATTINAINDIVARADVMERKTAEKEEAEKLAAAKETA
KKHIDELKHLADKTKELAKRDIDSATTIDAINDIVARADVMERKLSEKET
PEPEEEVTIKANLIFADGSTQNAEFKGTFAKAVSDAYAYADALKKDNGEY
TVDVADKGLTLNIKFAGKKEKPEEPKEEVTIKVNLIFADGKTQTAEFKGT
FEEATAKAYAYADLLAKENGEYTADLEDGGNTINIKFAGKETPETPEEPK
EEVTIKVNLIFADGKIQTAEFKGTFEEATAKAYAYANLLAKENGEYTADL
EDGGNTINIKFAGKETPETPEEPKEEVTIKVNLIFADGKTQTAEFKGTFE
EATAEAYRYADLLAKVNGEYTADLEDGGYTINIKFAGKEQPGENPGITID
EWLLKNAKEEAIKELKEAGITSDLYFSLINKAKTVEGVEALKNEILKAHA
GEETPELKDGYATYEEAEAAAKEALKNDDVNNAYEIVQGADGRYYYVLKI
EVADEEEPGEDTPEVQEGYATYEEAEAAAKEALKEDKVNNAYEVVQGADG
RYYYVLKIEDKEDEQPGEEPGENPGITIDEWLLKNAKEDAIKELKEAGIS
SDIYFDAINKAKTVEGVEALKNEILKAHAEKPGENPGITIDEWLLKNAKE
AAIKELKEAGITAEYLFNLINKAKTVEGVESLKNEILKAHAEKPGENPGI
TIDEWLLKNAKEDAIKELKEAGITSDIYFDAINKAKTIEGVEALKNEILK
AHKKDEEPGKKPGEDKKPEDKKPGEDKKPEDKKPGEDKKPEDKKPGKTDK
DSPNKKKKAKLPKAGSEAEILTLAAAALSTAAGAYVSLKKRK

In some embodiments, purification methods based on Protein L are used as described in WO 2020/154475. This method is referred to as “kappatization” because it involves the modification of an immunoglobulin light chain structure that is not from a kappa light chain (e.g., a lambda light chain) such that its similarity to a kappa light chain is increased in order to provide the functionality of higher affinity binding to Protein L.

In some embodiments, the Ig binding domain is Protein G from group C and G Streptococcal bacteria, or Protein A from S. aureus, or derivatives and binding domain fragments of any of the foregoing.

In some embodiments, a purification method comprises contacting a binding molecule comprising an Ig binding domain epitope with an Ig binding domain (e.g., protein L or a fragment or derivative thereof).

In some embodiments, the method comprises three steps: a binding step, a washing step, and an elution step. In the binding step, a protein comprising a chimeric immunoglobulin binding domain is contacted with protein L immobilized on a matrix. The matrix can be any solid support such as a bead, a resin, etc. In some embodiments, the matrix can be packed into a column or into a cartridge.

In the washing step, the matrix is washed to remove impurities. The washing step can be repeated, for example at least 2 times, at least 3 times, at least 4 times, or at least 5 times, until substantially all impurities are removed. In some embodiments, the washing step is repeated until at least 80%, at least 85%, at least 90%, at least 95%, at least 96%, at least 97%, at least 98%, or at least 99% of impurities are removed.

In the elution step, the protein comprising the chimeric immunoglobulin binding domain is eluted from the protein L-matrix. The protein can be eluted using, for example, a high salt wash solution (e.g., 1 M NaCl) or a change in pH. After elution, the protein can be collected and further purified and/or desalted as appropriate, according to standard methods.

In some embodiments, a method for purifying a CTLA-4 binding molecule from an expression system composition comprising the CTLA-4 binding molecule and at least one other biomolecule comprises (i) contacting the expression system composition with a bacterial protein L to create a CTLA-4 binding molecule-protein L complex, and (ii) recovering the CTLA-4 binding molecule-protein L complex. In some embodiments, the expression system composition is a cellular lysate. In some embodiments, the protein L is isolated or derived from F. magna. In some embodiments, the protein L is conjugated to a resin.

In some embodiments, a method of making a binding molecule comprises culturing a host cell under conditions wherein the binding molecule is expressed, and recovering the protein. In some embodiments, a method of purifying a binding molecule comprises contacting the binding molecule with a bacterial protein L. In some embodiments, the protein L is isolated or derived from F. magna. In some embodiments, the protein L is conjugated to a resin.

### VI. Compositions

Also provided herein are compositions comprising a binding molecule. In some embodiments, the compositions are pharmaceutical compositions. In some embodiments, the compositions are useful for treatment or prophylaxis of cancer, or conditions, diseases, or symptoms associated therewith.

Pharmaceutical compositions comprising a binding molecule, or an acceptable salt or solvate thereof, can also comprise a pharmaceutically acceptable carrier, excipient, surfactant, stabilizer, antioxidant, vehicle, etc. Such agents should be non-toxic and should not interfere with the stability or efficacy of the binding molecule. Illustrative pharmaceutically acceptable buffers include histidine-butters, citrate-butters, succinate-buffers, acetate-buffers and phosphate-buffers or mixtures thereof. Exemplary stabilizing agents include sugars or sugar alcohols (e.g., mannitol, dextrose, glucose, trehalose, and/or sucrose). Inorganic salts (e.g., sodium chloride (NaCl), sodium sulfate (Na₂SO₄), sodium thiocyanate (NaSCN), magnesium chloride (MgCl), magnesium sulfate (MgSO₄), ammonium thiocyanate (NH₄SCN), ammonium sulfate ((NH₄)₂SO₄), ammonium chloride (NH₄Cl), calcium chloride (CaCl₂), calcium sulfate (CaSO₄), zinc chloride (ZnCl₂)) may also be used as stabilizers. Illustrative surfactants include poloxamers, polysorbates, polyoxy ethylene alkyl ethers (Brij), alkylphenylpolyoxyethylene ethers (Triton-X) or sodium dodecyl sulphate (SDS). Suitable tonicity agents include but are not limited to salts, amino acids and sugars (e.g., sodium chloride, trehalose, sucrose or arginine). Antioxidants include but are not limited to EDTA, citric acid, ascorbic add, butylated hydroxytoluene (BHT), butylated hydroxy anisole (BHA), sodium sulfite, p-amino benzoic acid, glutathione, propyl gallate, cysteine, methionine, ethanol and N-acetyl cysteine. Chelating agents, reactive oxygen scavengers and chain terminators can also be used. Additional suitable carriers, diluents, excipients, stabilizers, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA), Remington's Pharmaceutical Sciences, 20th edition, pub. Lippincott, Williams & Wilkins, 2000; and Handbook of Pharmaceutical Excipients, 2nd edition, 1994. The precise nature of the carrier or other agent will depend on the route of administration, which may be oral, or by injection, e.g., cutaneous, subcutaneous, or intravenous.

In some embodiments, the compositions comprising binding molecules described herein are useful for intravenous infusion. In some embodiments, the binding molecules are formulated in an aqueous buffer solution containing a cryogenic protectant and a surfactant.

Pharmaceutical compositions can conveniently be presented in unit dosage form and can be prepared by any of the methods well known in the art of pharmacy. In such form, the composition is divided into unit doses containing appropriate quantities of the active component. Compositions can be formulated for any suitable route and means of administration.

In some embodiments, a pharmaceutical composition comprising a CTLA-4 binding molecule as described herein, and at least one pharmaceutically acceptable excipient or carrier In some embodiments, a pharmaceutical composition comprises: a binding molecule comprising a (i) Shiga toxin A subunit effector polypeptide and (ii) a binding region capable of specifically binding CTLA-4 on the surface of an immunosuppressive immune cell; and (ii) a pharmaceutically acceptable carrier, excipient or buffer.

In some embodiments, the composition comprising the CTLA-4 binding molecule of the disclosure (e.g., the composition for administering to the subject) comprises about 0.1 mg/mL to about 100.0 mg/mL of the CTLA-4 binding molecule. In some embodiments, the composition comprises about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, about 10 mg/mL, about 15.0 mg/mL, about 20.0 mg/mL, about 25.0 mg/mL, about 30.0 mg/mL, about 35.0 mg/mL, about 40.0 mg/mL, about 45.0 mg/mL, about 50.0 mg/mL, about 55.0 mg/mL, about 60.0 mg/mL, about 65.0 mg/mL, about 70.0 mg/mL, about 75.0 mg/mL, about 80.0 mg/mL, about 85.0 mg/mL, about 90.0 mg/mL, about 95.0 mg/mL, or about 100.0 mg/mL of the CTLA-4 binding molecule. In some embodiments, the composition comprises about 0.5 mg/mL of the CTLA-4 binding molecule. In some embodiments, the composition comprises about 1 mg/mL of the CTLA-4 binding molecule.

In some embodiments, the pharmaceutical composition comprising the CTLA-4 binding molecule of the disclosure (e.g., the composition for administering to the subject) comprises a buffer comprising one or more salts, sugars, and/or poloxamers. In some embodiments, the pharmaceutical composition comprising the CTLA-4 binding molecule of the disclosure (e.g., the composition for administering to the subject) comprises a buffer comprising sodium acetate, sucrose, sodium chloride, and a poloxamer. In some embodiments, the composition comprising the CTLA-4 binding molecule of the disclosure (e.g., the composition for administering to the subject) comprises a buffer comprising one or more of sodium acetate, sucrose, sodium chloride, and a poloxamer. In some embodiments, the composition comprising the CTLA-4 binding molecule of the disclosure (e.g., the composition for administering to the subject) comprises a buffer comprising one or more of sodium acetate, sucrose, sodium chloride, and poloxamer 188. In some embodiments, the composition comprises a buffer comprising sodium acetate. In some embodiments, the composition comprises a buffer comprising sucrose. In some embodiments, the composition comprises a buffer comprising sodium chloride. In some embodiments, the composition comprises a buffer comprising poloxamer 188. In some embodiments, the composition comprises a buffer comprising sodium acetate, sucrose, sodium chloride, and poloxamer 188. In some embodiments, the composition comprises about 0.1 mg/mL to about 1.0 mg/mL, about 0.2 mg/mL to about 0.8 mg/mL, about 0.3 mg/mL to about 0.7 mg/mL, or about 0.4 mg/mL to about 0.6 mg/mL, of the CTLA-4 binding molecule. In some embodiments, the composition comprises about 0.5 mg/mL of the CTLA-4 binding molecule.

In some embodiments, the composition comprising the CTLA-4 binding molecule of the disclosure (e.g., the composition for administering to the subject) comprises a buffer comprising sodium acetate. In some embodiments, the composition comprises about 1 mM to about 50 mM sodium acetate. In some embodiments, the composition comprises about 1 mM, about 1.2 mM, about 1.4 mM, about 1.7 mM, about 2 mM, about 2.4 mM, about 2.7 mM, about 3 mM, about 3.5 mM, about 4 mM, about 4.5 mM, about 5 mM, about 6 mM, about 7 mM, about 8 mM, about 9 mM, about 10 mM, about 12 mM, about 14 mM, about 17 mM, about 20 mM, about 24 mM, about 27 mM, about 30 mM, about 35 mM, about 40 mM, about 45 mM, or about 50 mM, sodium acetate. In some embodiments, the composition comprises about 1 mM to about 2 mM, about 2 mM to about 5 mM, about 5 mM to about 10 mM, about 10 mM to about 15 mM, about 15 mM to about 20 mM, about 20 mM to about 25 mM, about 25 mM to about 30 mM, about 30 mM to about 40 mM, about 40 mM to about 50 mM, about 1 mM to about 5 mM, about 2 mM to about 10 mM, about 5 mM to about 15 mM, about 10 mM to about 20 mM, about 15 mM to about 25 mM, about 20 mM to about 30 mM, about 25 mM to about 40 mM, about 30 mM to about 50 mM, about 1 mM to about 10 mM, about 2 mM to about 15 mM, about 5 mM to about 20 mM, about 10 mM to about 25 mM, about 15 mM to about 30 mM, about 20 mM to about 40 mM, about 25 mM to about 50 mM, about 1 mM to about 15 mM, about 2 mM to about 20 mM, about 5 mM to about 25 mM, about 10 mM to about 30 mM, about 15 mM to about 40 mM, about 20 mM to about 50 mM, about 1 mM to about 20 mM, about 2 mM to about 25 mM, about 5 mM to about 30 mM, about 10 mM to about 40 mM, about 15 mM to about 50 mM, about 1 mM to about 25 mM, about 2 mM to about 30 mM, about 5 mM to about 40 mM, about 10 mM to about 50 mM, about 1 mM to about 30 mM, about 2 mM to about 40 mM, about 5 mM to about 50 mM, about 1 mM to about 40 mM, or about 2 mM to about 50 mM, sodium acetate, including all ranges and subranges in between. In some embodiments, the composition comprises about 5 mM to about 35 mM, about 10 mM to about 30 mM, or about 15 mM to about 25 mM, sodium acetate, including all ranges and subranges in between. In some embodiments, the composition comprises about 20 mM sodium acetate. In some embodiments, the composition comprises about 0.1 mg/mL to about 1.0 mg/mL, about 0.2 mg/mL to about 0.8 mg/mL, about 0.3 mg/mL to about 0.7 mg/mL, or about 0.4 mg/mL to about 0.6 mg/mL, of the CTLA-4 binding molecule. In some embodiments, the composition comprises about 0.5 mg/mL of the CTLA-4 binding molecule.

In some embodiments, the composition comprising the CTLA-4 binding molecule of the disclosure (e.g., the composition for administering to the subject) comprises a buffer comprising sucrose. In some embodiments, the composition comprises about 1% w/v to about 10% w/v sucrose. In some embodiments, the composition comprises about 1% w/v, about 1.2% w/v, about 1.4% w/v, about 1.7% w/v, about 2% w/v, about 2.4% w/v, about 2.7% w/v, about 3% w/v, about 3.5% w/v, about 4% w/v, about 4.5% w/v, about 5% w/v, about 5.5% w/v, about 6% w/v, about 6.5% w/v, about 7% w/v, about 8% w/v, about 9% w/v, or about 10% w/v, sucrose. In some embodiments, the composition comprises about 1% w/v to about 2% w/v, about 2% w/v to about 3% w/v, about 3% w/v to about 4% w/v, about 4% w/v to about 5% w/v, about 5% w/v to about 6% w/v, about 6% w/v to about 7% w/v, about 7% w/v to about 8% w/v, about 8% w/v to about 9% w/v, about 9% w/v to about 10% w/v, about 1% w/v to about 3% w/v, about 2% w/v to about 4% w/v, about 3% w/v to about 5% w/v, about 4% w/v to about 6% w/v, about 5% w/v to about 7% w/v, about 6% w/v to about 8% w/v, about 7% w/v to about 9% w/v, about 8% w/v to about 10% w/v, about 1% w/v to about 4% w/v, about 2% w/v to about 5% w/v, about 3% w/v to about 6% w/v, about 4% w/v to about 7% w/v, about 5% w/v to about 8% w/v, about 6% w/v to about 9% w/v, about 7% w/v to about 10% w/v, about 1 w/v to about 5% w/v, about 2% w/v to about 6% w/v, about 3% w/v to about 7% w/v, about 4% w/v to about 8% w/v, about 5% w/v to about 9% w/v, about 6% w/v to about 10% w/v, about 1% w/v to about 6% w/v, about 2% w/v to about 7% w/v, about 3% w/v to about 8% w/v, about 4% w/v to about 9% w/v, about 5% w/v to about 10% w/v, about 1% w/v to about 7% w/v, about 2% w/v to about 8% w/v, about 3% w/v to about 9% w/v, about 4% w/v to about 10% w/v, about 1% w/v to about 8% w/v, about 2% w/v to about 9% w/v, about 3% w/v to about 10% w/v, about 1% w/v to about 9% w/v, or about 2% w/v to about 10% w/v, including all ranges and subranges in between, sucrose. In some embodiments, the composition comprises about 2% w/v to about 10% w/v, about 3% w/v to about 9% w/v, about 4% w/v to about 8% w/v, about 5% w/v to about 7% w/v, or about 5.5% w/v to about 6.5% w/v, including all ranges and subranges in between, sucrose. In some embodiments, the composition comprises about 6% w/v sucrose. In some embodiments, the composition comprises about 0.1 mg/mL to about 1.0 mg/mL, about 0.2 mg/mL to about 0.8 mg/mL, about 0.3 mg/mL to about 0.7 mg/mL, or about 0.4 mg/mL to about 0.6 mg/mL, of the CTLA-4 binding molecule. In some embodiments, the composition comprises about 0.5 mg/mL of the CTLA-4 binding molecule.

In some embodiments, the composition comprising the CTLA-4 binding molecule of the disclosure (e.g., the composition for administering to the subject) comprises a buffer comprising sodium chloride. In some embodiments, the composition comprises about 50 mM to about 100 mM sodium chloride. In some embodiments, the composition comprises about 50 mM, about 55 mM, about 60 mM, about 65 mM, about 70 mM, about 75 mM, about 80 mM, about 85 mM, about 90 mM, about 95 mM, or about 100 mM, sodium chloride. In some embodiments, the composition comprises about 50 mM to about 60 mM, about 60 mM to about 70 mM, about 70 mM to about 80 mM, about 80 mM to about 90 mM, about 90 mM to about 100 mM, about 50 mM to about 70 mM, about 60 mM to about 80 mM, about 70 mM to about 90 mM, about 80 mM to about 100 mM, about 50 mM to about 80 mM, about 60 mM to about 90 mM, about 70 mM to about 100 mM, about 50 mM to about 90 mM, about 60 mM to about 100 mM, including all ranges and subranges in between, sodium chloride. In some embodiments, the composition comprises about 60 mM to about 90 mM, about 65 mM to about 85 mM, or about 70 mM to about 80 mM, including all ranges and subranges in between, sodium chloride. In some embodiments, the composition comprises about 75 mM sodium chloride. In some embodiments, the composition comprises about 0.1 mg/mL to about 1.0 mg/mL, about 0.2 mg/mL to about 0.8 mg/mL, about 0.3 mg/mL to about 0.7 mg/mL, or about 0.4 mg/mL to about 0.6 mg/mL, of the CTLA-4 binding molecule. In some embodiments, the composition comprises about 0.5 mg/mL of the CTLA-4 binding molecule.

In some embodiments, the pharmaceutical composition comprising the CTLA-4 binding molecule of the disclosure (e.g., the composition for administering to the subject) comprises a buffer comprising at least one poloxamer. In some embodiments, the poloxamer is poloxamer-105, poloxamer-108, poloxamer-122, poloxamer-123, poloxamer-124, poloxamer-182, poloxamer-183, poloxamer-184, poloxamer-185, poloxamer-188, poloxamer-212, poloxamer-215, poloxamer-217, poloxamer-234, poloxamer-235, poloxamer-237, poloxamer-238, poloxamer-288, poloxamer-333, poloxamer-334, poloxamer-335, poloxamer-338, poloxamer-402, poloxamer-403, or poloxamer-407. In some embodiments, the pharmaceutical composition comprising the CTLA-4 binding molecule of the disclosure (e.g., the composition for administering to the subject) comprises a buffer comprising poloxamer-188.

In some embodiments, the composition comprising the CTLA-4 binding molecule of the disclosure (e.g., the composition for administering to the subject) comprises a buffer comprising a poloxamer. In some embodiments, the composition comprises about 0.01% w/v to about 1% w/v of poloxamer. In some embodiments, the composition comprises about 0.01% w/v, about 0.012% w/v, about 0.014% w/v, about 0.017% w/v, about 0.02% w/v, about 0.024% w/v, about 0.027% w/v, about 0.03% w/v, about 0.035% w/v, about 0.04% w/v, about 0.045% w/v, about 0.05% w/v, about 0.06% w/v, about 0.07% w/v, about 0.08% w/v, about 0.09% w/v, about 0.1% w/v, about 0.12% w/v, about 0.14% w/v, about 0.17% w/v, about 0.2% w/v, about 0.24% w/v, about 0.27% w/v, about 0.3% w/v, about 0.35% w/v, about 0.4% w/v, about 0.45% w/v, about 0.5% w/v, about 0.6% w/v, about 0.7% w/v, about 0.8% w/v, about 0.9% w/v, or about 1% w/v of poloxamer. In some embodiments, the composition comprises about 0.01% w/v to about 0.02% w/v, about 0.02% w/v to about 0.04% w/v, about 0.04% w/v to about 0.06% w/v, about 0.06% w/v to about 0.08% w/v, about 0.08% w/v to about 0.1% w/v, about 0.1% w/v to about 0.13% w/v, about 0.13% w/v to about 0.17% w/v, about 0.17% w/v to about 0.2% w/v, about 0.2% w/v to about 0.3% w/v, about 0.3% w/v to about 0.5% w/v, about 0.5% w/v to about 0.7% w/v, about 0.7% w/v to about 1% w/v, about 0.01% w/v to about 0.04% w/v, about 0.02% w/v to about 0.06% w/v, about 0.04% w/v to about 0.08% w/v, about 0.06% w/v to about 0.1% w/v, about 0.08% w/v to about 0.13% w/v, about 0.1% w/v to about 0.17% w/v, about 0.13% w/v to about 0.2% w/v, about 0.17% w/v to about 0.3% w/v, about 0.2% w/v to about 0.5% w/v, about 0.3% w/v to about 0.7% w/v, about 0.5% w/v to about 1% w/v, about 0.01% w/v to about 0.06% w/v, about 0.02% w/v to about 0.08% w/v, about 0.04% w/v to about 0.1% w/v, about 0.06% w/v to about 0.13% w/v, about 0.08% w/v to about 0.17% w/v, about 0.1% w/v to about 0.2% w/v, about 0.13% w/v to about 0.3% w/v, about 0.17% w/v to about 0.5% w/v, about 0.2% w/v to about 0.7% w/v, about 0.3% w/v to about 1% w/v, about 0.01% w/v to about 0.08% w/v, about 0.02% w/v to about 0.1% w/v, about 0.04% w/v to about 0.13% w/v, about 0.06% w/v to about 0.17% w/v, about 0.08% w/v to about 0.2% w/v, about 0.1% w/v to about 0.3% w/v, about 0.13% w/v to about 0.5% w/v, about 0.17% w/v to about 0.7% w/v, about 0.2% w/v to about 1% w/v, about 0.01% w/v to about 0.1% w/v, about 0.02% w/v to about 0.13% w/v, about 0.04% w/v to about 0.17% w/v, about 0.06% w/v to about 0.2% w/v, about 0.08% w/v to about 0.3% w/v, about 0.1% w/v to about 0.5% w/v, about 0.13% w/v to about 0.7% w/v, about 0.17% w/v to about 1% w/v, about 0.01% w/v to about 0.13% w/v, about 0.02% w/v to about 0.17% w/v, about 0.04% w/v to about 0.2% w/v, about 0.06% w/v to about 0.3% w/v, about 0.08% w/v to about 0.5% w/v, about 0.1% w/v to about 0.7% w/v, about 0.13% w/v to about 1% w/v, about 0.01% w/v to about 0.17% w/v, about 0.02% w/v to about 0.2% w/v, about 0.04% w/v to about 0.3% w/v, about 0.06% w/v to about 0.5% w/v, about 0.08% w/v to about 0.7% w/v, about 0.1% w/v to about 1% w/v, about 0.01% w/v to about 0.2% w/v, about 0.02% w/v to about 0.3% w/v, about 0.04% w/v to about 0.5% w/v, about 0.06% w/v to about 0.7% w/v, about 0.08% w/v to about 1% w/v, about 0.01% w/v to about 0.3% w/v, about 0.02% w/v to about 0.5% w/v, about 0.04% w/v to about 0.7% w/v, about 0.06% w/v to about 1% w/v, about 0.01% w/v to about 0.5% w/v, about 0.02% w/v to about 0.7% w/v, about 0.04% w/v to about 1% w/v, about 0.01% w/v to about 0.7% w/v, or about 0.02% w/v to about 1% w/v, including all ranges and subranges in between, of poloxamer. In some embodiments, the composition comprises about 0.01% w/v to about 0.2% w/v, about 0.02% w/v to about 0.18% w/v, about 0.04% w/v to about 0.16% w/v, about 0.06% w/v to about 0.14% w/v, about 0.08% w/v to about 0.12% w/v, or about 0.09% w/v to about 0.11% w/v, including all ranges and subranges in between, of poloxamer. In some embodiments, the composition comprises about 0.1% w/v of poloxamer. In some embodiments, the composition comprises about 0.1 mg/mL to about 1.0 mg/mL, about 0.2 mg/mL to about 0.8 mg/mL, about 0.3 mg/mL to about 0.7 mg/mL, or about 0.4 mg/mL to about 0.6 mg/mL, of the CTLA-4 binding molecule. In some embodiments, the composition comprises about 0.5 mg/mL of the CTLA-4 binding molecule.

In some embodiments, the composition comprising the CTLA-4 binding molecule of the disclosure (e.g., the composition for administering to the subject) comprises a buffer comprising poloxamer 188. In some embodiments, the composition comprises about 0.01% w/v to about 1% w/v poloxamer 188. In some embodiments, the composition comprises about 0.01% w/v, about 0.012% w/v, about 0.014% w/v, about 0.017% w/v, about 0.02% w/v, about 0.024% w/v, about 0.027% w/v, about 0.03% w/v, about 0.035% w/v, about 0.04% w/v, about 0.045% w/v, about 0.05% w/v, about 0.06% w/v, about 0.07% w/v, about 0.08% w/v, about 0.09% w/v, about 0.1% w/v, about 0.12% w/v, about 0.14% w/v, about 0.17% w/v, about 0.2% w/v, about 0.24% w/v, about 0.27% w/v, about 0.3% w/v, about 0.35% w/v, about 0.4% w/v, about 0.45% w/v, about 0.5% w/v, about 0.6% w/v, about 0.7% w/v, about 0.8% w/v, about 0.9% w/v, or about 1% w/v of poloxamer 188. In some embodiments, the composition comprises about 0.01% w/v to about 0.02% w/v, about 0.02% w/v to about 0.04% w/v, about 0.04% w/v to about 0.06% w/v, about 0.06% w/v to about 0.08% w/v, about 0.08% w/v to about 0.1% w/v, about 0.1% w/v to about 0.13% w/v, about 0.13% w/v to about 0.17% w/v, about 0.17% w/v to about 0.2% w/v, about 0.2% w/v to about 0.3% w/v, about 0.3% w/v to about 0.5% w/v, about 0.5% w/v to about 0.7% w/v, about 0.7% w/v to about 1% w/v, about 0.01% w/v to about 0.04% w/v, about 0.02% w/v to about 0.06% w/v, about 0.04% w/v to about 0.08% w/v, about 0.06% w/v to about 0.1% w/v, about 0.08% w/v to about 0.13% w/v, about 0.1% w/v to about 0.17% w/v, about 0.13% w/v to about 0.2% w/v, about 0.17% w/v to about 0.3% w/v, about 0.2% w/v to about 0.5% w/v, about 0.3% w/v to about 0.7% w/v, about 0.5% w/v to about 1% w/v, about 0.01% w/v to about 0.06% w/v, about 0.02% w/v to about 0.08% w/v, about 0.04% w/v to about 0.1% w/v, about 0.06% w/v to about 0.13% w/v, about 0.08% w/v to about 0.17% w/v, about 0.1% w/v to about 0.2% w/v, about 0.13% w/v to about 0.3% w/v, about 0.17% w/v to about 0.5% w/v, about 0.2% w/v to about 0.7% w/v, about 0.3% w/v to about 1% w/v, about 0.01% w/v to about 0.08% w/v, about 0.02% w/v to about 0.1% w/v, about 0.04% w/v to about 0.13% w/v, about 0.06% w/v to about 0.17% w/v, about 0.08% w/v to about 0.2% w/v, about 0.1% w/v to about 0.3% w/v, about 0.13% w/v to about 0.5% w/v, about 0.17% w/v to about 0.7% w/v, about 0.2% w/v to about 1% w/v, about 0.01% w/v to about 0.1% w/v, about 0.02% w/v to about 0.13% w/v, about 0.04% w/v to about 0.17% w/v, about 0.06% w/v to about 0.2% w/v, about 0.08% w/v to about 0.3% w/v, about 0.1% w/v to about 0.5% w/v, about 0.13% w/v to about 0.7% w/v, about 0.17% w/v to about 1% w/v, about 0.01% w/v to about 0.13% w/v, about 0.02% w/v to about 0.17% w/v, about 0.04% w/v to about 0.2% w/v, about 0.06% w/v to about 0.3% w/v, about 0.08% w/v to about 0.5% w/v, about 0.1% w/v to about 0.7% w/v, about 0.13% w/v to about 1% w/v, about 0.01% w/v to about 0.17% w/v, about 0.02% w/v to about 0.2% w/v, about 0.04% w/v to about 0.3% w/v, about 0.06% w/v to about 0.5% w/v, about 0.08% w/v to about 0.7% w/v, about 0.1% w/v to about 1% w/v, about 0.01% w/v to about 0.2% w/v, about 0.02% w/v to about 0.3% w/v, about 0.04% w/v to about 0.5% w/v, about 0.06% w/v to about 0.7% w/v, about 0.08% w/v to about 1% w/v, about 0.01% w/v to about 0.3% w/v, about 0.02% w/v to about 0.5% w/v, about 0.04% w/v to about 0.7% w/v, about 0.06% w/v to about 1% w/v, about 0.01% w/v to about 0.5% w/v, about 0.02% w/v to about 0.7% w/v, about 0.04% w/v to about 1% w/v, about 0.01% w/v to about 0.7% w/v, or about 0.02% w/v to about 1% w/v, including all ranges and subranges in between of poloxamer 188. In some embodiments, the composition comprises about 0.01% w/v to about 0.2% w/v, about 0.02% w/v to about 0.18% w/v, about 0.04% w/v to about 0.16% w/v, about 0.06% w/v to about 0.14% w/v, about 0.08% w/v to about 0.12% w/v, or about 0.09% w/v to about 0.11% w/v, including all ranges and subranges in between of poloxamer 188. In some embodiments, the composition comprises about 0.1% w/v of poloxamer 188. In some embodiments, the composition comprises about 0.1 mg/mL to about 1.0 mg/mL, about 0.2 mg/mL to about 0.8 mg/mL, about 0.3 mg/mL to about 0.7 mg/mL, or about 0.4 mg/mL to about 0.6 mg/mL, of the CTLA-4 binding molecule. In some embodiments, the composition comprises about 0.5 mg/mL of the CTLA-4 binding molecule.

In some embodiments, the composition comprising the CTLA-4 binding molecule of the disclosure (e.g., the composition for administering to the subject) has a pH of about 3.0 to about 7.0. In some embodiments, the pH of the composition is about 3.0, about 3.5, about 4.0, about 4.5, about 5.0, about 5.5, about 6.0, about 6.5, or about 7.0. In some embodiments, the pH of the composition is about 3 to about 3.4, about 3.4 to about 3.8, about 3.8 to about 4.2, about 4.2 to about 4.6, about 4.6 to about 5, about 5 to about 5.4, about 5.4 to about 5.8, about 5.8 to about 6.2, about 6.2 to about 6.6, about 6.6 to about 7, about 3 to about 3.8, about 3.4 to about 4.2, about 3.8 to about 4.6, about 4.2 to about 5, about 4.6 to about 5.4, about 5 to about 5.8, about 5.4 to about 6.2, about 5.8 to about 6.6, about 6.2 to about 7, about 3 to about 4.2, about 3.4 to about 4.6, about 3.8 to about 5, about 4.2 to about 5.4, about 4.6 to about 5.8, about 5 to about 6.2, about 5.4 to about 6.6, about 5.8 to about 7, about 3 to about 4.6, about 3.4 to about 5, about 3.8 to about 5.4, about 4.2 to about 5.8, about 4.6 to about 6.2, about 5 to about 6.6, about 5.4 to about 7, about 3 to about 5, about 3.4 to about 5.4, about 3.8 to about 5.8, about 4.2 to about 6.2, about 4.6 to about 6.6, about 5 to about 7, about 3 to about 5.4, about 3.4 to about 5.8, about 3.8 to about 6.2, about 4.2 to about 6.6, about 4.6 to about 7, about 3 to about 5.8, about 3.4 to about 6.2, about 3.8 to about 6.6, about 4.2 to about 7, about 3 to about 6.2, about 3.4 to about 6.6, about 3.8 to about 7, about 3 to about 6.6, or about 3.4 to about 7, including all ranges and subranges in between. In some embodiments, the pH of the composition is about 4.0 to about 6.0, about 4.2 to about 5.8, about 4.4 to about 5.6, about 4.6 to about 5.4, about 4.8 to about 5.2, or about 4.9 to about 5.1, including all ranges and subranges in between. In some embodiments, the pH of the composition is about 5.0. In some embodiments, the composition comprises about 0.1 mg/mL to about 1.0 mg/mL, about 0.2 mg/mL to about 0.8 mg/mL, about 0.3 mg/mL to about 0.7 mg/mL, or about 0.4 mg/mL to about 0.6 mg/mL, of the CTLA-4 binding molecule. In some embodiments, the composition comprises about 0.5 mg/mL of the CTLA-4 binding molecule.

In some embodiments, the composition comprising the CTLA-4 binding molecule of the disclosure (e.g., the composition for administering to the subject) comprises:
(i) about 0.25-1.0 mg/mL of a CTLA-4 binding molecule;
(ii) about 10-40 mM sodium acetate;
(iii) about 3-12% w/v sucrose;
(iv) about 30-150 mM sodium chloride; and
(v) about 0.05-0.2% poloxamer 188;
wherein the composition has a pH of about 4.0-6.0.
   In some embodiments, the CTLA-4 binding molecule comprises an amino acid sequence of SEQ ID NO: 329.

In some embodiments, the composition comprising the CTLA-4 binding molecule of the disclosure (e.g., the composition for administering to the subject) comprises:
(i) about 0.3-0.75 mg/mL of a CTLA-4 binding molecule;
(ii) about 12-30 mM sodium acetate;
(iii) about 4-9% w/v sucrose;
(iv) about 50-120 mM sodium chloride; and
(v) about 0.07-0.15% poloxamer 188;
wherein the composition has a pH of about 4.5-5.5.
   In some embodiments, the CTLA-4 binding molecule comprises an amino acid sequence of SEQ ID NO: 329.

In some embodiments, the composition comprising the CTLA-4 binding molecule of the disclosure (e.g., the composition for administering to the subject) comprises:
(i) about 0.4-0.6 mg/mL of a CTLA-4 binding molecule;
(ii) about 15-25 mM sodium acetate;
(iii) about 5-7% w/v sucrose;
(iv) about 60-90 mM sodium chloride; and
(v) about 0.08-0.13% poloxamer 188;
wherein the composition has a pH of about 4.7-5.3.
   In some embodiments, the CTLA-4 binding molecule comprises an amino acid sequence of SEQ ID NO: 329.

In some embodiments, the composition comprising the CTLA-4 binding molecule of the disclosure (e.g., the composition for administering to the subject) comprises:
(i) about 0.45-0.55 mg/mL of a CTLA-4 binding molecule;
(ii) about 18-22 mM sodium acetate;
(iii) about 5.5-6.5% w/v sucrose;
(iv) about 70-80 mM sodium chloride; and
(v) about 0.09-0.11% poloxamer 188;
wherein the composition has a pH of about 4.9-5.1.
   In some embodiments, the CTLA-4 binding molecule comprises an amino acid sequence of SEQ ID NO: 329.

In some embodiments, the composition comprising the CTLA-4 binding molecule of the disclosure (e.g., the composition for administering to the subject) comprises:
(i) about 0.5 mg/mL of a CTLA-4 binding molecule;
(ii) about 20 mM sodium acetate;
(iii) about 6% w/v sucrose;
(iv) about 75 mM sodium chloride; and
(v) about 0.1% poloxamer 188;
wherein the composition has a pH of about 5.0.
   In some embodiments, the CTLA-4 binding molecule comprises an amino acid sequence of SEQ ID NO: 329.

In some embodiments, the pharmaceutical composition is contained in a container closure system. In some embodiments, the container closure system containing the pharmaceutical composition comprises a glass vial with an elastomeric stopper and an aluminum seal with a flip-off cap.

The pharmaceutical composition can be formulated for administration systemically or locally. In some embodiments, the pharmaceutical composition may be formulated for administration orally, parenterally, sublingually, transdermally, rectally, transmucosally, topically, via inhalation, via buccal administration, intrapleurally, intravenously, intraarterially, intragastrically, nasally, intraperitoneally, subcutaneously, intramuscularly, intranasally intrathecally, and intraarticularly or combinations thereof. In some embodiments, the pharmaceutical composition is formulated for intravenous administration.

In some embodiments, the pharmaceutical composition is sterile. In some embodiments, the sterility of the pharmaceutical composition is confirmed by the absence of microbial growth using standard methodology known to those skilled in the art. In some embodiments, the pharmaceutical composition comprises no detectable microbial growth.

Diagnostic compositions can comprise a binding molecule and at least one detection promoting agent. When producing or manufacturing a diagnostic composition, a binding molecule can be directly or indirectly linked to at least one detection promoting agent. There are numerous standard techniques known to the skilled worker for incorporating, affixing, and/or conjugating various detection promoting agents to proteins or proteinaceous components of molecules, especially to immunoglobulins and immunoglobulin-derived domains.

There are numerous detection promoting agents known to the skilled worker, such as isotopes, dyes, colorimetric agents, contrast enhancing agents, fluorescent agents, bioluminescent agents, and magnetic agents, which can be operably linked to the polypeptides or binding molecules for information gathering methods, such as for diagnostic and/or prognostic applications to diseases or conditions of an organism (see e.g. Cai W et al., J Nucl Med 48: 304-10 (2007); Nayak T, Brechbiel M, Bioconjug Chem 20: 825-41 (2009); Paudyal P et al., Oncol Rep 22: 115-9 (2009); Qiao J et al., PLoS ONE 6: e18103 (2011); Sano K et al., Breast Cancer Res 14: R61 (2012)). The incorporation of the agent is in such a way to enable the detection of the presence of the diagnostic composition in a screen, assay, diagnostic procedure, and/or imaging technique.

Similarly, there are numerous imaging approaches known to the skilled worker, such as non-invasive in vivo imaging techniques commonly used in the medical arena, for example: computed tomography imaging (CT scanning), optical imaging (including direct, fluorescent, and bioluminescent imaging), magnetic resonance imaging (MRI), positron emission tomography (PET), single-photon emission computed tomography (SPECT), ultrasound, and x-ray computed tomography imaging.

### VII. Methods for Treating

Also provided herein are methods for treating a subject in need thereof, the methods comprising administering to the subject an effective amount of (i) a binding molecule, (ii) a nucleic acid encoding the binding molecule, or (iii) a composition comprising a binding molecule or nucleic acid encoding the same.

As used herein, the term “subject” refers to any organism, commonly a mammalian subject, such as a humans or non-human animal. The terms “subject” and “patient” are used interchangeably. In some embodiments, the subject can be a mammal, such as a primate (e.g., a human or non-human primate), a livestock animal (e.g. cow, horse, pig, sheep, goat, etc.), a companion animal (e.g. cat, dog, etc.) or a laboratory animal (e.g. mouse, rabbit, rat, etc.). In some embodiments, the subject presents one or more symptoms, signs, and/or indications of at least one form of cancer.

As used herein, the terms “treat,” “treating,” or “treatment”, and grammatical variants thereof, have the same meaning as commonly understood by those of ordinary skill in the art. In some embodiments, these terms can refer to an approach for obtaining beneficial or desired clinical results. The terms can refer to slowing the onset or rate of development of a condition, disorder or disease, reducing or alleviating symptoms associated with it, generating a complete or partial regression of the condition, or some combination of any of the above. As described herein, beneficial or desired clinical results include, but are not limited to, reduction or alleviation of symptoms, diminishment of extent of disease, stabilization (e.g. not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject (e.g. a human) in need of treatment can thus be a subject already afflicted with the disease or disorder in question. The terms “treat,” “treating,” or “treatment” includes inhibition or reduction of an increase in severity of a pathological state or symptoms relative to the absence of treatment and is not necessarily meant to imply complete cessation of the relevant disease or condition.

As used herein, the terms “prevent,” “preventing,” “prevention” and grammatical variants thereof refer to an approach for preventing the development of, or altering the pathology of, a condition or disease. Accordingly, “prevention” can refer to prophylactic or preventive measures. As described herein, beneficial or desired clinical results include, but are not limited to, prevention or slowing of symptoms, progression or development of a disease, whether detectable or undetectable. A subject (e.g. a human) in need of prevention can thus be a subject not yet afflicted with the disease or disorder in question. The term “prevention” includes slowing the onset of disease relative to the absence of treatment and is not necessarily meant to imply permanent prevention of the relevant disease, disorder or condition. Thus “preventing” or “prevention” of a condition can in certain contexts refer to reducing the risk of developing the condition, or preventing or delaying the development of symptoms associated with the condition.

As used herein, an “effective amount” is an amount effective for treating and/or preventing a disease, disorder, or condition as disclosed herein. In some embodiments, an effective amount is an amount or dose of a composition (e.g., a therapeutic composition, compound, or agent) that produces at least one desired therapeutic effect in a subject, such as preventing or treating a target condition or beneficially alleviating a symptom associated with the condition. The most desirable effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic composition (including activity, pharmacokinetics, pharmacodynamics, and bioavailability), the physiological condition of the subject (including age, sex, disease type, disease stage, general physical condition, responsiveness to a given dosage, and type of medication), the nature of the pharmaceutically acceptable carrier or carriers in the formulation, and the route of administration. One skilled in the clinical and pharmacological arts will be able to determine an effective amount through routine experimentation, namely by monitoring a subject's response to administration of a composition and adjusting the dosage accordingly (see e.g., Remington: The Science and Practice of Pharmacy (Gennaro A, ed., Mack Publishing Co., Easton, Pa., U.S., 19th ed., 1995)).

In some embodiments, the effective amount of the CTLA-4 binding molecule is a dose in the range of about 0.01 μg/kg to about 2000 μg/kg body weight. In some embodiments, the effective amount of the CTLA-4 binding molecule is a dose in the range of about 1 μg/kg to about 250 μg/kg body weight. In some embodiments, the effective amount of the CTLA-4 binding molecule is a dose of about 1 μg/kg, about 2 μg/kg, about 4 μg/kg, about 8 μg/kg, about 16 μg/kg, about 32 μg/kg, about 64 μg/kg, about 128 μg/kg, about 192 μg/kg, or about 250 μg/kg body weight. In some embodiments, the effective amount of the CTLA-4 binding molecule is about 0.01 μg/kg, about 0.02 μg/kg, about 0.03 μg/kg, about 0.04 μg/kg, about 0.05 μg/kg, about 0.06 μg/kg, about 0.07 μg/kg, about 0.08 μg/kg, about 0.09 μg/kg, about 0.1 μg/kg, about 0.2 μg/kg, about 0.3 μg/kg, about 0.4 μg/kg, about 0.5 μg/kg, about 0.6 μg/kg, about 0.7 μg/kg, about 0.8 μg/kg, about 0.9 μg/kg, about 1 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 125 μg/kg, about 150 μg/kg, about 175 μg/kg, about 200 μg/kg, about 250 μg/kg, about 300 μg/kg, about 350 μg/kg, about 400 μg/kg, about 450 μg/kg, about 500 μg/kg, about 600 μg/kg, about 700 μg/kg, about 800 μg/kg, about 900 μg/kg, about 1000 μg/kg, about 1100 μg/kg, about 1200 μg/kg, about 1300 μg/kg, about 1400 μg/kg, about 1500 μg/kg, about 1600 μg/kg, about 1700 μg/kg, about 1800 μg/kg, about 1900 μg/kg, or about 2000 μg/kg body weight. In some embodiments, the effective amount of the CTLA-4 binding molecule is about 0.01 μg/kg, about 0.012 μg/kg, about 0.014 μg/kg, about 0.017 μg/kg, about 0.02 μg/kg, about 0.024 μg/kg, about 0.027 μg/kg, about 0.03 μg/kg, about 0.035 μg/kg, about 0.04 μg/kg, about 0.045 μg/kg, about 0.05 μg/kg, about 0.06 μg/kg, about 0.07 μg/kg, about 0.08 μg/kg, about 0.09 μg/kg, about 0.1 μg/kg, about 0.12 μg/kg, about 0.14 μg/kg, about 0.17 μg/kg, about 0.2 μg/kg, about 0.24 μg/kg, about 0.27 μg/kg, about 0.3 μg/kg, about 0.35 μg/kg, about 0.4 μg/kg, about 0.45 μg/kg, about 0.5 μg/kg, about 0.6 μg/kg, about 0.7 μg/kg, about 0.8 μg/kg, about 0.9 μg/kg, about 1 μg/kg, about 1.2 μg/kg, about 1.4 μg/kg, about 1.7 μg/kg, about 2 μg/kg, about 2.4 μg/kg, about 2.7 μg/kg, about 3 μg/kg, about 3.3 μg/kg, about 3.7 μg/kg, about 4 μg/kg, about 4.5 μg/kg, about 5 μg/kg, about 6 μg/kg, about 7 μg/kg, about 8 μg/kg, about 9 μg/kg, about 10 μg/kg, about 11 μg/kg, about 12 μg/kg, about 14 μg/kg, about 17 μg/kg, about 20 μg/kg, about 24 μg/kg, about 27 μg/kg, about 30 μg/kg, about 35 μg/kg, about 40 μg/kg, about 45 μg/kg, about 50 μg/kg, about 60 μg/kg, about 70 μg/kg, about 80 μg/kg, about 90 μg/kg, about 100 μg/kg, about 110 μg/kg, about 120 μg/kg, about 130 μg/kg, about 140 μg/kg, about 150 μg/kg, about 160 μg/kg, about 170 μg/kg, about 180 μg/kg, about 190 μg/kg, about 200 μg/kg, about 210 μg/kg, about 220 μg/kg, about 230 μg/kg, about 240 μg/kg, about 250 μg/kg, about 260 μg/kg, about 270 μg/kg, about 280 μg/kg, about 290 μg/kg, about 300 μg/kg, about 350 μg/kg, about 400 μg/kg, about 450 μg/kg, about 500 μg/kg, about 550 μg/kg, about 600 μg/kg, about 650 μg/kg, about 700 μg/kg, about 750 μg/kg, about 800 μg/kg, about 850 μg/kg, about 900 μg/kg, about 950 μg/kg, or about 1000 μg/kg, body weight. In some embodiments, the effective amount of the CTLA-4 binding molecule is a dose in the range of about 0.01 μg/kg to about 0.02 μg/kg, about 0.02 μg/kg to about 0.05 μg/kg, about 0.05 μg/kg to about 0.1 μg/kg, about 0.1 μg/kg to about 0.2 μg/kg, about 0.2 μg/kg to about 0.5 μg/kg, about 0.5 μg/kg to about 1 μg/kg, about 1 μg/kg to about 2 μg/kg, about 2 μg/kg to about 5 μg/kg, about 5 μg/kg to about 10 μg/kg, about 10 μg/kg to about 20 μg/kg, about 20 μg/kg to about 50 μg/kg, about 50 μg/kg to about 100 μg/kg, about 100 μg/kg to about 200 μg/kg, about 200 μg/kg to about 500 μg/kg, about 500 μg/kg to about 1000 μg/kg, about 0.01 μg/kg to about 0.05 μg/kg, about 0.02 μg/kg to about 0.1 μg/kg, about 0.05 μg/kg to about 0.2 μg/kg, about 0.1 μg/kg to about 0.5 μg/kg, about 0.2 μg/kg to about 1 μg/kg, about 0.5 μg/kg to about 2 μg/kg, about 1 μg/kg to about 5 μg/kg, about 2 μg/kg to about 10 μg/kg, about 5 μg/kg to about 20 μg/kg, about 10 μg/kg to about 50 μg/kg, about 20 μg/kg to about 100 μg/kg, about 50 μg/kg to about 200 μg/kg, about 100 μg/kg to about 500 μg/kg, about 200 μg/kg to about 1000 μg/kg, about 0.01 μg/kg to about 0.1 μg/kg, about 0.02 μg/kg to about 0.2 μg/kg, about 0.05 μg/kg to about 0.5 μg/kg, about 0.1 μg/kg to about 1 μg/kg, about 0.2 μg/kg to about 2 μg/kg, about 0.5 μg/kg to about 5 μg/kg, about 1 μg/kg to about 10 μg/kg, about 2 μg/kg to about 20 μg/kg, about 5 μg/kg to about 50 μg/kg, about 10 μg/kg to about 100 μg/kg, about 20 μg/kg to about 200 μg/kg, about 50 μg/kg to about 500 μg/kg, about 100 μg/kg to about 1000 μg/kg, about 0.01 μg/kg to about 0.2 μg/kg, about 0.02 μg/kg to about 0.5 μg/kg, about 0.05 μg/kg to about 1 μg/kg, about 0.1 μg/kg to about 2 μg/kg, about 0.2 μg/kg to about 5 μg/kg, about 0.5 μg/kg to about 10 μg/kg, about 1 μg/kg to about 20 μg/kg, about 2 μg/kg to about 50 μg/kg, about 5 μg/kg to about 100 μg/kg, about 10 μg/kg to about 200 μg/kg, about 20 μg/kg to about 500 μg/kg, about 50 μg/kg to about 1000 μg/kg, about 0.01 μg/kg to about 0.5 μg/kg, about 0.02 μg/kg to about 1 μg/kg, about 0.05 μg/kg to about 2 μg/kg, about 0.1 μg/kg to about 5 μg/kg, about 0.2 μg/kg to about 10 μg/kg, about 0.5 μg/kg to about 20 μg/kg, about 1 μg/kg to about 50 μg/kg, about 2 μg/kg to about 100 μg/kg, about 5 μg/kg to about 200 μg/kg, about 10 μg/kg to about 500 μg/kg, about 20 μg/kg to about 1000 μg/kg, about 0.01 μg/kg to about 1 μg/kg, about 0.02 μg/kg to about 2 μg/kg, about 0.05 μg/kg to about 5 μg/kg, about 0.1 μg/kg to about 10 μg/kg, about 0.2 μg/kg to about 20 μg/kg, about 0.5 μg/kg to about 50 μg/kg, about 1 μg/kg to about 100 μg/kg, about 2 μg/kg to about 200 μg/kg, about 5 μg/kg to about 500 μg/kg, about 10 μg/kg to about 1000 μg/kg, about 0.01 μg/kg to about 2 μg/kg, about 0.02 μg/kg to about 5 μg/kg, about 0.05 μg/kg to about 10 μg/kg, about 0.1 μg/kg to about 20 μg/kg, about 0.2 μg/kg to about 50 μg/kg, about 0.5 μg/kg to about 100 μg/kg, about 1 μg/kg to about 200 μg/kg, about 2 μg/kg to about 500 μg/kg, about 5 μg/kg to about 1000 μg/kg, about 0.01 μg/kg to about 5 μg/kg, about 0.02 μg/kg to about 10 μg/kg, about 0.05 μg/kg to about 20 μg/kg, about 0.1 μg/kg to about 50 μg/kg, about 0.2 μg/kg to about 100 μg/kg, about 0.5 μg/kg to about 200 μg/kg, about 1 μg/kg to about 500 μg/kg, about 2 μg/kg to about 1000 μg/kg, about 0.01 μg/kg to about 10 μg/kg, about 0.02 μg/kg to about 20 μg/kg, about 0.05 μg/kg to about 50 μg/kg, about 0.1 μg/kg to about 100 μg/kg, about 0.2 μg/kg to about 200 μg/kg, about 0.5 μg/kg to about 500 μg/kg, about 1 μg/kg to about 1000 μg/kg, about 0.01 μg/kg to about 20 μg/kg, about 0.02 μg/kg to about 50 μg/kg, about 0.05 μg/kg to about 100 μg/kg, about 0.1 μg/kg to about 200 μg/kg, about 0.2 μg/kg to about 500 μg/kg, about 0.5 μg/kg to about 1000 μg/kg, about 0.01 μg/kg to about 50 μg/kg, about 0.02 μg/kg to about 100 μg/kg, about 0.05 μg/kg to about 200 μg/kg, about 0.1 μg/kg to about 500 μg/kg, about 0.2 μg/kg to about 1000 μg/kg, about 0.01 μg/kg to about 100 μg/kg, about 0.02 μg/kg to about 200 μg/kg, about 0.05 μg/kg to about 500 μg/kg, about 0.1 μg/kg to about 1000 μg/kg, about 0.01 μg/kg to about 200 μg/kg, about 0.02 μg/kg to about 500 μg/kg, about 0.05 μg/kg to about 1000 μg/kg, about 0.01 μg/kg to about 500 μg/kg, or about 0.02 μg/kg to about 1000 μg/kg body weight, including all ranges and subranges in between. In some embodiments, the effective amount of the CTLA-4 binding molecule is a dose in the range of about 1 μg/kg to about 2 μg/kg, about 2 μg/kg to about 4 μg/kg, about 4 μg/kg to about 8 μg/kg, about 8 μg/kg to about 16 μg/kg, about 16 μg/kg to about 24 μg/kg, about 24 μg/kg to about 32 μg/kg, about 32 μg/kg to about 40 μg/kg, about 40 μg/kg to about 48 μg/kg, about 48 μg/kg to about 56 μg/kg, about 56 μg/kg to about 64 μg/kg, about 64 μg/kg to about 96 μg/kg, about 96 μg/kg to about 128 μg/kg, about 128 μg/kg to about 160 μg/kg, about 160 μg/kg to about 192 μg/kg, about 192 μg/kg to about 224 μg/kg, about 224 μg/kg to about 250 μg/kg, about 1 μg/kg to about 4 μg/kg, about 2 μg/kg to about 8 μg/kg, about 4 μg/kg to about 16 μg/kg, about 8 μg/kg to about 24 μg/kg, about 16 μg/kg to about 32 μg/kg, about 24 μg/kg to about 40 μg/kg, about 32 μg/kg to about 48 μg/kg, about 40 μg/kg to about 56 μg/kg, about 48 μg/kg to about 64 μg/kg, about 56 μg/kg to about 96 μg/kg, about 64 μg/kg to about 128 μg/kg, about 96 μg/kg to about 160 μg/kg, about 128 μg/kg to about 192 μg/kg, about 160 μg/kg to about 224 μg/kg, about 192 μg/kg to about 250 μg/kg, about 1 μg/kg to about 8 μg/kg, about 2 μg/kg to about 16 μg/kg, about 4 μg/kg to about 24 μg/kg, about 8 μg/kg to about 32 μg/kg, about 16 μg/kg to about 40 μg/kg, about 24 μg/kg to about 48 μg/kg, about 32 μg/kg to about 56 μg/kg, about 40 μg/kg to about 64 μg/kg, about 48 μg/kg to about 96 μg/kg, about 56 μg/kg to about 128 μg/kg, about 64 μg/kg to about 160 μg/kg, about 96 μg/kg to about 192 μg/kg, about 128 μg/kg to about 224 μg/kg, about 160 μg/kg to about 250 μg/kg, about 1 μg/kg to about 16 μg/kg, about 2 μg/kg to about 24 μg/kg, about 4 μg/kg to about 32 μg/kg, about 8 μg/kg to about 40 μg/kg, about 16 μg/kg to about 48 μg/kg, about 24 μg/kg to about 56 μg/kg, about 32 μg/kg to about 64 μg/kg, about 40 μg/kg to about 96 μg/kg, about 48 μg/kg to about 128 μg/kg, about 56 μg/kg to about 160 μg/kg, about 64 μg/kg to about 192 μg/kg, about 96 μg/kg to about 224 μg/kg, about 128 μg/kg to about 250 μg/kg, about 1 μg/kg to about 24 μg/kg, about 2 μg/kg to about 32 μg/kg, about 4 μg/kg to about 40 μg/kg, about 8 μg/kg to about 48 μg/kg, about 16 μg/kg to about 56 μg/kg, about 24 μg/kg to about 64 μg/kg, about 32 μg/kg to about 96 μg/kg, about 40 μg/kg to about 128 μg/kg, about 48 μg/kg to about 160 μg/kg, about 56 μg/kg to about 192 μg/kg, about 64 μg/kg to about 224 μg/kg, about 96 μg/kg to about 250 μg/kg, about 1 μg/kg to about 32 μg/kg, about 2 μg/kg to about 40 μg/kg, about 4 μg/kg to about 48 μg/kg, about 8 μg/kg to about 56 μg/kg, about 16 μg/kg to about 64 μg/kg, about 24 μg/kg to about 96 μg/kg, about 32 μg/kg to about 128 μg/kg, about 40 μg/kg to about 160 μg/kg, about 48 μg/kg to about 192 μg/kg, about 56 μg/kg to about 224 μg/kg, about 64 μg/kg to about 250 μg/kg, about 1 μg/kg to about 40 μg/kg, about 2 μg/kg to about 48 μg/kg, about 4 μg/kg to about 56 μg/kg, about 8 μg/kg to about 64 μg/kg, about 16 μg/kg to about 96 μg/kg, about 24 μg/kg to about 128 μg/kg, about 32 μg/kg to about 160 μg/kg, about 40 μg/kg to about 192 μg/kg, about 48 μg/kg to about 224 μg/kg, about 56 μg/kg to about 250 μg/kg, about 1 μg/kg to about 48 μg/kg, about 2 μg/kg to about 56 μg/kg, about 4 μg/kg to about 64 μg/kg, about 8 μg/kg to about 96 μg/kg, about 16 μg/kg to about 128 μg/kg, about 24 μg/kg to about 160 μg/kg, about 32 μg/kg to about 192 μg/kg, about 40 μg/kg to about 224 μg/kg, about 48 μg/kg to about 250 μg/kg, about 1 μg/kg to about 56 μg/kg, about 2 μg/kg to about 64 μg/kg, about 4 μg/kg to about 96 μg/kg, about 8 μg/kg to about 128 μg/kg, about 16 μg/kg to about 160 μg/kg, about 24 μg/kg to about 192 μg/kg, about 32 μg/kg to about 224 μg/kg, about 40 μg/kg to about 250 μg/kg, about 1 μg/kg to about 64 μg/kg, about 2 μg/kg to about 96 μg/kg, about 4 μg/kg to about 128 μg/kg, about 8 μg/kg to about 160 μg/kg, about 16 μg/kg to about 192 μg/kg, about 24 μg/kg to about 224 μg/kg, about 32 μg/kg to about 250 μg/kg, about 1 μg/kg to about 96 μg/kg, about 2 μg/kg to about 128 μg/kg, about 4 μg/kg to about 160 μg/kg, about 8 μg/kg to about 192 μg/kg, about 16 μg/kg to about 224 μg/kg, about 24 μg/kg to about 250 μg/kg, about 1 μg/kg to about 128 μg/kg, about 2 μg/kg to about 160 μg/kg, about 4 μg/kg to about 192 μg/kg, about 8 μg/kg to about 224 μg/kg, about 16 μg/kg to about 250 μg/kg, about 1 μg/kg to about 160 μg/kg, about 2 μg/kg to about 192 μg/kg, about 4 μg/kg to about 224 μg/kg, about 8 μg/kg to about 250 μg/kg, about 1 μg/kg to about 192 μg/kg, about 2 μg/kg to about 224 μg/kg, about 4 μg/kg to about 250 μg/kg, about 1 μg/kg to about 224 μg/kg, or about 2 μg/kg to about 250 μg/kg body weight, including all ranges and subranges in between. In some embodiments, the effective amount of the CTLA-4 binding molecule is a dose in the range of about 8 μg/kg to about 32 μg/kg, about 16 μg/kg to about 36 μg/kg, about 20 μg/kg to about 40 μg/kg, about 24 μg/kg to about 44 μg/kg, about 28 μg/kg to about 48 μg/kg, about 32 μg/kg to about 56 μg/kg, about 16 μg/kg to about 32 μg/kg, about 20 μg/kg to about 36 μg/kg, about 24 μg/kg to about 40 μg/kg, about 28 μg/kg to about 44 μg/kg, about 32 μg/kg to about 48 μg/kg, about 20 μg/kg to about 32 μg/kg, about 24 μg/kg to about 36 μg/kg, about 28 μg/kg to about 40 μg/kg, about 32 μg/kg to about 44 μg/kg, about 24 μg/kg to about 32 μg/kg, about 28 μg/kg to about 36 μg/kg, about 32 μg/kg to about 40 μg/kg, about 28 μg/kg to about 32 μg/kg, or about 32 μg/kg to about 36 μg/kg body weight, including all ranges and subranges in between. In some embodiments, the effective amount of the CTLA-4 binding molecule is a dose of about 32 μg/kg body weight.

In some embodiments, the CTLA-4 binding molecule is administered to the subject at a dose in the range of about 0.1 mg to about 2000 mg. In some embodiments, the CTLA-4 binding molecule is administered to the subject at a dose of about 0.1 mg, about 0.12 mg, about 0.14 mg, about 0.17 mg, about 0.2 mg, about 0.24 mg, about 0.27 mg, about 0.3 mg, about 0.35 mg, about 0.4 mg, about 0.45 mg, about 0.5 mg, about 0.6 mg, about 0.7 mg, about 0.8 mg, about 0.9 mg, about 1 mg, about 1.2 mg, about 1.4 mg, about 1.7 mg, about 2 mg, about 2.4 mg, about 2.7 mg, about 3 mg, about 3.3 mg, about 3.7 mg, about 4 mg, about 4.5 mg, about 5 mg, about 6 mg, about 7 mg, about 8 mg, about 9 mg, about 10 mg, about 11 mg, about 12 mg, about 14 mg, about 17 mg, about 20 mg, about 24 mg, about 27 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg. In some embodiments, the CTLA-4 binding molecule is administered to the subject at a dose of about 120 mg, about 240 mg, about 360 mg, about 480 mg, about 600 mg, about 720 mg, about 840 mg, or about 960 mg. In some embodiments, the CTLA-4 binding molecule is administered to the subject at a dose in the range of about 1 mg to about 2 mg, about 2 mg to about 5 mg, about 5 mg to about 10 mg, about 10 mg to about 20 mg, about 20 mg to about 50 mg, about 50 mg to about 100 mg, about 100 mg to about 200 mg, about 200 mg to about 500 mg, about 500 mg to about 1000 mg, about 1000 mg to about 2000 mg, about 1 mg to about 5 mg, about 2 mg to about 10 mg, about 5 mg to about 20 mg, about 10 mg to about 50 mg, about 20 mg to about 100 mg, about 50 mg to about 200 mg, about 100 mg to about 500 mg, about 200 mg to about 1000 mg, about 500 mg to about 2000 mg, about 1 mg to about 10 mg, about 2 mg to about 20 mg, about 5 mg to about 50 mg, about 10 mg to about 100 mg, about 20 mg to about 200 mg, about 50 mg to about 500 mg, about 100 mg to about 1000 mg, about 200 mg to about 2000 mg, about 1 mg to about 20 mg, about 2 mg to about 50 mg, about 5 mg to about 100 mg, about 10 mg to about 200 mg, about 20 mg to about 500 mg, about 50 mg to about 1000 mg, about 100 mg to about 2000 mg, about 1 mg to about 50 mg, about 2 mg to about 100 mg, about 5 mg to about 200 mg, about 10 mg to about 500 mg, about 20 mg to about 1000 mg, about 50 mg to about 2000 mg, about 1 mg to about 100 mg, about 2 mg to about 200 mg, about 5 mg to about 500 mg, about 10 mg to about 1000 mg, about 20 mg to about 2000 mg, about 1 mg to about 200 mg, about 2 mg to about 500 mg, about 5 mg to about 1000 mg, about 10 mg to about 2000 mg, about 1 mg to about 500 mg, about 2 mg to about 1000 mg, about 5 mg to about 2000 mg, about 1 mg to about 1000 mg, about 2 mg to about 2000 mg, or about 1 mg to about 2000 mg, including all ranges and subranges in between. In some embodiments, the CTLA-4 binding molecule is administered to the subject at a dose in the range of about 120 mg to about 840 mg, about 240 mg to about 720 mg, about 360 mg to about 600 mg, about 400 mg to about 560 mg, about 440 mg to about 520 mg, or about 460 mg to about 500 mg, including all ranges and subranges in between.

In some embodiments, the CTLA-4 binding molecule is administered to the subject over a period of about 10 minutes to about 1 hour (i.e., 60 minutes). In some embodiments, the CTLA-4 binding molecule is administered to the subject over a period of about 10 minutes, about 12 minutes, about 14 minutes, about 17 minutes, about 20 minutes, about 24 minutes, about 27 minutes, about 30 minutes, about 35 minutes, about 40 minutes, about 45 minutes, about 50 minutes, about 55 minutes, or about 60 minutes. In some embodiments, the CTLA-4 binding molecule is administered to the subject over a period of about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, about 55 minutes to about 60 minutes, about 10 minutes to about 20 minutes, about 15 minutes to about 25 minutes, about 20 minutes to about 30 minutes, about 25 minutes to about 35 minutes, about 30 minutes to about 40 minutes, about 35 minutes to about 45 minutes, about 40 minutes to about 50 minutes, about 45 minutes to about 55 minutes, about 50 minutes to about 60 minutes, about 10 minutes to about 25 minutes, about 15 minutes to about 30 minutes, about 20 minutes to about 35 minutes, about 25 minutes to about 40 minutes, about 30 minutes to about 45 minutes, about 35 minutes to about 50 minutes, about 40 minutes to about 55 minutes, about 45 minutes to about 60 minutes, about 10 minutes to about 30 minutes, about 15 minutes to about 35 minutes, about 20 minutes to about 40 minutes, about 25 minutes to about 45 minutes, about 30 minutes to about 50 minutes, about 35 minutes to about 55 minutes, about 40 minutes to about 60 minutes, about 10 minutes to about 35 minutes, about 15 minutes to about 40 minutes, about 20 minutes to about 45 minutes, about 25 minutes to about 50 minutes, about 30 minutes to about 55 minutes, about 35 minutes to about 60 minutes, about 10 minutes to about 40 minutes, about 15 minutes to about 45 minutes, about 20 minutes to about 50 minutes, about 25 minutes to about 55 minutes, about 30 minutes to about 60 minutes, about 10 minutes to about 45 minutes, about 15 minutes to about 50 minutes, about 20 minutes to about 55 minutes, about 25 minutes to about 60 minutes, about 10 minutes to about 50 minutes, about 15 minutes to about 55 minutes, about 20 minutes to about 60 minutes, about 10 minutes to about 55 minutes, or about 15 minutes to about 60 minutes, including all ranges and subranges in between. In some embodiments, the CTLA-4 binding molecule is administered to the subject over a period of about 15 minutes to about 45 minutes, about 20 minutes to about 40 minutes, or about 25 minutes to about 35 minutes, including all ranges and subranges in between. In some embodiments, the CTLA-4 binding molecule is administered to the subject over a period of about 30 minutes.

In some embodiments, the CTLA-4 binding molecule is administered to the subject once.

In some embodiments, the CTLA-4 binding molecule is administered to the subject more than once. In some embodiments, the CTLA-4 binding molecule is administered to the subject about 2, 3, 4, 5, 6, 7, 8, 9, 10, 11, 12, 13, 14, 15, 16, 17, 18, 19, or 20 times. In some embodiments, the CTLA-4 binding molecule is administered to the subject at least about 2, 3, 4, 5, 6, 7, 8, 9, 10, 11, 12, 13, 14, 15, 16, 17, 18, 19, or 20 times. In some embodiments, the CTLA-4 binding molecule is administered to the subject no more than about 2, 3, 4, 5, 6, 7, 8, 9, 10, 11, 12, 13, 14, 15, 16, 17, 18, 19, or 20 times.

In some embodiments, the CTLA-4 binding molecule is administered to the subject once every about 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the CTLA-4 binding molecule is administered to the subject once every about 1, 2, 3, 4, 5, 6, 7, 8, 9, 10, 11, 12, 13, 14, 15, 16, 17, 18, 19, or 20 weeks. In some embodiments, the CTLA-4 binding molecule is administered to the subject once every about 1, 2, 3, 4, 5, 6, 7, 8, 9, 10, 11, or 12 months. In some embodiments, the CTLA-4 binding molecule is administered to the subject once every 1-2 days, once every 2-4 days, once every 4-8 days, once every 8-16 days, once every 16-32 days, once every 32-64 days, once every 64-128 days, once every 128-256 days, once every 1-4 days, once every 2-8 days, once every 4-16 days, once every 8-32 days, once every 16-64 days, once every 32-128 days, once every 64-256 days, once every 1-8 days, once every 2-16 days, once every 4-32 days, once every 8-64 days, once every 16-128 days, once every 32-256 days, once every 1-16 days, once every 2-32 days, once every 4-64 days, once every 8-128 days, once every 16-256 days, once every 1-32 days, once every 2-64 days, once every 4-128 days, once every 8-256 days, once every 1-64 days, once every 2-128 days, once every 4-256 days, once every 1-128 days, once every 2-256 days, or once every 1-256 days, including all ranges and subranges in between. In some embodiments, the CTLA-4 binding molecule is administered to the subject once every 3-5 days, once every 5-7 days, once every 7-9 days, once every 9-11 days, once every 11-13 days, once every 13-15 days, once every 15-17 days, once every 17-19 days, once every 19-21 days, once every 3-7 days, once every 5-9 days, once every 7-11 days, once every 9-13 days, once every 11-15 days, once every 13-17 days, once every 15-19 days, once every 17-21 days, once every 3-9 days, once every 5-11 days, once every 7-13 days, once every 9-15 days, once every 11-17 days, once every 13-19 days, once every 15-21 days, once every 3-11 days, once every 5-13 days, once every 7-15 days, once every 9-17 days, once every 11-19 days, once every 13-21 days, once every 3-13 days, once every 5-15 days, once every 7-17 days, once every 9-19 days, once every 11-21 days, once every 3-15 days, once every 5-17 days, once every 7-19 days, once every 9-21 days, once every 3-17 days, once every 5-19 days, once every 7-21 days, once every 3-19 days, once every 5-21 days, or once every 3-21 days, including all ranges and subranges in between. In some embodiments, the CTLA-4 binding molecule is administered to the subject once every 10-18 days, once every 11-17 days, once every 12-16 days, or once every 13-15 days, including all ranges and subranges in between. In some embodiments, the CTLA-4 binding molecule is administered to the subject once every about 14 days. In some embodiments, the CTLA-4 binding molecule is administered to the subject once every 3-11 days, once every 4-10 days, once every 5-9 days, or once every 6-8 days. In some embodiments, the CTLA-4 binding molecule is administered to the subject once every about 7 days.

In some embodiments, the CTLA-4 binding molecule is administered to the subject over an about 14, 15, 16, 17, 18, 19, 20, 21, 22, 23, 24, 25, 26, 27, 28, 29, 30, 31, 32, 33, 34, 35, 36, 37, 38, 39, 40, 41, or 42 day cycle. In some embodiments, the CTLA-4 binding molecule is administered to the subject over an about 14, 21, 28, 35, or about 42 day cycle. In some embodiments, the CTLA-4 binding molecule is administered to the subject over an about 35 day cycle. In some embodiments, the CTLA-4 binding molecule is administered to the subject over an about 28 day cycle. In some embodiments, the CTLA-4 binding molecule is administered to the subject over an about 21 day cycle. In some embodiments, the CTLA-4 binding molecule is administered to the subject over an about 14 day cycle. In some embodiments, the CTLA-4 binding molecule is administered to the subject over 1, 2, 3, 4, 5, 6, 7, 8, 9, or 10 cycles.

In some embodiments, the CTLA-4 binding molecule is administered to the subject on days 1, 8, 15, and 22 of a 28 day cycle. In some embodiments, the CTLA-4 binding molecule is administered to the subject on days 1 and 15 of the 28 day cycle. In some embodiments, the CTLA-4 binding molecule is administered to the subject on days 1 and 8 of the 28 day cycle. In some embodiments, the CTLA-4 binding molecule is administered to the subject on days 1 and 22 of the 28 day cycle. In some embodiments, the CTLA-4 binding molecule is administered to the subject on days 8 and 22 of the 28 day cycle. In some embodiments, the CTLA-4 binding molecule is administered to the subject on days 15 and 22 of the 28 day cycle. In some embodiments, the CTLA-4 binding molecule is administered to the subject on days 8 and 15 of the 28 day cycle. In some embodiments, the CTLA-4 binding molecule is administered to the subject on days 1, 8, and 15 of the 28 day cycle. In some embodiments, the CTLA-4 binding molecule is administered to the subject on days 8, 15, and 22 of the 28 day cycle In some embodiments, the CTLA-4 binding molecule is administered to the subject on days 1, 5, 9, 13, 17, 21, and 25 of the 28 day cycle. In some embodiments, the CTLA-4 binding molecule is administered to the subject on days 1, 3, 5, 7, 9, 11, 13, 15, 17, 19, 21, 23, 25, and 27 of the 28-day cycle. In some embodiments, the CTLA-4 binding molecule is administered to the subject on any one of days 1, 2, 3, 4, 5, 6, 7, 8, 9, 10, 11, 12, 13, 14, 15, 16, 17, 18, 19, 20, 21, 22, 23, 24, 25, 26, 27, and 28 of the 28 day cycle.

In some embodiments, the subject receives the CTLA-4 binding molecule in combination with a PD-1 inhibitor. In some embodiments, the PD-1 inhibitor is administered to the subject at a dose in the range of about 0.1 mg/kg to about 2000 mg/kg body weight. In some embodiments, the PD-1 inhibitor is administered to the subject at a dose of about 0.1 mg/kg, about 0.12 mg/kg, about 0.14 mg/kg, about 0.17 mg/kg, about 0.2 mg/kg, about 0.24 mg/kg, about 0.27 mg/kg, about 0.3 mg/kg, about 0.35 mg/kg, about 0.4 mg/kg, about 0.45 mg/kg, about 0.5 mg/kg, about 0.6 mg/kg, about 0.7 mg/kg, about 0.8 mg/kg, about 0.9 mg/kg, about 1 mg/kg, about 1.2 mg/kg, about 1.4 mg/kg, about 1.7 mg/kg, about 2 mg/kg, about 2.4 mg/kg, about 2.7 mg/kg, about 3 mg/kg, about 3.3 mg/kg, about 3.7 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 11 mg/kg, about 12 mg/kg, about 14 mg/kg, about 17 mg/kg, about 20 mg/kg, about 24 mg/kg, about 27 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 210 mg/kg, about 220 mg/kg, about 230 mg/kg, about 240 mg/kg, about 250 mg/kg, about 260 mg/kg, about 270 mg/kg, about 280 mg/kg, about 290 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1000 mg/kg, about 1100 mg/kg, about 1200 mg/kg, about 1300 mg/kg, about 1400 mg/kg, about 1500 mg/kg, about 1600 mg/kg, about 1700 mg/kg, about 1800 mg/kg, about 1900 mg/kg, or about 2000 mg/kg, body weight. In some embodiments, the PD-1 inhibitor is administered to the subject at a dose in the range of about 0.1 mg/kg to about 0.2 mg/kg, about 0.2 mg/kg to about 0.5 mg/kg, about 0.5 mg/kg to about 1 mg/kg, about 1 mg/kg to about 2 mg/kg, about 2 mg/kg to about 5 mg/kg, about 5 mg/kg to about 10 mg/kg, about 10 mg/kg to about 20 mg/kg, about 20 mg/kg to about 50 mg/kg, about 50 mg/kg to about 100 mg/kg, about 0.1 mg/kg to about 0.5 mg/kg, about 0.2 mg/kg to about 1 mg/kg, about 0.5 mg/kg to about 2 mg/kg, about 1 mg/kg to about 5 mg/kg, about 2 mg/kg to about 10 mg/kg, about 5 mg/kg to about 20 mg/kg, about 10 mg/kg to about 50 mg/kg, about 20 mg/kg to about 100 mg/kg, about 0.1 mg/kg to about 1 mg/kg, about 0.2 mg/kg to about 2 mg/kg, about 0.5 mg/kg to about 5 mg/kg, about 1 mg/kg to about 10 mg/kg, about 2 mg/kg to about 20 mg/kg, about 5 mg/kg to about 50 mg/kg, about 10 mg/kg to about 100 mg/kg, about 0.1 mg/kg to about 2 mg/kg, about 0.2 mg/kg to about 5 mg/kg, about 0.5 mg/kg to about 10 mg/kg, about 1 mg/kg to about 20 mg/kg, about 2 mg/kg to about 50 mg/kg, about 5 mg/kg to about 100 mg/kg, about 0.1 mg/kg to about 5 mg/kg, about 0.2 mg/kg to about 10 mg/kg, about 0.5 mg/kg to about 20 mg/kg, about 1 mg/kg to about 50 mg/kg, about 2 mg/kg to about 100 mg/kg, about 0.1 mg/kg to about 10 mg/kg, about 0.2 mg/kg to about 20 mg/kg, about 0.5 mg/kg to about 50 mg/kg, about 1 mg/kg to about 100 mg/kg, about 0.1 mg/kg to about 20 mg/kg, about 0.2 mg/kg to about 50 mg/kg, about 0.5 mg/kg to about 100 mg/kg, about 0.1 mg/kg to about 50 mg/kg, about 0.2 mg/kg to about 100 mg/kg, or about 0.1 mg/kg to about 100 mg/kg, including all ranges and subranges in between. In some embodiments, the PD-1 inhibitor is administered to the subject at a dose in the range of about 1 mg/kg to about 50 mg/kg, about 2 mg/kg to about 40 mg/kg, about 3 mg/kg to about 30 mg/kg, about 4 mg/kg to about 20 mg/kg, about 5 mg/kg to about 15 mg/kg, or about 6 mg/kg to about 10 mg/kg, including all ranges and subranges in between. In some embodiments, the PD-1 inhibitor is an anti-PD-1 antibody. In some embodiments, the anti-PD-1 antibody is nivolumab.

In some embodiments, the PD-1 inhibitor is administered to the subject at a dose in the range of about 0.1 mg to about 2000 mg. In some embodiments, the PD-1 inhibitor is administered to the subject at a dose of about 0.1 mg, about 0.12 mg, about 0.14 mg, about 0.17 mg, about 0.2 mg, about 0.24 mg, about 0.27 mg, about 0.3 mg, about 0.35 mg, about 0.4 mg, about 0.45 mg, about 0.5 mg, about 0.6 mg, about 0.7 mg, about 0.8 mg, about 0.9 mg, about 1 mg, about 1.2 mg, about 1.4 mg, about 1.7 mg, about 2 mg, about 2.4 mg, about 2.7 mg, about 3 mg, about 3.3 mg, about 3.7 mg, about 4 mg, about 4.5 mg, about 5 mg, about 6 mg, about 7 mg, about 8 mg, about 9 mg, about 10 mg, about 11 mg, about 12 mg, about 14 mg, about 17 mg, about 20 mg, about 24 mg, about 27 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg. In some embodiments, the PD-1 inhibitor is administered to the subject at a dose of about 120 mg, about 240 mg, about 360 mg, about 480 mg, about 600 mg, about 720 mg, about 840 mg, or about 960 mg. In some embodiments, the PD-1 inhibitor is administered to the subject at a dose in the range of about 1 mg to about 2 mg, about 2 mg to about 5 mg, about 5 mg to about 10 mg, about 10 mg to about 20 mg, about 20 mg to about 50 mg, about 50 mg to about 100 mg, about 100 mg to about 200 mg, about 200 mg to about 500 mg, about 500 mg to about 1000 mg, about 1000 mg to about 2000 mg, about 1 mg to about 5 mg, about 2 mg to about 10 mg, about 5 mg to about 20 mg, about 10 mg to about 50 mg, about 20 mg to about 100 mg, about 50 mg to about 200 mg, about 100 mg to about 500 mg, about 200 mg to about 1000 mg, about 500 mg to about 2000 mg, about 1 mg to about 10 mg, about 2 mg to about 20 mg, about 5 mg to about 50 mg, about 10 mg to about 100 mg, about 20 mg to about 200 mg, about 50 mg to about 500 mg, about 100 mg to about 1000 mg, about 200 mg to about 2000 mg, about 1 mg to about 20 mg, about 2 mg to about 50 mg, about 5 mg to about 100 mg, about 10 mg to about 200 mg, about 20 mg to about 500 mg, about 50 mg to about 1000 mg, about 100 mg to about 2000 mg, about 1 mg to about 50 mg, about 2 mg to about 100 mg, about 5 mg to about 200 mg, about 10 mg to about 500 mg, about 20 mg to about 1000 mg, about 50 mg to about 2000 mg, about 1 mg to about 100 mg, about 2 mg to about 200 mg, about 5 mg to about 500 mg, about 10 mg to about 1000 mg, about 20 mg to about 2000 mg, about 1 mg to about 200 mg, about 2 mg to about 500 mg, about 5 mg to about 1000 mg, about 10 mg to about 2000 mg, about 1 mg to about 500 mg, about 2 mg to about 1000 mg, about 5 mg to about 2000 mg, about 1 mg to about 1000 mg, about 2 mg to about 2000 mg, or about 1 mg to about 2000 mg, including all ranges and subranges in between. In some embodiments, the PD-1 inhibitor is administered to the subject at a dose in the range of about 120 mg to about 840 mg, about 240 mg to about 720 mg, about 360 mg to about 600 mg, about 400 mg to about 560 mg, about 440 mg to about 520 mg, or about 460 mg to about 500 mg, including all ranges and subranges in between. In some embodiments, the PD-1 inhibitor is administered to the subject at a dose of about 480 mg. In some embodiments, the PD-1 inhibitor is an anti-PD-1 antibody. In some embodiments, the anti-PD-1 antibody is nivolumab.

In some embodiments, the PD-1 inhibitor is administered to the subject once.

In some embodiments, the PD-1 inhibitor is administered to the subject more than once. In some embodiments, the PD-1 inhibitor is administered to the subject about 2, 3, 4, 5, 6, 7, 8, 9, 10, 11, 12, 13, 14, 15, 16, 17, 18, 19, or 20 times. In some embodiments, the PD-1 inhibitor is administered to the subject at least about 2, 3, 4, 5, 6, 7, 8, 9, 10, 11, 12, 13, 14, 15, 16, 17, 18, 19, or 20 times. In some embodiments, the PD-1 inhibitor is administered to the subject no more than about 2, 3, 4, 5, 6, 7, 8, 9, 10, 11, 12, 13, 14, 15, 16, 17, 18, 19, or 20 times. In some embodiments, the PD-1 inhibitor is an anti-PD-1 antibody. In some embodiments, the anti-PD-1 antibody is nivolumab.

In some embodiments, the PD-1 inhibitor is administered to the subject once in a treatment cycle. In some embodiments, the PD-1 inhibitor is administered to the subject over at least one treatment cycle. In some embodiments, the PD-1 inhibitor is administered to the subject over the second treatment cycle. In some embodiments, the treatment cycle is a 14, 15, 16, 17, 18, 19, 20, 21, 22, 23, 24, 25, 26, 27, 28, 29, 30, 31, 32, 33, 34, 35, 36, 37, 38, 39, 40, 41, or 42 day cycle. In some embodiments, the treatment cycle is a 28 day cycle. In some embodiments, the PD-1 inhibitor is administered to the subject on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of the treatment cycle. In some embodiments, the PD-1 inhibitor is administered to the subject on day 1 of the treatment cycle. In some embodiments, the PD-1 inhibitor is administered to the subject on any one of days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle. In some embodiments, the PD-1 inhibitor is administered to the subject on day 1 of a 28 day cycle. In some embodiments, the 28 day cycle is the second treatment cycle. In some embodiments, the PD-1 inhibitor is an anti-PD-1 antibody. In some embodiments, the anti-PD-1 antibody is nivolumab.

In some embodiments, methods for reducing the immunosuppressive activity of an immune cell in a subject in need thereof comprise administering to the subject an effective amount of (i) a binding molecule, (ii) a nucleic acid encoding the binding molecule (e.g., an expression vector), or (iii) a composition comprising the binding molecule or the nucleic acid encoding the same.

In some embodiments, methods for treating or preventing cancer comprise administering to a subject in need thereof an effective amount of (i) a binding molecule, (ii) a nucleic acid encoding the binding molecule (e.g., an expression vector), or (iii) a composition comprising the binding molecule or the nucleic acid encoding the same.

In some embodiments, the binding molecule binds to CTLA-4 that is present on the surface of an immunosuppressive immune cell in the subject but is not present on the surface of the subject's cancer cells. In some embodiments, the binding molecule directly kills the immunosuppressive immune cell, but does not directly kill the subject's cancer cells.

In some embodiments, the binding molecule binds to CTLA-4 that is present on the surface of an immunosuppressive cell in the subject, and the subject's cancer cells. In some embodiments, the binding molecule directly kills the immunosuppressive immune cell and the subject's cancer cells.

In some embodiments, the binding molecule binds to CTLA-4, but does not block the interaction between CTLA-4 and one or more of its ligands. For example, in some embodiments, a CTLA-4 binding molecule does not block the interaction between CTLA-4 and CD80 (B7-1). In some embodiments, a CTLA-4 binding molecule does not block the interaction between CTLA-4 and CD86 (B7-2). In some embodiments, a CTLA-4 binding molecule does not block the interaction between CTLA-4 and either CD80 (B7-1) or CD86 (B7-2).

In some embodiments, the binding molecule binds to CTLA-4 and also blocks the interaction between CTLA-4 and one or more of its ligands. For example, in some embodiments, a CTLA-4 binding molecule blocks the interaction between CTLA-4 and CD80 (B7-1). In some embodiments, a CTLA-4 binding molecule blocks the interaction between CTLA-4 and CD86 (B7-2). In some embodiments, a CTLA-4 binding molecule blocks the interaction between CTLA-4 and both of CD80 (B7-1) or CD86 (B7-2).

In some embodiments, the subject has cancer. In some embodiments, the cancer is characterized by the presence of at least one immunosuppressive cell, for example in the tumor microenvironment. In some embodiments, the cancer is characterized by a high mutational burden (TMB) and/or a high frequency of indels. Mutational burden can be analyzed by various methods, including hybrid-based next-generation sequencing, and is reported as the total number of sequence variants or mutations per tumor genomic region analyzed (e.g., mutations per megabase). Cancers can be classified as having a “high” mutational burden if they have greater than or equal to 20 mutations per megabase. High mutational burden is typical of cancers developed as a consequence of exposure to powerful carcinogens, such as tobacco smoke and polycyclic aromatic hydrocarbons (e.g., in lung and bladder cancers), as well as exposure to mutagens (e.g., UV light in melanoma). Indels (insertions and deletions) are one type of mutation commonly seen in cancer cells. Indels that produce frameshift mutations can generate highly immunogenic tumor neoantigens. Therefore, the presence of a high frequency of indels can lead to better response the therapeutic approaches described herein.

In some embodiments, the cancer is a solid tumor. In some embodiments, the cancer is a blood cancer. In some embodiments, the cancer is metastatic.

In some embodiments, the cancer is any one of the following: bladder cancer (e.g., urothelial carcinoma), breast cancer (e.g., HER2 positive breast cancer, triple negative breast cancer), cervical cancer (cervical carcinoma), colon cancer (e.g., colorectal cancer such as metastatic microsatellite instability-high or mismatch repair deficient colorectal cancer), endometrial cancer, esophageal cancer (esophageal squamous cell carcinoma), microsatellite instability-high (MSI-H) or mismatch repair deficient (dMMR) cancers, fallopian tube cancer, gastrointestinal cancer (e.g., gastric carcinoma, biliary tract neoplasm, gastroesophageal junction carcinoma), glioblastoma, glioma, head and neck cancer (e.g., squamous cell carcinoma of the head and neck), kidney cancer (e.g., renal cell carcinoma, advanced renal cell carcinoma), liver cancer (e.g., hepatocellular carcinoma), lung cancer (e.g., non-small cell lung cancer (e.g., metastatic non-small cell lung cancer), small-cell lung cancer), lymphoma (e.g., diffuse large B-cell lymphoma, Hodgkin lymphoma, non-Hodgkin lymphoma, primary mediastinal large B-cell lymphoma), Merkel cell carcinoma, mesothelioma (e.g., pleural mesothelioma, malignant pleural mesothelioma, unresectable malignant pleural mesothelioma), myeloma (e.g., multiple myeloma), nasopharyngeal neoplasm, ovarian cancer, pancreatic cancer, peritoneal neoplasm, prostate cancer, soft tissue sarcoma, skin cancer (e.g., squamous cell cancer of the skin, melanoma (e.g., unresectable melanoma or metastatic melanoma), transitional cell carcinoma, or urothelial cancer.

In some embodiments, the cancer is cervical cancer, and the cervical cancer is cervical carcinoma.

In some embodiments, the cancer is bladder cancer, and the bladder cancer is urothelial carcinoma.

In some embodiments, the cancer is breast cancer, and the breast cancer is HER2 positive breast cancer or triple negative breast cancer.

In some embodiments, the cancer is colon cancer, and the colon cancer is colorectal cancer.

In some embodiments, the cancer is esophageal cancer, and the esophageal cancer is esophageal squamous cell carcinoma.

In some embodiments, the cancer is gastrointestinal cancer, and the gastrointestinal cancer is gastric cancer, biliary tract neoplasm, or gastroesophageal junction cancer.

In some embodiments, the cancer is MSI-H or dMMR cancer.

In some embodiments, the cancer is glioma, and the glioma is glioblastoma.

In some embodiments, the cancer is head and neck cancer, and the head and neck cancer is squamous cell carcinoma of the head and neck.

In some embodiments, the cancer is kidney cancer, and the kidney cancer is renal cell carcinoma.

In some embodiments, the cancer is liver cancer, and the liver cancer is hepatocellular carcinoma.

In some embodiments, the cancer is lung cancer, and the lung cancer is non-small cell lung cancer or small-cell lung cancer. In some embodiments, the non-small cell lung cancer is metastatic non-small cell lung cancer.

In some embodiments, the cancer is lymphoma, and the lymphoma is Hodgkin lymphoma, non-Hodgkin lymphoma, primary mediastinal large B-cell lymphoma, or diffuse large B-cell lymphoma.

In some embodiments, the cancer is mesothelioma, and the mesothelioma is pleural mesothelioma. In some embodiments, the mesothelioma is malignant pleural mesothelioma.

In some embodiments, the cancer the cancer is myeloma, and the myeloma is multiple myeloma.

In some embodiments, the cancer is skin cancer, and the skin cancer is squamous cell cancer of the skin or melanoma. In some embodiments, the melanoma is unresectable melanoma or metastatic melanoma.

In some embodiments, the subject received at least one line or regimen of prior treatment, before administration with a binding molecule. In some embodiments, subject has cancer, and the cancer is relapsed or refractory to at least one prior treatment, such as checkpoint inhibitor therapy. In some embodiments, the cancer is relapsed or refractory to ipilimumab, nivolumab, pembrolizumab, atezolizumab, durvalumab, avelumab, tremelimumab, cemiplimab, relatlimab, tiragolumab, ociperlimab, vibostolimab, domvanalimab, sacituzumab, sacituzumab govitecan, datopotamab, or datopotamab deruxtecan. In some embodiments, the cancer is one of the cancers listed in Table 8, below, and is relapsed or refractory to at least one prior treatment marked with an “X” in the table.

TABLE 8
Cancers treatable with a binding molecule of the disclosure that can be relapsed or refractory to prior treatments

| Cancer | Ipilimumab | Nivolumab | Pembroizumab | Atezolizumab | Durvalumab | Avelumab | Cemiplimab |
|---|---|---|---|---|---|---|---|
| Melanoma | X | X | X | | | | |
| Merkel Cell | | | X | | | X | |
| Cutaneous | | | | | | | X |
| Squamous | | | | | | | |
| Cell | | | | | | | |
| Carcinoma | | | | | | | |
| Non-small | | X | X | X | X | | |
| cell lung | | | | | | | |
| cancer | | | | | | | |
| Small cell | | X | X | X | | | |
| lung cancer | | | | | | | |
| Squamous | | X | X | | X | | |
| cell | | | | | | | |
| carcinoma | | | | | | | |
| of the head | | | | | | | |
| and neck | | | | | | | |
| Esophageal | | | X | | | | |
| cancer | | | | | | | |
| Gastric | | X | X | | | | |
| cancer | | | | | | | |
| Colorectal | | X | X | | | | |
| cancer | | | | | | | |
| Hepatocellular | | X | | | | | |
| carcinoma | | | | | | | |
| Bladder | | X | X | X | X | X | |
| cancer | | | | | | | |
| Renal Cell | X | X | X | | | X | |
| Carcinoma | | | | | | | |

In some embodiments, the subject receives at least one pre-medication prior to administration of the CTLA-4 binding molecule. In some embodiments, the at least one pre-medication is an H1/H2 blocker-containing agent and/or anti-pyrectic agent.

In some embodiments, a method for treating cancer comprises administering to the subject in need thereof an effective amount a nucleic acid or an expression vector as described herein, e.g., a nucleic acid or an expression vector encoding a CTLA-4 binding molecule or a fragment or variant thereof.

The binding molecules or pharmaceutical compositions described herein can be administered alone or in combination with other therapeutic or diagnostic agents. A combination therapy can include a binding molecule, or pharmaceutical composition thereof, combined with at least one other therapeutic agent selected based on the particular subject, disease or condition to be treated. Examples of other such agents include, inter alia, a cytotoxic, anti-cancer or chemotherapeutic agent, a checkpoint inhibitor, an anti-inflammatory or anti-proliferative agent, an antimicrobial or antiviral agent, growth factors, cytokines, an analgesic, a therapeutically active small molecule or polypeptide, a single chain antibody, a classical antibody or fragment thereof, or a nucleic acid molecule which modulates signaling pathways, and similar modulating therapeutic molecules which can complement or otherwise be beneficial in a therapeutic or prophylactic treatment regimen. In some embodiments, the anti-cancer agent is an antimetabolite, alkylating agent, anti-tumor antibiotic, vinca alkaloid, taxane, podophyllotoxin, and/or camptothecin. In some embodiments, the anti-cancer agent is a small molecule. In some embodiments, the anti-cancer agent is a platinum-based agent, such as, for example, cisplatin, carboplatin, oxaliplatin, nedaplatin, lobaplatin, heptaplatin, miriplatin, or a combination thereof. In some embodiments, the CTLA-4 binding molecule is administered in combination with cisplatin. In some embodiments, the CTLA-4 binding molecule is administered in combination with carboplatin. In some embodiments, the CTLA-4 binding molecule is administered in combination with oxaliplatin. In some embodiments, the CTLA-4 binding molecule is administered in combination with cisplatin, carboplatin, oxaliplatin, or a combination thereof.

In some embodiments, the CTLA4 binding molecule is administered as part of a combination therapy. Provided herein is a method of treating cancer, the method comprising administering to a subject in need thereof: (i) an effective amount of a CTLA-4 binding molecule comprising a Shiga toxin A subunit effector polypeptide and a binding region capable of specifically binding an extracellular part of CTLA-4, wherein the binding region comprises a VHH domain comprising a HCDR1, a HCDR2, and a HCDR3; and (ii) an additional anti-cancer agent, wherein the anti-cancer agent is an inhibitor of PD-1, PD-L1, or CTLA-4.

Further provided herein is a method of treating cancer, the method comprising administering to a subject in need thereof: (i) an effective amount of a CTLA-4 binding molecule comprising a Shiga toxin A subunit effector polypeptide and a binding region capable of specifically binding an extracellular part of CTLA-4; wherein the binding region comprises a first VHH domain comprising a first HCDR1, a first HCDR2, and a first HCDR3 and a second VHH domain comprising a second HCDR1, a second HCDR2, and a second HCDR3; and (ii) an additional anti-cancer agent, wherein the anti-cancer agent is an inhibitor of PD-1, PD-L1, or CTLA-4.

In some embodiments, the inhibitor of PD-1 is an anti-PD-1 antibody or an anti-PD-1 antibody-drug conjugate (ADC). In some embodiments, the anti-PD-1 antibody is nivolumab, pembrolizumab, dostarlimab, tislelizumab, or cemiplimab. In some embodiments, the anti-PD-1 antibody is nivolumab.

In some embodiments, the inhibitor of PD-L1 is an anti-PD-L1 antibody or an anti-PD-L1 ADC. In some embodiments, the anti-PD-L1 antibody is atezolizumab, durvalumab, or avelumab.

In some embodiments, the inhibitor of CTLA-4 is an anti-CTLA-4 antibody or an anti-CTLA-4 ADC. In some embodiments, the anti-CTLA-4 antibody is ipilimumab or tremelimumab.

In some embodiments of the methods disclosed herein, the CTLA-4 binding molecule is administered to the subject before the additional anti-cancer agent. In some embodiments of the methods disclosed herein, the CTLA-4 binding molecule is administered to the subject after the additional anti-cancer agent. In some embodiments of the methods disclosed herein, the CTLA-4 binding molecule is administered to the subject at the same time as the additional anti-cancer agent.

In some embodiments, treatment of a subject with a binding molecule or pharmaceutical composition leads to cell death of targeted cells and/or the inhibition of growth of targeted cells, wherein the targeted cells can be immunosuppressive immune cells and/or tumor cells. As such, the disclosed binding molecules, and pharmaceutical compositions comprising them, will be useful in methods for treating a variety of pathological disorders in which killing or depleting target cells can be beneficial, such as, inter alia, cancer, tumors, and other growth abnormalities.

Also provided herein are methods for killing a CTLA-4 expressing cell, the methods comprising the step of contacting the cell with a CTLA-4 binding molecule or a pharmaceutical composition comprising the same.

### VIII. Kits

Also provided herein are kits comprising a binding molecule, and optionally, instructions for use, additional reagent(s), and/or pharmaceutical delivery device(s). The kit can comprise reagents and other tools for detecting a cell type (e.g., an immunosuppressive immune cell or a tumor cell) in a sample or in a subject.

In some embodiments, provided herein are a device comprising a binding molecule (e.g., in the form of a pharmaceutical composition or diagnostic composition), for delivery to a subject in need thereof. Thus, a delivery device comprising a composition as described herein can be used to administer to a subject a binding molecule by various delivery methods, including: intravenous, subcutaneous, intramuscular or intraperitoneal injection; or by other suitable means recognized by a person of skill in the art.

Also provided herein are kits comprising at least one composition of matter disclosed herein (e.g., a binding molecule), and optionally, packaging and instructions for use. Kits can be useful for drug administration and/or diagnostic information gathering. In some embodiments, a kit can optionally comprise at least one additional reagent (e.g., standards, markers and the like). Kits typically include a label indicating the intended use of the contents of the kit. The kit can further comprise reagents and other tools for detecting a cell type (e.g., a tumor cell) in a sample or in a subject, or for diagnosing whether a subject belongs to a group that responds to a therapeutic strategy which makes use of a compound, composition, or related method, e.g., such as a method described herein.

TABLE 9
Exemplary CTLA-4 binding molecules

| SEQ ID | | |
|---|---|---|
| NO | ETB ID | Amino Acid Sequence |
| 286 | 116983 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHHHHASAVAAQVKLVESGGGSVQAGGSLRLSCAASGDSYSVKYMGWFRRAPGKQRDQREEVASIYPTGGT |
| | | FYTDSVKGRFTISRDNAKSTLYLQMNSLKPEDTAMYYCAAGKWGTDYWGQGTQVTVSS |
| 287 | 116984 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSS |
| 288 | 117094 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSS |
| 289 | 117146 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMTALKPEDTAMYYCAAGKWGTDYWGQGTQVTVSSEAAAKQVQLVESGGGSVQAG |
| | | GSLRLSCAASGYYNRYCLGWFRQTPGKEREAVATIDTDGSTSYADSVKGRFTISFDNAKNTLYLQMNSLKPEDTAMY |
| | | YCAAGPNPRYCSGAVNTRGAEHYFGYWAQGTQVTVSS |
| 290 | 117147 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMTALKPEDTAMYYCAAGKWGTDYWGQGTQVTVSSEAAAKEAAAKEAAAKQVQLVE |
| | | SGGGSVQAGGSLRLSCAASGYYNRYCLGWFRQTPGKEREAVATIDTDGSTSYADSVKGRFTISFDNAKNTLYLQMNS |
| | | LKPEDTAMYYCAAGPNPRYCSGAVNTRGAEHYFGYWAQGTQVTVSS |
| 291 | 117148 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMTALKPEDTAMYYCAAGKWGTDYWGQGTQVTVSSRNSISSLSIPSTVPRARDPPVD |
| | | MASSEDVIKEFMRFKVRMEGSVNGHEFEIEGEGEGRPYEGTQTAKLKVTKGGPLPFAWDILSPQFQYGSKAYVKHPA |
| | | DIPDYLKLSFPEGFKWERVMNFEDGGVVTVTQDSSLQDGEFIYKVKLRGTNFPSDGPVMQKKTMGWEASTERMYPE |
| | | DGALKGEIKMRLKLKDGGHYDAEVKTTYMAKKPVQLPGAYKTDIKLDITSHNEDYTIVEQYERAEGRHSTGAGLYKQV |
| | | QLVESGGGSVQAGGSLRLSCAASGYYNRYCLGWFRQTPGKEREAVATIDTDGSTSYADSVKGRFTISFDNAKNTLYL |
| | | QMNSLKPEDTAMYYCAAGPNPRYCSGAVNTRGAEHYFGYWAQGTQVTVSS |
| 292 | 117178 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSSEAAAKQVQLVESGGGLVQPG |
| | | GSLRLSCAASGVNFSSYTMSWVRQAPGLGLEWVAYINSGGGTTSYADSVKGRFTISRDNAKNTLYLQMNSLLPEDTA |
| | | MYYCQGGLYRGQGTQVTVSS |
| 293 | 117179 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVQLVESGGGLVQPGGSLRLSCAASGVNFSSYTMSWVRQAPGLGLEWVAYINSGGGTTSYADS |
| | | VKGRFTISRDNAKNTLYLQMNSLLPEDTAMYYCQGGLYRGQGTQVTVSSEAAAKEVQLVESGGGLVQPGGSLRLSC |
| | | EGTGSIFSPNAMGWYRQGPGKQRELVASVTSSGTTNYSDSVKGRFTISMDNYKHLAYLQMNSLKPEDTAVYYCNLKE |
| | | LGTFYRRDFWGQGTQVTVSS |
| 294 | 117182 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSSEAAAKQVQLQESGGGLVQPG |
| | | GSLRLSCAASGSIRSSNTMGWYRQAPGKERDLIVSMSSGGFRSYVDSVKDRFTISRDNAKNTVYLQMNSLKSEDTAV |
| | | YYCRYLAPDVQSWGQGIQVTVSS |
| 295 | 117183 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVQLQESGGGLVQPGGSLRLSCAASGSIRSSNTMGWYRQAPGKERDLIVSMSSGGFRSYVDSV |
| | | KDRFTISRDNAKNTVYLQMNSLKSEDTAVYYCRYLAPDVQSWGQGIQVTVSSEAAAKEVQLVESGGGLVQPGGSLRL |
| | | SCEGTGSIFSPNAMGWYRQGPGKQRELVASVTSSGTTNYSDSVKGRFTISMDNYKHLAYLQMNSLKPEDTAVYYCNL |
| | | KELGTFYRRDFWGQGTQVTVSS |
| 296 | 117407 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTADALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSS |
| 297 | 117408 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSS |
| 298 | 117586 | MKEFTLDFSTAKTYVDSLNVIRSAIGTPLQTISSGGTSLLMIDSGIGDNLFAVDILGFDFTLGRFNNLRLIVERNNLSVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTADALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNUSDSV |
| | | KGRFTISMDNYKHLAYLQMNSLKPEDTAVYYCNLKELGTFYRRDFWGQGTQVTVSS |
| 299 | 117590 | MKEFTLDFSTAKTYVDSLNVIRSAIGTPLQTISSGGTSLLMIDSGIGDNLFAVDILGFDFTLGRFNNLRLIVERNNLSVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTADALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSS |
| 300 | 117709 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVQLVESGGGSVQAGGSLRLSCAASGYYNRYCLGWFRQTPGKEREAVATIDTDGSTSYADSVK |
| | | GRFTISFDNAKNTLYLQMNSLKPEDTAMYYCAAGPNPRYCSGAVNTRGAEHYFGYWAQGTQVTVSSEAAAKEVQLV |
| | | ESGGGLVQPGGSLRLSCEGTGSIFSPNAMGWYRQGPGKQRELVASVTSSGTTNYSDSVKGRFTISMDNYKHLAYLQ |
| | | MNSLKPEDTAVYYCNLKELGTFYRRDFWGQGTQVTVSS |
| 301 | 117710 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVQLVESGGGSVQAGGSLRLSCAASGYYNRYCLGWFRQTPGKEREAVATIDTDGSTSYADSVK |
| | | GRFTISFDNAKNTLYLQMNSLKPEDTAMYYCAAGPNPRYCSGAVNTRGAEHYFGYWAQGTQVTVSSGGGGSGGGS |
| | | EVQLVESGGGLVQPGGSLRLSCEGTGSIFSPNAMGWYRQGPGKQRELVASVTSSGTTNYSDSVKGRFTISMDNYKH |
| | | LAYLQMNSLKPEDTAVYYCNLKELGTFYRRDFWGQGTQVTVSS |
| 302 | 117713 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSSEAAAKEVQLVESGGGLVQPG |
| | | GSLRLSCEGTGSIFSPNAMGWYRQGPGKQRELVASVTSSGTTNYSDSVKGRFTISMDNYKHLAYLQMNSLKPEDTAV |
| | | YYCNLKELGTFYRRDFWGQGTQVTVSS |
| 303 | 117714 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSSGGGGSGGGSEVQLVESGGGL |
| | | VQPGGSLRLSCEGTGSIFSPNAMGWYRQGPGKQRELVASVTSSGTTNYSDSVKGRFTISMDNYKHLAYLQMNSLKP |
| | | EDTAVYYCNLKELGTFYRRDFWGQGTQVTVSS |
| 304 | 118123 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | KGRFTISMDNYKHLAYLQMNSLKPEDTAVYYCNLKELGTFYRRDFWGQGTQVTVSSGGGGSGGGSEVQLVESGGGL |
| | | VQPGGSLRLSCEGTGSIFSPNAMGWYRQGPGKQRELVASVTSSGTTNYSDSVKGRFTISMDNYKHLAYLQMNSLKP |
| | | EDTAVYYCNLKELGTFYRRDFWGQGTQVTVSSGGGGSLAEAKVLANRELDKYGVSDYYKNLINNAKTVEGVKALIDEI |
| | | LAALP |
| 305 | 118124 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | KGRFTISMDNYKHLAYLQMNSLKPEDTAVYYCNLKELGTFYRRDFWGQGTQVTVSSGGGGSGGGSEVQLVESGGGL |
| | | VQPGGSLRLSCEGTGSIFSPNAMGWYRQGPGKQRELVASVTSSGTTNYSDSVKGRFTISMDNYKHLAYLQMNSLKP |
| | | EDTAVYYCNLKELGTFYRRDFWGQGTQVTVSSGGGGSGGGGSGGGGSLAEAKVLANRELDKYGVSDYYKNLINNAK |
| | | TVEGVKALIDEILAALP |
| 306 | 118125 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAALAEAKVLANRELDKYGVSDYYKNLINNAKTVEGVKALIDEILAALPEVQLVESGGGLVQPGGSLRLS |
| | | CEGTGSIFSPNAMGWYRQGPGKQRELVASVTSSGTTNYSDSVKGRFTISMDNYKHLAYLQMNSLKPEDTAVYYCNLK |
| | | ELGTFYRRDFWGQGTQVTVSSGGGGSGGGSEVQLVESGGGLVQPGGSLRLSCEGTGSIFSPNAMGWYRQGPGKQ |
| | | RELVASVTSSGTTNYSDSVKGRFTISMDNYKHLAYLQMNSLKPEDTAVYYCNLKELGTFYRRDFWGQGTQVTVSS |
| 307 | 118128 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | KGRFTISMDNYKHLAYLQMNSLKPEDTAVYYCNLKELGTFYRRDFWGQGTQVTVSSGGGGSGGGSEVQLVESGGGL |
| | | VQPGGSLRLSCEGTGSIFSPNAMGWYRQGPGKQRELVASVTSSGTTNYSDSVKGRFTISMDNYKHLAYLQMNSLKP |
| | | EDTAVYYCNLKELGTFYRRDFWGQGTQVTVSSGGGGSGGGSEVQLVESGGGLVQPGNSLRLSCAASGFTFSSFGM |
| | | SWVRQAPGKGLEWVSSISGSGSDTLYADSVKGRFTISRDNAKTTLYLQMNSLRPEDTAVYYCTIGGSLSRSSQGTLVT |
| | | VSS |
| 308 | 118129 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSSGGGGSGGGSEVQLVESGGGL |
| | | VQPGNSLRLSCAASGFTFSSFGMSWVRQAPGKGLEWVSSISGSGSDTLYADSVKGRFTISRDNAKTTLYLQMNSLRP |
| | | EDTAVYYCTIGGSLSRSSQGTLVTVSSGGGGSGGGSEVQLVESGGGLVQPGGSLRLSCEGTGSIFSPNAMGWYRQG |
| | | PGKQRELVASVTSSGTTNYSDSVKGRFTISMDNYKHLAYLQMNSLKPEDTAVYYCNLKELGTFYRRDFWGQGTQVTV |
| | | SS |
| 309 | 118131 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSSGGGGSGGGSKEFTLDFSTAK |
| | | TYVDSLNVIRSAIGTPLQTISSGGTSLLMIDSGIGDNLFAVDILGFDFTLGRFNNLRLIVERNNLYVTGFVNRTNNVFYRF |
| | | ADFSHVTFPGTTAVTLSADSSYTTLQRVAGISRTGMQINRHSLTTSYLDLMSHSGTSLTQSVARAMLRFVTVTAEALRF |
| | | RQIQRGFRTTLDDLSGASYVMTAEDVDLTLNWGRLSSVLPDYHGQDSVRVGRISFGSINAILGSVALILNSHHHASAVA |
| | | AEVQLVESGGGLVQPGGSLRLSCEGTGSIFSPNAMGWYRQGPGKQRELVASVTSSGTTNYSDSVKGRFTISMDNYK |
| | | HLAYLQMNSLKPEDTAVYYCNLKELGTFYRRDFWGQGTQVTVSS |
| 310 | 118133 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSSGGSFRRGGGSKEFTLDFSTAK |
| | | TYVDSLNVIRSAIGTPLQTISSGGTSLLMIDSGIGDNLFAVDILGFDFTLGRFNNLRLIVERNNLYVTGFVNRTNNVFYRF |
| | | ADFSHVTFPGTTAVTLSADSSYTTLQRVAGISRTGMQINRHSLTTSYLDLMSHSGTSLTQSVARAMLRFVTVTAEALRF |
| | | RQIQRGFRTTLDDLSGASYVMTAEDVDLTLNWGRLSSVLPDYHGQDSVRVGRISFGSINAILGSVALILNSHHHASAVA |
| | | AEVQLVESGGGLVQPGGSLRLSCEGTGSIFSPNAMGWYRQGPGKQRELVASVTSSGTTNYSDSVKGRFTISMDNYK |
| | | HLAYLQMNSLKPEDTAVYYCNLKELGTFYRRDFWGQGTQVTVSS |
| 311 | 118136 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNCHHHASAVAAMASDEFPSMCPADEVQLVESGGGLVQPGGSLRLSCEGTGSIFSPNAMGWYRQGPGKQRELVAS |
| | | VTSSGTTNYSDSVKGRFTISMDNYKHLAYLQMNSLKPEDTAVYYCNLKELGTFYRRDFWGQGTQVTVSSGGGGSGG |
| | | GSEVQLVESGGGLVQPGGSLRLSCEGTGSIFSPNAMGWYRQGPGKQRELVASVTSSGTTNYSDSVKGRFTISMDNY |
| | | KHLAYLQMNSLKPEDTAVYYCNLKELGTFYRRDFWGQGTQVTVSS |
| 312 | 118137 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNCHHHASAVAAMASDEFPSMCPADGRVRGITHNKILWDSSTLGAILMRRTISSEVQLVESGGGLVQPGGSLRLSCE |
| | | GTGSIFSPNAMGWYRQGPGKQRELVASVTSSGTTNYSDSVKGRFTISMDNYKHLAYLQMNSLKPEDTAVYYCNLKEL |
| | | GTFYRRDFWGQGTQVTVSSGGGGSGGGSEVQLVESGGGLVQPGGSLRLSCEGTGSIFSPNAMGWYRQGPGKQRE |
| | | LVASVTSSGTTNYSDSVKGRFTISMDNYKHLAYLQMNSLKPEDTAVYYCNLKELGTFYRRDFWGQGTQVTVSS |
| 313 | 118140 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSSGGGGSGGGSEVQLVESGGGL |
| | | VQPGGSLRLSCEGTGSIFSPNAMGWYRQGPGKQRELVASVTSSGTTNYSDSVKGRFTISMDNYKHLAYLQMNSLKP |
| | | EDTAVYYCNLKELGTFYRRDFWGQGTQVTVSSGGGGSGGGSTRVDQTPRTATRETGESLTINCVLTDTSYPLYSTY |
| | | WYRKNPGSSNKEQISISGRYVESVNKGTKSFSLRIKDLTVADSATYICRAMGTNIWTGDGAGTVLTVN |
| 314 | 118141 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSSGGGGSGGGSTRVDQTPRTAT |
| | | RETGESLTINCVLTDTSYPLYSTYWYRKNPGSSNKEQISISGRYVESVNKGTKSFSLRIKDLTVADSATYICRAMGTNIW |
| | | TGDGAGTVLTVNGGGGSGGGSEVQLVESGGGLVQPGGSLRLSCEGTGSIFSPNAMGWYRQGPGKQRELVASVTSS |
| | | GTTNYSDSVKGRFTISMDNYKHLAYLQMNSLKPEDTAVYYCNLKELGTFYRRDFWGQGTQVTVSS |
| 315 | 118143 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEVQLVESGGGLVQPGGSLRLSCEGTGSIFSPNAMGWYRQGPGKQRELVASVTSSGTTNYSDSV |
| | | KGRFTISMDNYKHLAYLQMNSLKPEDTAVYYCNLKELGTFYRRDFWGQGTQVTVSSGGGGSGGGSEVQLVESGGGL |
| | | VQPGGSLRLSCEGTGSIFSPNAMGWYRQGPGKQRELVASVTSSGTTNYSDSVKGRFTISMDNYKHLAYLQMNSLKP |
| | | EDTAVYYCNLKELGTFYRRDFWGQGTQVTVSSGGGGSWWEQDRDWDFDVFGGGTP |
| 316 | 118305 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRQTPGKEREAVASIYPTGGTFYTDSVK |
| | | GRFTISRDNAKSTLYLQMNSLKPEDTAMYYCAAGKWGTDYWGQGTQVTVSS |
| 317 | 118306 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSSEAAAKQVQLVESGGNLVQPG |
| | | GSLRLSCAASGSIFSPNIMGWYRQPPGNERELVASVHSSGVTNYEDSAKGRFTISGDDAKYIWYLQMNGLKPEDTAA |
| | | YYCNVRELGVYFNRDFWGKGTLVTVSS |
| 318 | 118307 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRQTPGKEREAVASIYPTGGTFYTDSVK |
| | | GRFTISRDNAKSTLYLQMNSLKPEDTAMYYCAAGKWGTDYWGQGTQVTVSSEAAAKQVQLVESGGNLVQPGGSLRL |
| | | SCAASGSIFSPNIMGWYRQPPGNERELVASVHSSGVTNYEDSAKGRFTISGDDAKYIWYLQMNGLKPEDTAAYYCNV |
| | | RELGVYFNRDFWGKGTLVTVSS |
| 319 | 118308 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRQTPGKEREAVASIYPTGGTFYTDSVK |
| | | GRFTISRDNAKSTLYLQMNSLKPEDTAMYYCAAGKWGTDYWGQGTQVTVSSEAAAKQVQLVESGGNLVQPGGSLRL |
| | | SCAASGSIFSPNIMGWFRQPPGNERELVASVHSSGVTNYEDSAKGRFTISGDDAKYIWYLQMNGLKPEDTAAYYCNV |
| | | RELGVYFNRDFWGKGTLVTVSS |
| 320 | 118310 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWYRQPPGNERELVASIYPTGGTFYTDSVK |
| | | GRFTISRDNAKSTLYLQMNSLKPEDTAMYYCAAGKWGTDYWGQGTQVTVSS |
| 321 | 118311 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWYRQPPGNERELVASIYPTGGTFYTDSVK |
| | | GRFTISRDNAKSTLYLQMNSLKPEDTAMYYCAAGKWGTDYWGQGTQVTVSSEAAAKQVQLVESGGNLVQPGGSLRL |
| | | SCAASGSIFSPNIMGWYRQPPGNERELVASVHSSGVTNYEDSAKGRFTISGDDAKYIWYLQMNGLKPEDTAAYYCNV |
| | | RELGVYFNRDFWGKGTLVTVSS |
| 322 | 118414 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVQLVESGGGLVQPGGSLRLSCAASGVNFSSYTMSWYRQPPGNERELVAYINSGGGTTSYADS |
| | | VKGRFTISRDNAKNTLYLQMNSLKPEDTAMYYCQGGLYRGQGTQVTVSSEAAAKEVQLVESGGGLVQPGGSLRLSC |
| | | EGTGSIFSPNAMGWYRQGPGKQRELVASVTSSGTTNYSDSVKGRFTISMDNYKHLAYLQMNSLKPEDTAVYYCNLKE |
| | | LGTFYRRDFWGQGTQVTVSS |
| 323 | 118415 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVQLVESGGGLVQPGGSLRLSCAASGVNFSSYTMSWYRQPPGNERELVAYINSGGGTTSYADS |
| | | VKGRFTISRDNAKNTLYLQMNSLKPEDTAMYYCQGGLYRGQGTQVTVSSEAAAKQVQLVESGGGLVQPGGSLRLSC |
| | | EGTGSIFSPNAMGWYRQGPGKQRELVASVTSSGTTNYSDSVKGRFTISMDNYKHLAYLQMNSLKPEDTAVYYCNLKE |
| | | LGTFYRRDFWGQGTQVTVSS |
| 324 | 118416 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVQLVESGGGLVQPGGSLRLSCAASGVNFSSYTMSWYRQAPGKERELVAYINSGGGTTSYADSV |
| | | KGRFTISRDNAKNTLYLQMNSLKPEDTAMYYCQGGLYRGQGTQVTVSSEAAAKQVQLVESGGGLVQPGGSLRLSCE |
| | | GTGSIFSPNAMGWYRQGPGKQRELVASVTSSGTTNYSDSVKGRFTISMDNYKHLAYLQMNSLKPEDTAVYYCNLKEL |
| | | GTFYRRDFWGQGTQVTVSS |
| 325 | 118417 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSSEAAAKEVQLVESGGGLVQPG |
| | | GSLRLSCEGTGSIFSPNAMGWYRQGPGKQRELVASVTSSGTTNYSDSVKGRFTISMDNYKHLAYLQMNSLKPEDTAV |
| | | YYCNLKELGTFYRRDFWGQGTQVTVSS |
| 326 | 118418 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSSEAAAKQVQLVESGGGLVQPG |
| | | GSLRLSCEGTGSIFSPNAMGWYRQGPGKQRELVASVTSSGTTNYSDSVKGRFTISMDNYKHLAYLQMNSLKPEDTAV |
| | | YYCNLKELGTFYRRDFWGQGTQVTVSS |
| 327 | 118419 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEAAAK |
| | | QVQLVESGGGLVQPGGSLRLSCEGTGSIFSPNAMGWYRQGPGKQRELVASVTSSGTTNYSDSVKGRFTISMDNYKH |
| | | LAYLQMNSLKPEDTAVYYCNLKELGTFYRRDFWGQGTQVTVSS |
| 328 | 118420 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEAAAKEAAAKEAAAKQVKLVESGGGSVQAGGSLRLSCAASGDSYSVKYMGWFRRAPGKQRDQR |
| | | EEVASIYPTGGTFYTDSVKGRFTISRDNAKSTLYLQMNSLKPEDTAMYYCAAGKWGTDYWGQGTQVTVSSEAAAKQV |
| | | QLVESGGGLVQPGGSLRLSCEGTGSIFSPNAMGWYRQGPGKQRELVASVTSSGTTNYSDSVKGRFTISMDNYKHLA |
| | | YLQMNSLKPEDTAVYYCNLKELGTFYRRDFWGQGTQVTVSS |
| 329 | 118421 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCEGTGSIFSPNAMGWYRQGPGKQRELVASVTSSGTTNYSDSVKGRFTISM |
| | | DNYKHLAYLQMNSLKPEDTAVYYCNLKELGTFYRRDFWGQGTQVTVSS |
| 330 | 118422 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEAAAKEAAAKEAAAKQVKLVESGGGSVQAGGSLRLSCAASGDSYSVKYMGWFRRAPGKQRDQR |
| | | EEVASIYPTGGTFYTDSVKGRFTISRDNAKSTLYLQMNSLKPEDTAMYYCAAGKWGTDYWGQGTQVTVSSGGGGSG |
| | | GGSQVQLVESGGGLVQPGGSLRLSCEGTGSIFSPNAMGWYRQGPGKQRELVASVTSSGTTNYSDSVKGRFTISMDN |
| | | YKHLAYLQMNSLKPEDTAVYYCNLKELGTFYRRDFWGQGTQVTVSS |
| 331 | 118498 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSSGGGGSLAEAKVLANRELDKYG |
| | | VSDYYKNLINNAKTVEGVKALIDEILAALP |
| 332 | 118499 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSSGGGGSGGGGSGGGGSLAEA |
| | | KVLANRELDKYGVSDYYKNLINNAKTVEGVKALIDEILAALP |
| 333 | 118500 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSSGGGGSGGGGSGGGGSGGGG |
| | | SGGGGSLAEAKVLANRELDKYGVSDYYKNLINNAKTVEGVKALIDEILAALP |
| 334 | 118501 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSSEAAAKLAEAKVLANRELDKYG |
| | | VSDYYKNLINNAKTVEGVKALIDEILAALP |
| 335 | 118502 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSSEAAAKEAAAKEAAAKLAEAKVL |
| | | ANRELDKYGVSDYYKNLINNAKTVEGVKALIDEILAALP |
| 336 | 118503 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGGSGGGGSLAEAKVLANRELDKYGVSDYYKNLINNAKTVEGVKALIDEILAALP |
| 337 | 118504 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEAAAK |
| | | EAAAKEAAAKLAEAKVLANRELDKYGVSDYYKNLINNAKTVEGVKALIDEILAALP |
| 338 | 118505 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEAAAKEAAAKEAAAKQVKLVESGGGSVQAGGSLRLSCAASGDSYSVKYMGWFRRAPGKQRDQR |
| | | EEVASIYPTGGTFYTDSVKGRFTISRDNAKSTLYLQMNSLKPEDTAMYYCAAGKWGTDYWGQGTQVTVSSGGGGSG |
| | | GGGSGGGGSLAEAKVLANRELDKYGVSDYYKNLINNAKTVEGVKALIDEILAALP |
| 339 | 118506 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEAAAKEAAAKEAAAKQVKLVESGGGSVQAGGSLRLSCAASGDSYSVKYMGWFRRAPGKQRDQR |
| | | EEVASIYPTGGTFYTDSVKGRFTISRDNAKSTLYLQMNSLKPEDTAMYYCAAGKWGTDYWGQGTQVTVSSEAAAKEA |
| | | AAKEAAAKLAEAKVLANRELDKYGVSDYYKNLINNAKTVEGVKALIDEILAALP |
| 340 | 118507 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAAHHSEDPSSKAPKAPQVKLVESGGGSVQAGGSLRLSCAASGDSYSVKYMGWFRRAPGKQRDQ |
| | | REEVASIYPTGGTFYTDSVKGRFTISRDNAKSTLYLQMNSLKPEDTAMYYCAAGKWGTDYWGQGTQVTVSSGGGGS |
| | | GGGGSGGGGSLAEAKVLANRELDKYGVSDYYKNLINNAKTVEGVKALIDEILAALP |
| 341 | 118508 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAAHHSEDPSSKAPKAPQVKLVESGGGSVQAGGSLRLSCAASGDSYSVKYMGWFRRAPGKQRDQ |
| | | REEVASIYPTGGTFYTDSVKGRFTISRDNAKSTLYLQMNSLKPEDTAMYYCAAGKWGTDYWGQGTQVTVSSEAAAKE |
| | | AAAKEAAAKLAEAKVLANRELDKYGVSDYYKNLINNAKTVEGVKALIDEILAALP |
| 342 | 118509 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSS |
| 343 | 118510 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEAAAKEAAAKEAAAKQVKLVESGGGSVQAGGSLRLSCAASGDSYSVKYMGWFRRAPGKQRDQR |
| | | EEVASIYPTGGTFYTDSVKGRFTISRDNAKSTLYLQMNSLKPEDTAMYYCAAGKWGTDYWGQGTQVTVSS |
| 344 | 118511 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAAHHSEDPSSKAPKAPQVKLVESGGGSVQAGGSLRLSCAASGDSYSVKYMGWFRRAPGKQRDQ |
| | | REEVASIYPTGGTFYTDSVKGRFTISRDNAKSTLYLQMNSLKPEDTAMYYCAAGKWGTDYWGQGTQVTVSS |
| 345 | 118512 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSSGGGGSGGGGSGGGGSLAEA |
| | | KVLANRELDKYGVSDYYKNLINNAKTVEGVKALIDEILAALPGGGGS |
| 346 | 118513 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGGSGGGGSLAEAKVLANRELDKYGVSDYYKNLINNAKTVEGVKALIDEILAALPGGGGS |
| 347 | 118514 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAAHHSEDPSSKAPKAPQVKLVESGGGSVQAGGSLRLSCAASGDSYSVKYMGWFRRAPGKQRDQ |
| | | REEVASIYPTGGTFYTDSVKGRFTISRDNAKSTLYLQMNSLKPEDTAMYYCAAGKWGTDYWGQGTQVTVSSGGGGS |
| | | GGGGSGGGGSLAEAKVLANRELDKYGVSDYYKNLINNAKTVEGVKALIDEILAALPGGGGS |
| 348 | 118560 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVQLVESGGGLVQPGGSLRLSCEGTGSIFSPNAMGWYRQGPGKQREL |
| | | VASVTSSGTTNYSDSVKGRFTISMDNYKHLAYLQMNSLKPEDTAVYYCNLKELGTFYRRDFWGQGTQVTVSSGGGG |
| | | SGGGSQVKLVESGGGSVQAGGSLRLSCAASGDSYSVKYMGWFRRAPGKQRDQREEVASIYPTGGTFYTDSVKGRF |
| | | TISRDNAKSTLYLQMNSLKPEDTAMYYCAAGKWGTDYWGQGTQVTVSS |
| 349 | 118566 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEFPKP |
| | | STPPGSSGGAPLVEEPQNLIKQNCELFEQLGEYKFQNALLVRYTKKVPQVSTPTLVEVSRNLGKVGSKCCKHPEAKR |
| | | MPCAEDYLSVVLNQLCVLHEKTPVSDRVTKCCTESLVNRRPCFSALEVDETYVPKEFNAETFTFHADICTLSEKERQIK |
| | | KQTALVELVKHKPKATKEQLKAVMDDFAAFVEKCCKADDKETCFAEEGKKLVAASQAALGL |
| 350 | 118567 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEFPKP |
| | | STPPGSSGGAPLVEEPKNLVKTNCDLYEKLGEYGFQNAILVRYTQKAPQVSTPTLVEAARNLGRVGTKCCTLPEDQRL |
| | | PCVEDYLSAILNRVCLLHEKTPVSEHVTKCCSGSLVERRPCFSALTVDETYVPKEFKAETFTFHSDICTLPEKEKQIKKQ |
| | | TALAELVKHKPKATAEQLKTVMDDFAQFLDTCCKAADKDTCFSTEGPNLVTRCKDALA |
| 351 | 118568 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPLVEEPQNLIKQNCELFEQLGEYKFQNALLVRYTKKVPQVSTPTLVEVSRN |
| | | LGKVGSKCCKHPEAKRMPCAEDYLSVVLNQLCVLHEKTPVSDRVTKCCTESLVNRRPCFSALEVDETYVPKEFNAET |
| | | FTFHADICTLSEKERQIKKQTALVELVKHKPKATKEQLKAVMDDFAAFVEKCCKADDKETCFAEEGKKLVAASQAALGL |
| | | EFPKPSTPPGSSGGAPQVKLVESGGGSVQAGGSLRLSCAASGDSYSVKYMGWFRRAPGKQRDQREEVASIYPTGG |
| | | TFYTDSVKGRFTISRDNAKSTLYLQMNSLKPEDTAMYYCAAGKWGTDYWGQGTQVTVSS |
| 352 | 118569 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPLVEEPKNLVKTNCDLYEKLGEYGFQNAILVRYTQKAPQVSTPTLVEAARN |
| | | LGRVGTKCCTLPEDQRLPCVEDYLSAILNRVCLLHEKTPVSEHVTKCCSGSLVERRPCFSALTVDETYVPKEFKAETFT |
| | | FHSDICTLPEKEKQIKKQTALAELVKHKPKATAEQLKTVMDDFAQFLDTCCKAADKDTCFSTEGPNLVTRCKDALAEFP |
| | | KPSTPPGSSGGAPQVKLVESGGGSVQAGGSLRLSCAASGDSYSVKYMGWFRRAPGKQRDQREEVASIYPTGGTFY |
| | | TDSVKGRFTISRDNAKSTLYLQMNSLKPEDTAMYYCAAGKWGTDYWGQGTQVTVSS |
| 353 | 118570 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPEVQLVESGGGLVQAGGSLRLSCAASGLTFSSYAMGWFRQAPGKERER |
| | | VVSISRGGGYTYYADSVKGRFTISRDNAENTVYLQMNSLKPEDTAVYYCAAARYWATGSEYEFDYWGQGTLVTVSSG |
| | | GGGSGGGSQVKLVESGGGSVQAGGSLRLSCAASGDSYSVKYMGWFRRAPGKQRDQREEVASIYPTGGTFYTDSVK |
| | | GRFTISRDNAKSTLYLQMNSLKPEDTAMYYCAAGKWGTDYWGQGTQVTVSS |
| 354 | 118571 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSEVQLVESGGGLVQAGGSLRLSCAASGLTFSSYAMGWFRQAPGKERERVVSISRGGGYTYYADSVKGRFTIS |
| | | RDNAENTVYLQMNSLKPEDTAVYYCAAARYWATGSEYEFDYWGQGTLVTVSS |
| 355 | 118573 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCEGTGSIFSPNAMGWYRQGPGKQRELVASVTSSGTTNYSDSVKGRFTISM |
| | | DNYKHLAYLQMNSLKPEDTAVYYCNLKELGTFYRRDFWGQGTQVTVSSGGGGSGGGGSGGGGSLAEAKVLANREL |
| | | DKYGVSDYYKNLINNAKTVEGVKALIDEILAALP |
| 356 | 118574 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSLAEAKVLANRELDKYGVSDYYKNLINNAKTVEGVKALIDEILAALPGGGGSGGGSQVQLVESGGGLVQPGGS |
| | | LRLSCEGTGSIFSPNAMGWYRQGPGKQRELVASVTSSGTTNYSDSVKGRFTISMDNYKHLAYLQMNSLKPEDTAVYY |
| | | CNLKELGTFYRRDFWGQGTQVTVSS |
| 357 | 118575 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCEGTGSIFSPNAMGWYRQGPGKQRELVASVTSSGTTNYSDSVKGRFTISM |
| | | DNYKHLAYLQMNSLKPEDTAVYYCNLKELGTFYRRDFWGQGTQVTVSSEFPKPSTPPGSSGGAPLVEEPQNLIKQNC |
| | | ELFEQLGEYKFQNALLVRYTKKVPQVSTPTLVEVSRNLGKVGSKCCKHPEAKRMPCAEDYLSVVLNQLCVLHEKTPVS |
| | | DRVTKCCTESLVNRRPCFSALEVDETYVPKEFNAETFTFHADICTLSEKERQIKKQTALVELVKHKPKATKEQLKAVMD |
| | | DFAAFVEKCCKADDKETCFAEEGKKLVAASQAALGL |
| 358 | 118576 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCEGTGSIFSPNAMGWYRQGPGKQRELVASVTSSGTTNYSDSVKGRFTISM |
| | | DNYKHLAYLQMNSLKPEDTAVYYCNLKELGTFYRRDFWGQGTQVTVSSEFPKPSTPPGSSGGAPLVEEPKNLVKTNC |
| | | DLYEKLGEYGFQNAILVRYTQKAPQVSTPTLVEAARNLGRVGTKCCTLPEDQRLPCVEDYLSAILNRVCLLHEKTPVSE |
| | | HVTKCCSGSLVERRPCFSALTVDETYVPKEFKAETFTFHSDICTLPEKEKQIKKQTALAELVKHKPKATAEQLKTVMDD |
| | | FAQFLDTCCKAADKDTCFSTEGPNLVTRCKDALA |
| 359 | 118577 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEFPKP |
| | | STPPGSSGGAPLVEEPQNLIKQNCELFEQLGEYKFQNALLVRYTKKVPQVSTPTLVEVSRNLGKVGSKCCKHPEAKR |
| | | MPCAEDYLSVVLNQLCVLHEKTPVSDRVTKCCTESLVNRRPCFSALEVDETYVPKEFNAETFTFHADICTLSEKERQIK |
| | | KQTALVELVKHKPKATKEQLKAVMDDFAAFVEKCCKADDKETCFAEEGKKLVAASQAALGLEFPKPSTPPGSSGGAP |
| | | QVQLVESGGGLVQPGGSLRLSCEGTGSIFSPNAMGWYRQGPGKQRELVASVTSSGTTNYSDSVKGRFTISMDNYKH |
| | | LAYLQMNSLKPEDTAVYYCNLKELGTFYRRDFWGQGTQVTVSS |
| 360 | 118578 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEFPKP |
| | | STPPGSSGGAPLVEEPKNLVKTNCDLYEKLGEYGFQNAILVRYTQKAPQVSTPTLVEAARNLGRVGTKCCTLPEDQRL |
| | | PCVEDYLSAILNRVCLLHEKTPVSEHVTKCCSGSLVERRPCFSALTVDETYVPKEFKAETFTFHSDICTLPEKEKQIKKQ |
| | | TALAELVKHKPKATAEQLKTVMDDFAQFLDTCCKAADKDTCFSTEGPNLVTRCKDALAEFPKPSTPPGSSGGAPQVQ |
| | | LVESGGGLVQPGGSLRLSCEGTGSIFSPNAMGWYRQGPGKQRELVASVTSSGTTNYSDSVKGRFTISMDNYKHLAYL |
| | | QMNSLKPEDTAVYYCNLKELGTFYRRDFWGQGTQVTVSS |
| 361 | 118757 | MKEFTLDFSTAKTYVDSLNVIRSAIGTPLQTISSGGTSLLMIDSGSGDNLFAVDVRGIDPEEGRFNNLRLIVERNNLYVT |
| | | GFVNRTNNVFYRFADFSHVTFPGTTAVTLSGDSSYTTLQRVAGISRTGMQINRHSLTTSYLDLMSHSGTSLTQSVARA |
| | | MLRFVTVTAEALRFRQIQRGFRTTLDDLSGRSYVMTAEDVDLTLNWGRLSSVLPDYHGQDSVRVGRISFGSINAILGSV |
| | | ALILNCHHHASRVARQVKLVESGGGSVQAGGSLRLSCAASGDSYSVKYMGWFRRAPGKQRDQREEVASIYPTGGTF |
| | | YTDSVKGRFTISRDNAKSTLYLQMNSLKPEDTAMYYCAAGKWGTDYWGQGTQVTVSS |
| 362 | 118759 | MKEFTLDFSTAKTYVDSLNVIRSAIGTPLQTISSGGTSLLMIDSGSGDNLFAVDVRGIDPEEGRFNNLRLIVERNNLYVT |
| | | GFVNRTNNVFYRFADFSHVTFPGTTAVTLSGDSSYTTLQRVAGISRTGMQINRHSLTTSYLDLMSHSGTSLTQSVARA |
| | | MLRFVTVTAEALRFRQIQRGFRTTLDDLSGRSYVMTAEDVDLTLNWGRLSSVLPDYHGQDSVRVGRISFGSINAILGSV |
| | | ALILNCHHHASRVAREVQLVESGGGLVQPGGSLRLSCEGTGSIFSPNAMGWYRQGPGKQRELVASVTSSGTTNYSD |
| | | SVKGRFTISMDNYKHLAYLQMNSLKPEDTAVYYCNLKELGTFYRRDFWGQGTQVTVSS |
| 363 | 118761 | MKEFTLDFSTAKTYVDSLNVIRSAIGTPLQTISSGGTSLLMIDSGSGDNLFAVDVRGIDPEEGRFNNLRLIVERNNLYVT |
| | | GFVNRTNNVFYRFADFSHVTFPGTTAVTLSGDSSYTTLQRVAGISRTGMQINRHSLTTSYLDLMSHSGTSLTQSVARA |
| | | MLRFVTVTAEALRFRQIQRGFRTTLDDLSGRSYVMTAEDVDLTLNWGRLSSVLPDYHGQDSVRVGRISFGSINAILGSV |
| | | ALILNCHHHASRVAREFPKPSTPPGSSGGAPQVKLVESGGGSVQAGGSLRLSCAASGDSYSVKYMGWFRRAPGKQR |
| | | DQREEVASIYPTGGTFYTDSVKGRFTISRDNAKSTLYLQMNSLKPEDTAMYYCAAGKWGTDYWGQGTQVTVSSGGG |
| | | GSGGGSQVQLVESGGGLVQPGGSLRLSCEGTGSIFSPNAMGWYRQGPGKQRELVASVTSSGTTNYSDSVKGRFTIS |
| | | MDNYKHLAYLQMNSLKPEDTAVYYCNLKELGTFYRRDFWGQGTQVTVSS |
| 364 | 118765 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSS |
| 365 | 118831 | MKEFTLDFcTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCEGTGSIFSPNAMGWYRQGPGKQRELVASVTSSGTTNYSDSVKGRFTISM |
| | | DNYKHLAYLQMNSLKPEDTAVYYCNLKELGTFYRRDFWGQGTQVTVSS |
| 366 | 118844 | MKEFTLDFSTAKTYVDSLNVIRSAIGTPLQTISSGGTSLLMIDSGIGDNLFAVDILGFDFTLGRFNNLRLIVERNNLSVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTADALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCEGTGSIFSPNAMGWYRQGPGKQRELVASVTSSGTTNYSDSVKGRFTISM |
| | | DNYKHLAYLQMNSLKPEDTAVYYCNLKELGTFYRRDFWGQGTQVTVSS |
| 367 | 118845 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SQVQLVESGGGLVQPGGSLRLSCEGTGSIFSPNAMGWYRQGPGKQRELVASVTSSGTTNYSDSVKGRFTISMDNYK |
| | | HLAYLQMNSLKPEDTAVYYCNLKELGTFYRRDFWGQGTQVTVSS |
| 368 | 118846 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGGSGGGGSGGGGSGGGGSQVQLVESGGGLVQPGGSLRLSCEGTGSIFSPNAMGWYRQGPGKQRELVASVT |
| | | SSGTTNYSDSVKGRFTISMDNYKHLAYLQMNSLKPEDTAVYYCNLKELGTFYRRDFWGQGTQVTVSS |
| 369 | 118847 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | IEFPKPSTPPGSSGGAPQVKLVESGGGSVQAGGSLRLSCAASGDSYSVKYMGWFRRAPGKQRDQREEVASIYPTGG |
| | | TFYTDSVKGRFTISRDNAKSTLYLQMNSLKPEDTAMYYCAAGKWGTDYWGQGTQVTVSSGGGGSGGGSQVQLVES |
| | | GGGLVQPGGSLRLSCEGTGSIFSPNAMGWYRQGPGKQRELVASVTSSGTTNYSDSVKGRFTISMDNYKHLAYLQMN |
| | | SLKPEDTAVYYCNLKELGTFYRRDFWGQGTQVTVSS |
| 370 | 118848 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVQLVESGGGLVQPGGSLRLSCEGTGSIFSPNAMGWYRQGPGKQREL |
| | | VASVTSSGTTNYSDSVKGRFTISMDNYKHLAYLQMNSLKPEDTAVYYCNLKELGTFYRRDFWGQGTQVTVSS |
| 371 | 118898 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCEGTGSIFSPNAMGWYRQGPGKQRELVASVTSSGTTNYSDSVKGRFTISM |
| | | DNYKHLAYLQMNSLKPEDTAVYYCNLKELGTFYRRDFWGQGTQVTVSS |
| 372 | 119009 | MKEFTLDFcTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSS |
| 373 | 119028 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SQVKLVESGGGSVQAGGSLRLSCAASGDSYSVKYMGWFRRAPGKQRDQREEVASIYPTGGTFYTDSVKGRFTISRD |
| | | NAKSTLYLQMNSLKPEDTAMYYCAAGKWGTDYWGQGTQVTVSS |
| 374 | 119029 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVKLVESGGGSVQAGGSLRLSCAASGDSYSVKYMGWFRRAPGKQRDQREEVASIYPTGGTFYTDSVKGRF |
| | | TISRDNAKSTLYLQMNSLKPEDTAMYYCAAGKWGTDYWGQGTQVTVSS |
| 375 | 119030 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGGSGGGGSQVKLVESGGGSVQAGGSLRLSCAASGDSYSVKYMGWFRRAPGKQRDQREEVASIYPTGGTFYT |
| | | DSVKGRFTISRDNAKSTLYLQMNSLKPEDTAMYYCAAGKWGTDYWGQGTQVTVSS |
| 376 | 119031 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGGSGGGGSGGGGSGGGGSQVKLVESGGGSVQAGGSLRLSCAASGDSYSVKYMGWFRRAPGKQRDQREEVA |
| | | SIYPTGGTFYTDSVKGRFTISRDNAKSTLYLQMNSLKPEDTAMYYCAAGKWGTDYWGQGTQVTVSS |
| 377 | 119032 | MKEFTLDFCTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSS |
| 378 | 119049 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCAGTGSIFSPNAMGWYRQGPGKQRELVASVTSSGTTNYSDSVKGRFTISM |
| | | DNYKHLAYLQMNSLKPEDTAVYYCNLKELGTFYRRDFWGQGTQVTVSS |
| 379 | 119050 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCAATGSIFSPNAMGWYRQGPGKQRELVASVTSSGTTNYSDSVKGRFTISM |
| | | DNYKHLAYLQMNSLKPEDTAVYYCNLKELGTFYRRDFWGQGTQVTVSS |
| 380 | 119051 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCAGTGSIFSPNAMGWYRQGPGKQRELVASVTSSGTTNYSDSVKGRFTISM |
| | | DNYKHLAYLQMNSLKPEDTAVYYCALKELGTFYRRDFWGQGTQVTVSS |
| 381 | 119052 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCAATGSIFSPNAMGWYRQGPGKQRELVASVTSSGTTNYSDSVKGRFTISM |
| | | DNYKHLAYLQMNSLKPEDTAVYYCALKELGTFYRRDFWGQGTQVTVSS |
| 382 | 119053 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEAAAK |
| | | GGGGSQVQLVESGGGLVQPGGSLRLSCEGTGSIFSPNAMGWYRQGPGKQRELVASVTSSGTTNYSDSVKGRFTIS |
| | | MDNYKHLAYLQMNSLKPEDTAVYYCNLKELGTFYRRDFWGQGTQVTVSS |
| 383 | 119054 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEAAAK |
| | | EAAAKGGGGSQVQLVESGGGLVQPGGSLRLSCEGTGSIFSPNAMGWYRQGPGKQRELVASVTSSGTTNYSDSVKG |
| | | RFTISMDNYKHLAYLQMNSLKPEDTAVYYCNLKELGTFYRRDFWGQGTQVTVSS |
| 384 | 119055 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEAAAK |
| | | GGGGSGGGSQVQLVESGGGLVQPGGSLRLSCEGTGSIFSPNAMGWYRQGPGKQRELVASVTSSGTTNYSDSVKG |
| | | RFTISMDNYKHLAYLQMNSLKPEDTAVYYCNLKELGTFYRRDFWGQGTQVTVSS |
| 385 | 119056 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEGKS |
| | | SGSGSESKSTQVQLVESGGGLVQPGGSLRLSCEGTGSIFSPNAMGWYRQGPGKQRELVASVTSSGTTNYSDSVKG |
| | | RFTISMDNYKHLAYLQMNSLKPEDTAVYYCNLKELGTFYRRDFWGQGTQVTVSS |
| 386 | 119057 | MKEFTLDFSTAKTYVDSLNVIRSAIGTPLQTISSGGTSLLMIDSGIGDNLFAVDILGFDFTLGRENNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSKESG |
| | | SVSSEQLAQFRSLDQVQLVESGGGLVQPGGSLRLSCEGTGSIFSPNAMGWYRQGPGKQRELVASVTSSGTTNYSDS |
| | | VKGRFTISMDNYKHLAYLQMNSLKPEDTAVYYCNLKELGTFYRRDFWGQGTQVTVSS |
| 387 | 119058 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEAAAK |
| | | GGGGSQVQLVESGGGLVQPGGSLRLSCAGTGSIFSPNAMGWYRQGPGKQRELVASVTSSGTTNYSDSVKGRFTIS |
| | | MDNYKHLAYLQMNSLKPEDTAVYYCNLKELGTFYRRDFWGQGTQVTVSS |
| 388 | 119059 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEAAAK |
| | | EAAAKGGGGSQVQLVESGGGLVQPGGSLRLSCAGTGSIFSPNAMGWYRQGPGKQRELVASVTSSGTTNYSDSVKG |
| | | RFTISMDNYKHLAYLQMNSLKPEDTAVYYCNLKELGTFYRRDFWGQGTQVTVSS |
| 389 | 119060 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEAAAK |
| | | GGGGSGGGSQVQLVESGGGLVQPGGSLRLSCAGTGSIFSPNAMGWYRQGPGKQRELVASVTSSGTTNYSDSVKG |
| | | RFTISMDNYKHLAYLQMNSLKPEDTAVYYCNLKELGTFYRRDFWGQGTQVTVSS |
| 390 | 119061 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSEGKS |
| | | SGSGSESKSTQVQLVESGGGLVQPGGSLRLSCAGTGSIFSPNAMGWYRQGPGKQRELVASVTSSGTTNYSDSVKG |
| | | RFTISMDNYKHLAYLQMNSLKPEDTAVYYCNLKELGTFYRRDFWGQGTQVTVSS |
| 391 | 119062 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSKESG |
| | | SVSSEQLAQFRSLDQVQLVESGGGLVQPGGSLRLSCAGTGSIFSPNAMGWYRQGPGKQRELVASVTSSGTTNYSDS |
| | | VKGRFTISMDNYKHLAYLQMNSLKPEDTAVYYCNLKELGTFYRRDFWGQGTQVTVSS |
| 392 | 119063 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPqVQLVESGGGLVQPGGSLRLSCaGTGSIFSPNAMGWYRQGPGKQRELV |
| | | ASVTSSGTTNYSDSVKGRFTISMDNYKHLAYLQMNSLKPEDTAVYYCNLKELGTFYRRDFWGQGTQVTVSS |
| 393 | 119064 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPqVQLVESGGGLVQPGGSLRLSCaaTGSIFSPNAMGWYRQGPGKQRELV |
| | | ASVTSSGTTNYSDSVKGRFTISMDNYKHLAYLQMNSLKPEDTAVYYCNLKELGTFYRRDFWGQGTQVTVSS |
| 394 | 119065 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPqVQLVESGGGLVQPGGSLRLSCaGTGSIFSPNAMGWYRQGPGKQRELV |
| | | ASVTSSGTTNYSDSVKGRFTISMDNYKHLAYLQMNSLKPEDTAVYYCaLKELGTFYRRDFWGQGTQVTVSS |
| 395 | 119066 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPqVQLVESGGGLVQPGGSLRLSCaaTGSIFSPNAMGWYRQGPGKQRELV |
| | | ASVTSSGTTNYSDSVKGRFTISMDNYKHLAYLQMNSLKPEDTAVYYCaLKELGTFYRRDFWGQGTQVTVSS |
| 396 | 119067 | MKEFTLDFSTAKTYVDSLNVIRSAIGTPLQTISSGGTSLLMIDSGIGDNLFAVDILGFDFTLGRFNNLRLIVERNNLYVTGF |
| | | VNRTNNVFYRFADFSHVTFPGTTAVTLSADSSYTTLQRVAGISRTGMQINRHSLTTSYLDLMSHSGTSLTQSVARAML |
| | | RFVTVTAEALRFRQIQRGFRTTLDDLSGASYVMTAEDVDLTLNWGRLSSVLPDYHGQDSVRVGRISFGSINAILGSVAL |
| | | ILNSHHHASAVAAEFPKPSTPPGSSGGAPQVKLVESGGGSVQAGGSLRLSCAASGDSYSVKYMGWFRRAPGKQRD |
| | | QREEVASIYPTGGTFYTDSVKGRFTISRDNAKSTLYLQMNSLKPEDTAMYYCAAGKWGTDYWGQGTQVTVSSGGGG |
| | | SGGGSQVQLVESGGGLVQPGGSLRLSCEGTGSIFSPNAMGWYRQGPGKQRELVASVTSSGTTNYSDSVKGRFTISM |
| | | DNYKHLAYLQMNSLKPEDTAVYYCNLKELGTFYRRDFWGQGTQVTVSSHHHHHH |

### EXAMPLES

The present invention is further illustrated by the following non-limiting examples.

### Example 1: Binding Domain Selection

Binding domains are derived from known human or murine antibodies directed against CTLA-4. Initially, antibodies are selected based on their ability to bind to CTLA-4 with high affinity, avidity, and specificity. Murine antibody candidates are optionally humanized using recombinant methods to make murine/human chimeric sequences, and the resulting chimeric antibodies are screened for binding affinity. For certain candidate antibodies, amino acid residues identified as putative post-translational modification sites and/or potentially disadvantageous to manufacturing, such as noncanonical or unpaired cysteine residues and N-glycosylation sites, are altered or removed. If greater CTLA-4 binding affinity is desired, then mutagenesis of complementarity determining regions (CDRs) is performed.

Binding domains are generated in various formats, such as VHH and scFvs, using the CDRs, VH and/or VL sequences of the known CTLA-4 antibodies.

### Example 2: Binding Domain Discovery

Binding domains are also derived from antibodies generated de novo. Transgenic murine B-cells expressing anti-CTLA-4 antibodies are obtained from mice immunized, subcutaneously on a prime/boost schedule, using a recombinant CTLA-4 extracellular domain (or a fragment or variant thereof) as the immunogen. Lymphoid tissue samples and bone marrow are harvested and pooled, and B-cells expressing monoclonal antibodies (mAbs) on the cell surface are sorted and screened for binding to CTLA-4. The variable regions from monoclonal antibodies of certain B-cell clones are identified and cloned as chimeric mAbs. Candidate mAbs are characterized for CTLA-4 binding. Binding domains are also derived from llama or chicken immunization, followed by phage display, panning, screening and selection for clones that specifically bind CTLA-4.

Binding domains are generated in various formats, such as VHH and scFvs, using the CDRs, VH and/or VL sequences of the identified CTLA-4 antibodies.

### Example 3: Exemplary CTLA-4 Binding Molecule Exhibits Activity In Vitro

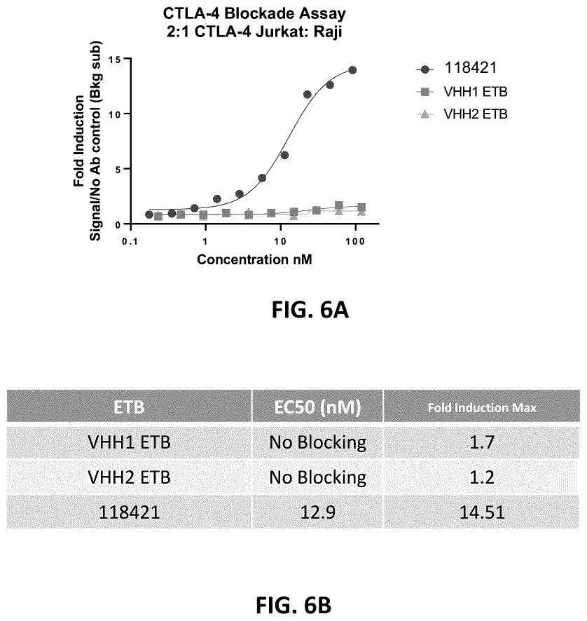

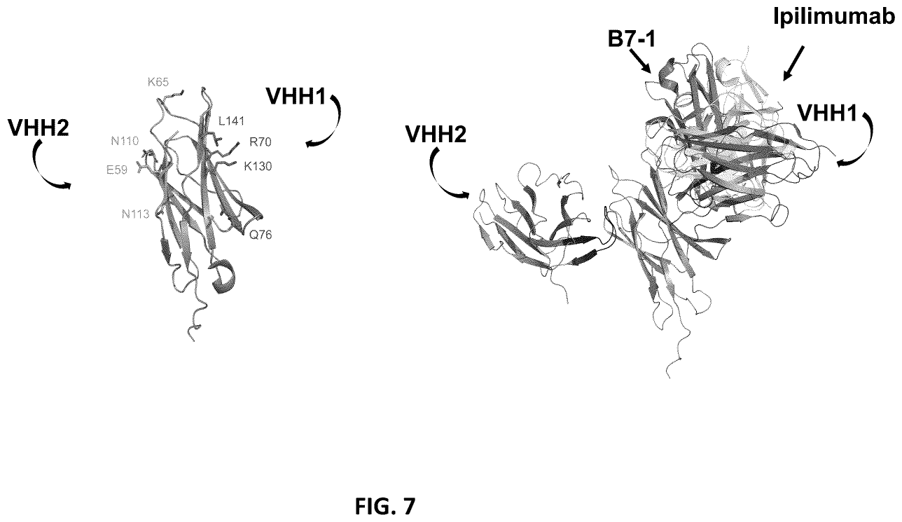

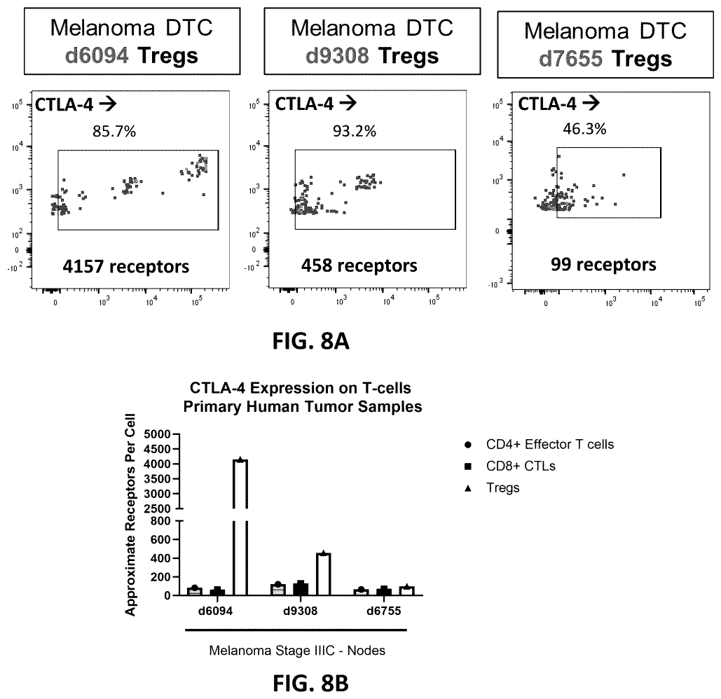

An exemplary CTLA-4 binding molecule comprising two anti-CTLA-4 VHH domains was prepared (CTLA-4 ETB 118421; Table 5). CTLA-4 ETB 118421 is of biparatopic nature, composed of two unique VHH domains in tandem (FIG. 5A). Each VHH binds human and non-human primate (NHP) CTLA-4 with similar affinities (within 3-fold) as measured by ELISA (FIG. 5B). CTLA-4 ETB 118421 binds with higher affinity compared to the single VHH CTLA-4 binding molecules. For the ELISA, Fc-CTLA-4 protein from each species was coated on the plate. The next day, a CTLA-4 binding molecule is added in a dilution series, washed, and detected with an anti-DI SLTA-HRP antibody.

CTLA-4 ETB 118421 blocks protein synthesis. The A subunit of Shiga-like toxins is known to depurinate ribosomal RNA, which leads to protein synthesis inhibition and apoptotic cell death. The direct cell kill mechanism of action of ETBs is thus dependent on the protein synthesis inhibition activity of the DI SLTA subunit. To demonstrate that 118421 retains the ribosomal inhibitory properties of the DI SLTA domain, a cell free protein expression assay was used (TnT® kit, Promega). FIG. 5C shows that CTLA-4 ETB 118421 and the positive control DI SLTA demonstrated comparable inhibition of protein synthesis with IC50 values of 14.3 pM and 14.8 pM, respectively. In contrast, the inactive control ETB, containing two point mutations within the DI SLTA subunit, did not inhibit ribosome activity.

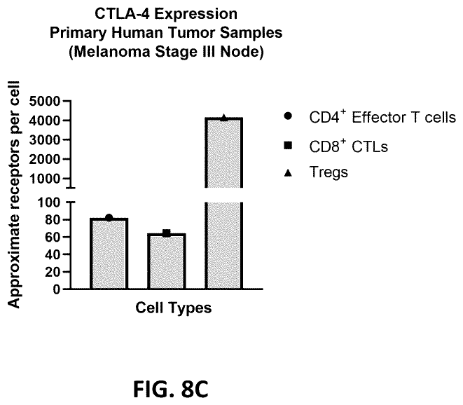

CTLA-4 ETB 118421 blocks CTLA-4:B7 interactions in a cellular system. CTLA-4 Blockade Bioassay (Promega) was used to measure the ability of exemplary CTLA-4 binding molecules to block the interaction of CTLA-4 with its ligands in a Jurkat:Raji cell system. CTLA-4 binding molecules were added to CTLA-4-Jurkat cells, aAPC/Raji cells were added, then the signal was read after an 8h incubation. CTLA-4 ETB 118421 is of biparatopic nature, composed of two unique VHH domains in tandem. CTLA-4 binding molecules containing only one of the VHH domains did not block CTLA4 interaction with B7 (FIG. 6A; FIG. 6B). However, CTLA-4 ETB 118421 did induce a robust blockade signal.

VHH1 and VHH2 critical CTLA-4 contact residues were identified through shotgun mutagenesis and high-throughput flow cytometry (Integral Molecular). The docked structure supports that VHH1 competes with Ipilimumab for a similar epitope region, while VHH2 does not compete with ipilimumab (FIG. 7).

### Example 4: Potency of CTLA-4 Binding Molecules Depends on Level of CTLA-4 Expression

The objective of this study was to examine cytotoxicity of CTLA-4 binding molecules in cell lines expressing various levels of CTLA-4.

Studies were first conducted to determine whether CTLA-4 expression differed between T cell subsets in the tumor microenvironment of subjects with cancer. CTLA-4 expression on T cells from tumor samples of melanoma patients was analyzed. Three different stage III melanoma samples were dissociated and vialed at Discovery life sciences. These samples were thawed and stained with a multicolor immunophenotyping panel to distinguish the various T cell subtypes and measure CTLA-4 expression. BD Quantibrite Beads were used to convert gMFI of CTLA-4 expression to receptors per cell.

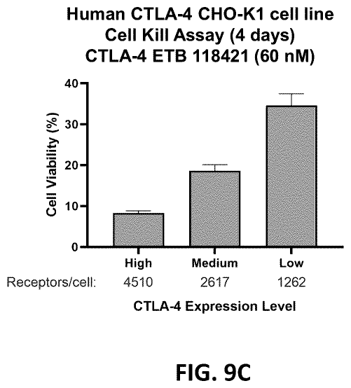

Results are shown in FIG. 8A, FIG. 8B, and FIG. 8C. Tregs from primary tumor samples of melanoma patients exhibited the highest level of CTLA-4 expression compared to CD4+ effector T cells and CD8+ cytotoxic T lymphocytes (CTLs).

Gain-of-function human CHO-K1 cell lines were then generated to express different CTLA-4 levels representative of human tumor Treg expression. The cell lines were different subclones of the same parental hCTLA-4-CHOK1 monoclonal cell line; each subclone was selected to represent a different range of CTLA-4 expression. These cell lines were grown and maintained in Hams F-12K media supplemented with 10% FBS and 100U/10014 Pen/Strep per mL.

CTLA-4 expressing cells for each expression level were plated into 384 well microplates at a concentration of 500 cells/well. A dilution series of the CTLA-4 ETB 118421 was prepared in PBS then added in triplicate to each cell line for a final concentration range of 180.0-0.001 nM (12, 3-fold dilutions). CTLA-4 expressing cells treated with ETBs were incubated for 4 days at 37° C. in a tissue culture incubator supplemented with 5% CO₂. On Day 4, 50 μL of CTG2.0 was added and incubated for ˜2 minutes with gentle agitation followed by a 10-minute incubation at room temperature in the dark. Luminescence (RLU) was measured on Spectramax iD3 multimode plate reader at 100 ms and exported to Excel for data analysis. Viability of various cell lines was measured 96 hours after CTLA-4 ETB addition to cells using Cell Titer-Glo® (Promega). Cell viability was normalized to cells-only control wells and values were analyzed via non-linear regression curve fit on log transformed concentration values (log(inhibitor) vs. response (three parameters)) to determine IC50 values. IC50 values were reported in nM.

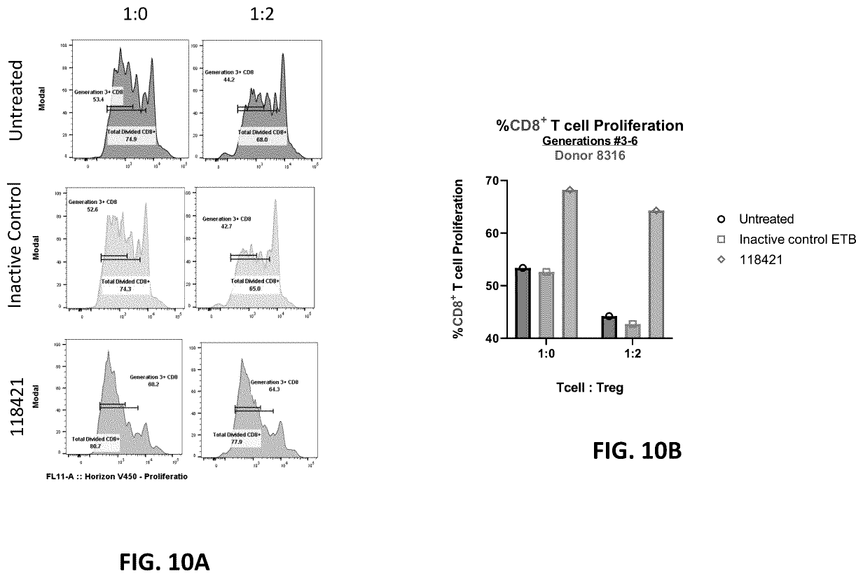

Results are shown in FIG. 9A, FIG. 9B, and FIG. 9C, and show that CTLA-4 ETB 118421 cell death was dependent on CTLA-4 receptor density.

### Example 5: CTLA-4 Binding Molecules Reduce Treg-Mediated Suppression of Effector T Cells

The objective of this study was to determine whether CTLA-4 binding molecules reduce T reg-mediated suppression of effector T cells.

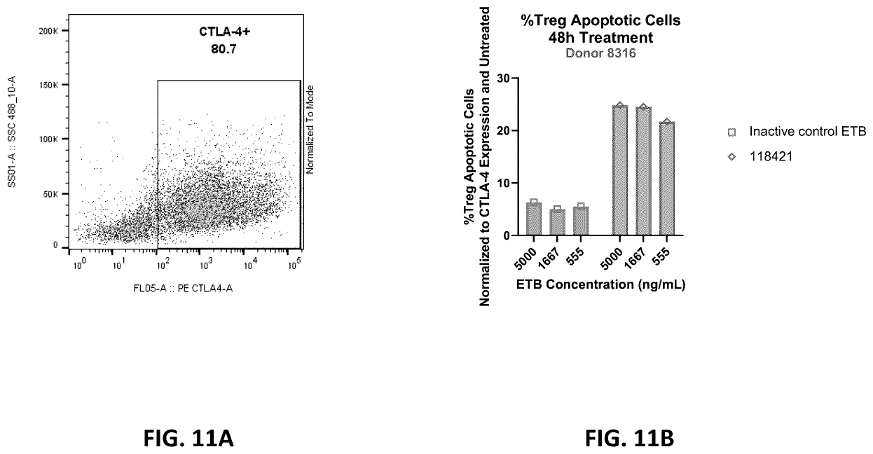

Tregs and CD8+ T cells were magnetically isolated from peripheral blood mononuclear cells (PBMC) of a healthy donor. The CD8+ T cells were stained with Violet proliferation dye and then co-cultured with autologous Tregs at various ratios and were either treated with or without CTLA-4 ETB 118421 for 4 days. The cells were plated with antiCD3/CD28 beads to stimulate the proliferation of CD8+ T cells. After 4 days of incubation, the antiCD3/CD28 beads were removed from the cells and the cells were surface stained with antiCD4 and antiCD8 antibody and acquired on a flow cytometer. Results are shown in FIG. 10A and FIG. 10B. Co-cultures treated with CTLA-4 ETB 118421 exhibited significantly increased CD8+ T cell proliferation compared to enzymatically inactive CTLA-4 ETB control or untreated cells.

### Example 6: CTLA-4 Binding Molecules Show Cytotoxicity on CTLA-4-Expressing Ex Vivo Expanded Primary Tregs

The objective of this study was to determine whether CTLA-4 binding molecules induce apoptosis of primary human Tregs.

Primary Tregs were isolated from frozen PBMC vials of a normal, healthy donor using EasySep™ Human CD4⁺CD127^{low}CD49d⁻ Regulatory T cell Enrichment kit based on negative selection as per manufacturer's instructions. The isolated cells were counted and plated at 0.5e6 cells/mL in a 24 well plate in IMDM media containing 10% human serum, 100 U/100 ug Pen/Strep per mL, 500 IU/mL recombinant human IL-2 and human T cell activator αCD3/αCD28 Dynabeads™ (1:1 ratio; Gibco). After 5 days in culture, the cells were harvested, washed once with cell staining buffer (1×PBS+1% BSA) and stained with αCTLA-4 antibody or isotype control to confirm the expression of CTLA-4 on expanded Tregs.

Expanded Tregs (5e4 cells) were transferred to each well of a 96 well round bottom plate. A dilution series of each ETB was prepared in 1×PBS then added to experimental wells for a final concentration range of 400.0-0.39 nM (6, 4-fold dilutions) and incubated for 48 hours at 37° C. in a tissue culture incubator supplemented with 5% CO₂. After 48 hours of incubation, the Tregs were transferred to their respective wells in a 96 well V-bottom plate, washed two times with 1×PBS and stained in the dark at 4° C. for 30 minutes with Zombie Violet dye diluted 1:1000 in 1×PBS. Following incubation, the cells were washed 2 times with 1×PBS before proceeding with Apotracker™ Green staining.

Apotracker™ dye was reconstituted with 100 μL dimethyl sulfoxide (DMSO) as directed by the product sheet (100 μL DMSO for 100 test vial). The reconstituted dye was diluted 1:10 with cell staining buffer (1×PBS+1% BSA). 0.5 μL of diluted reagent was mixed with 99.5 μL cell staining buffer (100 μL staining volume) to stain Tregs. The cells were incubated with Apotracker™ Green in the dark for 15 minutes at RT. Following incubation, the cells were washed twice with cell staining buffer. The cells were then resuspended in 200-250 μL cell staining buffer and acquired on BioRad ZE5 flow cytometer. The raw fcs files were analyzed on FlowJo software (BD Biosciences).

Tregs at day 6 post isolation were treated with different concentrations of CTLA-4 ETB 118421 for 48h. The cells were stained with Apotracker green to measure apoptotic cells. The percentage of apoptotic cells was normalized to the untreated control and adjusted to account for the CTLA-4 positivity.

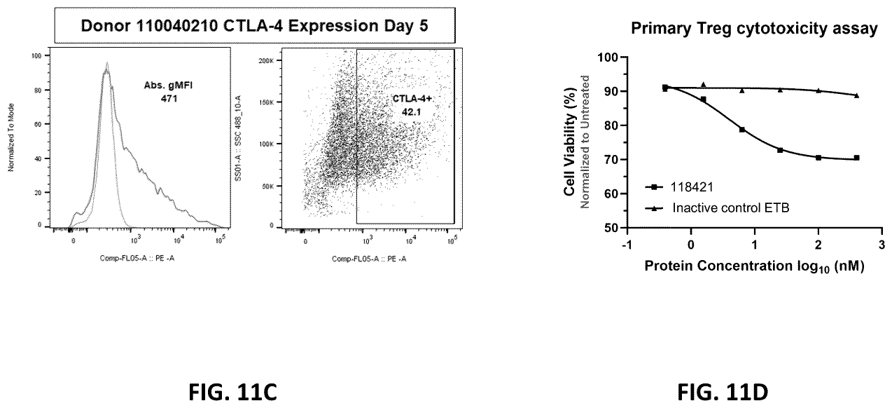

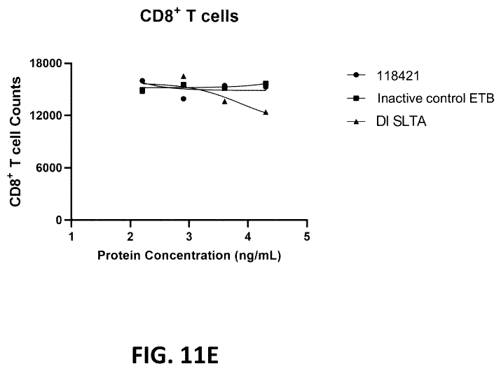

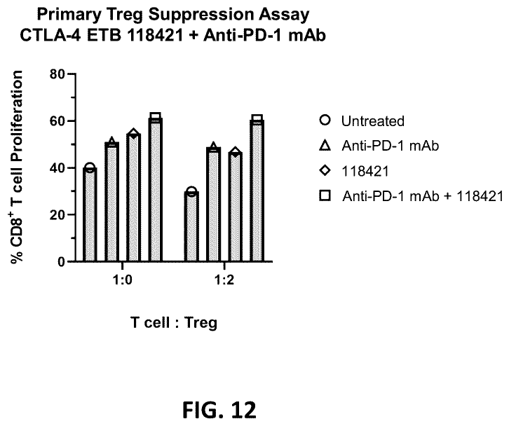

Results are shown in FIG. 11A to FIG. 11D. FIG. 11A and FIG. 11C show phenotyping of ex-vivo expanded Tregs from healthy donor 8316 and 110040210, respectively. FIG. 11B and FIG. 11D shows the cytotoxicity assays analyzing CTLA-4 ETB 118421 induction of apoptosis using the ex-vivo expanded primary Tregs. CTLA-4 ETB 118421 induced apoptosis of primary Tregs in a dose dependent manner. Tregs treated with inactive ETB exhibited no reduction in cell viability.

Regulatory T cells (Treg) constitutively express CTLA-4 while resting T cells do not express CTLA-4 but can upregulate CTLA-4 expression upon activation. Further experiments were performed to determine whether CTLA-4 binding molecules were cytotoxic to CD8+ effector T cells from PBMCs isolated from healthy subjects. CD8+ T cells in the periphery have low to no expression of CTLA-4.

PBMCs from a healthy donor were washed in IMDM containing 10% human serum and 1% penicillin/streptomycin and plated at 1×10⁶ cells/well in a 24 well plate. The cells were treated with different concentrations (160-20000 ng/mL; 4-point, 5-fold dilution series) of either CTLA-4 ETB 118421, enzymatically inactive CTLA-4 ETB, or deimmunized (DI) SLTA and incubated for 48h at 37° C. with 5% CO2. After 48 h, the cells were stained with a panel of surface antibodies and acquired on a flow cytometer. The data was analyzed using FlowJo software (BD Biosciences).

The results from the cytotoxicity assay on primary CD8⁺ T cells are shown in FIG. 11E. There was no effect of CTLA-4 ETB 118421 on primary CD8⁺ T cells from healthy donors. Enzymatically inactive CTLA-4 ETB did not show any change in CD8⁺ T cell numbers and DI-SLTA showed minimal non-specific kill only at the highest concentration tested. This has been seen in overexpressing systems where saturating concentrations of DI-SLTA show non-specific cytotoxicity (data not shown). CD8⁺ T cells were negative for CTLA-4 expression (data not shown). This data supports the specificity and potency of CTLA-4 ETB 118421 on CTLA-4 expressing cells.

### Example 7: CTLA-4 Binding Molecules, Alone or in Combination with Anti-PD-1 Antibody, Inhibit Treg-Mediated Suppression of Effector T Cells

The objective of this study was to determine whether CTLA-4 binding molecules and/or anti-PD-1 antibody reduce T reg-mediated suppression of effector T cells.

Primary Tregs were isolated from frozen PBMC vials of a normal, healthy donor using EasySep™ Human CD4⁺CD127^{low}CD49d⁻ Regulatory T cell Enrichment kit based on negative selection as per manufacturer's instructions. Autologous T cells were isolated from the same donor using EasySep™ Human T cell isolation kit. The isolated cells were washed once with PBMC media, 10% human serum, 100U/100 μg Pen/Strep per mL and counted on a BioRad TC20 cell counter. After counting, 200,000 T cells were kept aside and were used as unstained cells. The remaining T cells were resuspended at 10-30 million cells/mL in 1×PBS and stained with 1 μM VPD450 for 10 minutes at 37° C. The staining of T cells was quenched after 10 minutes by addition of 9 times the original volume of 1×PBS counted. The cells were centrifuged at 350 g for 5 minutes and then washed with PBMC media. The stained T cells were counted and plated in a round bottom 96 well plate at a concentration of 2 million cells/mL (100,000 T cells per well) in PBMC media.

The stained T cells were co-cultured with autologous Tregs at a 1:2 ratio and αCD3/αCD28 activator Dynabeads™ were used to drive the proliferation of T cells. In addition, cells were treated with anti-PD-1 antibody (nivolumab, 20 μg/mL) for approximately 20h followed by CTLA-4 ETB 118421 (72 nM) to evaluate the combination effect. T cells cultured without beads were used as a negative control. Other treatment groups included anti-PD-1 antibody alone (nivolumab, 20 μg/mL), CTLA-4 ETB 118421 alone (72 nM), and untreated cells. Cells were incubated for 4 days at 37° C. in a tissue culture incubator supplemented with 5% CO₂.

After 4 days of incubation, the αCD3/αCD28 beads were removed from the cells using a magnet and washed 1× with cell staining buffer (1×PBS+1% BSA). The cells were surface stained with αCD8 antibody in cell staining buffer for 30 minutes at 4° C. in the dark. Following incubation, cells were washed twice with cell staining buffer and acquired on BioRad ZE5 flow cytometer.

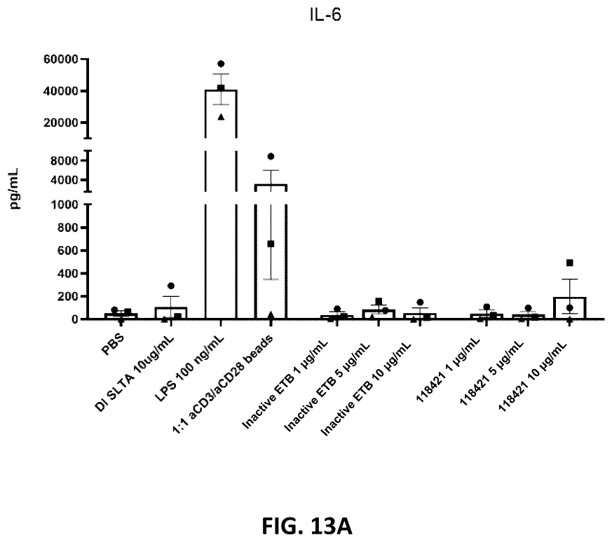

Results are shown in FIG. 12. Selective depletion of CTLA-4 expressing Tregs using CTLA-4 ETB 118421 promoted proliferation of CD8+ T cells. Treatment with CTLA-4 binding molecule and anti-PD-1 antibody further enhanced CD8+ T cell proliferation better than either treatment alone.

### Example 8: CTLA-4 Binding Molecules do not Result in Pro-Inflammatory Cytokine Release

Cytokine release assays (CRA) are commonly used as a pre-clinical in vitro risk assessment and prediction tool for new biotherapeutic candidates to potentially elicit adverse pro-inflammatory cytokine responses in patients. Although severe cytokine release syndrome (CRS) is relatively rare in the clinic, in some cases the massive release of potent pro-inflammatory cytokines can lead to multi organ damage and death. Thus, in vitro evaluation of novel therapeutics, including ETBs, to predict potential CRS in vivo is critical to preclinical safety testing.

PBMCs were isolated from healthy donors (n=3) and were plated at a density of 5e4 cells per well in PBMC media in a 96 well round bottom plate. The cells were allowed to rest for 2 h at 37° C. in an incubator. The cells were then treated in triplicate with the following agents: LPS (100 ng), DI-SLTA, Dynabeads™ human T cell activator CD3/CD28 (Thermo Scientific), CTLA-4 ETB 118421 (10000 ng/mL, 5000 ng/mL, or 1000 ng/mL), and enzymatically inactive CTLA-4 ETB (10000 ng/mL, 5000 ng/mL, 1000 ng/mL). The cells were left in an incubator for 24 h. After the incubation, 60 μL supernatant was collected and stored at −20° C. until running it on Flex Map 3D Luminex (Luminex Corporation). To run on Luminex, samples were thawed at room temperature for 30 minutes and centrifuged to remove any cells or cell debris. The Human XL Cytokine Luminex Performance Premixed 10-plex kit was used for running this assay (R&D Systems). Samples were diluted 1:2 with the supplied diluent and the assay was run according to the manufacturer's instructions.

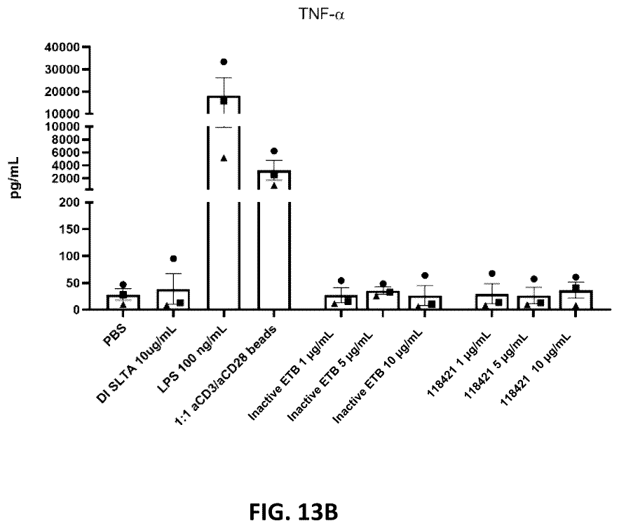

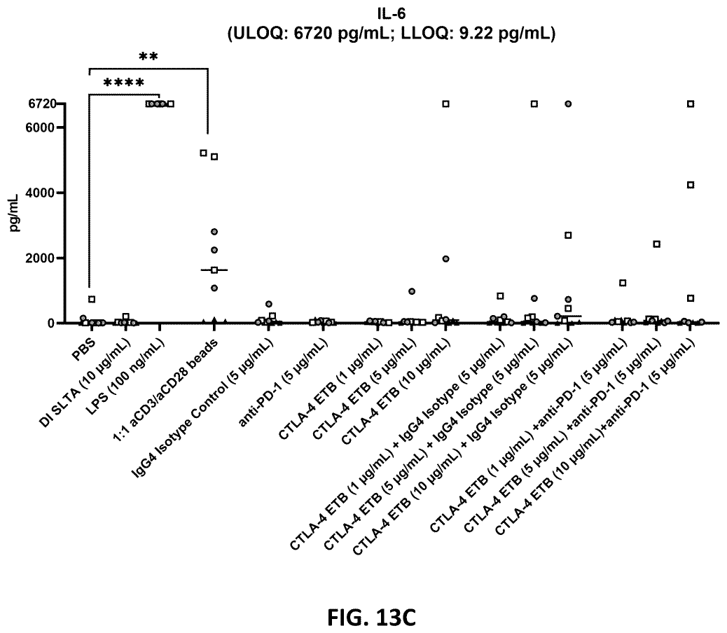

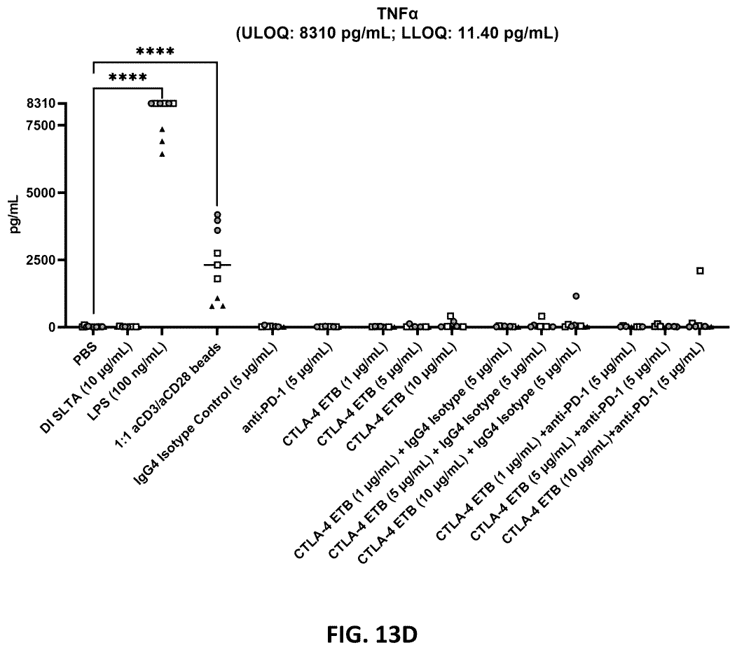

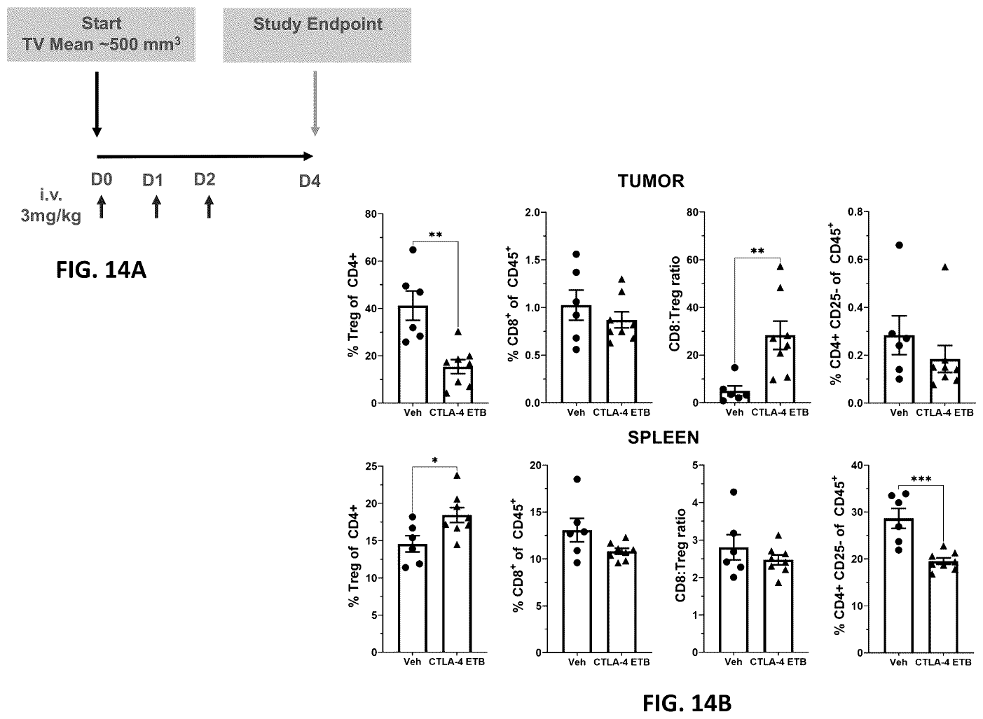

Results are shown in FIG. 13A and FIG. 13B. No major changes in IL-6 or TNF-α were observed upon treatment with either CTLA-4 ETB 118421, enzymatically inactive CTLA-4 ETB or DI-SLTA. Anti-CD3/CD28 beads were used as a positive control for activated T cells and resulted in robust IL-6 and TNFα release. LPS was used as a positive control for cytokine release resulting from innate immune cell activation and resulted in cytokine release as expected. The levels of these cytokines released in the supernatant were similar to the baseline levels in PBS treated cells. IL-6 increase has been shown to correlate with αCTLA-4 mAb toxicity and drive tumor growth (Hailemichael et al., Cancer Cell, 2022; 40:509-523). Absence of IL-6 release by CTLA-4 ETB 118421 could be attributed to a different mechanism of action of ETB versus a monoclonal antibody against CTLA-4. In summary, this data supports no general innate immune activation and cytokine secretion after treatment of PBMCs with CTLA-4 ETB 118421.

Additional experiments were performed to determine whether there would be adverse pro-inflammatory cytokine responses in patients treated with CTLA-4 ETB 118421 in combination with anti-PD-1. In general, the CTLA-4 ETB 118421 in combination with anti-PD-1 did not induce significant cytokine release across three healthy donor PBMCs tested using the cytokine release assay (FIG. 13C and FIG. 13D). These data support a favorable safety profile and indicate that the CTLA-4 ETB 118421 in combination with anti-PD-1 would not be expected to induce significant cytokine release syndrome nor severe adverse events in patients.

### Example 9: CTLA-4 Binding Molecules Selectively Deplete Tregs in Syngeneic Humanized Mouse Model

The objective of this study was to determine whether CTLA-4 ETB 118421 are capable of depleting Tregs in the tumor microenvironment in vivo.

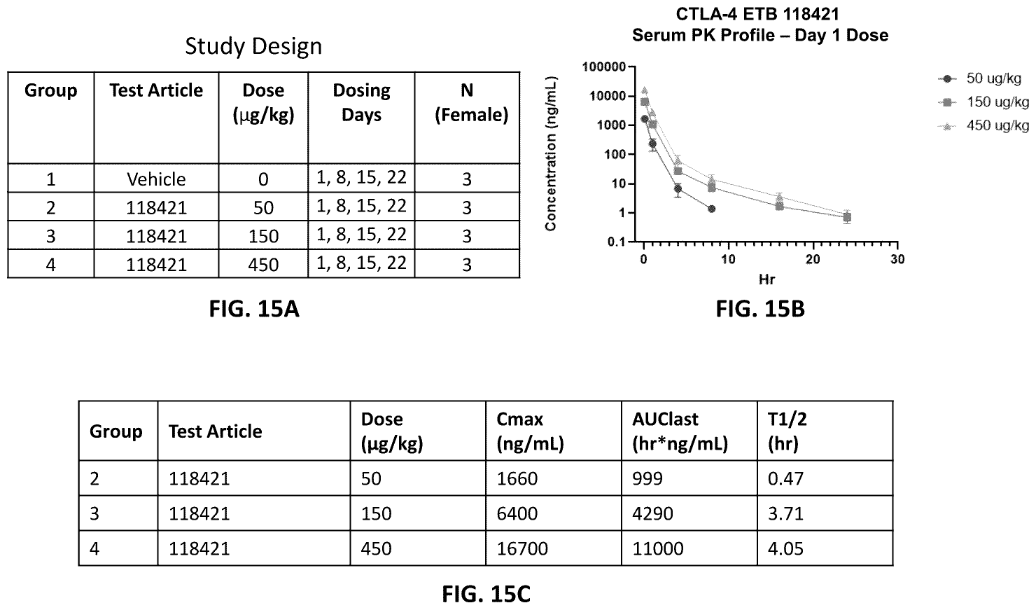

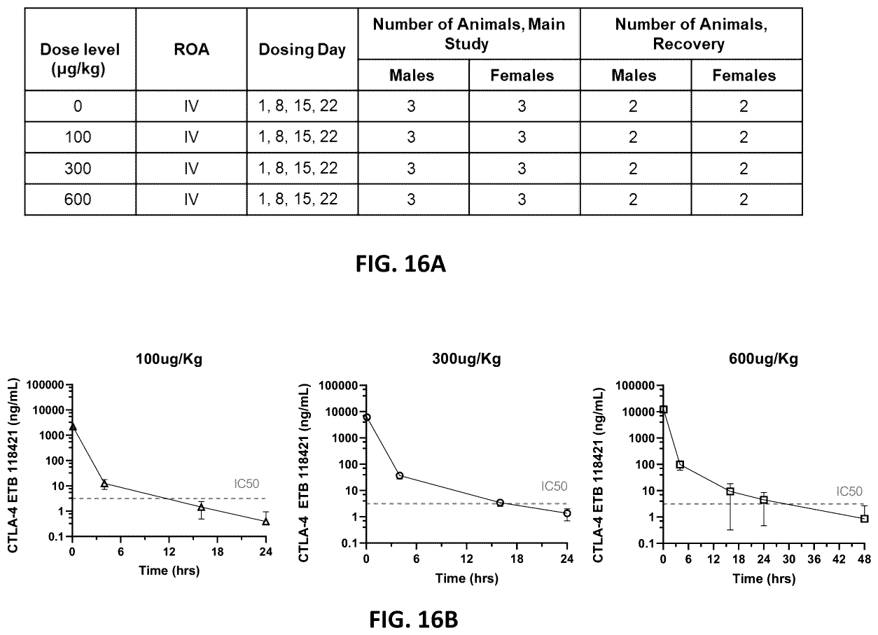

Human CTLA-4 knock-in HuGEMM mice (B-hCTLA-4 C57/BL6) (Biocytogen) were inoculated with MC38 tumors. When the tumors reached 500 mm³, CTLA-4 ETB 118421 was administered at 3 mg/kg for 3 consecutive days. On day 4, the tumors and spleens were harvested and processed for immunophenotyping (FIG. 14A).

The percentages of CD4+ effectors, CD8+ CTLs and Tregs from the tumor and spleen are displayed on the graphs in FIG. 14B. CTLA-4 ETB 118421 significantly depleted CD4+ Tregs from the tumor microenvironment relative to vehicle control but did not significantly deplete CD8+ T-cells from the tumor microenvironment. A statistical increase in the CD8 to Treg ratio was observed in the tumor microenvironment with CTLA-4 ETB 118421, primarily driven by the reduction in Tregs in the tumor microenvironment. CTLA-4 ETB 118421 did not show the same effect outside the tumor microenvironment. CTLA-4 ETB 118421 resulted in an increase in Tregs in the spleen of the animals and the CD8 to Treg ratio in the spleen was not significantly altered by CTLA-4 ETB 118421.

### Example 10: Toxicology and Pharmacokinetics of CTLA-4 Binding Molecules in Non-Human Primates

The objective of these studies was to determine the toxicology and pharmacokinetics of CTLA-4 binding molecules using non-human primates.

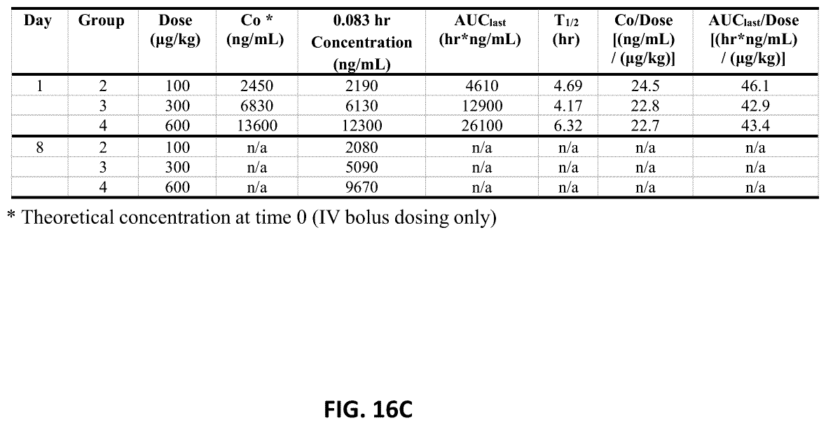

FIG. 15A shows the study design. FIG. 15B depicts a graph showing serum concentration-time profiles following intravenous administration of CTLA-4 ETB 118421 in non-human primates. FIG. 15C is a table summarizing individual pharmacokinetic parameters following intravenous administration of CTLA-4 ETB 118421 in non-human primates.

No mortality was observed. Clinical observations included mild flaking/sloughing of the skin of the face, of the right hindlimb, and of both hindlimbs in one animal at 150 μg/kg; mildly decreased food consumption in all animals at 450 μg/kg. Minimally decreased albumin and mildly to markedly increased C-reactive protein in animals administered ≥50 μg/kg was observed. These results demonstrated that CTLA-4 ETB 118421 was well-tolerated at 450 μg/kg (highest dose tested) administered intravenously once weekly for 4-weeks and did not significantly alter the CTLA-4 low/null peripheral T cell populations.

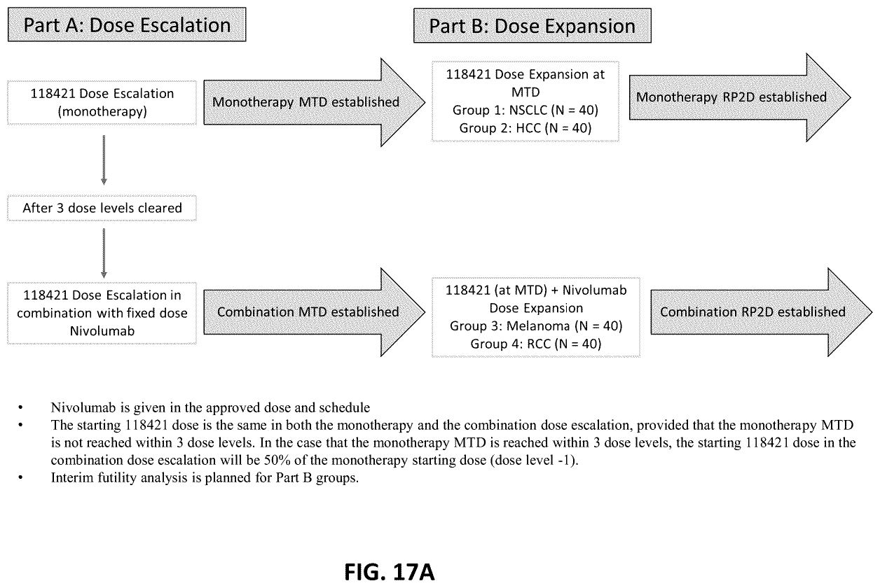

CTLA-4 ETB 118421 pharmacokinetics was analyzed in male and female non-human primates following a bolus injection of 0, 100, 300, or 600 μg/kg CTLA-4 ETB 118421 once weekly for four weeks (FIG. 16A). Serum concentrations of CTLA-4 ETB 118421 was determined using a Meso Scale Discovery (MSD) based sandwich immunoassay developed at BioAgilytix (Boston, Mass.). Briefly, biotinylated CTLA-4 was bound to streptavidin-coated MSD plates to capture CTLA-4 ETB 118421. Samples were diluted and incubated in pre-coated MSD plates. After the detection antibody (SULFO-TAG™-labeled anti-diSLTA mAb) binds to CTLA-4 ETB 118421, an ECL signal was measured using was read using MSD Sector Imager S120.

Results are shown in FIG. 16B and FIG. 16C. On day 1, mean CTLA-4 ETB 118421 Coincreased proportionally to dose (FIG. 16C). C₀ was hundreds of fold above the IC50 of primary Tregs. No accumulation of CTLA-4 ETB 118421 occurred on day 8 (based on the 0.083 hr timepoint).

### Example 11: A Phase 1 Open-Label, Dose-ranging Study to Investigate Safety, Tolerability, Efficacy, Pharmacokinetics, Pharmacodynamics, and Immunogenicity of 118421 as a Monotherapy and in Combination with Nivolumab in Patients with Advanced Solid Cancer

The CTLA-4 ETB 118421will be evaluated as a monotherapy and in combination with nivolumab in a phase 1 open-label dose-ranging study in subjects with advanced solid cancer.

### I. Investigational Plan

A. Study Design:

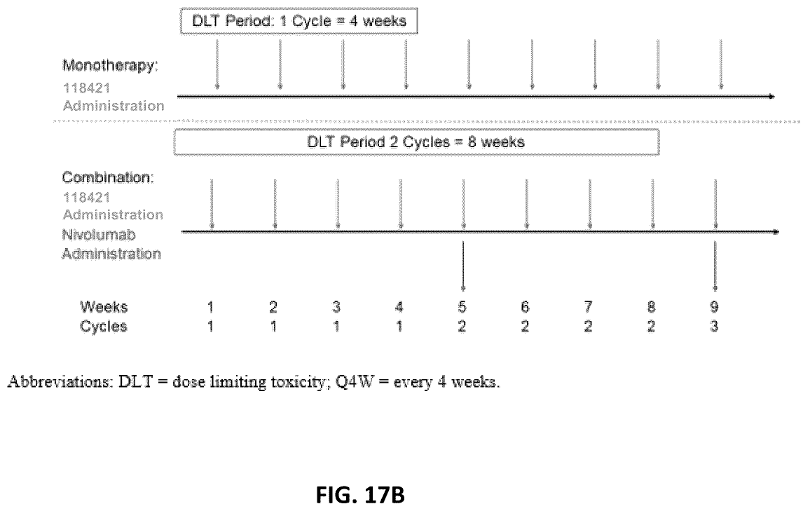

This study will be conducted in two sequential parts: Part A and Part B. The primary objective of Part A is to evaluate the safety and tolerability of 118421 as monotherapy and in combination with nivolumab in patients with selected advanced solid tumor types and estimate the maximum tolerated dose (MTD). The primary objective for Part B is to identify the dose(s) to be studied as the recommended phase 2 dose (RP2D) of 118421 as monotherapy and in combination with nivolumab in patients with selected advanced solid tumor types. The study design of both Part A and Part B will be open-label, parallel dose cohorts in patients with advanced solid tumors. Schematics of the study design are shown in FIG. 17A and FIG. 17B.

The study will have approximately 190 subjects: 24 to 30 subjects in Part A monotherapy, 12-24 subjects in Part A combination therapy with nivolumab, and 160 subjects in Part B (40 per arm).

(1) Part A Study Design:

The starting 118421 dose will be no higher than ⅙ of the highest non-severely toxic dose (HNSTD) in the relevant animal species, non-human primates. If the first monotherapy cohort, Cohort 1, dose proves intolerable, the next dose cohort, Cohort −1, will enroll patients at 50% of the dose as Cohort 1 used. After the first 3 monotherapy dose cohorts have been observed to have tolerable safety profiles, parallel combination dose escalation cohorts will be initiated, using the lowest safe monotherapy starting dose of weekly 118421 and 480 mg nivolumab every 4 weeks. If the monotherapy MTD is reached within 3 dose levels, the starting 118421 dose in the combination dose escalation will be 50% of the monotherapy starting dose (dose level −1). If the monotherapy MTD is dose level −1, the combination with nivolumab will first be studied with 118421 at a dose 50% of dose level −1. The 118421 dose in each combination escalation cohort must be at least one dose level lower than the dose level in the concurrent 118421 monotherapy escalation cohort until the MTD has been declared. Once the 118421 MTD has been declared, it will be permissible to evaluate the safety of 118421 at its MTD with nivolumab, provided the lower 118421 doses in combination with nivolumab have been tolerable.

Part A will enroll patients with tumors where CTLA-4 inhibitors have been proven to provide benefit and in other select tumor types known to frequently have an immune rich tumor microenvironment. This will include melanoma, hepatocellular carcinoma (HCC), non-small cell lung cancer (NSCLC), renal cell carcinoma (RCC), Microsatellite Instability-High (MSI-H)/Mismatch Repair Deficient (dMMR) cancer, mesothelioma, esophageal squamous cell carcinoma (ESCC), squamous cell carcinoma of the head and neck (SCCHN), urothelial carcinoma, and cervical carcinoma.

The Dose Limiting Toxicity (DLT) period in the monotherapy dose escalation arm will be 1 Cycle (4 weeks). The DLT period in the combination dose escalation arm will be 2 Cycles (8 weeks). The first 4 weeks of the DLT period in the combination arm will assess the safety profile of 118421 as a monotherapy.

Part A of this study will employ a modified Toxicity Probability Interval (mTPI-2) design to guide dose escalation and find the MTDs. The mTPI-2 design is implemented in a fashion similar to the traditional 3+3 design but is more flexible and possesses superior operating characteristics that are comparable to those of the more complex model-based designs, such as the continual reassessment method (CRM). If adverse events such as cytokine release syndrome are observed with frequency particularly after the first 1-2 doses, a “step-up” dosing strategy may be used, wherein doses lower than the target dose are administered on cycle 1, days 1 and 8, before administering the target dose on cycle 1, day 15 and weekly thereafter.

Based on the experience gained during dose escalation, the dosing and frequency of 118421 may be modified or additional dose escalation cohorts will be included.

(2) Part B Study Design:

After the monotherapy and combination MTDs have been determined in Part A of the study, Part B of the study will enroll up to 40 additional patients in each treatment cohort at or below the MTD from Part A to further explore safety and efficacy and determine RP2D of 118421 as a monotherapy and in combination with nivolumab. Patients will be enrolled into one of 4 treatment cohorts defined by tumor indication.

Part B will enroll into parallel cohorts of patients with advanced disease not amenable to standard of care treatment. The patients must have received a programmed death ligand-1 PD-(L)1 inhibitor with or without a CTLA-4 inhibitor: Monotherapy Cohort 1: NSCLC; Monotherapy Cohort 2: HCC; Combination therapy Cohort 3: Melanoma; and Combination therapy Cohort 4: RCC.

B. Objectives and Endpoints:

The primary objectives are shown in Table 10 below.

TABLE 10

| OBJECTIVES | ENDPOINTS |
|---|---|
| PART A: To evaluate the safety and | incidence of AEs, including dose limiting |
| tolerability of 118421 as monotherapy | toxicities (DLTs), inclusive of physical exam findings, |
| and in combination with nivolumab in | laboratory abnormalities, and/or patient reported |
| patients with selected advanced solid | symptoms |
| tumor types and estimate the maximum | |
| tolerated dose (MTD). | |
| PART B: To confirm the | incidence of AEs |
| recommended phase 2 dose (RP2D) | objective response using Response Evaluation |
| of 118421 as monotherapy and in | Criteria in Solid Tumors (RECIST1.1) criteria |
| combination with nivolumab in | |
| patients with selected advanced solid | |
| tumor types | |
| To evaluate efficacy of 118421 as | |
| monotherapy and in combination with | |
| nivolumab in patients with selected | |
| advanced solid tumor types by using | |
| Objective Response Rate (ORR) | |

The secondary objectives are shown in Table 11 below.

TABLE 11

| OBJECTIVES | ENDPOINTS |
|---|---|
| Part A only: | ORR by RECIST 1.1 |
| To assess preliminary efficacy | duration of response (DOR) |
| | time to response (TTR) |
| | PFS |
| | disease control rate (DCR) |
| Part A and B: | PK parameters: |
| To characterize the PK profile of | maximum observed plasma concentration (Cₘₐₓ) |
| 118421 given as monotherapy and in | half-life |
| combination with nivolumab in | time of maximum observed plasma concentration |
| patients with selected advanced solid | (tₘₐₓ) |
| tumor types | the area under the concentration-time curve |
| | (AUC) from time zero to the last measurable |
| | concentration (AUC₀₋ₜ), total exposure (AUC_{0-∞}) |
| | clearance (CL) |
| | volume of distribution at steady-state (Vₛₛ) |
| Part B only: | DOR |
| To assess additional efficacy | TTR |
| parameters | PFS |
| | DCR |
| Part A and B: | ADA |
| To evaluate the immunogenicity of | neutralizing antibodies (Nab) |
| 118421 in patients with selected | |
| advanced solid tumors. | |

The exploratory objectives are shown in Table 12 below.

TABLE 12

| OBJECTIVES | ENDPOINTS |
|---|---|
| To explore the immune response to | change from baseline in immune cell subsets in the |
| 118421 treatment | periphery |
| If warranted by the study results, to | PK, pharmacodynamic, safety, and tumor response |
| evaluate the exposure-response | variables |
| relationship for 118421 | |
| To explore the Pharmacodynamic and | Serum cytokine levels, PK and efficacy data |
| Pharmacokinetic relationship of | |
| circulating biomarkers with tumor | |
| response | |
| To correlate the pharmacodynamic | Changes in the tumor microenvironment in biopsy |
| markers of cancer under study with the | tissue, correlating with tumor response |
| tumor response to 118421 | |

C. Dose Modification Criteria:

Refer to Table 18 for guidance on dose modification for Treatment Emergent Adverse Events (TEAEs).

The following actions are recommended after any TEAE:
(1) The Investigator should monitor the subject and if necessary, perform unscheduled diagnostic procedures and therapeutic interventions, including those not specified in the study protocol.
(2) If a subject experienced Grade 2 IRR, or another Grade 2 hypersensitivity event, Grade 2 CRS or Grade 2 CLS, then the Investigator should obtain serum samples for cytokines, complement, and histamine analyses as soon as possible after the TEAE onset. In case of SIR syndrome, Grade 2 CRS or Grade 2 CLS the subject should be hospitalized, if clinically appropriate.
(3) 118421 dose may be reduced at the Investigator's discretion after discussion with the Medical Monitor.
(4) 118421 treatment may be restarted if the TEAE that previously led to treatment delay has resolved to Grade 1 within 14 days. Treatment re-start after longer than 14 days delay needs to be approved by the Medical Monitor.
(5) After the resolution of the TEAE, the 118421 dose for continued treatment may be the same or reduced, after discussion with the Medical Monitor.

A TEAE that fulfills a DLT criterion will be declared as a DLT if the TEAE occurred during Cycle 1 (4 weeks) of 118421 therapy monotherapy OR, for the combination cohorts, during the first cycle of combination therapy after the start of 118421 infusion of dose 1 on cycle 1 day 1 (within the first 8 weeks).

If a TEAE that fulfills a DLT criterion is observed in subsequent treatment cycles of Part A, then this event will be considered in the overall determination of treatment tolerability after consultation with the investigator and medical monitor.

All AEs regardless of attribution should be considered for DLT except those events clearly due to the underlying disease or extraneous causes. The type and severity of TEAEs that could qualify as DLTs are presented below. The severity of TEAEs potentially qualifying as DLTs will be graded according to the Common Terminology Criteria for Adverse Events (CTCAE) V5.0. If the AE is not listed in the CTCAE V5.0, then the highest intensity level reached according to the scale in Table 19.

(1) Hematological TEAEs:
Grade ≥3 febrile neutropenia (absolute neutrophil count [ANC] defined as ANC <1000/4 and a single body temperature reading of >38.3° C. [101° F.] or a sustained body temperature of ≥38° C. [100.4° F.] for more than 1 hour);
Grade 4 neutropenia (ANC <500/4);
Grade 3 thrombocytopenia (<50,000/4 and 25,000/4)>7 days;
Grade 3 thrombocytopenia with bleeding; and
Grade 4 thrombocytopenia (<25,000/4) with or without bleeding.

(2) Non-hematological TEAEs:
Grade ≥3 non-hematologic adverse events regardless of duration with the following exceptions: (1) Grade 3 nausea/vomiting or diarrhea for less than 72 hours with adequate antiemetic and other supportive care; (2) Grade 3 fatigue for less than 1 week; (3) Grade 3 or higher electrolyte abnormality that lasts up to 72 hours, is not clinically significant, and resolves spontaneously or responds to conventional medical interventions; (4) Grade 3 or higher amylase or lipase that is not associated with symptoms or clinical manifestations of pancreatitis; and (5) Grade 3 skin reactions that improve to Grade ≤2 within 72 hours (with or without systemic corticosteroid treatment);
LVEF decrease of ≥10% and the value is <50% (consult with medical monitor to determine if patient can continue study treatment);
Aspartate aminotransferase (AST) and/or alanine aminotransferase (ALT) increase >5.0×Upper Limit of Normal (ULN) and ≤8×ULN for patients who enroll with AST and ALT <3×ULN; the increase must endure for ≥14 days;
AST and/or ALT increase ≥3.0×ULN with concomitant increase in total bilirubin >2×ULN and alkaline phosphatase <2×ULN and there is no other explanation for the combination of laboratory abnormalities (i.e., patients meeting criteria for Hy's Law)
AST or ALT >8.0×ULN regardless of duration;
Any death not clearly due to the underlying disease or extraneous causes;
Any dose delays of >14 days due to an adverse event that has not resolved or reduced to grade 1; and

Any other toxicity regardless of attribution should be considered for DLT (except for those events clearly due to the underlying disease or extraneous causes), irrespective of the type or severity, as determined by the investigator, medical monitor, and sponsor, considering the severity, duration, poor response to remedial therapy and/or inadequate resolution.

D. Dose Escalation/De-Escalation Guidelines:

(1) Dose Escalation Guidelines Between Cohorts:

If 1 DLT occurred, and the cohort is eligible for escalation: 1.33(X) μg/kg once per week (QW) of a 28-day cycle.

If no DLT occurred and if at least 1 subject experiences a Grade 2 118421-related non-DLT: 1.5(X) μg/kg QW of a 28-day cycle; if no subjects experience a Grade ≥2 118421-related non-DLT (i.e., if no 118421-related AEs occur or if all 118421-related AEs are Grade 1): 2(X) μg/kg QW of a 28-day cycle.

Lower dose increases (i.e., 25% increase) may be considered based on the frequency and severity of non-DLT AEs.

Following 25% or 33% dose escalations, a higher dose escalation increment (i.e., 50%) may be re-instituted if no Grade ≥3 118421-related non-DLT AEs occur in the 2 subsequent (consecutive) cohorts

Doses higher than 160 μg/kg may be explored only with maximal incremental increases of 33%.

(2) Dose De-Escalation Guidelines Between Cohorts:

The timing of the DLT in the cycle may influence the decision to dose reduce or change the dosing schedule.

Dose-limiting toxicity occur during Weeks 1 or 2—dose is reduced with the same schedule once per 4 weeks (Q4W).

Dose-limiting toxicity occur during Week 3—dose schedule is modified to every 2 weeks (Q2W).

Dose-limiting toxicity occur during Week 4—dose schedule is modified to once per week×3 weeks (QW×3) of a 28-day cycle

Dose-limiting toxicity does not clearly fit to a pattern or 2 DLTs occur at different time points—Safety Committee decides on the dose level and/or schedule change. If the first dose cohort is intolerable, a “dose minus 1” dose cohort will enroll patients at 50% of the cohort 1 dose (32 μg/kg). If the “dose minus 1” dose cohort is tolerable, then a new cohort will open at a further 50% reduction (8 μg/kg) in combination with nivolumab.

E. Treatment Beyond Disease Progression:

Treatment should be discontinued in all subjects who exhibit symptomatic disease progression per RECIST 1.1.

Subjects who continue treatment beyond radiographic disease progression per RECIST 1.1 should be closely monitored clinically and with a follow-up scan in 4 to 6 weeks after the initial determination of progression. Treatment should be discontinued at any time if clinical deterioration due to disease progression occurs, or if persistent disease growth is confirmed in a follow-up scan. In addition, subjects should be discontinued for unacceptable toxicity or for symptoms of deterioration attributed to disease progression as determined by the Investigator after an integrated assessment of radiographic data and clinical status.

118421 will be administered until disease progression, unacceptable toxicity, death, withdrawal of consent, or another reason for withdrawal. Subjects with clinical benefit (confirmed complete response [CR], partial response [PR] for at least 12 weeks, or stable disease [SD] for at least 24 weeks) may suspend treatment for up to 8 weeks and later resume treatment if the Investigator determines it is in the subject's best interest after discussion with the Medical Monitor. The subject needs to attend the scheduled imaging assessment during the drug holiday.

### II. Selection and Withdrawal of Subjects

A. Subject Inclusion Criteria:

Subjects meeting ALL the following inclusion criteria will be eligible for participation in the study.
(1) Subjects must be at least 18 years old at the time of informed consent.
(2) Subjects must have histologically confirmed, unresectable, locally advanced or metastatic melanoma, HCC, NSCLC, RCC, MSI-H/dMMR malignancies, urothelial carcinoma, mesothelioma, SCCHN, or cervical carcinoma are also eligible not amenable to standard treatment, or standard treatment is not available, or in the investigator's opinion, the standard treatment would not be in the patient's best interest. Part A only: evaluable or measurable disease according to RECIST 1.1. Part B only: at least 1 measurable tumor lesion according to RECIST 1.1.
(3) Eastern Cooperative Oncology Group (ECOG) performance score of 0 or 1.
(4) Prior treatment must include a PD-1 or PD-L1 inhibitor. Prior treatment CTLA-4 inhibitor is not required.
(5) Adequate bone marrow function as determined by: (a) ANC ≥1,500/μL (should not have received growth factors within 2 weeks prior to screening); (b) Platelet count ≥75,000/μL; (c) Hemoglobin 8 g/dL (no red blood cell transfusion within 2 weeks of study treatment start is allowed).
(6) Adequate renal function, based on estimated creatinine clearance (eCrCl) 50 mL/min, calculated by the Cockcroft-Gault equation. At the investigator's discretion, the eCrCl result <50 mL/min may be verified by the measured creatinine clearance (mCrCl) based on the 24-hour urine collection. Patients with mCrCl≥50 mL/min will be eligible irrespective of the eCrCl result calculated by the Cockcroft-Gault equation.
(7) Adequate hepatic function, as determined by: (a) Total bilirubin ≤1.5×ULN, or ≤2×ULN direct bilirubin for patients with Gilbert's Syndrome; (b) AST ≤3×ULN (or ≤5×ULN if liver metastasis or HCC); (c) ALT ≤3×ULN (or ≤5×ULN if liver metastasis or HCC); and (d) Adequate serum albumin (albumin ≥2.5 g/dL).
(8) Availability of a lesion which can be biopsied with acceptable risk.
(9) Subjects capable of bearing children must have a negative highly sensitive pregnancy test within 72 hours before the start of treatment. Subjects who are postmenopausal (>1 year since last menstrual cycle) or permanently sterilized (eg, bilateral tubal occlusion, hysterectomy, bilateral salpingectomy) may be considered as not of reproductive potential.
(10) Subjects of reproductive potential must agree either to abstain continuously from heterosexual intercourse or to use a highly effective birth control method from signing the informed consent until 30 days after the last dose of 118421 for subjects capable of bearing children and until 90 days after the last dose of 118421 for subjects capable of fathering a child.

B. Subject Exclusion Criteria:
(1) Unwilling or unable to provide an archived tumor biopsy sample.
(2) Unwilling or unable to undergo two sets or 3-6 core tumor biopsies: one set at study baseline (prior to first dose of study drug), one set between weeks 6-8. A final optional set will be collected at the end of treatment
(3) Received approved or investigational treatment for the disease under study (except ipilimumab where exclusion criterion 3 applies, requiring a washout which will be dependent upon prior ipilimumab dose and planned 118421 dose) within 4 weeks before the start of treatment. For small molecules (MW<0.9 kDa), the washout is 5 half-lives, but at least 2 weeks.
(4) Received tremelimumab within 18 days or ipilimumab 3 mg/kg within 60 days or ipilimumab 1 mg/kg within 30 days before the start of treatment. Once dose cohort of 32 μg/kg 118421 has been closed in dose escalation, a 30-day washout of ipilimumab is acceptable irrespective of prior dosing.
(5) Any concurrent cancer treatment, apart from local treatment of non-evaluable lesions for palliative intent (e.g., local surgery or radiotherapy).
(6) History or current evidence of another neoplastic disease, except cervical carcinoma in situ, superficial noninvasive bladder tumors, curatively treated, Stage I to II non-melanoma skin cancer, prostate cancer managed by active surveillance, or any previous cancer curatively treated <2 years before the start of treatment.
(7) Active autoimmune disease that required systemic treatment in the past. Patients who have not required systemic treatment for at least two years may be enrolled if permission is provided after discussion with the Medical Monitor (replacement therapy, e.g., thyroxine, insulin, or physiologic corticosteroid replacement therapy for adrenal or pituitary insufficiency is allowed).
(8) Evidence of active noninfectious Grade 2 pneumonitis or current evidence of ≥Grade 3 other underlying pulmonary disease.
(9) Ongoing >Grade 1 immune related toxicity caused by prior CPI therapy (i.e., PD-1 inhibitors, PD-L1 inhibitors, or CTLA-4 inhibitors). Subjects with stable endocrinological AEs, e.g., hypothyroidism, adrenal insufficiency, hypopituitarism, or diabetes mellitus, must have been on a stable dose of supplemental therapy for at least 2 weeks before screening to be eligible for this study. History of repeat Grade 2 pneumonitis or myocarditis on previous CPI and/or Grade 3 irAE on previous CPI treatment.
(10) Current evidence of new or growing CNS metastases during screening. Patients with known CNS metastases will be eligible if they meet all the following criteria: (a) received radiotherapy or another appropriate therapy for the CNS metastases, if clinically indicated; (b) have stable CNS disease on the computed tomography (CT) or magnetic resonance imaging (MRI) scan within 4 weeks before signing the informed consent compared with prior neuroimaging.
(11) History or current evidence of significant cardiovascular disease before the start of treatment including but not limited to the following conditions: (a) angina pectoris requiring anti-anginal medication (chest pain: CTCAE Grade ≥2); (b) clinically significant valvular disease; (c) myocardial infarction within 12 months prior to the start of treatment; (d) arterial thrombosis or pulmonary embolism within 3 months before the start of treatment; (e) current Grade ≥2 symptomatic congestive heart failure (CHF) or New York Heart Association (NYHA) criteria Class ≥II; (f) left ventricular ejection fraction (LVEF) below normal (per institutional standard), assessed, preferably by Echo or multiple-gated acquisition (MUGA) scan if Echo is not available, within 1 month before starting study treatment; (g) high-risk uncontrolled arrhythmias (i.e., atrial tachycardia with a heart rate >100/min at rest and upon repeat testing, significant ventricular arrhythmia (CTCAE grade ≥2 [ventricular tachycardia], or higher-grade atrioventricular [AV]-block [second degree AV-block Type 2 [Mobitz 2] or third-degree AV-block]) or left ventricular bundle branch block. Patients receiving digoxin, calcium channel blockers, or beta-adrenergic blockers are eligible at the investigator's discretion after consultation with medical monitor if the dose has been stable for ≥2 weeks before the start of treatment with 118421; (h) any of the following within 3 months before the start of treatment: pericarditis (any CTCAE grade), pericardial effusion (CTCAE Grade ≥3), non-malignant pleural effusion (CTCAE Grade ≥2) or malignant pleural effusion (CTCAE Grade ≥3) (patients with pleural effusion that is manageable and stable >3 months prior to study are eligible); (i) QTcF ≥470 ms (average from 3 QTcF values on the triplicate 12-lead ECG) at screening.
(12) Current evidence of active, uncontrolled hepatitis B virus, hepatitis C virus, Human Immunodeficiency Virus (HIV) [evidenced by detectable viral load by Polymerase Chain Reaction (PCR)] or Acquired Immunodeficiency Syndrome (AIDS)-related illness: (a) Serology and virology measurements are not required to be performed at screening, but any previously reported results should be used for eligibility purposes. Investigators will test per their discretion; (b) Patients with a history of treated hepatitis C and non-quantifiable hepatitis C virus-RNA may be enrolled; (c) Patients on treatment for hepatitis B, hepatitis C, and/or HIV will be eligible if they have undetectable viral load (patients with HIV with CD4+ T cell [CD4+] counts ≥350 cells/μL may be enrolled).
(13) Patients with unintentional weight loss greater than 10% of their body weight over the preceding 2 months or less prior to Cycle 1 Day 1.

C. Subject Withdrawal Criteria:

Subjects must be withdrawn from the study if requested or at the request of their legal representative. The subject has the right to withdraw from the study at any time for any reason, without the need to justify withdrawal. The subject will not suffer any disadvantage because of the withdrawal.

Subjects must also be withdrawn if the serum beta human chorionic gonadotropin (β-HCG) pregnancy test indicates that they are pregnant at any time from signing the consent until the end of treatment (EoT) Visit.

The subject may be withdrawn from the study at the discretion of the Investigator due to: (a) safety concerns; (b) lack of clinical benefit (radiographical disease progression is not documented but the Investigator determines that the subject requires alternative anti-cancer treatment); and/or (c) non-compliance with study procedures to the extent that precludes the assessment of study objectives.

All subjects who permanently discontinue study treatment for any reason should have an EoT Visit performed.

The reason for any discontinuation from the study will be documented in the subject's medical record and recorded on the appropriate eCRF.

### III. Treatment of Subjects:

A. 118421:

A description of the study drug is described in Table 13 below.

TABLE 13

| | Investigational Product |
|---|---|
| Product Name: | 118421 |
| Dosage Form: | 2 mL glass vial containing 2 mL of 118421 at 0.5 mg/mL |
| Unit Dose | 1.0 mg per vial |
| Route of Administration | Intravenous |
| Physical Description | Clear colorless solution, may contain white or translucent particles |

All packaging and labeling operations for 118421 will be performed according to good manufacturing practice (GMP) and good clinical practice (GCP) guidelines, as well as the national regulatory requirements which apply to this clinical investigation.

118421 will be visually inspected for discoloration prior to use.

118421 will be administered over 30 minutes (±5 min) IV syringe infusion prior to the infusion of nivolumab, if used. 118421 will be administered through an intravenous line containing a sterile, non-pyrogenic, low protein binding in-line filter.

118421 will be administered on days 1, 8, 15, and 22 of a 28-day cycle. 118421 doses may be administered within a 2-day window if deemed appropriate by the investigator, however a minimum of 18 days is required for all 4 infusions.

The dose of 118421 will be calculated based on the patient's baseline body weight. The body weight will be measured before the first dose of 118421 in each cycle. If the body weight has changed by greater than 10% from the baseline value (pre-dose on C1D1), this will require recalculation of 118421 dose. The new body weight will serve as a new baseline.

The dose of 118421 in Part A (dose ranging) will depend on the cohort. The starting dose of 118421 in Part B (dose expansion) will be at or below the MTD determined in Part A for 118421 as a monotherapy and at or below the MTD for 118421 in combination with nivolumab.

H1/H2 blocker-containing agents and anti-pyrectics are required before each dose in Cycle 1, and the anti-pyretic should continue for 24 hours post-dose of Cycle 1, Day 1. From Cycle 2 on, the investigator may decide to change the premedication (e.g., reduce dose or skip) if deemed appropriate.

118421 will be administered until disease progression, unacceptable toxicity, death, withdrawal of consent or another reason for withdrawal. For patients with clinical benefit (clinical benefit is considered to be any of the following: confirmed complete response (CR); partial response (PR) for at least 12 weeks; or SD for at least 24 weeks) the treatment can be suspended for a period if it is in the patient's best interest.

If deemed necessary, in the combination arm the investigator may decide to terminate the nivolumab treatment while continuing 118421 treatment. In case the investigator decides to stop 118421 but intends to continue the nivolumab treatment, the patient needs to be withdrawn from study treatment.

B. Nivolumab (if Applicable):

Nivolumab will be visually inspected for particulate matter and discoloration prior to use.

Nivolumab infusion will be completed prior to initiating the 118421 infusion.

There will be separate infusion bags and filters used for each infusion. The intravenous line will be flushed at end of infusion. Other drugs will not be co-administered through the same intravenous line.

C. Concomitant Medications:

New medication [both prescription and Over-the-Counter (OTC) medications and supplements, including herbals], or change in medication reported by the subject or subject's medical records between the screening medical history and the start of treatment should be reported as concomitant medication. During the treatment and up to the short-term follow-up (STFU) Visit 2, concomitant medications will be reported by verbal probes at every visit to the clinic or at every phone contact; in addition, subject's spontaneous reports will be captured.

Except where expressly prohibited, the use of concomitant medications is permitted at the Investigator's discretion for management of toxicities.

Concomitant use of blood products and growth factors are permitted for supportive care at the Investigator's discretion after the DLT period, or if the patient experiences a hematologic DLT necessitating growth factor or blood product support.

Palliative therapy, such as radiotherapy or minor surgery, to non-target lesions, is allowed at the Investigator's discretion after consultation with the Medical Monitor. Lesions which are irradiated or managed with substantive surgical or other invasive treatment likely to alter their size will be considered non-evaluable for response following the intervention. However, if a substantively treated lesion demonstrates growth after procedure completion, disease progression must be considered.

Following enrollment, patients may not receive: (a) other investigational drugs; (b) corticosteroids except to treat immune-related AEs or to provide prophylaxis of AE's such as IRR's; and/or (c) cancer drugs other than clinical trial materials.

### IV. Study and Safety Assessments:

Schedules of study and safety assessments are provided in Tables 14-17 below.

TABLE 14
Schedule of Assessments

| | Treatment Period | | | | | | | | |
|---|---|---|---|---|---|---|---|---|---|
| | (28-day cycles) | | | | | | STFU | | |
| SCR¹ | D1 | D2 | D8 | D15 | D22 | EoT⁴ | 1&2² | LTFU³ | Notes |

Study Treatment-All assessments are performed prior to 118421 infusion unless otherwise indicated

| | | | | | |
|---|---|---|---|---|---|
| 118421 infusion | X | X | X | X | In case of a dosing schedule |
| | | | | | change, the subject visit |
| | | | | | days will change accordingly |
| | | | | | (D1, D15; OR D1, D8, |
| | | | | | D15). Assessments are the |
| | | | | | same as described in this |
| | | | | | SOA. |
| Nivolumab infusion | (X) | | | | (X): 480 mg nivolumab is |
| | | | | | administered on D1 of each |
| | | | | | cycle starting with Cycle 2 for |
| | | | | | all combination dose escalation |
| | | | | | and dose expansion cohorts. |

General and Safety Assessments

| Informed consent | X | | | | | | | | A separate informed consent |
|---|---|---|---|---|---|---|---|---|---|
| | | | | | | | | | form is required to be signed |
| | | | | | | | | | prior to treatment continuing |
| | | | | | | | | | beyond progression. |
| Eligibility criteria | X | | | | | | | | |
| Demography | X | | | | | | | | |
| Physical exam | X | X | | | | X | | | Complete physical exam at |
| | | | | | | | | | screening and EoT. Abbreviated |
| | | | | | | | | | exam D1 of every cycle. |
| Height | X | | | | | | | | |
| Weight | X | X | X | X | X | X | X | | Body weight measured before |
| | | | | | | | | | the start of treatment on C1D1 |
| | | | | | | | | | will be used to calculate the |
| | | | | | | | | | 118421 dose in all subsequent |
| | | | | | | | | | cycles. The dose must be re- |
| | | | | | | | | | calculated when the body weight |
| | | | | | | | | | has changed by ≥10% from the |
| | | | | | | | | | baseline C1D1 value; or according |
| | | | | | | | | | to institutional policies. |
| Medical history and | X | | | | | | | | |
| prior cancer therapy | | | | | | | | | |
| Prior medication | X | | | | | | | | Any medication (prescription, |
| | | | | | | | | | OTC, and supplements) used within |
| | | | | | | | | | 4 weeks prior to screening. |
| NYHA | X | (X) | | | | X | | | Only applicable to subjects with |
| | | | | | | | | | heart failure. |
| | | | | | | | | | (X): Only collected D1 every |
| | | | | | | | | | odd cycle, starting with Cycle 3. |
| ECOG | X | X | | | | X | | | |
| Triplicate 12-lead ECG | Refer to Table 15 for detailed time point collection. | | | | | | | | |
| Vital signs | X | X | X | X | X | X | X | | Measured after at least 5 minutes |
| (BP, HR, RR, | | | | | | | | | in a sitting or semi-recumbent |
| body temperature) | | | | | | | | | position. |
| | | | | | | | | | Collect at the following times on |
| | | | | | | | | | dosing days (±10 min): (a) pre-dose |
| | | | | | | | | | (any time before start of infusion); |
| | | | | | | | | | (b) at the end of infusion then 30, |
| | | | | | | | | | 60 and 120 minutes after the end of |
| | | | | | | | | | the infusion; (c) C1D1: also collect |
| | | | | | | | | | 3 and 4 hours after start of infusion |
| LVEF | X | (X) | | | | | | | A pre-study LVEF assessment is |
| | | | | | | | | | acceptable if obtained within 28 |
| | | | | | | | | | days before starting study treatment. |
| | | | | | | | | | (X): Collected within 2 weeks |
| | | | | | | | | | before D1 dosing of every odd |
| | | | | | | | | | cycle, starting with Cycle 3. |
| AE review | X | [symbol] (X) | | | | | | | (X) AE review through STFU Visit 1. |
| | | | | | | | | | Events which occurring screening |
| | | | | | | | | | are reported in medical history. |
| Concomitant | X | [symbol] | | | | | | | Any medication used within 4 weeks |
| medication | | | | | | | | | prior to the first dose of investigational |
| review | | | | | | | | | product until 30 d (+7 d) following |
| | | | | | | | | | the EOT. |
| LTFU Visit | | | | | | | | X | Every 3 months (±30 days) |

Laboratory Assessments (if not otherwise specified, the safety labs are taken pre-dose)

| | | | | | | | | | |
|---|---|---|---|---|---|---|---|---|---|
| Viral serology | X | | | | | | | | Serology for HIV, HBV, and HCV |
| | | | | | | | | | is not required if seronegativity is |
| | | | | | | | | | documented in medical history and |
| | | | | | | | | | there are no clinical signs suggestive |
| | | | | | | | | | of HIV or hepatitis infections, or |
| | | | | | | | | | suspected exposure. |
| Urinalysis (Dipstick) | (X) | X | | X | X | X | X | X | Analysis of urine micro-sediment |
| | | | | | | | | | may be performed at the |
| | | | | | | | | | Investigator's discretion by the |
| | | | | | | | | | local lab. |
| | | | | | | | | | (X): At screening, collected within |
| | | | | | | | | | 14 days of the start of treatment, |
| | | | | | | | | | if applicable |
| Pregnancy test | X | X | | | | | X | (X) | Performed within 72 hr before |
| | | | | | | | | | dosing on D1. |
| | | | | | | | | | (X) required only at STFU Visit 1. |
| Hematology | X | X | (X) | X | X | X | X | X | At screening, collected within 14 |
| | | | | | | | | | days of the start of treatment. |
| | | | | | | | | | (X): Collected during Cycle 1 only, |
| | | | | | | | | | unless deemed necessary due to |
| | | | | | | | | | emerging data. |
| Chemistry | X | (X) | | X | X | X | X | X | Includes creatinine clearance. |
| | | | | | | | | | At screening, collected within 14 |
| | | | | | | | | | days of the start of treatment. |
| | | | | | | | | | (X) HBA1c assessed on D1 of |
| | | | | | | | | | every odd cycle starting at Cycle 3. |
| | | | | | | | | | Coagulation will be tested prior |
| | | | | | | | | | to the first dose of every cycle. |
| Adrenal function | | X | | | | | | | Cortisol will be measured prior to |
| | | | | | | | | | the first dose of every cycle |
| Thyroid function | X | X | | | | | X | | |
| hs-troponin T | X | X | X | X | X | X | X | X | Collected pre-infusion (all cycles) |
| | | | | | | | | | and 3 to 4 hours post-infusion up |
| | | | | | | | | | to C6 or as symptoms indicate. |
| | | | | | | | | | At screening, collected within 14 |
| | | | | | | | | | days of the start of treatment |
| NT-pro-BNP | | X | | X | X | X | | | Collected up to C6 or as symptoms |
| | | | | | | | | | indicate. |

| | |
|---|---|
| PK Collection | See PK Collection Table (Table 15) for details. |
| Biomarker Collection | See Biomarker Collection Table (Table 16) for details. |

Efficacy Assessments

| | | | |
|---|---|---|---|
| Radiological | X | Every 8 weeks (±1 week) | Scans may be obtained before the |
| assessment | | until disease progression, death, or lost to follow-up | signing of informed consent if |
| | | AND | collected for SOC, up to 6 weeks |
| | | For subjects without response or stable disease at EoT: | before the start of treatment. |
| | | within 7 days of the EoT Visit (only if the previous tumor | Radiological assessments need to |
| | | scan has been performed >4 weeks before the EoT Visit) | follow the original schedule, |
| | | | regardless of dose delays. Patients |
| | | | with radiographic CR, PR, or SD |
| | | | for at least 24 weeks may have scans |
| | | | performed every 12 weeks. |
| | | | Assessments during LTFU should |
| | | | be collected every 3 months. |

¹Screening procedures will be performed within 28 days before the start of treatment on C1D1, except safety labs which must be within 14 days before treatment start and the radiographical disease assessment which may be performed within 6 weeks before C1D1.
²STFU Visits should occur 30 days (±7 days) and 90 days (±7 days) after the last dose of 118421. Can be skipped only for subjects who withdrew consent and refused further data collection, started new anticancer therapy or another investigational drug, or were lost to follow-up. STFU Visits should be performed in the clinic, where possible but may be performed via a telephone call if subject cannot attend a clinic visit.
3LTFU visits (phone calls or site visits) should occur every 3 months (±30 days) after the STFU Visit 2 for up to 24 months. Subjects who discontinue the study treatment for radiographical disease progression or who started new anticancer therapy or another investigational drug, will be followed only for OS (collect information about death if applicable). Subjects who discontinue the study treatment for reasons other than radiographical disease progression, will be followed for PFS (collect information also on tumor status and any new anti-cancer therapy since the last study visit/phone call) and radiological assessments will be performed per local SOC and symptoms.
⁴The EOT Visit will be performed at the end of the treatment period. EOT Visit should occur within 14 days after the last dose of 118421, and before start of new therapy except for subjects who withdrew consent and objected to further data collection or were lost to follow-up. EOT Visit should be performed in the clinic, where possible, and it can be completed on the same day as another study visit. EOT Visit may be performed by telephone call if a subject cannot attend a clinic visit. In such instances, missed assessments [e.g., laboratory assessments, physical examinations PEs)], are not considered deviations.

TABLE 15
Schedule of Pharmacokinetic and Triplicate ECG Sampling

| | | Cycle 1 | | | | | | | | |
|---|---|---|---|---|---|---|---|---|---|---|
| | | Day 1 | | | Day 8 | | Day 15 | | Day 22 | |
| | Triplicate | Triplicate | | Day 2 | Triplicate | | Triplicate | | Triplicate | |
| | ECG | ECG | PK | PK | ECG | PK | ECG | PK | ECG | PK |
| Screening | X | | | | | | | | | |
| (within 28 | | | | | | | | | | |
| days of | | | | | | | | | | |
| C1D1) | | | | | | | | | | |
| Predose ≤4 h | | X | X | | X | X | X | X | X | X |
| prior SOI | | | | | | | | | | |
| 5 min after | | X | X | | X | X | X | X | X | X |
| EOI (±1 m) | | | | | | | | | | |
| 1 hr after | | | X | | | X | | | | |
| EOI (±5 m) | | | | | | | | | | |
| 2 hr after | | X | X | | X | X | X | | X | |
| EOI (±30 m) | | | | | | | | | | |
| 4 hr after | | X | X | | X | X | X | X | X | X |
| EOI (±30 m) | | | | | | | | | | |
| 6 hr after | | | X | | | X | | | | |
| EOI (±1 hr) | | | | | | | | | | |
| 24 hr after | | | | X | | | | | | |
| EOI (±2 hr) | | | | | | | | | | |
| | Cycle 2 | | | | | | | | Cycle 3+ | |
| | Day 1 | | Day 8 | | Day 15 | | Day 22 | | Day 1 | |
| | Triplicate | | Triplicate | | Triplicate | | Triplicate | | Triplicate | |
| | ECG | PK | ECG | PK | ECG | PK | ECG | PK | ECG | PK |
| Screening | | | | | | | | | | |
| (within 28 | | | | | | | | | | |
| days of | | | | | | | | | | |
| C1D1) | | | | | | | | | | |
| Predose ≤4 h | X | X | X | | X | | X | | X | X |
| prior SOI | | | | | | | | | | |
| 5 min after | X | X | X | | X | | X | | X | X |
| EOI (±1 m) | | | | | | | | | | |
| 1 hr after | | | | | | | | | | |
| EOI (±5 m) | | | | | | | | | | |
| 2 hr after | X | | X | | X | | X | | X | |
| EOI (±30 m) | | | | | | | | | | |
| 4 hr after | X | X | X | | X | | X | | | X |
| EOI (±30 m) | | | | | | | | | | |
| 6 hr after | | | | | | | | | | |
| EOI (±1 hr) | | | | | | | | | | |
| 24 hr after | | | | | | | | | | |
| EOI (±2 hr) | | | | | | | | | | |

C1D1 = Cycle 1 Day 1, ECG = electrocardiogram; EOI = end of infusions; PK = pharmacokinetic; SOI = start of infusion.
If a 118421 dose is rescheduled, then the PK sampling needs to follow the new dosing schedule. Unscheduled assessments may be performed at any time at the Investigator's discretion. Samples for PK must be drawn from either a different line than that used for 118421 administration or from the same line after flushing with saline and with 10 mL of blood discarded. If not medically feasible, on an individual basis the study site staff must consult with the sponsor on alternative collection options.

TABLE 16
Biomarker Sample Collection

| | Screening¹ | Cycle 1 | | | | | | | | | Cycle 2 | |
|---|---|---|---|---|---|---|---|---|---|---|---|---|
| | Within 28 | Day 1 | | Day 2 | Day 8 | | Day 15 | | Day 22 | | Day 1 | |
| | Days of | Pre- | Post- | Post- | Pre- | Post- | Pre- | Post- | Pre- | Post- | Pre- | Post- |
| | C1D1 | dose | dose | dose | dose | dose | dose | dose | dose | dose | dose | dose |
| Immunophenotyping: | | X | X | X | X | X | X | X | X | X | X | X |
| TBNKs, T-Regs | | | | | | | | | | | | |
| Complement³ | | X | | | | | | | | | | |
| Histamine³ | | X | | | | | | | | | | |
| Circulating | | X | X | X | X | X | X | X | X | X | X | X |
| Biomarkers³ | | | | | | | | | | | | |
| 118421 ADA/Nab | | X | | | X | | X | | X | | X | |
| Tumor Tissue | X | | | | | | X | | | | | |
| Biopsy⁴ | | | | | | | | | | | | |

| | Cycle 2 | | | | | | Cycle 3+ | | | |
|---|---|---|---|---|---|---|---|---|---|---|
| | Day 8 | | Day 15 | | Day 22 | | Day 1 | | | |
| | Pre- | Post- | Pre- | Post- | Pre- | Post- | Pre- | Post- | | |
| | dose | dose | dose | dose | dose | dose | dose | dose | EOT⁵ | STFU² |
| Immunophenotyping: | X | X | X | X | X | X | X | X | X | X |
| TBNKs, T-Regs | | | | | | | | | | |
| Complement³ | | | | | | | | | | |
| Histamine³ | | | | | | | | | | |
| Circulating | X | X | X | X | X | X | X | X | X | X |
| Biomarkers³ | | | | | | | | | | |
| 118421 ADA/Nab | | | | | | X | | | X | X |
| Tumor Tissue | | | | | | | | | X⁶ | |
| Biopsy⁴ | | | | | | | | | | |

Pre dose, Post dose = 4 hours ±30 mins after EOI; Post dose on day 2 = 24 hours ±2 hours after EOI
¹Screening procedures will be performed within 28 days before the start of treatment on C1D1, except safety labs which must be within 14 days before treatment start and the radiographical disease assessment which may be performed within 6 weeks before C1D1.
²STFU Visits should occur 30 days (±7 days) and 90 days (±7 days) after the last dose of 118421. Can be skipped only for subjects who withdrew consent and objected to further data collection, started new anticancer therapy or another investigational drug, or were lost to follow-up. STFU Visits should be performed in the clinic, where possible, but may be performed via a telephone call if subject cannot attend a clinic visit.
³Also collected if a subject experiences a Grade ≥2 or repeat Grade ≥2 IRR/CLS/CRS or other hypersensitivity reaction.
⁴Collected during screening (after a patient has been determined to be eligible), after 6-8 weeks on treatment, and at EOT (optional).
⁵The EOT Visit will be performed at the end of the treatment period. EOT Visit should occur within 14 days after the last dose of 118421, and before start of new therapy except for subjects who withdrew consent and objected to further data collection or were lost to follow-up. EOT Visit should be performed in the clinic, where possible, and it can be completed on the same day as another study visit. EOT Visit may be performed by telephone call if a subject cannot attend a clinic visit. In such instances, missed assessments [e.g., laboratory assessments, physical examinations PEs)], are not considered deviations.
⁶The final tumor tissue biopsy will take place at the time of progression.
ADA = anti-drug antibodies, CLS = capillary leak syndrome, CRS = cytokine release syndrome, EOI = end of infusion, IRR = infusion related reaction, Nab = neutralizing antibodies

TABLE 17
Laboratory Safety Assessments

| | |
|---|---|
| Assessment | |
| Category | Specific Assessment |

Local

| | | |
|---|---|---|
| Hematology | Hematocrit | RBC¹ |
| | Hemoglobin | WBC |
| | Platelet count | Complete WBC Differential² |
| | Neutrophil count | |
| Pregnancy | Serum or urine β-human chorionic | |
| | gonadotropin. | |
| Coagulation | aPTT | INR or PT |
| Chemistry | Albumin | Creatinine |
| | ALP | eCrCl (Cockcroft Gault)³ |
| | ALT (SGPT) | GGT |
| | Amylase | Glucose |
| | AST (SGOT) | HbA1c |
| | β-HCG³ | LDH |
| | Bicarbonate | Lipase |
| | Bilirubin | Magnesium, phosphorous, |
| | BUN or urea | potassium |
| | Calcium | Protein (total) |
| | Chloride | Sodium |
| | CPK (including isoenzymes if > ULN) | Uric acid |
| Cardiac | Troponin T or I | NT-pro-BNP |
| Urinalysis (dipstick) | Glucose | pH |
| | Ketones | Protein |
| | Leukocytes | Specific gravity |
| | Nitrites | Urobilinogen |
| | Occult blood | |
| Urinalysis | Bacteria | Epithelial cells |
| (microscopic) | Casts | Mucous threads |
| | Crystals | RBC, WBC |
| Adrenal function | Cortisol | |
| Thyroid function | Free T4 | TSH |
| Viral serology (if | Anti-HIV-1 antibody | HCV-RNA quantitation (if |
| applicable)⁴ | Anti-HIV-2 antibody | applicable) |
| | HBsAg, anti-HBsAg antibody, anti- | HBV-RNA quantitation (if |
| | HBcAg-antibody | applicable) |
| | | anti-HCV antibody |

Central

| | | |
|---|---|---|
| Chemistry | CRP | |
| Cardiac | hs-Troponin T | NT-proBNP |
| Markers of | Cytokines (serum) | Complement |
| Inflammation³ | Histamine | |
| Other | ADA/Nab PK | |

ALP = alkaline phosphatase;
ALT = alanine aminotransferase;
aPTT = activated partial thromboplastin time;
AST = aspartate aminotransferase;
β-HCG = beta human chorionic gonadotropin;
BUN = blood urea nitrogen;
CPK = creatine phosphokinase;
CRP = C-reactive protein;
eCrCl = estimated creatinine clearance;
freeT4 = free thyroxin;
GGT = gamma glutamyl transferase;
HbA1c = glycated hemoglobin;
Ig = immunoglobulin;
HBcAg = hepatitis B core antigen;
HBsAg = hepatitis B surface antigen;
HBV = hepatitis B virus;
HCV = hepatitis C virus;
HIV = human immunodeficiency virus;
INR = international normalized ratio;
LDH = lactate dehydrogenase;
NTpro-BNP = N-terminal prohormone brain natriuretic peptide;
PT = prothrombin time;
RBC = red blood cell;
SGOT = serum glutamic-oxaloacetic transaminase;
SGPT = serum glutamic-pyruvic transaminase;
TSH = thyroid-stimulating hormone;
ULN = upper limit of normal;
WBC = white blood cell.
1RBC indices (mean corpuscular volume [MCV], mean corpuscular hemoglobin [MCH], mean corpuscular hemoglobin concentration [MCHC]) and distribution widths (red cell)
²WBC with differential (including neutrophils, basophils, eosinophils, lymphocytes, monocytes) reported as percentage or absolute values.
³See TBD for analyte specific guidance.
⁴The serology for individual virus(es) may be omitted at the Investigator's discretion if seronegativity has beenpreviously documented and there are no signs of the corresponding viral infection.

TABLE 18
118421 Dose Modification Guidance and Specific Management of Toxicities

| | | |
|---|---|---|
| Toxicity | Specific Management | 118421 Dose Modification ^{a} |

Neutropenia or Thrombocytopenia

| | | | |
|---|---|---|---|
| ANC | >500/μl or < 500/μl for < | According to local standard of | No change |
| AND | 5 days | care. Colony stimulating factors are | |
| Platelet | ≥25,000/μl | allowed. Steroids should not be used to | |
| ANC | <500/μl for ≥ 5 days OR | manage cytopenia. | Decrease by 1 dose level |
| AND/OR | febrile neutropenia^{b} | | |
| Platelet | <25,000/μl for ≥ 7 days | | |
| | with or without active | | |
| | bleeding, OR < 50,000/μl | | |
| | for ≥ 7 days with clinically | | |
| | significant bleeding ^{c} | | |

Non-hematological TEAEs

| Grade ≥ 3 Immune-mediated toxicities | | Infusion must be stopped | Grade ≥ 3 immune mediated toxicities (once |
|---|---|---|---|
| (pneumonitis, colitis, endocrinopathies, nephritis, | | immediately. Steroids may be used to | confirmed) will result in the discontinuation of the |
| hepatitis and skin reactions) | | manage the acute symptoms of the | patient from the study. Other immune-mediated |
| | | event, and standard of care supportive | toxicities must improve to grade 1 or resolve before |
| | | measures should be given. | resuming treatment. |
| IRR | Grade ≥ 2 | Infusion must be interrupted. Any | For grade 2 IRR, treatment may be restarted or |
| OR | | repeat Grade 2 immune related event | permanently discontinued at the Investigator's |
| Hypersensitivity | | must result in the discontinuation of the | discretion. |
| reaction | | patient from the study. | If 118421 re-treatment after Grade 2 IRR is allowed, |
| | | | then it must occur: |
| | | | Only after the appropriate delay (ie, |
| | | | resolution of symptoms). |
| | | | At the reduced dose and/or reduced infusion |
| | | | rate, where both will be chosen at the |
| | | | Investigator's discretion. |
| | | | After the appropriate anti-allergic prophylaxis |
| | | | according to the premedication guidance in |
| | | | this protocol (see Section 8.2) or the |
| | | | institutional guideline. |
| | Grade ≥ 3 | | 118421 treatment should be permanently |
| | | | discontinued |
| Grade ≥ 2 CRS | | Subjects may be hospitalized and | 118421 treatment is discontinued or delayed until the |
| | | closely monitored for signs of CLS and | subject's full recovery. The treatment may be |
| | | CRS. This includes: | restarted at the same dose or at a reduced dose only |
| | | Monitoring of vital signs | after discussion with and agreement of the Medical |
| | | (temperature, heart rate, BP, | Monitor. |
| | | and respiration rate), body | |
| | | weight and clinical symptoms | |
| | | including headache, myalgia, | |
| | | muscle weakness, edema, | |
| | | neurological and gastro- | |
| | | intestinal symptoms, | |
| | | abdominal pain, and fatigue | |
| Grade ≥ 2 CLS | | Monitoring of laboratory | |
| | | parameters of hematology, | |
| | | albumin, kidney and liver | |
| | | function and cytokines in case | |
| | | of clinical symptoms indicative | |
| | | of CRS/CLS | |
| | | Investigators should consider | |
| | | supportive measures while on trial | |
| | | regardless of dosing or timing of | |
| | | dosing. If a subject experiences | |
| | | Grade 2 hypotension, orthostasis, | |
| | | edema, or hypoalbuminemia, normal | |
| | | saline and/or an albumin infusion may | |
| | | be given, as needed. | |

| | | |
|---|---|---|
| Decrease in albumin by ≥ 1 g/dL from baseline or | Fluid overload and CHF should be | May trigger a modification of 118421 treatment (dose |
| to < 2 g/dL, whichever comes first | ruled out. | interruption, dose delay, dose reduction or |
| | | permanent discontinuation) |
| SCr increase ≥ 1.5-3.0 times above baseline or 1.5- | Immune related nephritis should be | May trigger a modification of 118421 treatment (dose |
| 3.0 × ULN in the absence of dehydration or | ruled out, and other causes managed | interruption, dose delay, dose reduction or |
| bleeding | as per standard of care. Repeat | permanent discontinuation) |
| Any new Grade ≥ 3 electrolyte abnormality that does | immune mediated Grade > 3 nephritis | |
| not resolve, with or without intervention, to Grade < 2 | requires discontinuation of the patient | |
| within 72 hours | from the study. | |
| Any other Grade ≥ 3 non-hematological toxicity | Manage according to local standard of | May trigger a modification of 118421 treatment (dose |
| excluding the following: | care | interruption, dose delay, dose reduction or |
| Nausea, vomiting, or diarrhea, if | | permanent discontinuation) |
| manageable with antiemetic or antidiarrheal | | |
| agents within 7 days of onset | | |
| Fatigue lasting ≥ 72 hours | | |
| Laboratory abnormalities, even if | | |
| asymptomatic and without a clear clinical | | |
| correlate | | |
| Grade 3 or repeat Grade 2 immune mediated events | Manage as per immune mediated | Discontinue study drug(s) |
| including pancreatitis, myositis, pneumonitis, | events table | |
| myocarditis, hepatic toxicity | | |

Cardiac toxicity

| | | |
|---|---|---|
| hs-troponin elevation (above the ULN or more than | Provide medical support as clinically | Interrupt 118421 infusion or withhold planned dose |
| 10% increase compared to screening if the baseline | indicated. | while assessing event. |
| level was higher than the ULN) with no additional | Serial labs including protocol-specified | 118421 may be permanently discontinued or |
| clinical cardiac symptoms | hs-troponin, CPK, CPK-MB, CBC, | Irestarted, at the same or at a lower dose level |
| | chemistry panel, and cytokines should | |
| | be collected. Echocardiogram and/or | |
| | cardiac MRI, as indicated | |
| hs-troponin elevation (above the ULN or more than | Provide medical support as clinically | Discontinue study drug(s) |
| 10% increase compared to screening if the baseline | indicated. | after consultation with the MTEM Medical Monitor if |
| level was higher than ULN) with clinically significant | Serial labs including protocol-specified | causality is believed to be related to 118421. |
| EKG changes and/or clinical cardiac symptoms | hs-troponin, CPK, CPK-MB, CBC, | |
| Appearance of clinically significant new ECG | chemistry panel, and cytokines should | |
| abnormality (indicating ischemia or significant | be collected. | |
| conduction disorder or hemodynamically significant | Immune mediated myositis, | |
| arrhythmia) | myocarditis and thromboembolic | |
| Cardiac symptoms (e.g., chest pain, shortness of | events should be ruled out. | |
| breath, palpitation) | Depending on the abnormality and the | |
| Decrease of LVEF by at least 10% (and to 50% or | severity of symptoms, schedule | |
| below) or patient is symptomatic | cardiology consultation. | |
| Any other Grade ≥ 2 cardiac toxicity | Echocardiogram and/or cardiac MRI, | |
| | as indicated | |

Hepatotoxicity-Immune mediated toxicity and HY's Law should be ruled out

| | | |
|---|---|---|
| AST and/or ALT | ≤5 × ULN (isolated) | No change in treatment |
| OR | ≤1.5 × ULN (isolated) | Monitor subject |
| Bilirubin | | |
| AST and/or ALT | >5 × ULN-8 × ULN | Temporarily or permanently (see next row) |
| OR | (for subjects enrolled | discontinue treatment |
| Bilirubin | with AST/ALT < 3 × | Monitor subject |
| | ULN) | Continue at dose reduced by 1 dose level |
| | >1.5 × ULN-3 × ULN | after abnormal values resolve to Grade ≤ 1 or |
| | (in a patient with | baseline values |
| | Gilbert's syndrome, | |
| | direct bilirubin | |
| | elevations should be | |
| | evaluated) | |
| AST or ALT | >8 × ULN for any | Permanently discontinue treatment |
| | period of time (for any | |
| | subject) | |
| | >5 × ULN for more | |
| | than 2 weeks (for | |
| | subjects enrolled with | |
| | AST/ALT 3-5 × | |
| | ULN) | |
| AST and/or ALT | >3 × ULN (without | |
| AND | findings of cholestasis | |
| Bilirubin | defined as serum ALP | |
| | <2 × ULN) | |
| | >2 × ULN or INR > | |
| | 1.5 | |
| AST and/or ALT | >3 × ULN with the | Permanently discontinue treatment |
| OR | appearance of fatigue, | |
| Bilirubin | nausea, vomiting, | |
| | right upper quadrant | |
| | pain or tenderness, | |
| | fever, rash, and/or | |
| | eosinophilia (>5%) | |
| | >3 × ULN | |

The dose may be increased back to the subject's original starting dose after previous 118421 dose reduction. This decision needs agreement between the Investigator and the Medical Monitor

A. Pharmacokinetic Assessments

Blood samples will be collected prior to, during, and at specified times following 118421 infusion for determination of free 118421 drug concentrations in serum, using a validated assay method. Serum samples may be banked. Serum concentration time data from all eligible subjects will be subjected to noncompartmental assessment using the software package Phoenix WinNonlin (Certara, Princeton N.J.). Parameters will be stratified by dose group and summary statistics will be generated.

The following PK parameters will be evaluated in plasma after IV administration, if calculable: Cmax, tmax, AUCO-t, AUCO-∞, AUC from time zero to the end of the dosing interval (AUCtau), CL, Vss, volume of distribution during terminal phase (Vz), and accumulation ratios compared to Day 1 dosing (R). Dose-proportionality will be assessed based on dose-normalized Cmax and AUC values.

Serum samples collected for PK assessments can be stored and utilized for additional assay development or other future biological research.

Urine samples will be collected at specified times following 118421 infusion for urinalysis and pregnancy testing (refer to Table 14 above for specific time points).

B. Pharmacodynamic Assessments

Prior to and after treatment, peripheral blood will be collected at various time points to determine the effect of dosing on different immune cell subsets. In addition, circulating biomarkers, CRP, and hs troponin levels will be monitored during treatment.

Subjects must have an easily biopsiable lesion (biopsy sites of non-significant risk, in the opinion of the Investigator) and willingness to undergo biopsy.

Core tumor tissue biopsies at baseline and after 6-8 weeks on study are mandatory and at the time of disease progression (optional) and the tumor microenvironment of the samples will be evaluated to assess pharmacodynamic indicators of response and/or potential predictive indicators and biomarkers.

C. Immunogenicity Assessments

Antibodies to 118421 will be evaluated in serum samples collected from all subjects according to the schedule in Table 14.

The detection and characterization of antibodies to 118421 will be performed using a validated assay method. ADA positive samples will have the titer reported and screened for NAb. Impact of ADAs on 118421 serum concentration will also be evaluated.

### V. Assessment of Safety:

A. Safety Parameters

Planned time points for all safety assessments are provided in Table 14.

When the PK, ECG, and vital signs assessments are scheduled to occur at the same time point, the following order of assessments applies: (1) ECG, (2) vital signs, (3) PK sample collection.

The Investigator or delegate at the site evaluates the safety assessment results and categorizes them as “normal”, “abnormal, not clinically significant” (NCS) or “abnormal, clinically significant” (CS). Any clinically significant safety assessment results should be reported as AE.

Unscheduled safety assessment can be performed any time.

Information about date of birth/age, sex, race/ethnicity, detailed smoking history and alcohol history will be recorded during screening.

Medical history will be recorded at screening. This will include prior surgery, prior and concomitant illnesses, allergy history, prior antitumor chemotherapy, radiotherapy and tumor related biopsies or surgery.

Vital sign assessments (BP [systolic and diastolic], respiratory rate (RR), heart rate, body temperature) will be assessed at time points indicated in Table 14. Body temperature will be measured in Fahrenheit (° F.) or Celsius (° C.). The same body position and method of measurement of vital signs should be used whenever possible throughout a subject's participation in this study. The subject must rest in a sitting or semi-recumbent position for at least 5 minutes before and during the assessment.

Height (in meters [m]) and weight (in kilograms [kg]) will be measured; body-mass index (BMI) will then be calculated. Body weight measured before the start of treatment on Cycle 1 Day 1 (C1D1) will be used to calculate the 118421 dose in all subsequent cycles. The dose must be re-calculated when the body weight has changed by 10% from the baseline value; or according to institutional policies should they require adjustment for any change in body weight. If a subject's weight changes by 10% and a new dose is administered, this weight is considered the new baseline to assess for changes in weight for subsequent cycles.

At a minimum, the following aspects/body parts should be assessed during the complete physical examination: (a) general appearance; (b) skin (paleness, jaundice, redness/rash, acneiform changes); (c) extremities (petechial bleedings, ulcers, signs of thrombosis), hands and feet (signs of handfoot syndrome/palmar-plantar erythrodysesthesia); (d) ears, eyes (jaundice, inflammation), nose and throat (presence of petechial bleedings, gingival bleeding); (e) head and neck; (f) lungs; (g) heart; (h) abdomen (pain, tenderness, peristaltic, ascites, organomegaly); (i) lymph nodes; and (j) neurological examination. Other aspects/body parts or organ systems may be assessed at the Investigator's discretion.

At a minimum, the following aspects/body parts should be assessed during the abbreviated physical examination: (a) general appearance; (b) skin (paleness, jaundice, redness/rash, acneiform changes); (c) ears, eyes (jaundice, inflammation), nose and throat (presence of petechial bleedings, gingival bleeding); (d) lungs; (e) heart; (f) abdomen (pain, tenderness, peristaltic, ascites, organomegaly); (g) lymph nodes; and (h) abbreviated neurological examination. Other aspects/body parts or organ systems may be assessed at the Investigator's discretion.

ECG recordings will be obtained using standardized equipment supplied for this study. Standard resting 12-lead ECG assessments will be performed after the subject has rested for at least 5 minutes in supine or semi-recumbent position. Triplicate 12-lead ECG should be obtained as 3 standard ECGs recorded in close succession according to the operating manual provided by the central ECG vendor. If any of the ECG printouts in a triplicate is of poor quality, additional ECG(s) should be obtained as soon as possible at the same time point until 3 ECGs of adequate quality are obtained. The QT interval correction for heart rate using Fridericia's formula will be used for safety assessment. The 3 QTcF values from a triplicate ECG should be averaged to yield the mean QTcF value. In subjects with right bundle branch blocks, additional corrections will be performed to calculate the QT equivalent JT. Subjects with a new left bundle branch block should have treatment withheld and discussed with the Medical Monitor.

Refer to Table 14 for a list of laboratory assessments to be performed as well as timing and frequency. In the event of an unexplained clinically significant abnormal laboratory test value, the test should be repeated immediately and followed up until it has returned to the normal range and/or an adequate explanation of the abnormality is found. Laboratory test results will be graded according to the CTCAE v 5.0 criteria. In general, a confirmed Grade 3 or Grade 4 abnormal laboratory test result is considered to be an adverse event. All screening safety laboratory tests (hematology, coagulation, chemistry, hs-troponin, and urinalysis) must be collected within 14 days of start of treatment.

Creatine clearance will be derived from the serum creatine values obtained from the blood chemistry assessment. The eCrCl will be calculated using the Cockcroft-Gault formula:

eCrCl={((140−age)×weight)/(72×SCr)}×0.85 (if female)
where eCrCl=mL/minute; SCr=serum creatinine (mg/dL); age=years; and weight=kg.

In addition to the time points indicated in Table 14, if a subject experienced a Grade ≥2 IRR, or another Grade ≥2 hypersensitivity event, Grade ≥2 CRS or Grade ≥2 CLS, the Investigator should obtain serum samples for complement, histamine, and cytokines as soon as possible after the TEAE onset.

B. Adverse and Serious Adverse Events

An AE (referred to as an adverse event or adverse experience) includes any unfavorable sign (e.g., an abnormal laboratory finding), symptom, or clinical outcome temporally associated with the use of a test drug, active control, or placebo, regardless of whether the event is thought to be related to the drug.

An AE is any symptom, physical sign, syndrome, or disease that either emerges during the study or, if present at screening, worsens during the study, regardless of the suspected cause of the event. All AEs that occur in enrolled subjects during the AE reporting period specified in the protocol must be recorded, regardless of the relationship of the AE to study drug.

A serious adverse event (SAE) is an AE occurring during the reporting period that meets one or more of the following criteria: (a) results in death; (b) is immediately life-threatening; (c) requires in-patient hospitalization or prolongation of existing hospitalization; (d) results in persistent or significant disability or incapacity; (e) results in a congenital abnormality or birth defect; (f) is an important medical event that may jeopardize the patient or may require medical intervention to prevent one of the outcomes listed above.

AEs and SAEs will be collected from the first administration of study drug at Cycle 1 Day 2 through STFU visit 1 or until the start of new cancer therapy (unless the AE is related to 118421), whichever occurs first. Adverse events spontaneously reported by the patient/subject and/or in response to an open question from the study personnel or revealed by observation will be recorded in the clinical database. Laboratory, vital signs, and ECG abnormalities should also be recorded as AEs when considered clinically significant and representing a change from pretreatment baseline. Abnormal values resulting in discontinuation of study drug must be reported and recorded as an AE.

The AE term should be reported in standard medical terminology when possible. For each AE, the investigator will evaluate and report the onset (date and time), resolution (date and time), intensity, causality, action taken, serious outcome (if applicable), and whether or not it caused the patient to discontinue the study.

Intensity will be assessed according to the following scale when the AE cannot be identified by the Common Toxicity Criteria guidelines from the National Cancer Institute v5.0: (1) mild (awareness of sign or symptom, but easily tolerated); (2) moderate (discomfort sufficient to cause interference with normal activities); (3) severe (incapacitating, with inability to perform normal activities); (4) life threatening or disabling (puts subject life at risk); (5) fatal.

If the AE is not listed in the CTCAE v5.0, then the highest severity level reached according to the scale in Table 19 will be assigned. Every effort should be made to find the appropriate AE term and definitions of severity in the modified CTCAE v. 5.0.

TABLE 19
Classification of Adverse Events

| Grade | Definition |
|---|---|
| Grade 1 | An AE that is easily tolerated by the subject. It incurs only a minimum |
| (mild) | of discomfort and does not influence ordinary daily tasks. |
| Grade 2 | An AE that is of sufficient severity to have a negative |
| (moderate) | influence on ordinary daily tasks. |
| Grade 3 | An AE that effectively hinders ordinary |
| (Severe) | daily tasks, often requiring intervention. |
| Grade 4 | An AE that puts the subject's life at risk. |
| (life threatening or | |
| disabling) | |
| Grade 5 | Death related to an AE. |
| (fatal) | |

AE = adverse event.

Causality should also be assessed for 118421. The following should be considered when assessing causality: (1) temporal associations between the agent and the event; (2) effect of de-challenge and/or re-challenge; (3) pre-existing risk factors; (4) a plausible mechanism; and (5) concurrent illnesses. Causality will be classified as shown in Table 20 below.

TABLE 20
Classification of Adverse Events by Causality/Relationship to 118421

| Causality/Relatedness | Definition |
|---|---|
| Definitely related | Follows a reasonable temporal sequence from drug administration, abates upon |
| | discontinuation of the drug (de-challenge), is confirmed by reappearance of the |
| | reaction on repeat exposure (re-challenge). |
| Probably related | Follows a reasonable temporal sequence from drug administration, abates upon |
| | discontinuation of the drug, cannot be reasonably explained by the known |
| | characteristics of the subject's clinical state. |
| Possibly related | Follows a reasonable temporal sequence from drug administration, could have |
| | been produced by the subject's clinical state or by other modes of therapy |
| | administered to the subject. |
| Unlikely to be related | Does not follow a reasonable temporal sequence from drug administration, is |
| | readily explained by the subject's clinical state or by other modes of therapy |
| | administered to the subject. |
| Unrelated | The AE is definitely produced by the subject's clinical state or by other modes |
| | of therapy administered to the subject |

AE = adverse event.

An unexpected SAE that is at least possibly related to the study drug will be considered a suspected unexpected serious adverse reaction (SUSAR).

Any action with the study treatment during the AE management and follow-up should be documented using the following categories: (1) drug withdrawn; (2) drug interrupted; (3) infusion interrupted and resumed; (4) infusion stopped; (5) dose reduced; (6) dose not changed; (7) not applicable; or (8) unknown. The action “dose increased” is not available in this study.

The outcome of the AE should be documented as follows: (1) recovered/resolved; (2) recovered/resolved with sequelae; (3) not recovered/not resolved; (4) fatal; (5) unknown

### VI. Statistics

Summary statistics for continuous variables will include the mean, standard deviation, median, and range (minimum/maximum); categorical variables will be presented as frequency counts and percentages; and time-to-event variables will be summarized by Kaplan-Meier plots, medians and range. Where confidence limits are appropriate, the confidence level will be 95% (2-sided), unless otherwise stated.

A. Statistical Designs: Part A

Using the mTPI-2 method, with a target toxicity rate for the MTD of 33% and the equivalence interval of 28%-38%, approximately 24 DLT evaluable subjects are anticipated to enroll in Part A dose escalation. Subjects will enroll in cohorts of size 3-6 (DLT evaluable) and approximately 6 dose levels are expected. For the purposes of overdose control, doses j and higher levels will be eliminated from further examination if the probability of the event that, given data, the posterior probability of target toxicity rate for the MTD being greater than 0.33 is greater than 0.95, i.e., Pr(Pj >0.33 I data)>0.95 and at least 3 subjects have been treated at dose level j, where Pj is the true posterior DLT rate of dose level j, j=1, —, 6. The probability cutoff 0.95 is chosen to be consistent with the common practice that when the target DLT rate 1/6, a dose with 2/3 subjects having experienced a DLT is eliminated.

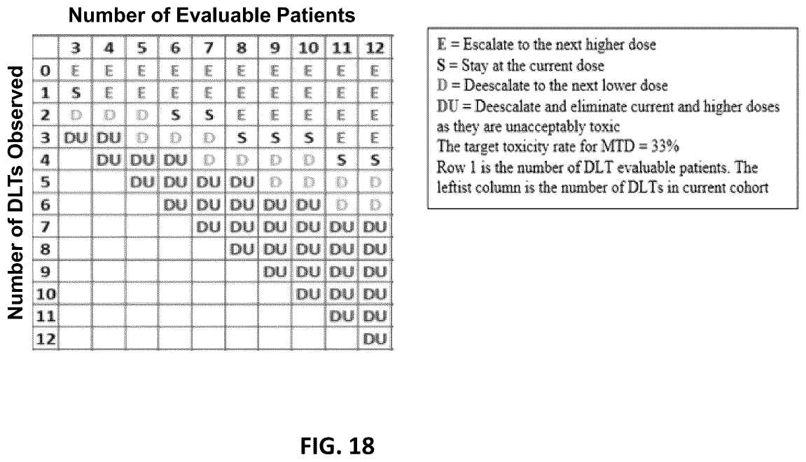

The dose escalation/de-escalation and elimination rule for Part A is displayed graphically in FIG. 18. Based on this display, the steps to implement the mTPI-2 design are as follows:
1. Subjects in the first cohort are treated at dose level 1; at least 3 subjects receive study treatment and become DLT evaluable before escalating to the next dose escalation.
2. If the first expansion dose in the combination cohort is deemed intolerable, the dosing level for the next, second, cohort will use 50% of the first dose cohort of 118421.
3. To assign a dose to the next cohort of subjects, dose escalation/de-escalation is conducted according to the algorithm displayed in FIG. 18. Note that: (a) “de-escalate and eliminate” means eliminate the current and higher doses from the trial to prevent treating any future patients at these doses because they are overly toxic; (b) if a dose is eliminated, automatically de-escalate the dose to the next lower level. When the lowest dose is eliminated, stop the trial for safety. In this case, no dose can be selected as the MTD; (c) if none of the preceding actions (i.e., escalation, de-escalation or elimination) is triggered, continue to treat the new patients at the current dose; (d) if the current dose is the highest dose and the rule indicates dose escalation, treat the new patients at the highest dose.
4. Repeat lines a to d in step 3 until the MTD is reached or until the number of patients treated at the current dose reaches 12, and the decision, according to FIG. 18, is to stay at the current dose. In the latter case, the current dose can be deemed to be MTD per totality of data.

B. Statistical Designs: Part B Expansion Cohorts

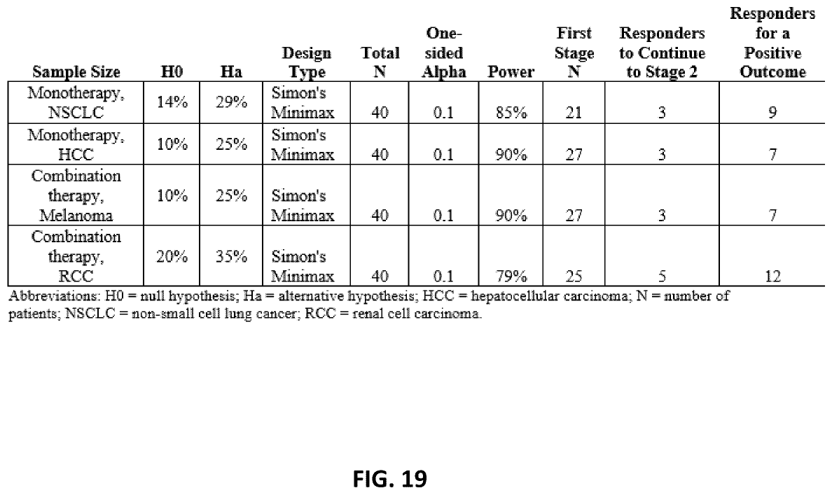

The sample size in Part B will be 40 for each expansion group based upon Simon's Two-Stage minimax design: 1) a null hypothesis based on the historical ORR of SoC for the specific tumor type versus an alternative hypothesis of a 15% greater ORR than historical SoC (the historical SoC ORR is estimated based on ORR with SoC in the same tumor type); 2) with one-sided type I error rate of 0.10; and 3) with power ranging from 79% to 90%. The statistical design for Part B is shown in FIG. 19.

C. Efficacy Analysis

The primary efficacy variable in Part B is the ORR per RECIST 1.1 (CR and PR confirmed within 4-6 weeks of the initial response). Other efficacy variables are Disease Control Rate (DCR), Duration of Response (DOR), Progression Free Survival (PFS); and Time to Response (TTR). All efficacy analyses will be performed based on the FAS population.

The study will include a Long Term Follow-Up contact every 3 months (±30 days) after the STFU Visit 2 for up to 24 months. Subjects who discontinue the study treatment for reasons other than disease progression will be followed for PFS.

DCR will be defined as proportion of subjects who have achieved CR, PR, and SD (SD defined as SD for 24 weeks or longer).

The DOR is defined for patients with confirmed CR or PR as the time from the first documented tumor response (CR or PR) to the date of radiographic PD per RECIST 1.1. Subjects who have not radiographically progressed or who have only clinical progression at the time of database lock will be censored at the date of their last tumor assessment. The DOR will be summarized descriptively using the Kaplan-Meier methods (KM median and corresponding 95% CI quartiles, number of events, number censored, Kaplan Meier figure).

The PFS is defined as the time from the start of treatment with 118421 on C1D1 to the date of radiographic PD per RECIST 1.1 or death from any cause, not including clinical progression. Subjects who have not radiographically progressed at the time of data cutoff or data base lock will be censored at the date of their last tumor assessment (see the censoring rules in the SAP). For PFS, those subjects who have no data on radiographic progression (e.g., subjects who discontinued the study due to reasons other than radiographic progression and who were not followed up until radiographic progression) and have no data on tumor assessment after baseline and are still alive will be censored at the subject's date of first dose.

TTR is defined as the time from the date of the first dose of the study treatment to the date of the first documentation of response (PR or better) per RECIST 1.1. Data listings will be created to support each analysis and to present all efficacy data.

### Example 12: Cytotoxicity of CTLA-4 ETB 118421 in the Presence of Ipilimumab

The objective of this study was to determine the cytotoxicity of the CTLA-4 ETB 118421 in the presence of the anti-CTLA-4 monoclonal antibody ipilimumab.

The direct cell kill mechanism of action exhibited by ETBs is dependent on selective targeting of antigen expressing cells via the binding domain of the ETB. 118421 is designed to target human CTLA-4 to elicit targeted cytotoxicity on immune suppressive regulatory T cells. 118421 is a biparatopic ETB containing two VHH targeting domains that bind to separate epitopes on the CTLA-4 antigen. One VHH domain interacts and binds with the same epitope of ipilimumab, and the second VHH domain interacts with an epitope separate from ipilimumab.

Ipilimumab is indicated for unresectable or metastatic melanoma at a dosage of 3 mg/kg. Clinical studies report that the Cmax of a 3 mg/kg dose of ipilimumab to range from 60 to 85 μg/mL with an estimated average half-life of 15.4 days. For this study, 80 μg/mL was used as an estimated representative Cmax for ipilimumab at 3 mg/kg and concentrations of ipilumumab estimated to be representative of 2 to 7 half-lives (30.8-107.8 days) post-dose were utilized in this study.

TABLE 21
Concentrations of Ipilimumab Used in Study

| Number of Half | Days | Estimated Serum |
|---|---|---|
| Lives Post- | Post-Dose | Concentration of |
| Dose (3 μg/kg | (3 μg/kg | Ipilimumab |
| ipilimumab) | ipilimumab) | (μg/mL) |
| 0 | 0 | 80 |
| 1 | 15.4 | 40 |
| 2 | 30.8 | 20 |
| 3 | 46.2 | 10 |
| 4 | 61.6 | 5 |
| 5 | 77 | 2.5 |
| 6 | 92.4 | 1.25 |
| 7 | 107.8 | 0.625 |

The cytotoxic activity of 118421 in the presence and absence of ipilimumab was evaluated using an in vitro CellTiter-Glo® 2.0 Assay. Briefly, CHO-K1 expression cell lines were grown and maintained in Hams F-12K media supplemented with 10% FBS and 100 U/100 μg Pen/Strep per mL. CTLA-4 expressing cells were plated into 384 well microplates at a concentration of 500 cells/well. A dilution series of ipilimumab was prepared in sterile DI-H20 then added in triplicate to plated cells and incubated at 37° C./5% CO₂ for 30 minutes. Concentration ranged from 135 nM to 4 nM. A dilution series of 118421 was prepared in PBS then added in triplicate to cells pre-treated with ipilimumab for a final concentration range of 100.0-0.0006 nM (12, 3-fold dilutions). CTLA-4 expressing cells treated with 118421 were incubated for 4 days at 37° C. in a tissue culture incubator supplemented with 5% CO₂. On Day 4, 50 μL of CellTiter-Glo® 2.0 was added and incubated for ˜2 minutes with gentle agitation followed by a 10-minute incubation at room temperature in the dark. Luminescence (RLU) was measured on Spectramax iD3 multimode plate reader at 100 ms and exported to Excel for data analysis.

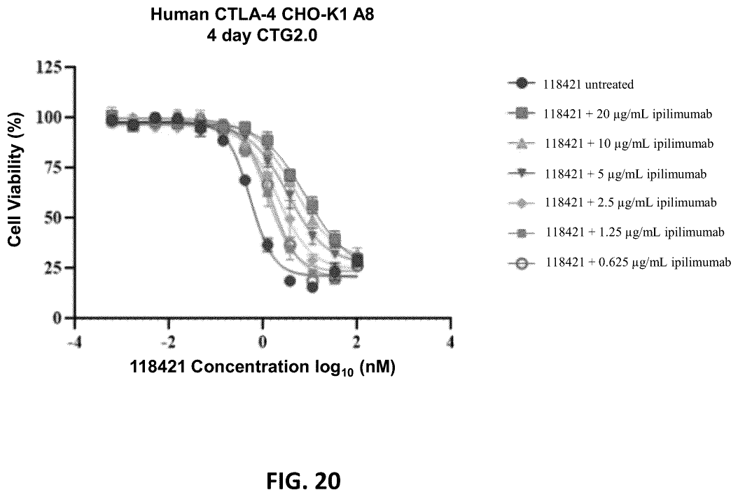

The results of the cytotoxicity assay are shown in FIG. 20 and Table 22 below. 118421 demonstrated targeted cell kill in the absence of Ipilimumab with an IC50 value of 0.55 nM. In the presence of ipilimumab, 118421 retained cytotoxicity but potency was decreased with IC50 values ranging from 8.51 nM at 20 μg/ml Ipilimumab down to 1.56 nM at 0.625 μg/ml ipilimumab.

TABLE 22

| Treatment Group | IC50 (nM) | R² |
|---|---|---|
| 118421 untreated | 0.531 | 0.98 |
| 118421 + 20 μg/mL ipilimumab | 8.21 | 0.971 |
| 118421 + 10 μg/mL ipilimumab | 4.61 | 0.978 |
| 118421 + 5 μg/mL ipilimumab | 3.29 | 0.978 |
| 118421 + 2.5 μg/mL ipilimumab | 2.01 | 0.963 |
| 118421 + 1.25 μg/mL ipilimumab | 1.19 | 0.973 |
| 118421 + 0.625 μg/mL ipilimumab | 1.49 | 0.984 |

Data is averaged from 3 biological replicate experiments

Overall, the CTLA-4 ETB 118421 displays targeted cytotoxicity against human CTLA-4 expressing cells. Toxicity of 118421 is retained but with a decrease in potency in the presence of Ipilimumab in a concentration-dependent manner. This data supports the ability of 118421 to retain targeted cytotoxicity of cells expressing human CTLA-4 in the presence of Ipilimumab.

In some embodiments of the invention have been described by way of illustration, it will be apparent that the invention can be put into practice with many modifications, variations and adaptations, and with the use of numerous equivalents or alternative solutions that are within the scope of persons skilled in the art, without departing from the spirit of the invention or exceeding the scope of the claims.

### Numbered Embodiments of the Invention

Notwithstanding the appended claims, the disclosure sets forth the following numbered embodiments:

### Section 1:

Embodiment 1. A CTLA-4 binding molecule comprising a Shiga toxin A subunit effector polypeptide and a binding region capable of specifically binding an extracellular part of CTLA-4; wherein the binding region comprises a VHH domain comprising a HCDR1, a HCDR2, and a HCDR3.

Embodiment 2. The CTLA-4 binding molecule of embodiment 1, wherein
(a) the HCDR1 comprises the amino acid sequence of SEQ ID NO: 23, the HCDR2 comprises the amino acid sequence of SEQ ID NO: 24, and first HCDR3 comprises the amino acid sequence of SEQ ID NO: 25; or
(b) the HCDR1 comprises the amino acid sequence of SEQ ID NO: 26, the HCDR2 comprises the amino acid sequence of SEQ ID NO: 27, and the HCDR3 comprises the amino acid sequence of SEQ ID NO: 28.

Embodiment 3. The CTLA-4 binding molecule of embodiment 1 or 2, wherein the VHH domain comprises the amino acid sequence of SEQ ID NO: 21 or SEQ ID NO: 22.

Embodiment 4. A CTLA-4 binding molecule comprising a Shiga toxin A subunit effector polypeptide and a binding region capable of specifically binding an extracellular part of CTLA-4; wherein the binding region comprises a first VHH domain comprising a first HCDR1, a first HCDR2, and a first HCDR3 and a second VHH domain comprising a second HCDR1, a second HCDR2, and a second HCDR3.

Embodiment 5. The CTLA-4 binding molecule of embodiment 4, comprising a linker that links the first VHH domain and the second VHH domain.

Embodiment 6. The CTLA-4 binding molecule of embodiment 4 or 5, wherein the first HCDR1 comprises the amino acid sequence of SEQ ID NO: 23, the first HCDR2 comprises the amino acid sequence of SEQ ID NO: 24, and the first HCDR3 comprises the amino acid sequence of SEQ ID NO: 25.

Embodiment 7. The CTLA-4 binding molecule of any one of embodiments 4-6, wherein the first VHH domain comprises the amino acid sequence of SEQ ID NO: 21.

Embodiment 8. The CTLA-4 binding molecule of any one of embodiments 4-7, wherein the second HCDR1 comprises the amino acid sequence of SEQ ID NO: 26, the second HCDR2 comprises the amino acid sequence of SEQ ID NO: 27, and the second HCDR3 comprises the amino acid sequence of SEQ ID NO: 28.

Embodiment 9. The CTLA-4 binding molecule of any one of embodiments 4-8, wherein the second VHH domain comprises the amino acid sequence of SEQ ID NO: 22.

Embodiment 10. The CTLA-4 binding molecule of any one of embodiments 5-9, wherein the linker comprises the amino acid sequence of SEQ ID NO: 29.

Embodiment 11. The CTLA-4 binding molecule of any one of embodiments 1-10, wherein the Shiga toxin A subunit effector polypeptide comprises a polypeptide having the sequence of:
(i) amino acids 1 to 251 of any one of SEQ ID NOs: 1-18; or
(ii) amino acids 1 to 261 of any one of SEQ ID NOs: 1-18;
or a polypeptide having a sequence that is at least 90% or at least 95% identical thereto.

Embodiment 12. The CTLA-4 binding molecule of any one of embodiments 1-10, wherein the Shiga toxin A subunit effector polypeptide comprises or consists of a polypeptide having the sequence of any one of SEQ ID NO: 40 to 68.

Embodiment 13. The CTLA-4 binding molecule of any one of embodiments 1-10, wherein the Shiga toxin A subunit effector polypeptide comprises the amino acid sequence of SEQ ID NO: 41.

Embodiment 14. The CTLA-4 binding molecule of any one of embodiments 1-13, wherein the CTLA-4 binding molecule comprises a linker that links the Shiga toxin A subunit effector polypeptide and the binding region.

Embodiment 15. The CTLA-4 binding molecule of embodiment 14, wherein the linker that links the Shiga toxin A subunit effector polypeptide and the binding region comprises the amino acid sequence of SEQ ID NO: 218.

Embodiment 16. The CTLA-4 binding molecule of embodiment 14 or 15, wherein the binding molecule comprises, from N-terminus to C-terminus or from C-terminus to N-terminus, the Shiga toxin A subunit effector polypeptide, the linker, and the binding region.

Embodiment 17. The CTLA-4 binding molecule of embodiment 14 or 15, wherein the binding molecule comprises, from N-terminus to C-terminus, the Shiga toxin A subunit effector polypeptide, the binding region linker, the first VHH domain, the linker, and the second VHH domain.

Embodiment 18. The CTLA-4 binding molecule of embodiment 17, wherein the binding molecule comprises the amino acid sequence of SEQ ID NO: 329.

Embodiment 19. The CTLA-4 binding molecule of any one of embodiments 1-18, wherein the binding molecule is a single continuous polypeptide.

Embodiment 20. The CTLA-4 binding molecule of any one of embodiments 1-18, wherein the CTLA-4 binding molecule comprises two polypeptides.

Embodiment 21. The CTLA-4 binding molecule of embodiment 20, wherein the polypeptides are non-covalently linked.

Embodiment 22. The CTLA-4 binding molecule of embodiment 20, wherein the polypeptides are covalently linked.

Embodiment 23. The CTLA-4 binding molecule of any one of embodiments 1-22, wherein the binding molecule is cytotoxic.

Embodiment 24. The CTLA-4 binding molecule of any one of embodiments 1-22, wherein the binding molecule is non-cytotoxic.

Embodiment 25. A pharmaceutical composition comprising the CTLA-4 binding molecule of any one of embodiments 1-24, and at least one pharmaceutically acceptable excipient or carrier.

Embodiment 26. A polynucleotide encoding the CTLA-4 binding molecule of any one of embodiments 1-24, or a complement thereof.

Embodiment 27. An expression vector comprising a polynucleotide according to embodiment 26.

Embodiment 28. A host cell comprising a polynucleotide according to embodiment 26 or an expression vector according to embodiment 27.

Embodiment 29. A method of killing a CTLA-4 expressing cell, the method comprising the step of contacting the cell with a CTLA-4 binding molecule according to any one of embodiments 1-24 or a pharmaceutical composition according to embodiment 25.

Embodiment 30. A method for making a CTLA-4 binding molecule, the method comprising (a) expressing a CTLA-4 binding molecule of any one of embodiments 1-24 and (b) recovering the CTLA-4 binding molecule.

Embodiment 31. The method of embodiment 30, wherein expressing the CTLA-4 binding molecule comprises culturing the host cell of embodiment 28 under conditions wherein the CTLA-4 binding molecule is expressed.

Embodiment 32. A method for purifying the CTLA-4 binding molecule of any one of embodiments 1-24 from an expression system composition comprising the CTLA-4 binding molecule and at least one other biomolecule, the method comprising (i) contacting the expression system composition with a bacterial protein L to create a CTLA-4 binding molecule-protein L complex, and (ii) recovering the CTLA-4 binding molecule-protein L complex.

Embodiment 33. The method of embodiment 32, wherein the expression system composition is a cellular lysate.

Embodiment 34. The method of embodiment 32 or 33, wherein the protein L is isolated or derived from F. magna.

Embodiment 35. The method of any one of embodiments 32-34, wherein the protein L is conjugated to a resin.

Embodiment 36. A method of treating cancer, the method comprising administering to a subject in need thereof an effective amount of the CTLA-4 binding molecule of any one of embodiments 1-24, or the pharmaceutical composition of embodiment 25.

Embodiment 37. The method of embodiment 36, wherein the cancer is a solid tumor.

Embodiment 38. The method of embodiment 36 or 37, wherein the cancer is bladder cancer, breast cancer, colon cancer, endometrial cancer, esophageal cancer, fallopian tube cancer, gastrointestinal cancer, glioma, head and neck cancer, kidney cancer, liver cancer, lung cancer, lymphoma, Merkel cell carcinoma, mesothelioma, myeloma, nasopharyngeal neoplasm, ovarian cancer, pancreatic cancer, peritoneal neoplasm, prostate cancer, skin cancer, transitional cell carcinoma, soft tissue sarcoma, or urothelial cancer.

Embodiment 39. The method of embodiment 36 or 37, wherein the cancer is bladder cancer, and the bladder cancer is urothelial carcinoma.

Embodiment 40. The method of embodiment 36 or 37, wherein the cancer is breast cancer, and the breast cancer is HER2 positive breast cancer or triple negative breast cancer.

Embodiment 41. The method of embodiment 36 or 37, wherein the cancer is colon cancer, and the colon cancer is colorectal cancer.

Embodiment 42. The method of embodiment 36 or 37, wherein the cancer is gastrointestinal cancer, and the gastrointestinal cancer is gastric cancer, biliary tract neoplasm, or gastroesophageal junction cancer.

Embodiment 43. The method of embodiment 36 or 37, wherein the cancer is glioma, and the glioma is glioblastoma.

Embodiment 44. The method of embodiment 36 or 37, wherein the cancer is head and neck cancer, and the head and neck cancer is squamous cell carcinoma of the head and neck.

Embodiment 45. The method of embodiment 36 or 37, wherein the cancer is kidney cancer, and the kidney cancer is renal cell carcinoma.

Embodiment 46. The method of embodiment 36 or 37, wherein the cancer is liver cancer, and the liver cancer is hepatocellular carcinoma.

Embodiment 47. The method of embodiment 36 or 37, wherein the cancer is lung cancer, and the lung cancer is non-small cell lung cancer or small-cell lung cancer.

Embodiment 48. The method of embodiment 47, wherein the non-small cell lung cancer is metastatic non-small cell lung cancer.

Embodiment 49. The method of embodiment 36 or 37, wherein the cancer is lymphoma, and the lymphoma is Hodgkin lymphoma, non-Hodgkin lymphoma, primary mediastinal large B-cell lymphoma, or diffuse large B-cell lymphoma.

Embodiment 50. The method of embodiment 36 or 37, wherein the cancer is mesothelioma, and the mesothelioma is pleural mesothelioma.

Embodiment 51. The method of embodiment 50, wherein the pleural mesothelioma is malignant pleural mesothelioma.

Embodiment 52. The method of embodiment 36 or 37, wherein the cancer is myeloma, and the myeloma is multiple myeloma.

Embodiment 53. The method of embodiment 36 or 37, wherein the cancer is skin cancer, and the skin cancer is squamous cell cancer of the skin or melanoma.

Embodiment 54. The method of embodiment 53, wherein the melanoma is unresectable melanoma or metastatic melanoma.

Embodiment 55. The method of any one of embodiments 36-54, wherein the cancer is relapsed or refractory to a treatment involving at least one of ipilimumab, nivolumab, pembrolizumab, atezolizumab, durvalumab, avelumab, tremelimumab and cemiplimab.

Embodiment 56. The method of any one of embodiments 36-55, wherein the cancer is metastatic.

Embodiment 57. The method of any one of embodiments 36-56, wherein the CTLA-4 binding molecule blocks the interaction between CTLA-4 and one or more of its ligands.

Embodiment 58. The method of any one of embodiments 36-57, wherein the CTLA-4 binding molecule blocks the interaction between CTLA-4 and CD80 (B7-1).

Embodiment 59. The method of any one of embodiments 36-58, wherein the CTLA-4 binding molecule blocks the interaction between CTLA-4 and CD86 (B7-2).

Embodiment 60. A CTLA-4 binding molecule comprising a Shiga toxin A subunit effector polypeptide and a binding region capable of specifically binding an extracellular part of CTLA-4; wherein the CTLA-4 binding molecule comprises the amino acid sequence of SEQ ID NO: 329, or an amino acid sequence at least 95% identical thereto.

Embodiment 61. The CTLA-4 binding molecule of embodiment 60, wherein the CTLA-4 binding molecule comprises the amino acid sequence of SEQ ID NO: 329.

Embodiment 62. A pharmaceutical composition comprising the CTLA-4 binding molecule of embodiment 60 or 61, and at least one pharmaceutically acceptable excipient or carrier.

Embodiment 63. A method of treating cancer, the method comprising administering to a subject in need thereof an effective amount of the CTLA-4 binding molecule of embodiment 60 or 61, or the pharmaceutical composition of embodiment 62.

Section 2:

Embodiment 1. A method of treating cancer, the method comprising administering to a subject in need thereof:
(i) an effective amount of a CTLA-4 binding molecule comprising a Shiga toxin A subunit effector polypeptide and a binding region capable of specifically binding an extracellular part of CTLA-4, wherein the binding region comprises a VHH domain comprising a HCDR1, a HCDR2, and a HCDR3; and
(ii) an additional anti-cancer agent, wherein the anti-cancer agent is an inhibitor of PD-1 or PD-L1.

Embodiment 2. A method of treating cancer, the method comprising administering to a subject in need thereof:
(i) an effective amount of a CTLA-4 binding molecule comprising a Shiga toxin A subunit effector polypeptide and a binding region capable of specifically binding an extracellular part of CTLA-4; wherein the binding region comprises a first VHH domain comprising a first HCDR1, a first HCDR2, and a first HCDR3 and a second VHH domain comprising a second HCDR1, a second HCDR2, and a second HCDR3; and
(ii) an additional anti-cancer agent, wherein the anti-cancer agent is an inhibitor of PD-1 or PD-L1.

Embodiment 3. The method of embodiment 1 or 2, wherein the inhibitor of PD-1 is an anti-PD-1 antibody or an anti-PD-1 antibody-drug conjugate (ADC).

Embodiment 4. The method of embodiment 3, wherein the anti-PD-1 antibody is nivolumab, pembrolizumab, dostarlimab, tislelizumab, or cemiplimab.

Embodiment 5. The method of embodiment 3, wherein the anti-PD-1 antibody is nivolumab.

Embodiment 6. The method of embodiment 1 or 2, wherein the inhibitor of PD-L1 is an anti-PD-L1 antibody or an anti-PD-L1 ADC.

Embodiment 7. The method of embodiment 6, wherein the anti-PD-L1 antibody is atezolizumab, durvalumab, or avelumab.

Embodiment 8. The method of any one of embodiments 1-7, wherein the CTLA-4 binding molecule is administered to the subject before the additional anti-cancer agent.

Embodiment 9. The method of any one of embodiments 1-7, wherein the CTLA-4 binding molecule is administered to the subject after the additional anti-cancer agent.

Embodiment 10. The method of any one of embodiments 1-7, wherein the CTLA-4 binding molecule is administered to the subject at the same time as the additional anti-cancer agent.

Embodiment 11. The method of any one of embodiments 1 and 3-10, wherein
(a) the HCDR1 comprises the amino acid sequence of SEQ ID NO: 23, the HCDR2 comprises the amino acid sequence of SEQ ID NO: 24, and first HCDR3 comprises the amino acid sequence of SEQ ID NO: 25; or
(b) the HCDR1 comprises the amino acid sequence of SEQ ID NO: 26, the HCDR2 comprises the amino acid sequence of SEQ ID NO: 27, and the HCDR3 comprises the amino acid sequence of SEQ ID NO: 28.

Embodiment 12. The method of any one of embodiments 1 and 3-10, wherein the VHH domain comprises the amino acid sequence of SEQ ID NO: 21 or SEQ ID NO: 22.

Embodiment 13. The method of any one of embodiments 2-10, wherein the CTLA-4 binding molecule comprises a linker that links the first VHH domain and the second VHH domain.

Embodiment 14. The method of any one of embodiments 2-10 and 13, wherein the first HCDR1 comprises the amino acid sequence of SEQ ID NO: 23, the first HCDR2 comprises the amino acid sequence of SEQ ID NO: 24, and the first HCDR3 comprises the amino acid sequence of SEQ ID NO: 25.

Embodiment 15. The method of any one of embodiments 2-10, 13, and 14, wherein the first VHH domain comprises the amino acid sequence of SEQ ID NO: 21.

Embodiment 16. The method of any one of embodiments 2-10 and 13-15, wherein the second HCDR1 comprises the amino acid sequence of SEQ ID NO: 26, the second HCDR2 comprises the amino acid sequence of SEQ ID NO: 27, and the second HCDR3 comprises the amino acid sequence of SEQ ID NO: 28.

Embodiment 17. The method of any one of embodiments 2-10 and 13-16, wherein the second VHH domain comprises the amino acid sequence of SEQ ID NO: 22.

Embodiment 18. The method of any one of embodiments 13-17, wherein the linker comprises the amino acid sequence of SEQ ID NO: 29.

Embodiment 19. The method of any one of embodiments 1-18, wherein the Shiga toxin A subunit effector polypeptide comprises a polypeptide having the sequence of:
(i) amino acids 1 to 251 of any one of SEQ ID NOs: 1-18; or
(ii) amino acids 1 to 261 of any one of SEQ ID NOs: 1-18;
or a polypeptide having a sequence that is at least 90% or at least 95% identical thereto.

Embodiment 20. The method of any one of embodiments 1-19, wherein the Shiga toxin A subunit effector polypeptide comprises or consists of a polypeptide having the sequence of any one of SEQ ID NO: 40 to 68.

Embodiment 21. The method of any one of embodiments 1-19, wherein the Shiga toxin A subunit effector polypeptide comprises the amino acid sequence of SEQ ID NO: 41.

Embodiment 22. The method of any one of embodiments 1-21, wherein the CTLA-4 binding molecule comprises a linker that links the Shiga toxin A subunit effector polypeptide and the binding region.

Embodiment 23. The method of embodiment 22, wherein the linker that links the Shiga toxin A subunit effector polypeptide and the binding region comprises the amino acid sequence of SEQ ID NO: 218.

Embodiment 24. The method of embodiment 22 or 23, wherein the CTLA-4 binding molecule comprises, from N-terminus to C-terminus or from C-terminus to N-terminus, the Shiga toxin A subunit effector polypeptide, the linker, and the binding region.

Embodiment 25. The method of embodiment 22 or 23, wherein the CTLA-4 binding molecule comprises, from N-terminus to C-terminus, the Shiga toxin A subunit effector polypeptide, the binding region linker, the first VHH domain, the linker, and the second VHH domain.

Embodiment 26. The method of embodiment 25, wherein the CTLA-4 binding molecule comprises the amino acid sequence of SEQ ID NO: 329, or an amino acid sequence at least 95% identical to SEQ ID NO: 329.

Embodiment 27. The method of any one of embodiments 1-26, wherein the CTLA-4 binding molecule is a single continuous polypeptide.

Embodiment 28. The method of any one of embodiments 1-26, wherein the CTLA-4 binding molecule comprises two polypeptides.

Embodiment 29. The method of embodiment 28, wherein the two polypeptides are non-covalently linked.

Embodiment 30. The method of embodiment 28, wherein the two polypeptides are covalently linked.

Embodiment 31. The method of any one of embodiments 1-30, wherein the CTLA-4 binding molecule is cytotoxic.

Embodiment 32. The method of any one of embodiments 1-30, wherein the CTLA-4 binding molecule is non-cytotoxic.

Embodiment 33. The method of any one of embodiments 1-32, wherein the cancer is a solid tumor.

Embodiment 34. The method of any one of embodiments 1-32, wherein the cancer is bladder cancer, breast cancer, colon cancer, endometrial cancer, esophageal cancer, fallopian tube cancer, gastrointestinal cancer, glioma, head and neck cancer, kidney cancer, liver cancer, lung cancer, lymphoma, Merkel cell carcinoma, mesothelioma, myeloma, nasopharyngeal neoplasm, ovarian cancer, pancreatic cancer, peritoneal neoplasm, prostate cancer, skin cancer, transitional cell carcinoma, soft tissue sarcoma, or urothelial cancer.

Embodiment 35. The method of any one of embodiments 1-34, wherein the cancer is bladder cancer, and the bladder cancer is urothelial carcinoma.

Embodiment 36. The method of any one of embodiments 1-34, wherein the cancer is breast cancer, and the breast cancer is HER2 positive breast cancer or triple negative breast cancer.

Embodiment 37. The method of any one of embodiments 1-34, wherein the cancer is colon cancer, and the colon cancer is colorectal cancer.

Embodiment 38. The method of any one of embodiments 1-34, wherein the cancer is gastrointestinal cancer, and the gastrointestinal cancer is gastric cancer, biliary tract neoplasm, or gastroesophageal junction cancer.

Embodiment 39. The method of any one of embodiments 1-34, wherein the cancer is glioma, and the glioma is glioblastoma.

Embodiment 40. The method of any one of embodiments 1-34, wherein the cancer is head and neck cancer, and the head and neck cancer is squamous cell carcinoma of the head and neck.

Embodiment 41. The method of any one of embodiments 1-34, wherein the cancer is kidney cancer, and the kidney cancer is renal cell carcinoma.

Embodiment 42. The method of any one of embodiments 1-34, wherein the cancer is liver cancer, and the liver cancer is hepatocellular carcinoma.

Embodiment 43. The method of any one of embodiments 1-34, wherein the cancer is lung cancer, and the lung cancer is non-small cell lung cancer or small-cell lung cancer.

Embodiment 44. The method of embodiment 43, wherein the non-small cell lung cancer is metastatic non-small cell lung cancer.

Embodiment 45. The method of any one of embodiments 1-34, wherein the cancer is lymphoma, and the lymphoma is Hodgkin lymphoma, non-Hodgkin lymphoma, primary mediastinal large B-cell lymphoma, or diffuse large B-cell lymphoma.

Embodiment 46. The method of any one of embodiments 1-34, wherein the cancer is mesothelioma, and the mesothelioma is pleural mesothelioma.

Embodiment 47. The method of embodiment 46, wherein the pleural mesothelioma is malignant pleural mesothelioma.

Embodiment 48. The method of any one of embodiments 1-34, wherein the cancer is myeloma, and the myeloma is multiple myeloma.

Embodiment 49. The method of any one of embodiments 1-34, wherein the cancer is skin cancer, and the skin cancer is squamous cell cancer of the skin or melanoma.

Embodiment 50. The method of embodiment 49, wherein the melanoma is unresectable melanoma or metastatic melanoma.

Embodiment 51. The method of any one of embodiments 1-50, wherein the cancer is relapsed or refractory to a treatment involving at least one of ipilimumab, nivolumab, pembrolizumab, atezolizumab, durvalumab, avelumab, tremelimumab, cemiplimab, relatlimab, tiragolumab, ociperlimab, vibostolimab, domvanalimab, sacituzumab, sacituzumab govitecan, datopotamab, or datopotamab deruxtecan.

Embodiment 52. The method of any one of embodiments 1-51, wherein the cancer is metastatic.

Embodiment 53. The method of any one of embodiments 1-52, wherein the CTLA-4 binding molecule blocks the interaction between CTLA-4 and one or more of its ligands.

Embodiment 54. The method of any one of embodiments 1-53, wherein the CTLA-4 binding molecule blocks the interaction between CTLA-4 and CD80 (B7-1).

Embodiment 55. The method of any one of embodiments 1-54, wherein the CTLA-4 binding molecule blocks the interaction between CTLA-4 and CD86 (B7-2).

Section 3:

Embodiment 1. A method of treating cancer, the method comprising administering to a subject in need thereof: an effective amount of a CTLA-4 binding molecule comprising a Shiga toxin A subunit effector polypeptide and a binding region capable of specifically binding an extracellular part of CTLA-4; wherein the binding region comprises a first VHH domain comprising a first HCDR1, a first HCDR2, and a first HCDR3 and a second VHH domain comprising a second HCDR1, a second HCDR2, and a second HCDR3; and wherein the effective amount of the CTLA-4 binding molecule is a dose in a range of about 1 μg/kg to about 250 μg/kg.

Embodiment 2. The method of embodiment 1, wherein the CTLA-4 binding molecule comprises a linker that links the first VHH domain and the second VHH domain.

Embodiment 3. The method of embodiment 1 or embodiment 2, wherein the first HCDR1 comprises the amino acid sequence of SEQ ID NO: 23, the first HCDR2 comprises the amino acid sequence of SEQ ID NO: 24, and the first HCDR3 comprises the amino acid sequence of SEQ ID NO: 25.

Embodiment 4. The method of any one of embodiments 1-3, wherein the first VHH domain comprises the amino acid sequence of SEQ ID NO: 21.

Embodiment 5. The method of any one of embodiments 1-4, wherein the second HCDR1 comprises the amino acid sequence of SEQ ID NO: 26, the second HCDR2 comprises the amino acid sequence of SEQ ID NO: 27, and the second HCDR3 comprises the amino acid sequence of SEQ ID NO: 28.

Embodiment 6. The method of any one of embodiments 1-5, wherein the second VHH domain comprises the amino acid sequence of SEQ ID NO: 22.

Embodiment 7. The method of any one of embodiments 1-6, wherein the linker comprises the amino acid sequence of SEQ ID NO: 29.

Embodiment 8. The method of any one of embodiments 1-7, wherein the Shiga toxin A subunit effector polypeptide comprises a polypeptide having the sequence of: (i) amino acids 1 to 251 of any one of SEQ ID NOs: 1-18; or (ii) amino acids 1 to 261 of any one of SEQ ID NOs: 1-18; or a polypeptide having a sequence that is at least 90% or at least 95% identical thereto.

Embodiment 9. The method of any one of embodiments 1-8, wherein the Shiga toxin A subunit effector polypeptide comprises or consists of a polypeptide having the sequence of any one of SEQ ID NOs: 40 to 68.

Embodiment 10. The method of any one of embodiments 1-9, wherein the Shiga toxin A subunit effector polypeptide comprises the amino acid sequence of SEQ ID NO: 41.

Embodiment 11. The method of any one of embodiments 1-10, wherein the CTLA-4 binding molecule comprises a linker that links the Shiga toxin A subunit effector polypeptide and the binding region.

Embodiment 12. The method of embodiment 11, wherein the linker that links the Shiga toxin A subunit effector polypeptide and the binding region comprises the amino acid sequence of SEQ ID NO: 218.

Embodiment 13. The method of embodiment 11 or embodiment 12, wherein the CTLA-4 binding molecule comprises, from N-terminus to C-terminus, the Shiga toxin A subunit effector polypeptide, the binding region linker, the first VHH domain, the linker, and the second VHH domain.

Embodiment 14. The method of embodiment 11 or 12, wherein the CTLA-4 binding molecule comprises an amino acid sequence of SEQ ID NO: 329, or an amino acid sequence at least 95% identical to SEQ ID NO: 329.

Embodiment 15. The method of any one of embodiments 1-14, wherein the CTLA-4 binding molecule is a single continuous polypeptide.

Embodiment 16. The method of any one of embodiments 1-14, wherein the CTLA-4 binding molecule comprises two polypeptides.

Embodiment 17. The method of embodiment 16, wherein the two polypeptides are non-covalently linked.

Embodiment 18. The method of embodiment 16, wherein the two polypeptides are covalently linked.

Embodiment 19. The method of any one of embodiments 1-18, wherein the dose is about 1 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 125 μg/kg, about 150 μg/kg, about 175 μg/kg, about 200 μg/kg, about 250 μg/kg, or any value therebetween.

Embodiment 20. The method of any one of embodiments 1-18, wherein the dose is about 32 μg/kg.

Embodiment 21. The method of any one of embodiments 1-20, wherein the CTLA-4 binding molecule is administered to the subject by intravenous, subcutaneous, or intramuscular injection.

Embodiment 22. The method of embodiment 21, wherein the CTLA-4 binding molecule is administered to the subject by intravenous injection.

Embodiment 23. The method of embodiment 22, wherein the CTLA-4 binding molecule is administered to the subject over a period of about 10 minutes to about 1 hour.

Embodiment 24. The method of embodiment 22, wherein the CTLA-4 binding molecule is administered to the subject over a period of about 30 minutes.

Embodiment 25. The method of any one of embodiments 1-24, wherein the CTLA-4 binding molecule is administered to the subject once.

Embodiment 26. The method of any one of embodiments 1-24, wherein the CTLA-4 binding molecule is administered to the subject more than once.

Embodiment 27. The method of embodiment 26, wherein the CTLA-4 binding molecule is administered to the subject once every seven days.

Embodiment 28. The method of embodiment 26, wherein the CTLA-4 binding molecule is administered to the subject over a 28 day cycle.

Embodiment The method of embodiment 28, wherein the CTLA-4 binding molecule is administered to the subject on days 1, 8, 15, and 22 of the 28 day cycle.

Embodiment 30. The method of embodiment 28, wherein the CTLA-4 binding molecule is administered to the subject on days 1 and 15 of the 28 day cycle.

Embodiment 31. The method of embodiment 28, wherein the CTLA-4 binding molecule is administered to the subject on days 1, 8, and 15 of the 28 day cycle.

Embodiment 32. The method of any one of embodiments 21-31, wherein the subject is administered a dose in the range of about 1 μg/kg to about 250 μg/kg at each administration.

Embodiment 33. The method of embodiment 32, wherein the subject is administered a dose of about 1 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 125 μg/kg, about 150 μg/kg, about 175 μg/kg, about 200 μg/kg, about 250 μg/kg, or any value therebetween at each administration.

Embodiment 34. The method of embodiment 32, wherein the subject is administered a dose of about 32 μg/kg at each administration.

Embodiment 35. The method of any one of embodiments 28-34, wherein the CTLA-4 binding molecule is administered to the subject over at least one 28 day cycle.

Embodiment 36. The method of any one of embodiments 28-34, wherein the CTLA-4 binding molecule is administered to the subject over one 28 day cycle, two 28 day cycles, three 28 day cycles, four 28 day cycles, five 28 day cycles, or six 28 day cycles.

Embodiment 37. The method of any one of embodiments 1-36, wherein the method comprises administering to the subject a composition comprising about 0.1 mg/mL to about 100.0 mg/mL of the CTLA-4 binding molecule.

Embodiment 38. The method of any one of embodiments 1-36, wherein the method comprises administering to the subject a composition comprising about 0.5 mg/mL of the CTLA-4 binding molecule.

Embodiment 39. The method of any one of embodiments 1-38, wherein the method comprises administering to the subject a composition comprising the CTLA-4 binding molecule in a buffer comprising one or more of sodium acetate, sucrose, sodium chloride, and poloxamer 188.

Embodiment 40. The method of embodiment 39, wherein the buffer has a pH of about 5.0.

Embodiment 41. The method of any one of embodiments 1-40, wherein the method comprises administering to the subject a composition comprising: (i) about 0.5 mg/mL of the CTLA-4 binding molecule; (ii) about 20 mM sodium acetate; (iii) about 6% w/v sucrose; (iv) about 75 mM sodium chloride; and (v) about 0.1% poloxamer 188; wherein the composition has a pH of about 5.0.

Embodiment 42. The method of any one of embodiments 1-41, wherein the method comprises administering to the subject a second anti-cancer agent.

Embodiment 43. The method of embodiment 42, wherein the second anti-cancer agent is a PD-1 inhibitor.

Embodiment 44. The method of embodiment 43, wherein the PD-1 inhibitor is an anti-PD-1 antibody.

Embodiment 45. The method of embodiment 44, wherein the anti-PD-1 antibody is nivolumab.

Embodiment 46. The method of any one of embodiments 43-45, wherein the PD-1 inhibitor is administered to the subject at a dose of about 250 mg to about 750 mg.

Embodiment 47. The method of any one of embodiments 43-45, wherein the PD-1 inhibitor is administered to the subject at a dose of about 480 mg.

Embodiment 48. The method of any one of embodiments 43-47, wherein the PD-1 inhibitor is administered to the subject by intravenous injection.

Embodiment 49. The method of any one of embodiments 43-48, wherein the PD-1 inhibitor is administered to the subject once.

Embodiment 50. The method of any one of embodiments 43-48, wherein the PD-1 inhibitor is administered to the subject more than once.

Embodiment 51. The method of embodiment 50, wherein the PD-1 inhibitor is administered to the subject once in a 28 day cycle.

Embodiment 52. The method of embodiment 51, wherein the PD-1 inhibitor is administered to the subject on day 1 of the 28 day cycle.

Embodiment 53. The method of any one of embodiments 50-52, wherein the PD-1 inhibitor is administered to the subject over at least one 28 day cycle.

Embodiment 54. The method of embodiment 53, wherein the PD-1 inhibitor is administered to the subject starting on day 1 of a second 28 day cycle.

Embodiment 55. The method of any one of embodiments 1-54, wherein the cancer is a solid tumor.

Embodiment 56. The method of any one of embodiments 1-54, wherein the cancer is bladder cancer, breast cancer, cervical cancer, colon cancer, endometrial cancer, esophageal cancer, fallopian tube cancer, gastrointestinal cancer, glioma, head and neck cancer, kidney cancer, liver cancer, lung cancer, lymphoma, Merkel cell carcinoma, mesothelioma, myeloma, nasopharyngeal neoplasm, ovarian cancer, pancreatic cancer, peritoneal neoplasm, prostate cancer, skin cancer, transitional cell carcinoma, soft tissue sarcoma, microsatellite instability-high (MSI-H) or mismatch repair deficient (dMMR) cancer, or urothelial cancer.

Embodiment 57. The method of any one of embodiments 1-56, wherein the cancer is cervical cancer, and the cervical cancer is cervical carcinoma.

Embodiment 58. The method of any one of embodiments 1-56, wherein the cancer is esophageal cancer, and the esophageal cancer is esophageal squamous cell carcinoma.

Embodiment 59. The method of any one of embodiments 1-56, wherein the cancer is bladder cancer, and the bladder cancer is urothelial carcinoma.

Embodiment 60. The method of any one of embodiments 1-56, wherein the cancer is breast cancer, and the breast cancer is HER2 positive breast cancer or triple negative breast cancer.

Embodiment 61. The method of any one of embodiments 1-56, wherein the cancer is colon cancer, and the colon cancer is colorectal cancer.

Embodiment 62. The method of any one of embodiments 1-56, wherein the cancer is gastrointestinal cancer, and the gastrointestinal cancer is gastric cancer, biliary tract neoplasm, or gastroesophageal junction cancer.

Embodiment 63. The method of any one of embodiments 1-56, wherein the cancer is MSI-H or dMMR cancer.

Embodiment 64. The method of any one of embodiments 1-56, wherein the cancer is glioma, and the glioma is glioblastoma.

Embodiment 65. The method of any one of embodiments 1-56, wherein the cancer is head and neck cancer, and the head and neck cancer is squamous cell carcinoma of the head and neck.

Embodiment 66. The method of any one of embodiments 1-56, wherein the cancer is kidney cancer, and the kidney cancer is renal cell carcinoma.

Embodiment 67. The method of any one of embodiments 1-56, wherein the cancer is liver cancer, and the liver cancer is hepatocellular carcinoma.

Embodiment 68. The method of any one of embodiments 1-56, wherein the cancer is lung cancer, and the lung cancer is non-small cell lung cancer or small-cell lung cancer.

Embodiment 69. The method of any one of embodiments 1-56, wherein the non-small cell lung cancer is metastatic non-small cell lung cancer.

Embodiment 70. The method of any one of embodiments 1-56, wherein the cancer is lymphoma, and the lymphoma is Hodgkin lymphoma, non-Hodgkin lymphoma, primary mediastinal large B-cell lymphoma, or diffuse large B-cell lymphoma.

Embodiment 71. The method of any one of embodiments 1-56, wherein the cancer is mesothelioma, and the mesothelioma is pleural mesothelioma.

Embodiment 72. The method of any one of embodiments 1-56, wherein the cancer is myeloma, and the myeloma is multiple myeloma.

Embodiment 73. The method of any one of embodiments 1-56, wherein the cancer is skin cancer, and the skin cancer is squamous cell cancer of the skin or melanoma.

Embodiment 74. The method of embodiment 73, wherein the melanoma is unresectable melanoma or metastatic melanoma.

Embodiment 75. The method of any one of embodiments 1-73, wherein the cancer is metastatic.

Embodiment 76. The method of any one of embodiments 1-75, wherein the cancer is relapsed or refractory to at least one other cancer therapy, or the subject is known to be intolerant of at least one other cancer therapy.

Embodiment 77. The method of embodiment 76, wherein the at least one other cancer therapy is ipilimumab, nivolumab, pembrolizumab, atezolizumab, durvalumab, avelumab, tremelimumab, cemiplimab, relatlimab, tiragolumab, ociperlimab, vibostolimab, domvanalimab, sacituzumab, sacituzumab govitecan, datopotamab, or datopotamab deruxtecan.

Embodiment 78. The method of any one of embodiments 1-77, wherein the subject receives at least one pre-medication prior to the administration of the CTLA-4 binding molecule.

Embodiment 79. The method of embodiment 78, wherein the at least one pre-medication is an H1/H2 blocker-containing agent and/or anti-pyrectic agent.

Embodiment 80. A composition comprising: (i) a CTLA-4 binding molecule; (ii) sodium acetate; (iii) sucrose; (iv) sodium chloride; and/or (v) poloxamer 188.

Embodiment 81. The composition of embodiment 80, wherein the CTLA-4 binding molecule comprises an amino acid sequence of SEQ ID NO: 329, or an amino acid sequence at least 95% identical to SEQ ID NO: 329.

Embodiment 82. The composition of embodiment 80 or embodiment 81, wherein the CTLA-4 molecule is at a concentration of about 0.1 mg/mL to about 10 mg/mL.

Embodiment 83. The composition of embodiment 82, wherein the CTLA-4 molecule is at a concentration of about 0.5 mg/mL.

Embodiment 84. The composition of any one of embodiments 80-83, wherein the sodium acetate is at a concentration of about 1 mM to about 50 mM.

Embodiment 85. The composition of embodiment 84, wherein the sodium acetate is at a concentration of about 20 mM.

Embodiment 86. The composition of any one of embodiments 80-85, wherein the sucrose is at a concentration of about 1% w/v to about 10% w/v.

Embodiment 87. The composition of embodiment 86, wherein the sucrose is at a concentration of about 6% w/v.

Embodiment 88. The composition of any one of embodiments 80-87, wherein the sodium chloride is at a concentration of about 50 mM to about 100 mM.

Embodiment 89. The composition of embodiment 88, wherein the sodium chloride is at a concentration of about 75 mM.

Embodiment 90. The composition of any one of embodiments 80-89, wherein the poloxamer 188 is at a concentration of about 0.01% w/v to about 1% w/v.

Embodiment 91. The composition of embodiment 90, wherein the poloxamer 188 is at a concentration of about 0.1% w/v.

Embodiment 92. The composition of any one of embodiments 80-91, wherein the pH of the composition is about 4.0 to about 7.0.

Embodiment 93. The composition of embodiment 92, wherein the pH of the composition is about 5.0.

Embodiment 94. The composition of embodiment 80 comprising: (i) about 0.5 mg/mL of a CTLA-4 binding molecule, wherein the CTLA-4 binding molecule comprises an amino acid sequence of SEQ ID NO: 329; (ii) about 20 mM sodium acetate; (iii) about 6% w/v sucrose; (iv) about 75 mM sodium chloride; and (v) about 0.1% poloxamer 188; wherein the composition has a pH of about 5.0.

Embodiment 95. A method of treating cancer, the method comprising administering to a subject in need thereof a composition comprising: (i) a CTLA-4 binding molecule; (ii) sodium acetate; (iii) sucrose; (iv) sodium chloride; and/or (v) poloxamer 188.

Embodiment 96. The method of embodiment 95, wherein the CTLA-4 binding molecule comprises an amino acid sequence of SEQ ID NO: 329, or an amino acid sequence at least 95% identical to SEQ ID NO: 329.

Embodiment 97. The method of embodiment 95 or embodiment 96, wherein the CTLA-4 molecule is at a concentration of about 0.1 mg/mL to about 10 mg/mL.

Embodiment 98. The method of embodiment 97, wherein the CTLA-4 molecule is at a concentration of about 0.5 mg/mL.

Embodiment 99. The method of any one of embodiments 95-98, wherein the sodium acetate is at a concentration of about 1 mM to about 50 mM.

Embodiment 100. The method of embodiment 99, wherein the sodium acetate is at a concentration of about 20 mM.

Embodiment 101. The method of any one of embodiments 95-100, wherein the sucrose is at a concentration of about 1% w/v to about 10% w/v.

Embodiment 102. The method of embodiment 101, wherein the sucrose is at a concentration of about 6% w/v.

Embodiment 103. The method of any one of embodiments 95-102, wherein the sodium chloride is at a concentration of about 50 mM to about 100 mM.

Embodiment 104. The method of embodiment 103, wherein the sodium chloride is at a concentration of about 75 mM.

Embodiment 105. The method of any one of embodiments 95-104, wherein the poloxamer 188 is at a concentration of about 0.01% w/v to about 1% w/v.

Embodiment 106. The method of embodiment 105, wherein the poloxamer 188 is at a concentration of about 0.1% w/v.

Embodiment 107. The method of any one of embodiments 95-106, wherein the pH of the composition is about 4.0 to about 7.0.

Embodiment 108. The method of embodiment 107, wherein the pH of the composition is about 5.0.

Embodiment 109. The method of embodiment 95, comprising: (i) about 0.5 mg/mL of a CTLA-4 binding molecule, wherein the CTLA-4 binding molecule comprises an amino acid sequence of SEQ ID NO: 329; (ii) about 20 mM sodium acetate; (iii) about 6% w/v sucrose; (iv) about 75 mM sodium chloride; and (v) about 0.1% poloxamer 188; wherein the composition has a pH of about 5.0.

Embodiment 110. The method of any one of embodiments 95-109, wherein the method comprises administering to the subject a second anti-cancer agent.

Embodiment 111. The method of embodiment 110, wherein the second anti-cancer agent is a PD-1 inhibitor.

Embodiment 112. The method of embodiment 111, wherein the PD-1 inhibitor is an anti-PD-1 antibody.

Embodiment 113. The method of embodiment 112, wherein the anti-PD-1 antibody is nivolumab.

Embodiment 114. The method of any one of embodiments 95-113, wherein the cancer is bladder cancer, breast cancer, cervical cancer, colon cancer, endometrial cancer, esophageal cancer, fallopian tube cancer, gastrointestinal cancer, glioma, head and neck cancer, kidney cancer, liver cancer, lung cancer, lymphoma, Merkel cell carcinoma, mesothelioma, myeloma, nasopharyngeal neoplasm, ovarian cancer, pancreatic cancer, peritoneal neoplasm, prostate cancer, skin cancer, transitional cell carcinoma, soft tissue sarcoma, microsatellite instability-high (MSI-H) or mismatch repair deficient (dMMR) cancer, or urothelial cancer.

Embodiment 115. A method of treating cancer, the method comprising administering to a subject in need thereof a composition comprising: (i) about 0.5 mg/mL of a CTLA-4 binding molecule, wherein the CTLA-4 binding molecule comprises an amino acid sequence of SEQ ID NO: 329; (ii) about 20 mM sodium acetate; (iii) about 6% w/v sucrose; (iv) about 75 mM sodium chloride; and (v) about 0.1% poloxamer 188; wherein the composition has a pH of about 5.0.

1. A CTLA-4 binding molecule comprising a Shiga toxin A subunit effector polypeptide and a binding region capable of specifically binding an extracellular part of CTLA-4; wherein the binding region comprises a VHH domain comprising a HCDR1, a HCDR2, and a HCDR3.

2. The CTLA-4 binding molecule of claim 1, wherein
(a) the HCDR1 comprises the amino acid sequence of SEQ ID NO: 23, the HCDR2 comprises the amino acid sequence of SEQ ID NO: 24, and the HCDR3 comprises the amino acid sequence of SEQ ID NO: 25; or
(b) the HCDR1 comprises the amino acid sequence of SEQ ID NO: 26, the HCDR2 comprises the amino acid sequence of SEQ ID NO: 27, and the HCDR3 comprises the amino acid sequence of SEQ ID NO: 28.

3. The CTLA-4 binding molecule of claim 1, wherein the VHH domain comprises the amino acid sequence of SEQ ID NO: 21 or SEQ ID NO: 22.

4. A CTLA-4 binding molecule comprising a Shiga toxin A subunit effector polypeptide and a binding region capable of specifically binding an extracellular part of CTLA-4; wherein the binding region comprises a first VHH domain comprising a first HCDR1, a first HCDR2, and a first HCDR3 and a second VHH domain comprising a second HCDR1, a second HCDR2, and a second HCDR3.

5. The CTLA-4 binding molecule of claim 4, comprising a linker that links the first VHH domain and the second VHH domain.

6. The CTLA-4 binding molecule of claim 4, wherein the first HCDR1 comprises the amino acid sequence of SEQ ID NO: 23, the first HCDR2 comprises the amino acid sequence of SEQ ID NO: 24, and the first HCDR3 comprises the amino acid sequence of SEQ ID NO: 25.

7. The CTLA-4 binding molecule of claim 4, wherein the first VHH domain comprises the amino acid sequence of SEQ ID NO: 21.

8. The CTLA-4 binding molecule of claims 4, wherein the second HCDR1 comprises the amino acid sequence of SEQ ID NO: 26, the second HCDR2 comprises the amino acid sequence of SEQ ID NO: 27, and the second HCDR3 comprises the amino acid sequence of SEQ ID NO: 28.

9. The CTLA-4 binding molecule of claim 4, wherein the second VHH domain comprises the amino acid sequence of SEQ ID NO: 22.

10. The CTLA-4 binding molecule of claim 5, wherein the linker comprises the amino acid sequence of SEQ ID NO: 29.

11. The CTLA-4 binding molecule of claim 4, wherein the Shiga toxin A subunit effector polypeptide comprises a polypeptide having the sequence of:
(i) amino acids 1 to 251 of any one of SEQ ID NOs: 1-18; or
(ii) amino acids 1 to 261 of any one of SEQ ID NOs: 1-18;
or a polypeptide having a sequence that is at least 90% or at least 95% identical thereto.

12. The CTLA-4 binding molecule of claim 4, wherein the Shiga toxin A subunit effector polypeptide comprises or consists of a polypeptide having the sequence of any one of SEQ ID NO: 40 to 68.

13. The CTLA-4 binding molecule of claims 4, wherein the Shiga toxin A subunit effector polypeptide comprises the amino acid sequence of SEQ ID NO: 41.

14. The CTLA-4 binding molecule of claim 4, wherein the CTLA-4 binding molecule comprises a linker that links the Shiga toxin A subunit effector polypeptide and the binding region.

15. The CTLA-4 binding molecule of claim 14, wherein the linker that links the Shiga toxin A subunit effector polypeptide and the binding region comprises the amino acid sequence of SEQ ID NO: 218.

16. The CTLA-4 binding molecule of claim 14, wherein the CTLA-4 binding molecule comprises, from N-terminus to C-terminus or from C-terminus to N-terminus, the Shiga toxin A subunit effector polypeptide, the linker, and the binding region.

17. The CTLA-4 binding molecule of claim 14, wherein the CTLA-4 binding molecule comprises, from N-terminus to C-terminus, the Shiga toxin A subunit effector polypeptide, the binding region linker, the first VHH domain, the linker, and the second VHH domain.

18. The CTLA-4 binding molecule of claim 17, wherein the CTLA-4 binding molecule comprises the amino acid sequence of SEQ ID NO: 329, or an amino acid sequence at least 95% identical to SEQ ID NO: 329.

19. The CTLA-4 binding molecule of claim 4, wherein the CTLA-4 binding molecule is a single continuous polypeptide.

20. The CTLA-4 binding molecule of claim 4, wherein the CTLA-4 binding molecule comprises two polypeptides.

21. The CTLA-4 binding molecule of claim 20, wherein the polypeptides are non-covalently linked.

22. The CTLA-4 binding molecule of claim 20, wherein the polypeptides are covalently linked.

23. The CTLA-4 binding molecule of claim 4, wherein the CTLA-4 binding molecule is cytotoxic.

24. The CTLA-4 binding molecule of claim 4, wherein the CTLA-4 binding molecule is non-cytotoxic.

25. A pharmaceutical composition comprising the CTLA-4 binding molecule of claim 4, and at least one pharmaceutically acceptable excipient or carrier.

26-38. (canceled)

39. A polynucleotide encoding the CTLA-4 binding molecule of claim 4, or a complement thereof.

40. An expression vector comprising a polynucleotide according to claim 39.

41. A host cell comprising a polynucleotide according to claim 39.

42. A method of treating cancer, the method comprising administering to a subject in need thereof an effective amount of the CTLA-4 binding molecule of claim 4.

43-106. (canceled)

107. A CTLA-4 binding molecule comprising a Shiga toxin A subunit effector polypeptide and a binding region capable of specifically binding an extracellular part of CTLA-4; wherein the CTLA-4 binding molecule comprises the amino acid sequence of SEQ ID NO: 329, or an amino acid sequence at least 95% identical thereto.

108-110. (canceled)


SEQUENCE LISTING (396 sequences)

SEQ ID NO: 1 - protein, 293 aa, synthetic construct
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 2 - protein, 293 aa, synthetic construct
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 3 - protein, 297 aa, synthetic construct
DEFTVDFSSQKSYVDSLNSIRSAISTPLGNISQGGVSVSVINHVLGGNYISLNVRGLDPYSERFNHLRLIMERNNLYVAGFINTETNIFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 4 - protein, 251 aa, synthetic construct
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVAR

SEQ ID NO: 5 - protein, 251 aa, synthetic construct
KEFTLDFSTAKKYVDSLNVIRSAIGTPLQTISSGGTSLLMIDSGTGDNLFAVDIMGLEPEEERFNNLRLIVERNNLYVTGFVNRTNNVFYRFADFSHVTFPGTRAVTLSGDSSYTTLQRVAGISRTGMQINRHSLTTSYLDLMSYSGTSLTQSVARAMLRFVTVTAEALRFRQIQRGFRTTLDDLSGRSYVMTAEDVDLTLNWGRLSSILPDYHGQDSVRVGRISFGSINAILGSVALILNCHHHASRVAR

SEQ ID NO: 6 - protein, 251 aa, synthetic construct
QDFTVDFSTAKKYVDSLNAIRSAIGTPLHSISSGGTSLLMIDNGTGDNLFAVDIRGLDPEEERFDNLRLIIERNNLYVTGFVNRTSNIFYRFADFSHVTFPGTRAVTLSGDSSYTTLQRVAGIGRTGMQINRHSLTTSYLDLMSYSGSSLTQPVARAMLRFVTVTAEALRFRQIQRGFRTTLDDVSGHSYTMTVEDVDLTLNWGRLSSVLPDYHGQDSVRVGRISFGGVNAILGSVALILNCHHHTSRVSR

SEQ ID NO: 7 - protein, 250 aa, synthetic construct
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 8 - protein, 250 aa, synthetic construct
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFAHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 9 - protein, 250 aa, synthetic construct
REFTIDFSTQQSYVSSLNSIRTEISTPLEHISQGTTSVSVINHTPPGSYFAVDIRGLDIYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 10 - protein, 250 aa, synthetic construct
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPSVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 11 - protein, 250 aa, synthetic construct
REFTIDFSTQQSYVSSLNSIRTEISTPLEHISQGTTSVSVINHTPPGSYFAVDIRGLDVYQARFDHLRLIIEQNNLYMAGFVNTATNTFYRFSDFTHISVPSVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 12 - protein, 250 aa, synthetic construct
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQVLSETAPVYTMTPGDVDLTLNWGRISNVLPEYRGEDGVRVGRISFNNISAILSTVAVILNCHHQGARSVR

SEQ ID NO: 13 - protein, 250 aa, synthetic construct
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFAHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVIPEYRGEDGVRVGRISFNNISAILGTVAVILNCHHQGARSVR

SEQ ID NO: 14 - protein, 250 aa, synthetic construct
REFMIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPEEVDLTLNWGRISNVLPEFRGEGGVRVGRISFNNISAILGTVAVILNCHHQGARSVR

SEQ ID NO: 15 - protein, 250 aa, synthetic construct
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVIPEYRGEDGVRVGRISFNNISAILSTVAVILNCHHQGARSVR

SEQ ID NO: 16 - protein, 250 aa, synthetic construct
QEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVR

SEQ ID NO: 17 - protein, 250 aa, synthetic construct
QEFTIDFSTQQSYVSSLNSIRTAISTPLEHISQGATSVSVINHTPPGSYISVGIRGLDVYQAHFDHLRLIIEQNNLYVAGFVNTATNTFYRFSDFAHISLPGVTTISMTTDSSYTTLQRVAALERSGMQISRHSLVSSYLALMEFSGNTMTREASRAVLRFVTVTAEALRFRQIQREFRQALSETAPVYTMTPEDVDLTLNWGRISNVLPEYRGEDGVRVGRISFNNISAILGTVAVILNCHHQGARSVR

SEQ ID NO: 18 - protein, 250 aa, synthetic construct
DEFTVDFSSQKSYVDSLNSIRSAISTPLGNISQGGVSVSVINHVPGGNYISLNVRGLDPYSERFNHLRLIMERNNLYVAGFINTETNTFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVR

SEQ ID NO: 19 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 20 - protein, 992 aa, Escherichia coli
MKINKKLLMAALAGAIVVGGGANAYAAEEDNTDNNLSMDEISDAYFDYHGDVSDSVDPVEEEIDEALAKALAEAKETAKKHIDSLNHLSETAKKLAKNDIDSATTINAINDIVARADVMERKTAEKEEAEKLAAAKETAKKHIDELKHLADKTKELAKRDIDSATTINAINDIVARADVMERKTAEKEEAEKLAAAKETAKKHIDELKHLADKTKELAKRDIDSATTIDAINDIVARADVMERKLSEKETPEPEEEVTIKANLIFADGSTQNAEFKGTFAKAVSDAYAYADALKKDNGEYTVDVADKGLTLNIKFAGKKEKPEEPKEEVTIKVNLIFADGKTQTAEFKGTFEEATAKAYAYADLLAKENGEYTADLEDGGNTINIKFAGKETPETPEEPKEEVTIKVNLIFADGKIQTAEFKGTFEEATAKAYAYANLLAKENGEYTADLEDGGNTINIKFAGKETPETPEEPKEEVTIKVNLIFADGKTQTAEFKGTFEEATAEAYRYADLLAKVNGEYTADLEDGGYTINIKFAGKEQPGENPGITIDEWLLKNAKEEAIKELKEAGITSDLYFSLINKAKTVEGVEALKNEILKAHAGEETPELKDGYATYEEAEAAAKEALKNDDVNNAYEIVQGADGRYYYVLKIEVADEEEPGEDTPEVQEGYATYEEAEAAAKEALKEDKVNNAYEVVQGADGRYYYVLKIEDKEDEQPGEEPGENPGITIDEWLLKNAKEDAIKELKEAGISSDIYFDAINKAKTVEGVEALKNEILKAHAEKPGENPGITIDEWLLKNAKEAAIKELKEAGITAEYLFNLINKAKTVEGVESLKNEILKAHAEKPGENPGITIDEWLLKNAKEDAIKELKEAGITSDIYFDAINKAKTIEGVEALKNEILKAHKKDEEPGKKPGEDKKPEDKKPGEDKKPEDKKPGEDKKPEDKKPGKTDKDSPNKKKKAKLPKAGSEAEILTLAAAALSTAAGAYVSLKKRK

SEQ ID NO: 21 - protein, 118 aa, synthetic construct
QVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 22 - protein, 119 aa, synthetic construct
QVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 23 - protein, 10 aa, synthetic construct
GDSYSVKYMG

SEQ ID NO: 24 - protein, 17 aa, synthetic construct
SIYPTGGTFYTDSVKGR

SEQ ID NO: 25 - protein, 8 aa, synthetic construct
AGKWGTDY

SEQ ID NO: 26 - protein, 5 aa, synthetic construct
PNAMG

SEQ ID NO: 27 - protein, 16 aa, synthetic construct
SVTSSGTTNYSDSVKG

SEQ ID NO: 28 - protein, 11 aa, synthetic construct
KELGTFYRRDF

SEQ ID NO: 29 - protein, 9 aa, synthetic construct
GGGGSGGGS

SEQ ID NO: 30-39 - skipped (10 entries)

SEQ ID NO: 40 - protein, 251 aa, synthetic construct
AEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLDDLSGRSYVMTAEDVDLTLNWGRLSSVLPDYHGQDSVRVGRISFGSINAILGSVALILNSHHHASAVAA

SEQ ID NO: 41 - protein, 251 aa, synthetic construct
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAA

SEQ ID NO: 42 - protein, 251 aa, synthetic construct
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLALMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAA

SEQ ID NO: 43 - protein, 251 aa, synthetic construct
KEFTLDFSTAKTYVDSLNVIRSAIGTPLQTISSGGTSLLMIDSGIGDNLFAVDILGFDFTLGRFNNLRLIVERNNLYVTGFVNRTNNAFYRFADFSHVTFPGTTAVTLSADSSYTTLQRVAGISRTGMQINRHSLTTSYLALMSHSGTSLTQSAARAMLRFVTVTAEALRFRQIQRGFRTTLDDLSGASYVMTAEDVDLTLNWGRLSSVLPDYHGQDSVRVGRISFGSINAILGSVALILNSHHHASAVAA

SEQ ID NO: 44 - protein, 251 aa, synthetic construct
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 45 - protein, 251 aa, synthetic construct
AEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTADALRFRQIQRGFRTTLDDLSGRSYVMTAEDVDLTLNWGRLSSVLPDYHGQDSVRVGRISFGSINAILGSVALILNSHHHASAVAA

SEQ ID NO: 46 - protein, 251 aa, synthetic construct
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAA

SEQ ID NO: 47 - protein, 251 aa, synthetic construct
KEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLALMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAA

SEQ ID NO: 48 - protein, 251 aa, synthetic construct
KEFTLDFSTAKTYVDSLNVIRSAIGTPLQTISSGGTSLLMIDSGIGDNLFAVDILGFDFTLGRFNNLRLIVERNNLYVTGFVNRTNNAFYRFADFSHVTFPGTTAVTLSADSSYTTLQRVAGISRTGMQINRHSLTTSYLALMSHSGTSLTQSAARAMLRFVTVTADALRFRQIQRGFRTTLDDLSGASYVMTAEDVDLTLNWGRLSSVLPDYHGQDSVRVGRISFGSINAILGSVALILNSHHHASAVAA

SEQ ID NO: 49 - protein, 251 aa, synthetic construct
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTADALRFRQIQRGFRTTLDDLSGRSYVMTAEDVDLTLNWGRLSSVLPDYHGQDSVRVGRISFGSINAILGSVALILNCHHHASAVAA

SEQ ID NO: 50 - protein, 251 aa, synthetic construct
KEFILRFSVAHKYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 51 - protein, 251 aa, synthetic construct
KEFTLDFSTAKTYVDSLNVIRSAIGTPLQTISSGGTSLLMIDNLVPMVATVVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 52 - protein, 251 aa, synthetic construct
KEFTLDFSTAKTYVDSLNVIRSAIGTPLQTISSGGTSLLMIDSNLVPMVATVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 53 - protein, 251 aa, synthetic construct
KEFTLDFSTAKTYVDSLNVIRSAIGTPLQTISSGGTSLLMIDSGILGFVFTLDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 54 - protein, 251 aa, synthetic construct
KEFTLDFSTAKTYVDSLNVIRSAIGTPLQTISSGGTSLLMIDSGSGDNLFAVGILGFDFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 55 - protein, 251 aa, synthetic construct
KEFTLDFSTAKTYVDSLNVIRSAIGTPLQTISSGGTSLLMIDSGSGDNLFAVDILGFVFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 56 - protein, 251 aa, synthetic construct
KEFTLDFSTAKTYVDSLNVIRSAIGTPLQTISSGGTSLLMIDSGSGDNLFAVDILGFDFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 57 - protein, 251 aa, synthetic construct
KEFTLDFSTAKTYVDSLNVIRSAIGTPLQTISSGGTSLLMIDSGSGDNLFAVGILGFVFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 58 - protein, 251 aa, synthetic construct
KEFILDFSTAKTYVDSLNVIRSAIGTPLQTISSGGTSLLMIDSGIGDNLFAVDVRGIAPIEARFNNLRLIVERNNLYVTGFVNRTNNVFYRFADFSHVTFPGTTAVTLSADSSYTTLQRVAGISRTGMQINRHSLTTSYLDLMSHSATSLTQSVARAMLRFVTVTAEALRFRQIQRGFRTTLAALSGASYVMTAEDVDLTLNWGRLSSVLPDYHGQDSVRVGRISFGSINAILGSVALILNSHHHASAVAA

SEQ ID NO: 59 - protein, 252 aa, synthetic construct
KEFTLDFSTAKTYVDSLNVIRSAIGTPLQTISSGGTSLLMIDSGSGDNLFAVGILGFVFTLEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 60 - protein, 251 aa, synthetic construct
KEFTLDFSTAKTYVDSLNVIRSAIGTPLQTISSGGTSLLMIDSGSGDNLFAVNLVPMVATVGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 61 - protein, 251 aa, synthetic construct
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTNLVPMVATVSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASAVAA

SEQ ID NO: 62 - protein, 251 aa, synthetic construct
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRGILGDVFTLSYVMTAEDVDLTLNWGRLSSVLPDYHGQDSVRVGRISFGSINAILGSVALILNSHHHASAVAA

SEQ ID NO: 63 - protein, 260 aa, synthetic construct
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHILRFSVAHKASAVAA

SEQ ID NO: 64 - protein, 262 aa, synthetic construct
KEFTLDFSTAKTYVDSLNVIRSAIGTPLQTISSGGTSLLMIDSGSGDNLFAVDILGFDFTLGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHARNLVPMVATVASAVAA

SEQ ID NO: 65 - protein, 251 aa, synthetic construct; identical to SEQ ID NO: 61

SEQ ID NO: 66 - protein, 251 aa, synthetic construct; identical to SEQ ID NO: 62

SEQ ID NO: 67 - protein, 260 aa, synthetic construct; identical to SEQ ID NO: 63

SEQ ID NO: 68 - protein, 262 aa, synthetic construct; identical to SEQ ID NO: 64

SEQ ID NO: 69 - protein, 7 aa, synthetic construct
QSVGSSY

SEQ ID NO: 70 - skipped

SEQ ID NO: 71 - protein, 9 aa, synthetic construct
QQYGSSPWT

SEQ ID NO: 72 - protein, 8 aa, synthetic construct
GFTFSSYT

SEQ ID NO: 73 - protein, 10 aa, synthetic construct
TFISYDGNNK

SEQ ID NO: 74 - protein, 11 aa, synthetic construct
ARTGWLGPFDY

SEQ ID NO: 75 - protein, 6 aa, synthetic construct
QSINSY

SEQ ID NO: 76 - skipped

SEQ ID NO: 77 - protein, 9 aa, synthetic construct
QQYYSTPFT

SEQ ID NO: 78 - protein, 8 aa, synthetic construct
GFTFSSYG

SEQ ID NO: 79 - protein, 10 aa, synthetic construct
AVIWYDGSNK

SEQ ID NO: 80 - protein, 18 aa, synthetic construct
ARDPRGATLYYYYYGMDV

SEQ ID NO: 81-186 - skipped (106 entries)

SEQ ID NO: 187 - protein, 8 aa, synthetic construct
  VARIANT 3..4: note = grouped amino acid residues may be present or absent
  VARIANT 5..6: note = grouped amino acid residues may be present or absent
  VARIANT 7..8: note = grouped amino acid residues may be present or absent
GSGSGSGS

SEQ ID NO: 188 - protein, 20 aa, synthetic construct
  VARIANT 6..10: note = grouped amino acid residues may be present or absent
  VARIANT 11..15: note = grouped amino acid residues may be present or absent
  VARIANT 16..20: note = grouped amino acid residues may be present or absent
GSGGSGSGGSGSGGSGSGGS

SEQ ID NO: 189 - protein, 20 aa, synthetic construct
  VARIANT 6..10: note = grouped amino acid residues may be present or absent
  VARIANT 11..15: note = grouped amino acid residues may be present or absent
  VARIANT 16..20: note = grouped amino acid residues may be present or absent
GGGGSGGGGSGGGGSGGGGS

SEQ ID NO: 190 - protein, 16 aa, synthetic construct
  VARIANT 5..8: note = grouped amino acid residues may be present or absent
  VARIANT 9..12: note = grouped amino acid residues may be present or absent
  VARIANT 13..16: note = grouped amino acid residues may be present or absent
GGGSGGGSGGGSGGGS

SEQ ID NO: 191-195 - skipped (5 entries)

SEQ ID NO: 196 - protein, 4 aa, synthetic construct
RVKR

SEQ ID NO: 197-198 - skipped (2 entries)

SEQ ID NO: 199 - protein, 27 aa, synthetic construct
SSGGGGSGGGGSGGGGSGGGGSGGGGS

SEQ ID NO: 200 - protein, 5 aa, synthetic construct
GSGSG

SEQ ID NO: 201 - protein, 6 aa, synthetic construct
ASGGPE

SEQ ID NO: 202 - protein, 54 aa, synthetic construct
  VARIANT 3..6: note = This region may encompass 2-4 residues
  VARIANT 8..11: note = This region may encompass 2-4 residues
  VARIANT 13..16: note = This region may encompass 2-4 residues
  VARIANT 18..21: note = This region may encompass 2-4 residues
  VARIANT 23..26: note = This region may encompass 2-4 residues
  VARIANT 28..31: note = This region may encompass 2-4 residues
  VARIANT 33..36: note = This region may encompass 2-4 residues
  VARIANT 38..41: note = This region may encompass 2-4 residues
  VARIANT 43..46: note = This region may encompass 2-4 residues
  VARIANT 48..51: note = This region may encompass 2-4 residues
  VARIANT 3..52: note = This region may encompass 1 to 6 'Gly(x) Ser' repeating units wherein x = 2 to 4
AMGGGGSGGGGSGGGGSGGGGSGGGGSGGGGSGGGGSGGGGSGGGGSGGGGSAM

SEQ ID NO: 203 - protein, 210 aa, synthetic construct
  VARIANT 1..6: note = This region may encompass 1-6 residues
  VARIANT 8..13: note = This region may encompass 1-6 residues
  VARIANT 15..20: note = This region may encompass 1-6 residues
  VARIANT 22..27: note = This region may encompass 1-6 residues
  VARIANT 29..34: note = This region may encompass 1-6 residues
  VARIANT 36..41: note = This region may encompass 1-6 residues
  VARIANT 43..48: note = This region may encompass 1-6 residues
  VARIANT 50..55: note = This region may encompass 1-6 residues
  VARIANT 57..62: note = This region may encompass 1-6 residues
  VARIANT 64..69: note = This region may encompass 1-6 residues
  VARIANT 71..76: note = This region may encompass 1-6 residues
  VARIANT 78..83: note = This region may encompass 1-6 residues
  VARIANT 85..90: note = This region may encompass 1-6 residues
  VARIANT 92..97: note = This region may encompass 1-6 residues
  VARIANT 99..104: note = This region may encompass 1-6 residues
  VARIANT 106..111: note = This region may encompass 1-6 residues
  VARIANT 113..118: note = This region may encompass 1-6 residues
  VARIANT 120..125: note = This region may encompass 1-6 residues
  VARIANT 127..132: note = This region may encompass 1-6 residues
  VARIANT 134..139: note = This region may encompass 1-6 residues
  VARIANT 141..146: note = This region may encompass 1-6 residues
  VARIANT 148..153: note = This region may encompass 1-6 residues
  VARIANT 155..160: note = This region may encompass 1-6 residues
  VARIANT 162..167: note = This region may encompass 1-6 residues
  VARIANT 169..174: note = This region may encompass 1-6 residues
  VARIANT 176..181: note = This region may encompass 1-6 residues
  VARIANT 183..188: note = This region may encompass 1-6 residues
  VARIANT 190..195: note = This region may encompass 1-6 residues
  VARIANT 197..202: note = This region may encompass 1-6 residues
  VARIANT 204..209: note = This region may encompass 1-6 residues
  VARIANT 1..210: note = This sequence may encompass 1 to 30 'Gly(x) Ser' repeating units wherein x = 1 to 6
GGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGSGGGGGGS

SEQ ID NO: 204 - protein, 210 aa, synthetic construct
  VARIANT 1..6: note = This region may encompass 1-6 residues
  VARIANT 8..13: note = This region may encompass 1-6 residues
  VARIANT 15..20: note = This region may encompass 1-6 residues
  VARIANT 22..27: note = This region may encompass 1-6 residues
  VARIANT 29..34: note = This region may encompass 1-6 residues
  VARIANT 36..41: note = This region may encompass 1-6 residues
  VARIANT 43..48: note = This region may encompass 1-6 residues
  VARIANT 50..55: note = This region may encompass 1-6 residues
  VARIANT 57..62: note = This region may encompass 1-6 residues
  VARIANT 64..69: note = This region may encompass 1-6 residues
  VARIANT 71..76: note = This region may encompass 1-6 residues
  VARIANT 78..83: note = This region may encompass 1-6 residues
  VARIANT 85..90: note = This region may encompass 1-6 residues
  VARIANT 92..97: note = This region may encompass 1-6 residues
  VARIANT 99..104: note = This region may encompass 1-6 residues
  VARIANT 106..111: note = This region may encompass 1-6 residues
  VARIANT 113..118: note = This region may encompass 1-6 residues
  VARIANT 120..125: note = This region may encompass 1-6 residues
  VARIANT 127..132: note = This region may encompass 1-6 residues
  VARIANT 134..139: note = This region may encompass 1-6 residues
  VARIANT 141..146: note = This region may encompass 1-6 residues
  VARIANT 148..153: note = This region may encompass 1-6 residues
  VARIANT 155..160: note = This region may encompass 1-6 residues
  VARIANT 162..167: note = This region may encompass 1-6 residues
  VARIANT 169..174: note = This region may encompass 1-6 residues
  VARIANT 176..181: note = This region may encompass 1-6 residues
  VARIANT 183..188: note = This region may encompass 1-6 residues
  VARIANT 190..195: note = This region may encompass 1-6 residues
  VARIANT 197..202: note = This region may encompass 1-6 residues
  VARIANT 204..209: note = This region may encompass 1-6 residues
  VARIANT 1..210: note = This sequence may encompass 1 to 30 'Ser(x) Gly' repeating units wherein x = 1 to 6
SSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSGSSSSSSG

SEQ ID NO: 205 - protein, 150 aa, synthetic construct
  VARIANT 1..150: note = sequence may encompass 1-30 'Gly Gly Gly Gly Ser' repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 206 - protein, 30 aa, synthetic construct
  VARIANT 1..30: note = sequence may encompass 1-30 residues
GGGGGGGGGGGGGGGGGGGGGGGGGGGGGG

SEQ ID NO: 207 - protein, 12 aa, synthetic construct
GKSSGSGSESKS

SEQ ID NO: 208 - protein, 14 aa, synthetic construct
EGKSSGSGSESKEF

SEQ ID NO: 209 - protein, 14 aa, synthetic construct
GSTSGSGKSSEGKG

SEQ ID NO: 210 - protein, 18 aa, synthetic construct
GSTSGSGKSSEGSGSTKG

SEQ ID NO: 211 - protein, 18 aa, synthetic construct
GSTSGSGKPGSGEGSTKG

SEQ ID NO: 212 - protein, 5 aa, synthetic construct
SRSSG

SEQ ID NO: 213 - protein, 5 aa, synthetic construct
SGSSC

SEQ ID NO: 214 - protein, 25 aa, synthetic construct
GGGGSGGGGSGGGGSGGGGSGGGGS

SEQ ID NO: 215 - protein, 20 aa, synthetic construct
GGGGSGGGGSGGGGSGGGGS

SEQ ID NO: 216 - protein, 18 aa, synthetic construct
GSTSGSGKPGSGEGSTKG

SEQ ID NO: 217 - protein, 5 aa, synthetic construct
GGGGS

SEQ ID NO: 218 - protein, 16 aa, synthetic construct
EFPKPSTPPGSSGGAP

SEQ ID NO: 219 - protein, 25 aa, synthetic construct
EFPKPSTPPGSSGGAPGILGFVFTL

SEQ ID NO: 220 - protein, 15 aa, synthetic construct
GSTSGSGKPGSGEGS

SEQ ID NO: 221 - protein, 16 aa, synthetic construct
SPSTPPTPSPSTPPAS

SEQ ID NO: 222 - protein, 15 aa, synthetic construct
AHHSEDPSSKAPKAP

SEQ ID NO: 223 - protein, 28 aa, synthetic construct
AMGRSGGGCAGNRVGSSLSCGGLNLQAM

SEQ ID NO: 224 - skipped

SEQ ID NO: 225 - protein, 4 aa, synthetic construct
GGGS

SEQ ID NO: 226 - protein, 5 aa, synthetic construct
GGGGS

SEQ ID NO: 227 - protein, 8 aa, synthetic construct
GGGGSGGG

SEQ ID NO: 228 - protein, 7 aa, synthetic construct
GGSGGGG

SEQ ID NO: 229 - protein, 18 aa, synthetic construct
GSTSGGGSGGGSGGGGSS

SEQ ID NO: 230 - protein, 18 aa, synthetic construct
GSTSGSGKPGSSEGSTKG

SEQ ID NO: 231 - protein, 4 aa, synthetic construct
KDEL

SEQ ID NO: 232 - protein, 4 aa, synthetic construct
HDEF

SEQ ID NO: 233 - protein, 4 aa, synthetic construct
HDEL

SEQ ID NO: 234 - protein, 4 aa, synthetic construct
RDEF

SEQ ID NO: 235 - protein, 4 aa, synthetic construct
RDEL

SEQ ID NO: 236 - protein, 4 aa, synthetic construct
WDEL

SEQ ID NO: 237 - protein, 4 aa, synthetic construct
YDEL

SEQ ID NO: 238 - protein, 4 aa, synthetic construct
HEEF

SEQ ID NO: 239 - protein, 4 aa, synthetic construct
HEEL

SEQ ID NO: 240 - protein, 4 aa, synthetic construct
KEEL

SEQ ID NO: 241 - protein, 4 aa, synthetic construct
REEL

SEQ ID NO: 242 - protein, 4 aa, synthetic construct
KAEL

SEQ ID NO: 243 - protein, 4 aa, synthetic construct
KCEL

SEQ ID NO: 244 - protein, 4 aa, synthetic construct
KFEL

SEQ ID NO: 245 - protein, 4 aa, synthetic construct
KGEL

SEQ ID NO: 246 - protein, 4 aa, synthetic construct
KHEL

SEQ ID NO: 247 - protein, 4 aa, synthetic construct
KLEL

SEQ ID NO: 248 - protein, 4 aa, synthetic construct
KNEL

SEQ ID NO: 249 - protein, 4 aa, synthetic construct
KQEL

SEQ ID NO: 250 - protein, 4 aa, synthetic construct
KREL

SEQ ID NO: 251 - protein, 4 aa, synthetic construct
KSEL

SEQ ID NO: 252 - protein, 4 aa, synthetic construct
KVEL

SEQ ID NO: 253 - protein, 4 aa, synthetic construct
KWEL

SEQ ID NO: 254 - protein, 4 aa, synthetic construct
KYEL

SEQ ID NO: 255 - protein, 4 aa, synthetic construct
KEDL

SEQ ID NO: 256 - protein, 4 aa, synthetic construct
KIEL

SEQ ID NO: 257 - protein, 4 aa, synthetic construct
DKEL

SEQ ID NO: 258 - protein, 4 aa, synthetic construct
FDEL

SEQ ID NO: 259 - protein, 4 aa, synthetic construct
KDEF

SEQ ID NO: 260 - protein, 4 aa, synthetic construct
KKEL

SEQ ID NO: 261 - protein, 4 aa, synthetic construct
HADL

SEQ ID NO: 262 - protein, 4 aa, synthetic construct
HAEL

SEQ ID NO: 263 - protein, 4 aa, synthetic construct
HIEL

SEQ ID NO: 264 - protein, 4 aa, synthetic construct
HNEL

SEQ ID NO: 265 - protein, 4 aa, synthetic construct
HTEL

SEQ ID NO: 266 - protein, 4 aa, synthetic construct
KTEL

SEQ ID NO: 267 - protein, 4 aa, synthetic construct
HVEL

SEQ ID NO: 268 - protein, 4 aa, synthetic construct
NDEL

SEQ ID NO: 269 - protein, 4 aa, synthetic construct
QDEL

SEQ ID NO: 270 - protein, 4 aa, synthetic construct
REDL

SEQ ID NO: 271 - protein, 4 aa, synthetic construct
RNEL

SEQ ID NO: 272 - protein, 4 aa, synthetic construct
RTDL

SEQ ID NO: 273 - protein, 4 aa, synthetic construct
RTEL

SEQ ID NO: 274 - protein, 4 aa, synthetic construct
SDEL

SEQ ID NO: 275 - protein, 4 aa, synthetic construct
TDEL

SEQ ID NO: 276 - protein, 4 aa, synthetic construct
SKEL

SEQ ID NO: 277 - protein, 4 aa, synthetic construct
STEL

SEQ ID NO: 278 - protein, 4 aa, synthetic construct
EDEL

SEQ ID NO: 279-284 - skipped (6 entries)

SEQ ID NO: 285 - protein, 5 aa, synthetic construct
EAAAK

SEQ ID NO: 286 - protein, 373 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 287 - protein, 370 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 288 - protein, 371 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 289 - protein, 504 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMTALKPEDTAMYYCAAGKWGTDYWGQGTQVTVSSEAAAKQVQLVESGGGSVQAGGSLRLSCAASGYYNRYCLGWFRQTPGKEREAVATIDTDGSTSYADSVKGRFTISFDNAKNTLYLQMNSLKPEDTAMYYCAAGPNPRYCSGAVNTRGAEHYFGYWAQGTQVTVSS

SEQ ID NO: 290 - protein, 514 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMTALKPEDTAMYYCAAGKWGTDYWGQGTQVTVSSEAAAKEAAAKEAAAKQVQLVESGGGSVQAGGSLRLSCAASGYYNRYCLGWFRQTPGKEREAVATIDTDGSTSYADSVKGRFTISFDNAKNTLYLQMNSLKPEDTAMYYCAAGPNPRYCSGAVNTRGAEHYFGYWAQGTQVTVSS

SEQ ID NO: 291 - protein, 750 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMTALKPEDTAMYYCAAGKWGTDYWGQGTQVTVSSRNSISSLSIPSTVPRARDPPVDMASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGAGLYKQVQLVESGGGSVQAGGSLRLSCAASGYYNRYCLGWFRQTPGKEREAVATIDTDGSTSYADSVKGRFTISFDNAKNTLYLQMNSLKPEDTAMYYCAAGPNPRYCSGAVNTRGAEHYFGYWAQGTQVTVSS

SEQ ID NO: 292 - protein, 488 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSEAAAKQVQLVESGGGLVQPGGSLRLSCAASGVNFSSYTMSWVRQAPGLGLEWVAYINSGGGTTSYADSVKGRFTISRDNAKNTLYLQMNSLLPEDTAMYYCQGGLYRGQGTQVTVSS

SEQ ID NO: 293 - protein, 488 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVQLVESGGGLVQPGGSLRLSCAASGVNFSSYTMSWVRQAPGLGLEWVAYINSGGGTTSYADSVKGRFTISRDNAKNTLYLQMNSLLPEDTAMYYCQGGLYRGQGTQVTVSSEAAAKEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 294 - protein, 491 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSEAAAKQVQLQESGGGLVQPGGSLRLSCAASGSIRSSNTMGWYRQAPGKERDLIVSMSSGGFRSYVDSVKDRFTISRDNAKNTVYLQMNSLKSEDTAVYYCRYLAPDVQSWGQGIQVTVSS

SEQ ID NO: 295 - protein, 491 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVQLQESGGGLVQPGGSLRLSCAASGSIRSSNTMGWYRQAPGKERDLIVSMSSGGFRSYVDSVKDRFTISRDNAKNTVYLQMNSLKSEDTAVYYCRYLAPDVQSWGQGIQVTVSSEAAAKEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 296 - protein, 370 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 297 - protein, 371 aa, synthetic construct; identical to SEQ ID NO: 288

SEQ ID NO: 298 - protein, 371 aa, synthetic construct
MKEFTLDFSTAKTYVDSLNVIRSAIGTPLQTISSGGTSLLMIDSGIGDNLFAVDILGFDFTLGRFNNLRLIVERNNLSVTGFVNRTNNVFYRFADFSHVTFPGTTAVTLSADSSYTTLQRVAGISRTGMQINRHSLTTSYLDLMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 299 - protein, 370 aa, synthetic construct
MKEFTLDFSTAKTYVDSLNVIRSAIGTPLQTISSGGTSLLMIDSGIGDNLFAVDILGFDFTLGRFNNLRLIVERNNLSVTGFVNRTNNVFYRFADFSHVTFPGTTAVTLSADSSYTTLQRVAGISRTGMQINRHSLTTSYLDLMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 300 - protein, 505 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVQLVESGGGSVQAGGSLRLSCAASGYYNRYCLGWFRQTPGKEREAVATIDTDGSTSYADSVKGRFTISFDNAKNTLYLQMNSLKPEDTAMYYCAAGPNPRYCSGAVNTRGAEHYFGYWAQGTQVTVSSEAAAKEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 301 - protein, 509 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVQLVESGGGSVQAGGSLRLSCAASGYYNRYCLGWFRQTPGKEREAVATIDTDGSTSYADSVKGRFTISFDNAKNTLYLQMNSLKPEDTAMYYCAAGPNPRYCSGAVNTRGAEHYFGYWAQGTQVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 302 - protein, 495 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSEAAAKEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 303 - protein, 499 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 304 - protein, 550 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSLAEAKVLANRELDKYGVSDYYKNLINNAKTVEGVKALIDEILAALP

SEQ ID NO: 305 - protein, 560 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGGSGGGGSLAEAKVLANRELDKYGVSDYYKNLINNAKTVEGVKALIDEILAALP

SEQ ID NO: 306 - protein, 545 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAALAEAKVLANRELDKYGVSDYYKNLINNAKTVEGVKALIDEILAALPEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 307 - protein, 623 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 308 - protein, 623 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 309 - protein, 750 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 310 - protein, 751 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGSFRRGGGSKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 311 - protein, 512 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNCHHHASAVAAMASDEFPSMCPADEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 312 - protein, 541 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNCHHHASAVAAMASDEFPSMCPADGRVRGITHNKILWDSSTLGAILMRRTISSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 313 - protein, 611 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSTRVDQTPRTATRETGESLTINCVLTDTSYPLYSTYWYRKNPGSSNKEQISISGRYVESVNKGTKSFSLRIKDLTVADSATYICRAMGTNIWTGDGAGTVLTVN

SEQ ID NO: 314 - protein, 611 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSTRVDQTPRTATRETGESLTINCVLTDTSYPLYSTYWYRKNPGSSNKEQISISGRYVESVNKGTKSFSLRIKDLTVADSATYICRAMGTNIWTGDGAGTVLTVNGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 315 - protein, 522 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSWWEQDRDWDFDVFGGGTP

SEQ ID NO: 316 - protein, 367 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRQTPGKEREAVASIYPTGGTFYTDSVKGRFTISRDNAKSTLYLQMNSLKPEDTAMYYCAAGKWGTDYWGQGTQVTVSS

SEQ ID NO: 317 - protein, 494 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKQVQLVESGGNLVQPGGSLRLSCAASGSIFSPNIMGWYRQPPGNERELVASVHSSGVTNYEDSAKGRFTISGDDAKYIWYLQMNGLKPEDTAAYYCNVRELGVYFNRDFWGKGTLVTVSS

SEQ ID NO: 318 - protein, 491 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRQTPGKEREAVASIYPTGGTFYTDSVKGRFTISRDNAKSTLYLQMNSLKPEDTAMYYCAAGKWGTDYWGQGTQVTVSSEAAAKQVQLVESGGNLVQPGGSLRLSCAASGSIFSPNIMGWYRQPPGNERELVASVHSSGVTNYEDSAKGRFTISGDDAKYIWYLQMNGLKPEDTAAYYCNVRELGVYFNRDFWGKGTLVTVSS

SEQ ID NO: 319 - protein, 491 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRQTPGKEREAVASIYPTGGTFYTDSVKGRFTISRDNAKSTLYLQMNSLKPEDTAMYYCAAGKWGTDYWGQGTQVTVSSEAAAKQVQLVESGGNLVQPGGSLRLSCAASGSIFSPNIMGWFRQPPGNERELVASVHSSGVTNYEDSAKGRFTISGDDAKYIWYLQMNGLKPEDTAAYYCNVRELGVYFNRDFWGKGTLVTVSS

SEQ ID NO: 320 - protein, 367 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWYRQPPGNERELVASIYPTGGTFYTDSVKGRFTISRDNAKSTLYLQMNSLKPEDTAMYYCAAGKWGTDYWGQGTQVTVSS

SEQ ID NO: 321 - protein, 491 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWYRQPPGNERELVASIYPTGGTFYTDSVKGRFTISRDNAKSTLYLQMNSLKPEDTAMYYCAAGKWGTDYWGQGTQVTVSSEAAAKQVQLVESGGNLVQPGGSLRLSCAASGSIFSPNIMGWYRQPPGNERELVASVHSSGVTNYEDSAKGRFTISGDDAKYIWYLQMNGLKPEDTAAYYCNVRELGVYFNRDFWGKGTLVTVSS

SEQ ID NO: 322 - protein, 488 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVQLVESGGGLVQPGGSLRLSCAASGVNFSSYTMSWYRQPPGNERELVAYINSGGGTTSYADSVKGRFTISRDNAKNTLYLQMNSLKPEDTAMYYCQGGLYRGQGTQVTVSSEAAAKEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 323 - protein, 488 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVQLVESGGGLVQPGGSLRLSCAASGVNFSSYTMSWYRQPPGNERELVAYINSGGGTTSYADSVKGRFTISRDNAKNTLYLQMNSLKPEDTAMYYCQGGLYRGQGTQVTVSSEAAAKQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 324 - protein, 488 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVQLVESGGGLVQPGGSLRLSCAASGVNFSSYTMSWYRQAPGKERELVAYINSGGGTTSYADSVKGRFTISRDNAKNTLYLQMNSLKPEDTAMYYCQGGLYRGQGTQVTVSSEAAAKQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 325 - protein, 494 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKEVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 326 - protein, 494 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 327 - protein, 510 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 328 - protein, 509 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEAAAKEAAAKEAAAKQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 329 - protein, 514 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 330 - protein, 513 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEAAAKEAAAKEAAAKQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 331 - protein, 421 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSLAEAKVLANRELDKYGVSDYYKNLINNAKTVEGVKALIDEILAALP

SEQ ID NO: 332 - protein, 431 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSLAEAKVLANRELDKYGVSDYYKNLINNAKTVEGVKALIDEILAALP

SEQ ID NO: 333 - protein, 441 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSGGGGSGGGGSLAEAKVLANRELDKYGVSDYYKNLINNAKTVEGVKALIDEILAALP

SEQ ID NO: 334 - protein, 421 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKLAEAKVLANRELDKYGVSDYYKNLINNAKTVEGVKALIDEILAALP

SEQ ID NO: 335 - protein, 431 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKEAAAKEAAAKLAEAKVLANRELDKYGVSDYYKNLINNAKTVEGVKALIDEILAALP

SEQ ID NO: 336 - protein, 447 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSLAEAKVLANRELDKYGVSDYYKNLINNAKTVEGVKALIDEILAALP

SEQ ID NO: 337 - protein, 447 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKEAAAKEAAAKLAEAKVLANRELDKYGVSDYYKNLINNAKTVEGVKALIDEILAALP

SEQ ID NO: 338 - protein, 446 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEAAAKEAAAKEAAAKQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSLAEAKVLANRELDKYGVSDYYKNLINNAKTVEGVKALIDEILAALP

SEQ ID NO: 339 - protein, 446 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEAAAKEAAAKEAAAKQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKEAAAKEAAAKLAEAKVLANRELDKYGVSDYYKNLINNAKTVEGVKALIDEILAALP

SEQ ID NO: 340 - protein, 446 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAAHHSEDPSSKAPKAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSLAEAKVLANRELDKYGVSDYYKNLINNAKTVEGVKALIDEILAALP

SEQ ID NO: 341 - protein, 446 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAAHHSEDPSSKAPKAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKEAAAKEAAAKLAEAKVLANRELDKYGVSDYYKNLINNAKTVEGVKALIDEILAALP

SEQ ID NO: 342 - protein, 386 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 343 - protein, 385 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEAAAKEAAAKEAAAKQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 344 - protein, 385 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAAHHSEDPSSKAPKAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 345 - protein, 436 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSLAEAKVLANRELDKYGVSDYYKNLINNAKTVEGVKALIDEILAALPGGGGS

SEQ ID NO: 346 - protein, 452 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSLAEAKVLANRELDKYGVSDYYKNLINNAKTVEGVKALIDEILAALPGGGGS

SEQ ID NO: 347 - protein, 451 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAAHHSEDPSSKAPKAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSLAEAKVLANRELDKYGVSDYYKNLINNAKTVEGVKALIDEILAALPGGGGS

SEQ ID NO: 348 - protein, 514 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGSQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 349 - protein, 608 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEFPKPSTPPGSSGGAPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 350 - protein, 607 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEFPKPSTPPGSSGGAPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 351 - protein, 608 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 352 - protein, 607 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 353 - protein, 518 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPEVQLVESGGGLVQAGGSLRLSCAASGLTFSSYAMGWFRQAPGKERERVVSISRGGGYTYYADSVKGRFTISRDNAENTVYLQMNSLKPEDTAVYYCAAARYWATGSEYEFDYWGQGTLVTVSSGGGGSGGGSQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 354 - protein, 518 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSEVQLVESGGGLVQAGGSLRLSCAASGLTFSSYAMGWFRQAPGKERERVVSISRGGGYTYYADSVKGRFTISRDNAENTVYLQMNSLKPEDTAVYYCAAARYWATGSEYEFDYWGQGTLVTVSS

SEQ ID NO: 355 - protein, 575 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSGGGGSGGGGSGGGGSLAEAKVLANRELDKYGVSDYYKNLINNAKTVEGVKALIDEILAALP

SEQ ID NO: 356 - protein, 569 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSLAEAKVLANRELDKYGVSDYYKNLINNAKTVEGVKALIDEILAALPGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 357 - protein, 736 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSEFPKPSTPPGSSGGAPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 358 - protein, 735 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSEFPKPSTPPGSSGGAPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 359 - protein, 743 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEFPKPSTPPGSSGGAPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEFPKPSTPPGSSGGAPQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 360 - protein, 742 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEFPKPSTPPGSSGGAPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAEFPKPSTPPGSSGGAPQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 361 - protein, 370 aa, synthetic construct
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 362 - protein, 371 aa, synthetic construct
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 363 - protein, 514 aa, synthetic construct
MKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAREFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 364 - protein, 386 aa, synthetic construct; identical to SEQ ID NO: 342

SEQ ID NO: 365 - protein, 514 aa, synthetic construct
MKEFTLDFCTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 366 - protein, 514 aa, synthetic construct
MKEFTLDFSTAKTYVDSLNVIRSAIGTPLQTISSGGTSLLMIDSGIGDNLFAVDILGFDFTLGRFNNLRLIVERNNLSVTGFVNRTNNVFYRFADFSHVTFPGTTAVTLSADSSYTTLQRVAGISRTGMQINRHSLTTSYLDLMSHSGTSLTQSVARAMLRFVTVTADALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 367 - protein, 510 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 368 - protein, 530 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSGGGGSGGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 369 - protein, 502 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALIEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 370 - protein, 387 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 371 - protein, 514 aa, synthetic construct; identical to SEQ ID NO: 329

SEQ ID NO: 372 - protein, 370 aa, synthetic construct
MKEFTLDFCTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 373 - protein, 509 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 374 - protein, 513 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 375 - protein, 519 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 376 - protein, 529 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGGSGGGGSGGGGSGGGGSQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 377 - protein, 386 aa, synthetic construct
MKEFTLDFCTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSS

SEQ ID NO: 378 - protein, 514 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCAGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 379 - protein, 514 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCAATGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 380 - protein, 514 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCAGTGSIFSPNAMGWYRQGPGKQRELVASVTSSGTTNYSDSVKGRFTISMDNYKHLAYLQMNSLKPEDTAVYYCALKELGTFYRRDFWGQGTQVTVSS

SEQ ID NO: 381 - protein, 514 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCAATGSIFSPNAMGWYRQGPGKQRELVASVTSSGTTNYSDSVKGRFTISMDNYKHLAYLQMNSLKPEDTAVYYCALKELGTFYRRDFWGQGTQVTVSS

SEQ ID NO: 382 - protein, 515 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKGGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 383 - protein, 520 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKEAAAKGGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 384 - protein, 519 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 385 - protein, 519 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEGKSSGSGSESKSTQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 386 - protein, 523 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSKESGSVSSEQLAQFRSLDQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 387 - protein, 515 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKGGGGSQVQLVESGGGLVQPGGSLRLSCAGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 388 - protein, 520 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKEAAAKGGGGSQVQLVESGGGLVQPGGSLRLSCAGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 389 - protein, 519 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEAAAKGGGGSGGGSQVQLVESGGGLVQPGGSLRLSCAGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 390 - protein, 519 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSEGKSSGSGSESKSTQVQLVESGGGLVQPGGSLRLSCAGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 391 - protein, 523 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSKESGSVSSEQLAQFRSLDQVQLVESGGGLVQPGGSLRLSCAGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 392 - protein, 387 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLVESGGGLVQPGGSLRLSCAGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 393 - protein, 387 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLVESGGGLVQPGGSLRLSCAATGSIFSPNAMGWYRQGPGKQRELVASVTSSGTTNYSDSVKGRFTISMDNYKHLAYLQMNSLKPEDTAVYYCNLKELGTFYRRDFWGQGTQVTVSS

SEQ ID NO: 394 - protein, 387 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLVESGGGLVQPGGSLRLSCAGTGSIFSPNAMGWYRQGPGKQRELVASVTSSGTTNYSDSVKGRFTISMDNYKHLAYLQMNSLKPEDTAVYYCALKELGTFYRRDFWGQGTQVTVSS

SEQ ID NO: 395 - protein, 387 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVQLVESGGGLVQPGGSLRLSCAATGSIFSPNAMGWYRQGPGKQRELVASVTSSGTTNYSDSVKGRFTISMDNYKHLAYLQMNSLKPEDTAVYYCALKELGTFYRRDFWGQGTQVTVSS

SEQ ID NO: 396 - protein, 520 aa, synthetic construct
MKEFTLDFSTAKTYVDSLNVIRSAIGTPLQTISSGGTSLLMIDSGIGDNLFAVDILGFDFTLGRFNNLRLIVERNNLYVTGFVNRTNNVFYRFADFSHVTFPGTTAVTLSADSSYTTLQRVAGISRTGMQINRHSLTTSYLDLMSHSGTSLTQSVARAMLRFVTVTAEALRFRQIQRGFRTTLDDLSGASYVMTAEDVDLTLNWGRLSSVLPDYHGQDSVRVGRISFGSINAILGSVALILNSHHHASAVAAEFPKPSTPPGSSGGAPQVKLVESGGGSVQAGGSLRLSCAASGDSYSVKYMGWFRRAPGKQRDQREEVASIYPTGGTFYTDSVKGRFTISRDNAKSTLYLQMNSLKPEDTAMYYCAAGKWGTDYWGQGTQVTVSSGGGGSGGGSQVQLVESGGGLVQPGGSLRLSCEGTGSIFSPNAMGWYRQGPGKQRELVASVTSSGTTNYSDSVKGRFTISMDNYKHLAYLQMNSLKPEDTAVYYCNLKELGTFYRRDFWGQGTQVTVSSHHHHHH